(19) United States
(12) Patent Application Publication
(10) Pub. No.: US 2026/0224748 A1
(43) Pub. Date: Aug. 6, 2026

(54) METHODS AND COMPOSITIONS FOR TREATING ALPHA-1 ANTITRYPSIN DEFICIENCY

(71) Applicant: Beam Therapeutics Inc., Cambridge, MA (US)

(72) Inventors: Michael Packer, Cambridge, MA (US); Brian Cafferty, Cambridge, MA (US); Yi Yu, Cambridge, MA (US)

(73) Assignee: Beam Therapeutics Inc., Cambridge, MA (US)

(21) Appl. No.: 19/649,581

(22) Filed: Apr. 16, 2026

Related U.S. Application Data
(63) Continuation of application No. 19/416,825, filed on Dec. 11, 2025.
Application No. 19/416,825 is a continuation of application No. PCT/US2024/034189, filed on Jun. 14, 2024.
(60) Provisional application No. 63/508,469, filed on Jun. 15, 2023.
(60) Provisional application No. 63/580,925, filed on Sep. 6, 2023.

(51) Int. Cl. A61K 48/00; A61K 38/57; A61P 1/16; C12N 5/071; C12N 9/22; C12N 9/78; C12N 15/11

(52) U.S. Cl. CPC A61K 48/005; A61K 38/57; A61P 1/16; C12N 5/067; C12N 9/226; C12N 9/78; C12N 15/111; C07K 2319/80; C12N 2310/20; C12N 2510/00; C12Y 305/04004

(57) ABSTRACT

Compositions and methods for editing deleterious mutations associated with Alpha-1 Antitrypsin Deficiency (A1AD). In particular embodiments, the invention provides methods for treating A1AD using a modified adenosine base editor with improved on-target editing and decreased off-target editing to correct mutations associated with A1AD.

### CROSS REFERENCE TO RELATED APPLICATIONS

This application is a continuation under 35 U.S.C. § 111 (a) of U.S. patent application Ser. No. 19/416,825, filed Dec. 11, 2025, which is a continuation of PCT International Patent Application No. PCT/US2024/034189, filed Jun. 14, 2024, designating the United States and published in English, which claims priority to and the benefit of U.S. Provisional Application No. 63/580,925, filed Sep. 6, 2023, and U.S. Provisional Application No. 63/508,469, filed Jun. 15, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Apr. 15, 2026, is named 180802-049713USCON_SL.xml, and is 1,109,611 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

### BACKGROUND

In healthy individuals, alpha-1 antitrypsin (A1AT) is produced by hepatocytes within the liver and secreted into systemic circulation where it functions as a protease inhibitor. It is a particularly good inhibitor of neutrophil elastase, thus protecting tissues and organs such as the lung from elastin degradation. In patients with Alpha-1 Antitrypsin Deficiency (A1AD), mutations in the gene that encodes A1AT lead to diminished protein production. Consequently, elastin in the lung is degraded more readily by neutrophil elastase, and over time the loss in lung elasticity develops into chronic obstructive pulmonary disease (COPD).

The most common pathogenic A1AT variant is a Guanine to Adenine mutation resulting in a glutamate to lysine substitution at amino acid 342 of the A1AT polypeptide. This substitution causes the protein to misfold and polymerize within hepatocytes, and ultimately the toxic aggregates can lead to liver injury and cirrhosis. While the liver toxicity could be addressed by a gene knockout (e.g., using CRISPR/ZFN/TALEN) or gene knockdown (e.g., using siRNA), neither approach addresses the pulmonary pathology. Although pulmonary pathology may be addressed with protein replacement therapy, this therapy also fails to address the liver toxicity. Gene therapy also would be inadequate to address the A1AT genetic defect. Because the liver of patients with A1AD is already under a severe disease burden caused by the endogenous A1AT, gene therapy that increases A1AT in the liver would be counterproductive.

Base editing technologies represent an approach that could be used to treat patients with A1AD that addresses both the lung pathology and the liver toxicity. In particular, base editing methods and compositions that are capable of correcting point mutations associated with A1AT with improved on-target and/or decreased off-target editing are needed.

### SUMMARY

As described below, the present invention features compositions and methods for editing deleterious mutations associated with Alpha-1 Antitrypsin Deficiency (A1AD). In particular embodiments, the invention provides methods for treating A1AD using a modified adenosine base editor with improved on-target editing and decreased off-target editing to correct mutations associated with A1AD.

In one aspect, the disclosure features base editor containing a nucleic acid programmable DNA binding protein (napDNAbp) domain and an adenosine deaminase domain. The polynucleotide programmable DNA binding domain variant contains an alteration selected from one or more of M1135L, E1250K, A1283D, Q1136Y, R1337K, R765A, and Q768A of an amino acid sequence, that is at least 80%, 85%, 90%, 95%, 96%, 97%, 98%, 99% or 100% identical to the following sequence, or a fragment thereof lacking an N-terminal methionine:

(SEQ ID NO: 554)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMVKRYDEHHQDLTLLKALVRQQLPEKYKEIFFDQSKNGYAGYIDGGASQEEF
YKFIKPILEKMDGTEELLVKLNREDLLRKQRTFDNGIIPHQIHLGELHAILRRQGDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRLRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGGHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKGNS
DKLIARKKDWDPKKYGGFMQPTVAYSVLVVAKVEKGKSKKLKSVKELLGITIMERSSFEKNP
IDFLEAKGYKEVKKDLIIKLPKYSLFELENGRKRMLASAKFLQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPRAFKYFDTTIARKEYRSTKEVLDATLIHQSITGLYETRIDLSQ
LGGD.

In another aspect, the disclosure features a base editor system containing the base editor of any aspect of the disclosure, or embodiments thereof, or one or more polynucleotides encoding the base editor, and a guide polynucleotide, or a polynucleotide encoding the guide polynucleotide.

In another aspect, the disclosure features a guide polynucleotide, or a polynucleotide encoding the guide polynucleotide. The guide polynucleotide contains a nucleotide sequence selected from one or more of: 5′-ACCAUCGACAAGAAAGGGACUGA-3′ (SEQ ID NO: 466); 5′-CCAUCGACAAGAAAGGGACUGA-3′ (SEQ ID NO: 559); 5′-CAUCGACAAGAAAGGGACUGA-3′ (SEQ ID NO: 560); 5′-AUCGACAAGAAAGGGACUGA-3′ (SEQ ID NO: 561); 5′-UCGACAAGAAAGGGACUGA-3′ (SEQ ID NO: 562); and 5′-CGACAAGAAAGGGACUGA-3′ (SEQ ID NO: 563).

In another aspect, the disclosure features a method of editing an alpha-1 antitrypsin polynucleotide containing a single nucleotide polymorphism (SNP) associated with alpha-1 antitrypsin deficiency. The method involves contacting the polynucleotide with one or more guide RNAs, or one or more polynucleotides encoding the one or more guide RNAs, and a base editor, or one or more polynucleotides encoding the base editor. The guide RNA targets the base editor to effect an alteration of the SNP associated with alpha-1 antitrypsin deficiency. The base editor is the base editor of any one of any aspect of the disclosure, or embodiments thereof. The one or more guide RNAs contain the guide polynucleotide of any aspect of the disclosure, or embodiments thereof.

In another aspect, the disclosure features a method of editing an alpha-1 antitrypsin polynucleotide containing a single nucleotide polymorphism (SNP) associated with alpha-1 antitrypsin deficiency. The method involves contacting an alpha-1 antitrypsin polynucleotide with one or more guide RNAs and a fusion protein containing the following amino acid sequence:

(SEQ ID NO: 588)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDSGGSSGGSSGSETPGTSESA
TPESSGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDA
ILLSDILRVNTEITKAPLSASMVKRYDEHHQDLTLLKALVRQQLPEKYKEIFFDQSKNGYAG
YIDGGASQEEFYKFIKPILEKMDGTEELLVKLNREDLLRKQRTFDNGIIPHQIHLGELHAIL
RRQGDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRLRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGGHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGF
SKESILPKGNSDKLIARKKDWDPKKYGGFLQPTVAYSVLVVAKVEKGKSKKLKSVKELLGIT
IMERSSFEKNPIDFLEAKGYKEVKKDLIIKLPKYSLFELENGRKRMLASAKFLQKGNELALP
SKYVNFLYLASHYEKLKGSPKDNEQKQLFVEQHKHYLDEIIEQISEFSKRVILDDANLDKVL
SAYNKHRDKPIREQAENIIHLFTLTNLGAPRAFKYFDTTIARKEYKSTKEVLDATLIHQSIT
GLYETRIDLSQLGGDEGADKRTADGSEFESPKKKRKV.

In another aspect, the disclosure features a polynucleotide or set of polynucleotides encoding the base editor of any aspect of the disclosure, or embodiments thereof.

In another aspect, the disclosure features a vector or set of vectors containing the polynucleotide or set of polynucleotides of any aspect of the disclosure, or embodiments thereof.

In another aspect, the disclosure features a cell produced by introducing into the cell, or a progenitor thereof the base editor of any any aspect of the disclosure, or embodiments thereof, or one or more polynucleotides encoding the base editor, and one or more guide polynucleotides, or one or more polynucleotides encoding the one or more guide polynucleotides. The one or more guide polynucleotides target the base editor to effect an A·T to G·C alteration of an SNP associated with alpha-1 antitrypsin deficiency.

In another aspect, the disclosure features a method of treating alpha-1 antitrypsin deficiency in a subject. The method involves administering to the subject a cell of any aspect of the disclosure, or embodiments thereof.

In another aspect, the disclosure features a method of producing a hepatocyte cell. The method involves (a) introducing into a hepatocyte progenitor containing an SNP associated with alpha-1 antitrypsin deficiency the base editor of any aspect of the disclosure, or embodiments thereof, or one or more polynucleotides encoding the base editor, and one or more guide polynucleotides, or one or more polynucleotides encoding the one or more guide polynucleotides. The one or more guide polynucleotides target the base editor to effect an A·T to G·C alteration of the SNP associated with alpha-1 antitrypsin deficiency. The method further involves (b) differentiating the hepatocyte progenitor into a hepatocyte.

In another aspect, the disclosure features an isolated cell or population of cells propagated or expanded from the cell of any aspect of the disclosure, or embodiments thereof.

In another aspect, the disclosure features a method of producing a hepatocyte cell. The method involves (a) introducing into a hepatocyte containing an SNP associated with alpha-1 antitrypsin deficiency the base editor of any aspect of the disclosure, or embodiments thereof, or one or more polynucleotides encoding the base editor; and one or more guide polynucleotides, or one or more polynucleotides encoding the one or more guide polynucleotides. The one or more guide polynucleotides target the base editor to effect an A·T to G·C alteration of the SNP associated with alpha-1 antitrypsin deficiency.

In another aspect, the disclosure features a method for treating alpha-1 antitrypsin deficiency (A1AD) in a subject. The method involves administering to the subject the base editor of any aspect of the disclosure, or embodiments thereof, or one or more polynucleotides encoding the base editor, and one or more guide polynucleotides that target the base editor to effect an A·T to G·C alteration of a single nucleotide polymorphism (SNP) associated with A1AD, thereby treating A1AD in the subject.

In another aspect, the disclosure features a pharmaceutical composition containing the base editor system of any aspect of the disclosure, or embodiments thereof, and a pharmaceutically acceptable carrier, vehicle, or excipient.

In another aspect, the disclosure features a pharmaceutical composition containing the cell of any aspect of the disclosure, or embodiments thereof, and a pharmaceutically acceptable carrier, vehicle, or excipient.

In another aspect, the disclosure features a kit containing a base editing system of any aspect of the disclosure, or embodiments thereof.

In another aspect, the disclosure features a kit containing the cell of any aspect of the disclosure, or embodiments thereof.

In another aspect, the disclosure features a TadA variant containing an amino acid sequence that is at least 80%, 85%, 90%, 95%, 96%, 97%, 98%, 99%, or 100% identical to a TadA*5 or the following amino acid sequence, or a fragment thereof that does not contain an N-terminal methionine:

Variant 12
(SEQ ID NO: 589)
MSEVEFSHEYWMRHALTLAKRARDEREVPVGAVLVHNNRVIGEGWNRAIG
LHDPTAHAEIMALRQGGLVMQNYRLYDATLYTTFEPCVMCAGAMIHSRIG
RVVFGVRNAKTGAAGSLMDVLHYPGMNHRVEITEGILADECAALLCTFFR
MPRSVFKAQKKAQSSTD.

The TadA variant further contains any of the amino acid substitutions or combinations of substitutions listed in any one of Tables 12, 14, or, 17.

In another aspect, the disclosure features a Cas9 variant containing an amino acid sequence that is at least 80%, 85%, 90%, 95%, 96%, 97%, 98%, 99%, or 100% identical to SpCas9 or the following amino acid sequence, or a fragment thereof that does not contain an N-terminal methionine:

(SEQ ID NO: 590)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMVKRYDEHHQDLTLLKALVRQQLPEKYKEIFFDQSKNGYAGYIDGGASQEEF
YKFIKPILEKMDGTEELLVKLNREDLLRKQRTFDNGIIPHQIHLGELHAILRRQGDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRLRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGGHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKGNS
DKLIARKKDWDPKKYGGFMQPTVAYSVLVVAKVEKGKSKKLKSVKELLGITIMERSSFEKNP
IDFLEAKGYKEVKKDLIIKLPKYSLFELENGRKRMLASAKFLQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPRAFKYFDTTIARKEYRSTKEVLDATLIHQSITGLYETRIDLSQ
LGGD,

further containing any of the amino acid substitutions or combinations of substitutions listed in any one of Tables 7, 8, 13, 15, 16, 17, or 18.

In any aspect of the disclosure, or embodiments thereof, the napDNAbp contains one, two, three, four, five or six amino acid alterations selected from one or more of M1135L, E1250K, A1283D, Q1136Y, R1337K, R765A, and Q768A. In any aspect of the disclosure, or embodiments thereof, the napDNAbp contains a combination of amino acid alterations selected from one or more of: R1337K; Q1136Y, and R1337K; M1135L, Q1136Y, and R1337K; Q1136Y, and R1337K; M1135L, Q1136Y, and R1337K; M1135L, A1283D, Q1136Y, and R1337K; M1135L, A1283D, E1250K, and R1337K; A1283D, E1250K, and Q1136Y; M1135L, A1283D, Q1136Y, and R1337K; M1135L, A1283D, Q1136Y, R1337K, R765A, and Q768A; and A1283D, E1250K, and Q1136Y. In any aspect of the disclosure, or embodiments thereof, the napDNAbp contains the alterations M1135L, A1283D, E1250K, and R1337K. In any aspect of the disclosure, or embodiments thereof, the napDNAbp contains of the following amino acid sequence:

Variant G
(SEQ ID NO: 555)
DKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDAILLSDILRVNTE
ITKAPLSASMVKRYDEHHQDLTLLKALVRQQLPEKYKEIFFDQSKNGYAGYIDGGASQEEFY
KFIKPILEKMDGTEELLVKLNREDLLRKQRTFDNGIIPHQIHLGELHAILRRQGDFYPFLKD
NREKIEKILTFRIPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKAIVDLLFKTNRKV
TVKQLKEDYFKKIECFDSVEISGVEDRFNASLGTYHDLLKIIKDKDFLDNEENEDILEDIVL
TLTLFEDREMIEERLKTYAHLFDDKVMKQLKRLRYTGWGRLSRKLINGIRDKQSGKTILDFL
KSDGFANRNFMQLIHDDSLTFKEDIQKAQVSGQGDSLHEHIANLAGSPAIKKGILQTVKVVD
ELVKVMGGHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKGNSD
KLIARKKDWDPKKYGGFLQPTVAYSVLVVAKVEKGKSKKLKSVKELLGITIMERSSFEKNPI
DFLEAKGYKEVKKDLIIKLPKYSLFELENGRKRMLASAKFLQKGNELALPSKYVNFLYLASH
YEKLKGSPKDNEQKQLFVEQHKHYLDEIIEQISEFSKRVILDDANLDKVLSAYNKHRDKPIR
EQAENIIHLFTLTNLGAPRAFKYFDTTIARKEYKSTKEVLDATLIHQSITGLYETRIDLSQL
GGD.

In any aspect of the disclosure, or embodiments thereof, the adenosine deaminase domain contains an amino acid sequence that is at least 80%, 85%, 90%, 95%, 96%, 97%, 98%, 99%, or 100% identical to the following amino acid sequence:

(SEQ ID NO: 426)
SEVEFSHEYWMRHALTLAKRARDEREVPVGAVLVHNNRVIGEGWNRAIGL
HDPTAHAEIMALRQGGLVMQNYRLYDATLYTTFEPCVMCAGAMIHSRIGR
VVFGVRNAKTGAAGSLMDVLHYPGMNHRVEITEGILADECAALLCTFFRM
PRSVFKAQKKAQSSTD.

In any aspect of the disclosure, or embodiments thereof, the adenosine deaminase domain is a TadA*7.10 variant that contains or only contains the following amino acid sequence, or a fragment thereof having adenosine deaminase activity:

(SEQ ID NO: 426)
SEVEFSHEYWMRHALTLAKRARDEREVPVGAVLVHNNRVIGEGWNRAIGL
HDPTAHAEIMALRQGGLVMQNYRLYDATLYTTFEPCVMCAGAMIHSRIGR
VVFGVRNAKTGAAGSLMDVLHYPGMNHRVEITEGILADECAALLCTFFRM
PRSVFKAQKKAQSSTD.

In any aspect of the disclosure, or embodiments thereof, the adenosine deaminase is a truncated TadA*7.10 variant that is missing 1, 2, 3, 4, 5, 6, 7, 8, 9, 10, 11, 12, 13, 14, 15, 6, 17, 18, 19, or 20 N-terminal amino acid residues relative to the following amino acid sequence: SEVEFSHEYWMRHALTLAKRARDEREVPVGAVLVHNNRVIGEGWNRAIGLHDPTAHAEIMAL RQGGLVMQNYRLYDATLYTTFEPCVMCAGAMIHSRIGRVVFGVRNAKTGAAGSLMDVLHYPG MNHRVEITEGILADECAALLCTFFRMPRSVFKAQKKAQSSTD (SEQ ID NO: 426). In any aspect of the disclosure, or embodiments thereof, the adenosine deaminase is a truncated TadA*7.10 variant that is missing 1, 2, 3, 4, 5, 6, 7, 8, 9, 10, 11, 12, 13, 14, 15, 6, 17, 18, 19, or 20 C-terminal amino acid residues relative to the following amino acid sequence:

(SEQ ID NO: 426)
SEVEFSHEYWMRHALTLAKRARDEREVPVGAVLVHNNRVIGEGWNRAIGL
HDPTAHAEIMALRQGGLVMQNYRLYDATLYTTFEPCVMCAGAMIHSRIGR
VVFGVRNAKTGAAGSLMDVLHYPGMNHRVEITEGILADECAALLCTFFRM
PRSVFKAQKKAQSSTD.

In any aspect of the disclosure, or embodiments thereof, the base editor further contains a linker between the adenosine deaminase and the napDNAbp. In embodiments, the linker contains one or more amino acids. In embodiments, the linker contains an amino acid sequence selected from those listed in Tables 9, 10, or 11. In embodiments, the linker contains an amino acid sequence selected from one or more of: SGGSSGGSSGSETPGTSESATPESSGGSSGGS (SEQ ID NO: 357), EGGSEEEEESGS (SEQ ID NO: 542), and KGPKPKKEESEK (SEQ ID NO: 439). In embodiments, the linker contains the following amino acid sequence: SGGSSGGSSGSETPGTSESATPESSGGSSGGS (SEQ ID NO: 357).

In any aspect of the disclosure, or embodiments thereof, the base editor further contains a nuclear localization sequence (NLS). In embodiments, the NLS contains the amino acid sequence EGADKRTADGSEFESPKKKRKV (SEQ ID NO: 438).

In any aspect of the disclosure, or embodiments thereof, the base editor contains an amino acid sequence that is at least 80%, 85%, 90%, 95%, 96%, 97%, 98%, 99%, or 100% identical to the following amino acid sequence:

Variant G
(SEQ ID NO: 557)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDSGGSSGGSSGSETPGTSESA
TPESSGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDA
ILLSDILRVNTEITKAPLSASMVKRYDEHHQDLTLLKALVRQQLPEKYKEIFFDQSKNGYAG
YIDGGASQEEFYKFIKPILEKMDGTEELLVKLNREDLLRKQRTFDNGIIPHQIHLGELHAIL
RRQGDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRLRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGGHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGF
SKESILPKGNSDKLIARKKDWDPKKYGGFLQPTVAYSVLVVAKVEKGKSKKLKSVKELLGIT
IMERSSFEKNPIDFLEAKGYKEVKKDLIIKLPKYSLFELENGRKRMLASAKFLQKGNELALP
SKYVNFLYLASHYEKLKGSPKDNEQKQLFVEQHKHYLDEIIEQISEFSKRVILDDANLDKVL
SAYNKHRDKPIREQAENIIHLFTLTNLGAPRAFKYFDTTIARKEYKSTKEVLDATLIHQSIT
GLYETRIDLSQLGGDEGADKRTADGSEFESPKKKRKV.

In any aspect of the disclosure, or embodiments thereof, the guide polynucleotide is a single guide RNA (sgRNA). In any aspect of the disclosure, or embodiments thereof, the guide polynucleotide contains a spacer containing a nucleotide sequence selected from one or more of: 5′-ACCAUCGACAAGAAAGGGACUGA-3′ (SEQ ID NO: 466); 5′-CCAUCGACAAGAAAGGGACUGA-3′ (SEQ ID NO: 559); 5′-CAUCGACAAGAAAGGGACUGA-3′ (SEQ ID NO: 560); 5′-AUCGACAAGAAAGGGACUGA-3′ (SEQ ID NO: 561); 5′-UCGACAAGAAAGGGACUGA-3′ (SEQ ID NO: 562); and 5′-CGACAAGAAAGGGACUGA-3′(SEQ ID NO: 563). In any aspect of the disclosure, or embodiments thereof, the guide polynucleotide contains a scaffold containing a nucleotide sequence that is at least 90%, 95%, 96%, 97%, 98%, 99%, or 100% identical to the following nucleotide sequence: 5′-GUUUUAGAGC UAGAAAUAGC AAGUUAAAAU AAGGCUAGUC CGUUAUCAAC UUGAAAAAGU GGCACCGAGU CGGUGCUUUU-3′ (SEQ ID NO: 324). In any aspect of the disclosure, or embodiments thereof, the guide polynucleotide contains a nucleotide sequence selected from one or more of: 5′-ACCAUCGACAAGAAAGGGACUGA GUUUUAGAGC UAGAAAUAGC AAGUUAAAAU AAGGCUAGUC CGUUAUCAAC UUGAAAAAGU GGCACCGAGU CGGUGCUUUU-3′ (SEQ ID NO: 558); 5′-CCAUCGACAAGAAAGGGACUGA GUUUUAGAGC UAGAAAUAGC AAGUUAAAAU AAGGCUAGUC CGUUAUCAAC UUGAAAAAGU GGCACCGAGU CGGUGCUUUU-3′ (SEQ ID NO: 564); 5′-CAUCGACAAGAAAGGGACUGA GUUUUAGAGC UAGAAAUAGC AAGUUAAAAU AAGGCUAGUC CGUUAUCAAC UUGAAAAAGU GGCACCGAGU CGGUGCUUUU-3′ (SEQ ID NO: 565); 5′-AUCGACAAGAAAGGGACUGA GUUUUAGAGC UAGAAAUAGC AAGUUAAAAU AAGGCUAGUC CGUUAUCAAC UUGAAAAAGU GGCACCGAGU CGGUGCUUUU-3′ (SEQ ID NO: 566); 5′-UCGACAAGAAAGGGACUGA GUUUUAGAGC UAGAAAUAGC AAGUUAAAAU AAGGCUAGUC CGUUAUCAAC UUGAAAAAGU GGCACCGAGU CGGUGCUUUU-3′ (SEQ ID NO: 567); and 5′-CGACAAGAAAGGGACUGA GUUUUAGAGC UAGAAAUAGC AAGUUAAAAU AAGGCUAGUC CGUUAUCAAC UUGAAAAAGU GGCACCGAGU CGGUGCUUUU-3′ (SEQ ID NO: 568). In any aspect of the disclosure, or embodiments thereof, the guide polynucleotide contains the following nucleotide sequence:

(SEQ ID NO: 566)
5′-AUCGACAAGAAAGGGACUGA GUUUUAGAGC UAGAAAUAGC
AAGUUAAAAU AAGGCUAGUC CGUUAUCAAC UUGAAAAAGU
GGCACCGAGU CGGUGCUUUU-3′.

In any aspect of the disclosure, or embodiments thereof, the guide polynucleotide contains one or more modified nucleotides. In any aspect of the disclosure, or embodiments thereof, the guide polynucleotide contains a nucleotide having a 2′-OMe modification, a 2′-fluoro (F) modification, and/or a phosphorothioate modification. In any aspect of the disclosure, or embodiments thereof, the guide polynucleotide contains a nucleotide sequence, from 5′ to 3′, selected from one or more of:

(SEQ ID NO: 569)
mAsmUsmCsGACAAGAAAGGGACUGAGUUUUAGAGCUAGAAAUAGCAAGU
UAAAAUAAGGCUAGUCCGUUAUCAACUUGAAAAAGUGGCACCGAGUCGGU
GCmUsmUsmUsU;

mAsmUsmCsGACAAGAAAGGGACUGAGUUUUAGAGCUAGAAAUAGCAAGUUAAAAUAAGGCU AGUCCGUUAUCAACUUGAAAAAGUGGCACCGAGUCGGUGCUsmUsmUsmU (SEQ ID NO: 570), where the guide is covalently linked at the 3′ end to a peptide with the amino acid sequence CKRTADGSEFESPKKKRKV (SEQ ID NO: 543);

```
  (SEQ ID NO: 571)
mAsmUsmCsmGmAmCmAmAmGmAfAfAfGfGGsAsfCfUGsmAmGUsUsUsfUfAmGmAmGm
CmUmAmGmAmAmAmUmAmGmCmAmAmGUUmAAmAmAUmAmAmGmGCUmAGUCmCGUUmAmUm
CmAmAmCmUmUmGmAmAmAmAmAmGmUmGmGmCmAmCmCmGmAmGmUmCmGmGmUmGmCsmU
smUsmU;
  (SEQ ID NOs: 572)
mAsmUsmCsGACAAGAAAGGGACUGAmGUUUUAGmAmGmCmUmAmGmAmAmAmUmAmGmCmA
mAGUUmAAmAAmUAmAmGmGmCmUmAGUmCmCGUUAmUmCAAmCmUmUmGmAmAmAmAmAmG
mUmGGmCmAmCmCmGmAmGmUmCmGmGmUmGmCmUsmUsmUsmU;
  (SEQ ID NOS: 573)
mCsmAsmUsCGACAAGAAAGGGACUGAGUUUUAGAGCUAGAAAUAGCAAGUUAAAAUAAGGC
UAGUCCGUUAUCAACUUGAAAAAGUGGCACCGAGUCGGUGCmUsmUsmUsU;
(SEQ ID NOs: 574)
mAsmUsmCsmGmAmCmAmAmGmAfAfAfGIGGsAsfCUGmAmGUSUUfUfAmGmAmGmCmUm
AmGmAmAmAmUmAmGmCmAmAmGUUmAAmAmAUmAmAmGmGCUmAGUCmCGUUmAmUmCmAm
AmCmUmUmGmAmAmAmAmAmGUGmGmCmAmCmCmGmAmGmUmCmGmGmUmGmCsmUsmUsmU;
  (SEQ ID NOs: 575)
mAsmUsmCsGACAAGAAAGGGACUGAmGUUUUAGmAmGmCmUmAmGmAmAmAmUmAmGmCmA
mAGUUmAAmAAmUAmAmGmGmCmUmAGUmCmCGUUAmUmCAAmCmUmUmGmAmAmAmAmAmG
UGGmCmAmCmCmGmAmGmUmCmGmGmUmGmCmUsmUsmUsmU;
  (SEQ ID NOs: 576)
5′mAsmUsmCsmGmAmCmAmAmGmAAAGGGACUGmAmGUUUUAmGmAmGmCmUmAmGmAmAmA
mUmAmGmCmAmAmGUUmAAmAmAUmAmAmGmGCUmAGUCmCGUUmAmUmCmAmAmCmUmUmG
mAmAmAmAmAmGUGmGmCmAmCmCmGmAmGmUmCmGmGmUmGmCsmUsmUsmU;
  (SEQ ID NOs: 577)
mAsmUsmCsmGmAmCmAmAmGmAAAGGGACUGAmGUUUUAGmAmGmCmUmAmGmAmAmAmUm
AmGmCmAmAGUUmAAmAAmUAmAmGmGmCmUmAGUmCmCGUUAmUmCAAmCmUmUmGmAmAm
AmAmAmGUGGmCmAmCmCmGmAmGmUmCmGmGmUmGmCmUsmUsmUsmU;
  (SEQ ID NOS: 578)
mCsmAsmUsmCmGmAmCmAmAmGmAAAGGGACUGAmGUUUUAGmAmGmCmUmAmGmAmAmAm
UmAmGmCmAmAGUUmAAmAAmUAmAmGmGmCmUmAGUmCmCGUUAmUmCAAmCmUmUmGmAm
AmAmAmAmGUGGmCmAmCmCmGmAmGmUmCmGmGmUmGmCmUsmUsmUsmU;
  (SEQ ID NOs: 579)
mAsmUsmCsmGmAmCmAmAmGmAmAAmGmGGAmCUGmAmGUUUUAGmAmGmCmUmAmGmAmA
mAmUmAmGmCmAmAGUUmAAmAAmUAmAmGmGmCmUmAGUmCmCGUUAmUmCAAmCmUmUmG
mAmAmAmAmAmGUGGmCmAmCmCmGmAmGmUmCmGmGmUmGmCmUsmUsmUsmU;
  (SEQ ID NOs: 580);
mCsmAsmUsmCmGmAmCmAmAmGmAmAAmGmGGAmCUGmAmGUUUUAGmAmGmCmUmAmGmA
mAmAmUmAmGmCmAmAGUUmAAmAAmUAmAmGmGmCmUmAGUmCmCGUUAmUmCAAmCmUmU
mGmAmAmAmAmAmGUGGmCmAmCmCmGmAmGmUmCmGmGmUmGmCmUsmUsmUsmU
  (SEQ ID NOs: 581)
mCsmAsmUsCGACAAGAAAGGGACUGAmGUUUUAGmAmGmCmUmAmGmAmAmAmUmAmGmCm
AmAGUUmAAmAAmUAmAmGmGmCmUmAGUmCmCGUUAmUmCAAmCmUmUmGmAmAmAmAmAm
GUGGmCmAmCmCmGmAmGmUmCmGmGmUmGmCmUsmUsmUsmU;
and
  (SEQ ID NOs: 582)
mAsmUsmCsGACAAGAAAGGGACUGAGUUUUAGAmGmCmCmGmGmCmGmGmAmAmAmCmGmC
mCmGmGmCAAGUUAAAAUAAGGCUAGUCCGUUAUCAmAmCmUmUmGmAmAmAmAmAmGmUmG
mGmCmAmCmCmGmAmGmUmCmGmGmUmGmCmUsmUsmUsmU;
  (SEQ ID NOs: 583)
mAsmUsmCsmGmAmCmAmAmGmAAAGGGACUGAGUUUUAGAmGmCmCmGmGmCmGmGmAmAm
AmCmGmCmCmGmGmCAAGUUAAAAUAAGGCUAGUCCGUUAUCAmAmCmUmUmGmAmAmAmAm
AmGmUmGmGmCmAmCmCmGmAmGmUmCmGmGmUmGmCmUsmUsmUsmU;
  (SEQ ID NOs: 584)
mAsmUsmCsGACAAGAAAGGGACUGAGUUUUAGAmGmCmUmAmGmAmAmAmUmAmGmCAAGU
UAAAAUAAGGCUAGUCCGUUAmUmCAAmCmUmUGGACUUCGGUCCmAmAmGmUmGGmCmAmC
mCmGmAmGmUmCmGmGmUmGmCmUsmUsmUsmU;
or
  (SEQ ID NO: 585)
mAsmUsmCsGACAAGAAAGGGACUGAGUUUUAGAmGmCmCmGmGmCmGmGmAmAmAmCmGmC
mCmGmGmCAAGUUAAAAUAAGGCUAGUCCGUUAmUmCAAmCmUmUGGACUUCGGUCCmAmAm
GmUmGGmCmAmCmCmGmAmGmUmCmGmGmUmGmCmUmUmUmU;
```

where “N” represents any nucleotide, “mN” indicates a 2′-OMe modification of the nucleotide “N”, fN indicates a 2′-fluoro (F) modification of the nucleotide “N,” and “Ns” indicates that the nucleotide “N” is linked to the following nucleotide by a phosphorothioate. In any aspect of the disclosure, or embodiments thereof, the guide polynucleotide contains the following nucleotide sequence, from 5′ to 3′:

(SEQ ID NO: 586)
mAsmUsmCsGACAAGAAAGGGACUGAmGUUUUAGmAmGmCmUmAmGmAm
AmAmUmAmGmCmAmAGUUmAAmAAmUAmAmGmGmCmUmAGUmCmCGUUA
mUmCAAmCmUmUmGmAmAmAmAmAmGUGGmCmAmCmCmGmAmGmUmCmG
mGmUmGmCmUsmUsmUsmU;

where “N” represents any nucleotide, “mN” indicates a 2′-OMe modification of the nucleotide “N”, and “Ns” indicates that the nucleotide “N” is linked to the following nucleotide by a phosphorothioate.

In any aspect of the disclosure, or embodiments thereof, the guide polynucleotide contains a nucleotide sequence that is at least 90%, 95%, 96%, 97%, 98%, 99%, or 100% identical to:

(SEQ ID NO: 324)
5′-GUUUUAGAGC UAGAAAUAGC AAGUUAAAAU AAGGCUAGUC
CGUUAUCAAC UUGAAAAAGU GGCACCGAGU CGGUGCUUUU-3′.

In any aspect of the disclosure, or embodiments thereof, the one or more guide RNAs is any of the guide polynucleotides of any aspect of the disclosure, or embodiments thereof.

In any aspect of the disclosure, or embodiments thereof, the base editor has a PAM specificity for the nucleotide sequence 5″-NGC-3′.

In any aspect of the disclosure, or embodiments thereof, the editing is in a cell. In any aspect of the disclosure, or embodiments thereof, the cell is in vivo or ex vivo.

In any aspect of the disclosure, or embodiments thereof, the alteration is a A·T to G·C alteration at the SNP associated with alpha-1 antitrypsin deficiency and changes a lysine at amino acid position 342 of an alpha-1 antitrypsin polypeptide encoded by the alpha-1 antitrypsin polynucleotide to glutamic acid. In any aspect of the disclosure, or embodiments thereof, the SNP associated with alpha-1 antitrypsin deficiency results in expression of an alpha-1 antitrypsin polypeptide having a lysine at amino acid position 342. In any aspect of the disclosure, or embodiments thereof, the SNP associated with alpha-1 antitrypsin deficiency results in the substitution of a glutamic acid amino acid with a lysine in the alpha-antitrypsin polypeptide encoded by the alpha-1 antitrypsin polynucleotide.

In any aspect of the disclosure, or embodiments thereof, the base editor is in complex with a single guide RNA (sgRNA) containing a nucleotide sequence complementary to an alpha-1 antitrypsin nucleotide sequence containing the SNP associated with alpha-1 antitrypsin deficiency.

In any aspect of the disclosure, or embodiments thereof, the one or more guide polynucleotides contain a guide polynucleotide of any aspect of the disclosure, or embodiments thereof.

In any aspect of the disclosure, or embodiments thereof, the cell produced is a hepatocyte or progenitor thereof. In any aspect of the disclosure, or embodiments thereof, the cell is from a subject having alpha-1 antitrypsin deficiency. In any aspect of the disclosure, or embodiments thereof, the cell is a mammalian cell or human cell.

In any aspect of the disclosure, or embodiments thereof, the A·T to G·C alteration at the SNP associated with alpha-1 antitrypsin deficiency results in the substitution of a lysine with a glutamic acid in an alpha-antitrypsin polypeptide encoded by an alpha-1 antitrypsin polynucleotide containing the SNP.

In any aspect of the disclosure, or embodiments thereof, the cell is selected for the A·T to G·C alteration of the SNP associated with alpha-1 antitrypsin deficiency.

In any aspect of the disclosure, or embodiments thereof, the one or more guide polynucleotides contain a CRISPR RNA (crRNA) and a trans-encoded small RNA (tracrRNA). The crRNA contains a nucleotide sequence complementary to an alpha-1 antitrypsin nucleic acid sequence containing the SNP associated with alpha-1 antitrypsin deficiency.

In any aspect of the disclosure, or embodiments thereof, the base editor and the one or more guide polynucleotides forms a complex in the cell.

In any aspect of the disclosure, or embodiments thereof, the base editor is in complex with a single guide RNA (sgRNA) containing a nucleotide sequence complementary to an alpha-1 antitrypsin nucleotide sequence containing the SNP associated with alpha-1 antitrypsin deficiency.

In any aspect of the disclosure, or embodiments thereof, the cell is autologous to the subject. In any aspect of the disclosure, or embodiments thereof, the cell is allogenic to the subject.

In any aspect of the disclosure, or embodiments thereof, the one or more guide polynucleotides is the guide polynucleotide of any aspect of the disclosure, or embodiments thereof.

In any aspect of the disclosure, or embodiments thereof, the hepatocyte or hepatocyte progenitor is a mammalian cell or human cell.

In any aspect of the disclosure, or embodiments thereof, the cell is selected for the A·T to G·C alteration of the SNP associated with alpha-1 antitrypsin deficiency.

In any aspect of the disclosure, or embodiments thereof, the base editor and the one or more guide polynucleotides forms a complex in the cell. In any aspect of the disclosure, or embodiments thereof, the base editor is in complex with a single guide RNA (sgRNA) containing a nucleotide sequence complementary to an alpha-1 antitrypsin nucleotide sequence containing the SNP associated with alpha-1 antitrypsin deficiency.

In any aspect of the disclosure, or embodiments thereof, the A·T to G·C alteration at the SNP associated with A1AD changes a lysine at amino acid position 342 of an alpha-1 antitrypsin polypeptide encoded by the alpha-1 antitrypsin polynucleotide to glutamic acid. In any aspect of the disclosure, or embodiments thereof, the SNP associated with A1AD results in expression of an alpha-1 antitrypsin polypeptide having a lysine at amino acid position 342.

In any aspect of the disclosure, or embodiments thereof, the method further involves effecting a deamination of the SNP associated with A1AD.

In any aspect of the disclosure, or embodiments thereof, the A·T to G·C alteration replaces a target nucleobase with a wild type nucleobase or with a non-wild type nucleobase, and where the replacing ameliorates symptoms of A1AD. In any aspect of the disclosure, or embodiments thereof, the A·T to G·C alteration results in the substitution of a glutamic acid amino acid with a lysine in an alpha-antitrypsin polypeptide encoded by an alpha-1 antitrypsin polynucleotide. In any aspect of the disclosure, or embodiments thereof, the A·T to G·C alteration is 1-20 nucleobases away from an NGC PAM sequence in a polynucleotide sequence targeted by the one or more guide polynucleotides. In any aspect of the disclosure, or embodiments thereof, the A·T to G·C alteration is 14 nucleobases upstream of the PAM sequence. In any aspect of the disclosure, or embodiments thereof, the PAM sequence is AGC.

In any aspect of the disclosure, or embodiments thereof, the subject is a non-human mammal or a human.

In any aspect of the disclosure, or embodiments thereof, the composition or pharmaceutical composition further contains a lipid. In embodiments, the lipid is a cationic lipid.

In any aspect of the disclosure, or embodiments thereof, the kit further contains a package insert with instructions for use.

In any aspect of the disclosure, or embodiments thereof, the variant does not contain an N-terminal methionine.

In any aspect provided herein, or embodiments thereof, the method is not a process for modifying the germline genetic identity of human beings.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenine” or “9H-Purin-6-amine” is meant a purine nucleobase with the molecular formula C₅H₅N₅, having the structure

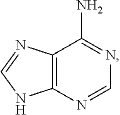

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2 (1H)-one” is meant an adenine molecule attached to a ribose sugar via a glycosidic bond, having the structure

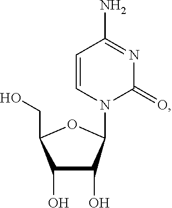

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, PCT/US2020/018195, and PCT/US2020/028568, the full contents of which are each incorporated herein by reference in their entireties for all purposes.

In some embodiments, the adenosine deaminase comprises at least one alteration in the following sequence:

(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD
(also termed TadA*7.10).

In the interest of clarity, residues L36, I76, V82, Y147, Q154, and N157 are indicated by bold and underlined text.

In some embodiments, TadA*7.10 comprises at least one amino acid alteration. In some embodiments, TadA*7.10 comprises an alteration in any one of amino acid residues L36, I76, V82, Y147, Q154, and N157 of TadA*7.10. In some embodiments, TadA*7.10 comprises any one of the amino acid alterations L36H, I76Y, V82T, Y147T, Q154S, and N157K of TadA*7.10.

In some embodiments, TadA*7.10 comprises the amino acid alterations L36H, I76Y, V82T, Y147T, Q154S, and N157K of TadA*7.10. For example, the TadA may have a sequence of

(SEQ ID NO: 426)
SEVEFSHEYWMRHALTLAKRARDEREVPVGAVLVHNNRVIGEGWNRAIGL
HDPTAHAEIMALRQGGLVMQNYRLYDATLYTTFEPCVMCAGAMIHSRIGR
VVFGVRNAKTGAAGSLMDVLHYPGMNHRVEITEGILADECAALLCTFFRM
PRSVFKAQKKAQSSTD.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE. By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, where such alterations are relative to the following reference sequence:

(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD,

or a corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, or polypeptide, or fragments thereof.

By “alpha-1 antitrypsin (A1AT) protein” or “Homo sapiens serpin family A member 1 (SERPINA1) polypeptide” is meant a polypeptide or fragment thereof having at least about 85% amino acid sequence identity to NCBI Ref Seq Accession No. NP_000286.3. In some embodiments, the A1AT protein has protease inhibition activity. In particular embodiments, an A1AT protein comprises one or more alterations (e.g., at E342) relative to the following reference sequence or a corresponding amino acid sequence. In some embodiments, the alteration reduces or eliminates A1AT protease inhibition activity and/or disrupts protein folding. In one particular embodiment, an A1AT protein associated with A1AD comprises an E342K mutation, where the position of the mutation is indicated relative to a mature A1AT protein that does not include the signal peptide underlined in the below sequence. An exemplary A1AT amino acid sequence is provided below, where a signal peptide is shown as plain underlined text, and position E342 is indicated in bold-underlined text.

>NP_000286.3 alpha-1-antitrypsin precursor [Homo sapiens]
(SEQ ID NO: 427)
MPSSVSWGILLLAGLCCLVPVSLAEDPQGDAAQKTDTSHHDQDHPTFNKITPNLAEFAFSLY
RQLAHQSNSTNIFFSPVSIATAFAMLSLGTKADTHDEILEGLNFNLTEIPEAQIHEGFQELL
RTLNQPDSQLQLTTGNGLFLSEGLKLVDKFLEDVKKLYHSEAFTVNFGDTEEAKKQINDYVE
KGTQGKIVDLVKELDRDTVFALVNYIFFKGKWERPFEVKDTEEEDFHVDQVTTVKVPMMKRL
GMFNI QHCKKLSSWVLLMKYLGNATAIFFLPDEGKLQHLENELTHDIITKFLENEDRRSASL
HLPKLSITGTYDLKSVLGQLGITKVFSNGADLSGVTEEAPLKLSKAVHKAVLTIDEKGTEAA
GAMFLEAIPMSIPPEVKFNKPFVFLMIEQNTKSPLFMGKVVNPTQK

In the above A1AT protein sequence, the first 24 amino acids constitute the signal peptide (underlined). Position 342 of the sequence, which is mutated in A1AD (i.e., E342K), is determined based on setting amino acid residue “E” following the signal sequence as amino acid “1”.

By “alpha-1 antitrypsin (A1AT) polynucleotide” or “Homo sapiens serpin family A member 1 (SERPINA1) polynucleotide” is meant a nucleic acid molecule encoding an A1AT polypeptide, as well as the introns, exons, 3′ untranslated regions, 5′ untranslated regions, and regulatory sequences associated with its expression, or fragments thereof. In embodiments, an A1AT polynucleotide is the genomic sequence, cDNA, mRNA, or gene associated with and/or required for A1AT expression. An exemplary A1AT nucleotide sequence from Homo Sapiens is provided below (NCBI Accession No. NM_NM_000295). A representative A1AT gene sequence is provided at ENSEMBL Accession No. ENSG00000197249, which is proved below. A further representative A1AT gene sequence is provided below corresponding to NCBI RefSeq Accession No. NM_000295.

>NM_000295.5:48-1304 Homo sapiens serpin family A member 1 (SERPINA1), transcript variant 1, mRNA. In embodiments, the methods of the disclosure involve deaminating a nucleobase corresponding to the nucleobase shown as bold-underlined text in the below sequence.

(SEQ ID NO: 428)
ATGCCGTCTTCTGTCTCGTGGGGCATCCTCCTGCTGGCAGGCCTGTGCTGCCTGGTCCCTGT
CTCCCTGGCTGAGGATCCCCAGGGAGATGCTGCCCAGAAGACAGATACATCCCACCATGATC
AGGATCACCCAACCTTCAACAAGATCACCCCCAACCTGGCTGAGTTCGCCTTCAGCCTATAC
CGCCAGCTGGCACACCAGTCCAACAGCACCAATATCTTCTTCTCCCCAGTGAGCATCGCTAC
AGCCTTTGCAATGCTCTCCCTGGGGACCAAGGCTGACACTCACGATGAAATCCTGGAGGGCC
TGAATTTCAACCTCACGGAGATTCCGGAGGCTCAGATCCATGAAGGCTTCCAGGAACTCCTC
CGTACCCTCAACCAGCCAGACAGCCAGCTCCAGCTGACCACCGGCAATGGCCTGTTCCTCAG
CGAGGGCCTGAAGCTAGTGGATAAGTTTTTGGAGGATGTTAAAAAGTTGTACCACTCAGAAG
CCTTCACTGTCAACTTCGGGGACACCGAAGAGGCCAAGAAACAGATCAACGATTACGTGGAG
AAGGGTACTCAAGGGAAAATTGTGGATTTGGTCAAGGAGCTTGACAGAGACACAGTTTTTGC
TCTGGTGAATTACATCTTCTTTAAAGGCAAATGGGAGAGACCCTTTGAAGTCAAGGACACCG
AGGAAGAGGACTTCCACGTGGACCAGGTGACCACCGTGAAGGTGCCTATGATGAAGCGTTTA
GGCATGTTTAACATCCAGCACTGTAAGAAGCTGTCCAGCTGGGTGCTGCTGATGAAATACCT
GGGCAATGCCACCGCCATCTTCTTCCTGCCTGATGAGGGGAAACTACAGCACCTGGAAAATG
AACTCACCCACGATATCATCACCAAGTTCCTGGAAAATGAAGACAGAAGGTCTGCCAGCTTA
CATTTACCCAAACTGTCCATTACTGGAACCTATGATCTGAAGAGCGTCCTGGGTCAACTGGG
CATCACTAAGGTCTTCAGCAATGGGGCTGACCTCTCCGGGGTCACAGAGGAGGCACCCCTGA
AGCTCTCCAAGGCCGTGCATAAGGCTGTGCTGACCATCGACGAGAAAGGGACTGAAGCTGCT
GGGGCCATGTTTTTAGAGGCCATACCCATGTCTATCCCCCCCGAGGTCAAGTTCAACAAACC
CTTTGTCTTCTTAATGATTGAACAAAATACCAAGTCTCCCCTCTTCATGGGAAAAGTGGTGA
ATCCCACCCAAAAATAA.

>ENSG00000197249; chromosome: GRCh38:14:94376147:94391293:−1 (exons are shown in bold text). In embodiments, the methods of the disclosure involve deaminating a nucleobase corresponding to the nucleobase shown as bold-underlined text in the below sequence. In embodiments, an NGC PAM sequence associated with deamination of the nucleobase according to the methods provided herein corresponds to the nucleotides shown as double-underlined plain text in the below sequence.

(SEQ ID NO: 429)
TGGTGCGTTTTTCCAGATTATCCTAGCCCTTCCTCCCAGGATGGATGTCCAGAGCAGGGCGG
GGGCTGAGCCTAGAGCCCTGCCAAAAGAGCAGGACCCCAAATTCTGAGCCCCTTACTTGCCT
CACCTGCTCCCACCCATGCTTTCTTCATTCCTCCTCCAAAAGCCCCAGCTCCCCACTGCAAT
CCCTTCTGCACCCAGCCAGGTCCTATGACACACACCTCCCCAGTGCACACAGACCTGCCCAA
CTGTGGGGCTGCCCACTGGGCATTTCATAGGTGGCTCAGTCCTCTTCCCTCTGCAGCTGGCC
CCAGAAACCTGCCAGTTATTGGTGCCAGGTCTGTGCCAGGAGGGCGAGGCCTGTCATTTCTA
GTAATCCTCTGGGCAGTGTGACTGTACCTCTTGCGGCAACTCAAAGGGAGAGGGTGACTTGT
CCCGGGTCACAGAGCTGAAAGGGCAGGTACAACAGGTGACATGCCGGGCTGTCTGAGTTTAT
GAGGGCCCAGTCTTGTGTCTGCCGGGCAATGAGCAAGGCTCCTTCCTGTCCAAGCTCCCCGC
CCCTCCCCAGCCTACTGCCTCCACCCGAAGTCTACTTCCTGGGTGGGCAGGAACTGGGCACT
GTGCCCAGGGCATGCACTGCCTCCACGCAGCAACCCTCAGAGTCCTGAGCTGAACCAAGAAG
GAGGAGGGGGTCGGGCCTCCGAGGAAGGCCTAGCCGCTGCTGCTGCCAGGAATTCCAGGTTG
GAGGGGCGGCAACCTCCTGCCAGCCTTCAGGCCACTCTCCTGTGCCTGCCAGAAGAGACAGA
GCTTGAGGAGAGCTTGAGGAGAGCAGGAAAGGTGGGACATTGCTGCTGCTGCTCACTCAGTT
CCACAGGTGGGAGGGACAGCAGGGCTTAGAGTGGGGGTCATTGTGCAGATGGGAAAACAAAG
GCCCAGAGAGGGGAAGAAATGCCCAGGAGCTACCGAGGGCAGGCGACCTCAACCACAGCCCA
GTGCTGGAGCTGTGAGTGGATGTAGAGCAGCGGAATATCCATTCAGCCAGCTCAGGGGAAGG
ACAGGGGCCCTGAAGCCAGGGGATGGAGCTGCAGGGAAGGGAGCTCAGAGAGAAGGGGAGGG
GAGTCTGAGCTCAGTTTCCCGCTGCCTGAAAGGAGGGTGGTACCTACTCCCTTCACAGGGTA
ACTGAATGAGAGACTGCCTGGAGGAAAGCTCTTCAAGTGTGGCCCACCCCACCCCAGTGACA
CCAGCCCCTGACACGGGGGAGGGAGGGCAGCATCAGGAGGGGCTTTCTGGGCACACCCAGTA
CCCGTCTCTGAGCTTTCCTTGAACTGTTGCATTTTAATCCTCACAGCAGCTCAACAAGGTAC
ATACCGTCACCATCCCCATTTTACAGATAGGGAAATTGAGGCTCGGAGCGGTTAAACAACTC
ACCTGAGGCCTCACAGCCAGTAAGTGGGTTCCCTGGTCTGAATGTGTGTGCTGGAGGATCCT
GTGGGTCACTCGCCTGGTAGAGCCCCAAGGTGGAGGCATAAATGGGACTGGTGAATGACAGA
AGGGGCAAAAATGCACTCATCCATTCACTCTGCAAGTATCTACGGCACGTACGCCAGCTCCC
AAGCAGGTTTGCGGGTTGCACAGCGGGCGATGCAATCTGATTTAGGCTTTTAAAGGGATTGC
AATCAAGTGGGGCCCCACTAGCCTCAACCCTGTACCTCCCCTCCCCTCCACCCCCAGCAGTC
TCCAAAGGCCTCCAACAACCCCAGAGTGGGGGCCATGTATCCAAAGAAACTCCAAGCTGTAT
ACGGATCACACTGGTTTTCCAGGAGCAAAAACAGAAACAGGCCTGAGGCTGGTCAAAATTGA
ACCTCCTCCTGCTCTGAGCAGCCTGGGGGGCAGACTAAGCAGAGGGCTGTGCAGACCCACAT
AAAGAGCCTACTGTGTGCCAGGCACTTCACCCGAGGCACTTCACAAGCATGCTTGGGAATGA
AACTTCCAACTCTTTGGGATGCAGGTGAAACAGTTCCTGGTTCAGAGAGGTGAAGCGGCCTG
CCTGAGGCAGCACAGCTCTTCTTTACAGATGTGCTTCCCCACCTCTACCCTGTCTCACGGCC
CCCCATGCCAGCCTGACGGTTGTGTCTGCCTCAGTCATGCTCCATTTTTCCATCGGGACCAT
CAAGAGGGTGTTTGTGTCTAAGGCTGACTGGGTAACTTTGGATGAGCGGTCTCTCCGCTCTG
AGCCTGTTTCCTCATCTGTCAAATGGGCTCTAACCCACTCTGATCTCCCAGGGCGGCAGTAA
GTCTTCAGCATCAGGCATTTTGGGGTGACTCAGTAAATGGTAGATCTTGCTACCAGTGGAAC
AGCCACTAAGGATTCTGCAGTGAGAGCAGAGGGCCAGCTAAGTGGTACTCTCCCAGAGACTG
TCTGACTCACGCCACCCCCTCCACCTTGGACACAGGACGCTGTGGTTTCTGAGCCAGGTACA
ATGACTCCTTTCGGTAAGTGCAGTGGAAGCTGTACACTGCCCAGGCAAAGCGTCCGGGCAGC
GTAGGCGGGCGACTCAGATCCCAGCCAGTGGACTTAGCCCCTGTTTGCTCCTCCGATAACTG
GGGTGACCTTGGTTAATATTCACCAGCAGCCTCCCCCGTTGCCCCTCTGGATCCACTGCTTA
AATACGGACGAGGACAGGGCCCTGTCTCCTCAGCTTCAGGCACCACCACTGACCTGGGACAG
TGAATCGTAAGTATGCCTTTCACTGCGAGAGGTTCTGGAGAGGCTTCTGAGCTCCCCATGGC
CCAGGCAGGCAGCAGGTCTGGGGCAGGAGGGGGGTTGTGGAGTGGGTATCCGCCTGCTGAGG
TGCAGGGCAGATGGAGAGGCTGCAGCTGAGCTCCTATTTTCATAATAACAGCAGCCATGAGG
GTTGTGTCCTGTTTCCCAGTCCTGCCCGGTCCCCCCTCGGTACCTCCTGGTGGATACACTGG
TTCCTGTAAGCAGAAGTGGATGAGGGTGTCTAGGTCTGCAGTCCTGGCACCCCAGGATGGGG
GACACCAGCCAAGATACAGCAACAGCAACAAAGCGCAGCCATTTCTTTCTGTTTGCACAGCT
CCTCTGTCTGTCGGGGGCTCCTGTCTGTTGTCTCCTATAAGCCTCACCACCTCTCCTACTGC
TTGGGCATGCATCTTTCTCCCCTTCTATAGATGAGGAGGTTAAGGTCCAGAGAGGGGTGGGG
AGGAACGCCGGCTCACATTCTCCATCCCCTCCAGATATGACCAGGAACAGACCTGTGCCAGG
CCTCAGCCTTACATCAAAATGGGCCTCCCCATGCACCGTGGACCTCTGGGCCCTCCTGTCCC
AGTGGAGGACAGGAAGCTGTGAGGGGCACTGTCACCCAGGGCTCAAGCTGGCATTCCTGAAT
AATCGCTCTGCACCAGGCCACGGCTAAGCTCAGTGCGTGATTAAGCCTCATAACCCTCCAAG
GCAGTTACTAGTGTGATTCCCATTTTACAGATGAGGAAGATGGGGACAGAGAGGTGAATAAC
TGGCCCCAAATCACACACCATCCATAATTCGGGCTCAGGCACCTGGCTCCAGTCCCCAAACT
CTTGAACCTGGCCCTAGTGTCACTGTTTCTCTTGGGTCTCAGGCGCTGGATGGGGAACAGGA
AACCTGGGCTGGACTTGAGGCCTCTCTGATGCTCGGTGACTTCAGACAGTTGCTCAACCTCT
CTGTTCTCTTGGGCAAAACATGATAACCTTTGACTTCTGTCCCCTCCCCTCACCCCACCCGA
CCTTGATCTCTGAAGTGTTGGAAGGATTTAATTTTTCCTGCACTGAGTTTTGGAGACAGGTC
AAAAAGATGACCAAGGCCAAGGTGGCCAGTTTCCTATAGAACGCCTCTAAAAGACCTGCAGC
AATAGCAGCAAGAACTGGTATTCTCGAGAACTTGCTGCGCAGCAGGCACTTCTTGGCATTTT
ATGTGTATTTAATTTCACAATAGCTCTATGACAAAGTCCACCTTTCTCATCTCCAGGAAACT
GAGGTTCAGAGAGGTTAAGTAACTTGTCCAAGGTCACACAGCTAATAGCAAGTTGACGTGGA
GCAATCTGGCCTCAGAGCCTTTAATTTTAGCCACAGACTGATGCTCCCCTCTTCATTTAGCC
AGGCTGCCTCTGAAGTTTTCTGATTCAAGACTTCTGGCTTCAGCTTTGTACACAGAGATGAT
TCAATGTCAGGTTTTGGAGTGAAATCTGTTTAATCCCAGACAAAACATTTAGGATTACATCT
CAGTTTTGTAAGCAAGTAGCTCTGTGATTTTTAGTGAGTTATTTAATGCTCTTTGGGGCTCA
ATTTTTCTATCTATAAAATAGGGCTAATAATTTGCACCTTATAGGGTAAGCTTTGAGGACAG
ATTAGATGATACGGTGCCTGTAAAACACCAGGTGTTAGTAAGTGTGGCAATGATGGTGACGC
TGAGGCTGATGTTTGCTTAGCATAGGGTTAGGCAGCTGGCAGGCAGTAAACAGTTGGATAAT
TTAATGGAAAATTTGCCAAACTCAGATGCTGTTCACTGCTGAGCAGGAGCCCCTTCCTGCTG
AAATGGTCCTGGGGAGTGCAGCAGGCTCTCCGGGAAGAAATCTACCATCTCTCGGGCAGGAG
CTCAACCTGTGTGCAGGTACAGGGAGGGCTTCCTCACCTGGTGCCCACTCATGCATTACGTC
AGTTATTCCTCATCCCTGTCCAAAGGATTCTTTTCTCCATTGTACAGCTATGAAGCTAGTGC
TCAAAGAAGTGAAGTCATTTACCCCAGGCCCCCTGCCAGTAAGTGACAGGGCCTGGTCACAC
TTGGGTTTATTTATTGCCCAGTTCAACAGGTTGTTTGACCATAGGCGAGATTCTCTTCCCTG
CACCCTGCCGGGTTGCTCTTGGTCCCTTATTTTATGCTCCCGGGTAGAAATGGTGTGAGATT
AGGCAGGGAGTGGCTCGCTTCCCTGTCCCTGGCCCCGCAAAGAGTGCTCCCACCTGCCCCGA
TCCCAGAAATGTCACCATGAAGCCTTCATTCTTTTGGTTTAAAGCTTGGCCTCAGTGTCCGT
ACACCATGGGGTACTTGGCCAGATGGCGACTTTCTCCTCTCCAGTCGCCCTCCCAGGCACTA
GCTTTTAGGAGTGCAGGGTGCTGCCTCTGATAGAAGGGCCAGGAGAGAGCAGGTTTTGGAGT
CCTGATGTTATAAGGAACAGCTTGGGAGGCATAATGAACCCAACATGATGCTTGAGACCAAT
GTCACAGCCCAATTCTGACATTCATCATCTGAGATCTGAGGACACAGCTGTCTCAGTTCATG
ATCTGAGTGCTGGGAAAGCCAAGACTTGTTCCAGCTTTGTCACTGACTTGCTGTATAGCCTC
AACAAGGCCCTGACCCTCTCTGGGCTTCAAACTCTTCACTGTGAAAGGAGGAAACCAGAGTA
GGTGATGTGACACCAGGAAAGATGGATGGGTGTGGGGGAATGTGCTCCTCCCAGCTGTCACC
CCCTCGCCACCCTCCCTGCACCAGCCTCTCCACCTCCTTTGAGCCCAGAATTCCCCTGTCTA
GGAGGGCACCTGTCTCATGCCTAGCCATGGGAATTCTCCATCTGTTTTGCTACATTGAACCC
AGATGCCATTCTAACCAAGAATCCTGGCTGGGTGCAGGGGCTCTCGCCTGTAACCCCAGCAC
TTTGGGAGGCCAAGGCAGGCGGATCAAGAGGTCAGGAGTTCAAGACCTGCCTGGCCAACACG
GTGAAACCTCAGCTCTACTAAAAATACAAAAATTAGCCAGGCGTGGTGGCACACGCCTGTAA
TCCCAGCTATTTGGGAAGCTGAGACAGAAGAATTTCTTGAACCCGGGAGGTGGAGGTTTCAG
TGAGCCGAGATCACGCCACTGCACTCCACCCTGGCAGATAAAGCGAGACTCTGTCTCAAAAA
AAACCCAAAAACCTATGTTAGTGTACAGAGGGCCCCAGTGAAGTCTTCTCCCAGCCCCACTT
TGCACAACTGGGGAGAGTGAGGCCCCAGGACCAGAGGATTCTTGCTAAAGGCCAAGTGGATA
GTGATGGCCCTGCCAGGGCTAGAAGCCACAACCTCTGGCCCTGAGGCCACTCAGCATATTTA
GTGTCCCCACCCTGCAGAGGCCCAACTCCCTCCTGACCACTGAGCCCTGTAATGATGGGGGA
ATTTCCATAAGCCATGAAGGACTGCACAAAGTTCAGTTGGGAAGTGAAAGAGAAATTAAAGG
GAGATGGAAATATACAGCACTAATTTTAGCACCGTCTTTAGTTCTAACAACACTAGCTAGCT
GAAGAAAAATACAAACATGTATTATGTAATGTGTGGTCTGTTCCATTTGGATTACTTAGAGG
CACGAGGGCCAGGAGAAAGGTGGTGGAGAGAAACCAGCTTTGCACTTCATTTGTTGCTTTAT
TGGAAGGAAACTTTTAAAAGTCCAAGGGGGTTGAAGAATCTCAATATTTGTTATTTCCAGCT
TTTTTTCTCCAGTTTTTCATTTCCCAAATTCAAGGACACCTTTTTCTTTGTATTTTGTTAAG
ATGATGGTTTTGGTTTTGTGACTAGTAGTTAACAATGTGGCTGCCGGGCATATTCTCCTCAG
CTAGGACCTCAGTTTTCCCATCTGTGAAGACGGCAGGTTCTACCTAGGGGGCTGCAGGCTGG
TGGTCCGAAGCCTGGGCATATCTGGAGTAGAAGGATCACTGTGGGGCAGGGCAGGTTCTGTG
TTGCTGTGGATGACGTTGACTTTGACCATTGCTCGGCAGAGCCTGCTCTCGCTGGTTCAGCC
ACAGGCCCCACCACTCCCTATTGTCTCAGCCCCGGGTATGAAACATGTATTCCTCACTGGCC
TATCACCTGAAGCCTTTGAATTTGCAACACCTGCCAACCCCTCCCTCAAAAGAGTTGCCCTC
TCAGATCCTTTTGATGTAAGGTTTGGTGTTGAGACTTATTTCACTAAATTCTCATACATAAA
CATCACTTTATGTATGAGGCAAAATGAGGACCAGGGAGATGAATGACTTGTCCTGGCTCATA
CACCTGGAAAGTGACAGAGTCAGATTAGATCCCAGGTCTATCTGAAGTTAAAAGAGGTGTCT
TTTCACTTCCCACCTCCTCCATCTACTTTAAAGCAGCACAAACCCCTGCTTTCAAGGAGAGA
TGAGCGTCTCTAAAGCCCCTGACAGCAAGAGCCCAGAACTGGGACACCATTAGTGACCCAGA
CGGCAGGTAAGCTGACTGCAGGAGCATCAGCCTATTCTTGTGTCTGGGACCACAGAGCATTG
TGGGGACAGCCCCGTCTCTTGGGAAAAAAACCCTAAGGGCTGAGGATCCTTGTGAGTGTTGG
GTGGGAACAGCTCCCAGGAGGTTTAATCACAGCCCCTCCATGCTCTCTAGCTGTTGCCATTG
TGCAAGATGCATTTCCCTTCTGTGCAGCAGTTTCCCTGGCCACTAAATAGTGGGATTAGATA
GAAGCCCTCCAAGGGCTTCCAGCTTGACATGATTCTTGATTCTGATCTGGCCCGATTCCTGG
ATAATCGTGGGCAGGCCCATTCCTCTTCTTGTGCCTCATTTTCTTCTTTTGTAAAACAATGG
CTGTACCATTTGCATCTTAGGGTCATTGCAGATGTAAGTGTTGCTGTCCAGAGCCTGGGTGC
AGGACCTAGATGTAGGATTCTGGTTCTGCTACTTCCTCAGTGACATTGAATAGCTGACCTAA
TCTCTCTGGCTTTGGTTTCTTCATCTGTAAAAGAAGGATATTAGCATTAGCACCTCACGGGA
TTGTTACAAGAAAGCAATGAATTAACACATGTGAGCACGGAGAACAGTGCTTGGCATATGGT
AAGCACTACGTACATTTTGCTATTCTTCTGATTCTTTCAGTGTTACTGATGTCGGCAAGTAC
TTGGCACAGGCTGGTTTAATAATCCCTAGGCACTTCCACGTGGTGTCAATCCCTGATCACTG
GGAGTCATCATGTGCCTTGACTCGGGGCCTGGCCCCCCCATCTCTGTCTTGCAGGACAATGC
CGTCTTCTGTCTCGTGGGGCATCCTCCTGCTGGCAGGCCTGTGCTGCCTGGTCCCTGTCTCC
CTGGCTGAGGATCCCCAGGGAGATGCTGCCCAGAAGACAGATACATCCCACCATGATCAGGA
TCACCCAACCTTCAACAAGATCACCCCCAACCTGGCTGAGTTCGCCTTCAGCCTATACCGCC
AGCTGGCACACCAGTCCAACAGCACCAATATCTTCTTCTCCCCAGTGAGCATCGCTACAGCC
TTTGCAATGCTCTCCCTGGGGACCAAGGCTGACACTCACGATGAAATCCTGGAGGGCCTGAA
TTTCAACCTCACGGAGATTCCGGAGGCTCAGATCCATGAAGGCTTCCAGGAACTCCTCCGTA
CCCTCAACCAGCCAGACAGCCAGCTCCAGCTGACCACCGGCAATGGCCTGTTCCTCAGCGAG
GGCCTGAAGCTAGTGGATAAGTTTTTGGAGGATGTTAAAAAGTTGTACCACTCAGAAGCCTT
CACTGTCAACTTCGGGGACACCGAAGAGGCCAAGAAACAGATCAACGATTACGTGGAGAAGG
GTACTCAAGGGAAAATTGTGGATTTGGTCAAGGAGCTTGACAGAGACACAGTTTTTGCTCTG
GTGAATTACATCTTCTTTAAAGGTAAGGTTGCTCAACCAGCCTGAGCTGTTCCCATAGAAAC
AAGCAAAAATATTCTCAAACCATCAGTTCTTGAACTCTCCTTGGCAATGCATTATGGGCCAT
AGCAATGCTTTTCAGCGTGGATTCTTCAGTTTTCTACACACAAACACTAAAATGTTTTCCAT
CATTGAGTAATTTGAGGAAATAATAGATTAAACTGTCAAAACTACTGACAGCTCTGCAGAAC
TTTTCAGAGCCTTTAATGTCCTTGTGTATACTGTATATGTAGAATATATAATGCTTAGAACT
ATAGAACAAATTGTAATACACTGCATAAAGGGATAGTTTCATGGAACATACTTTACACGACT
CTAGTGTCCCAGAATCAGTATCAGTTTTGCAATCTGAAAGACCTGGGTTCAAATCCTGCCTC
TAACACAATTAGCTTTTGACAAAAACAATGCATTCTACCTCTTTGAGGTGCTAATTTCTCAT
CTTAGCATGGACAAAATACCATTCTTGCTGTCAGGTTTTTTTAGGATTAAACAAATGACAAA
GACTGTGGGGATGGTGTGTGGCATACAGCAGGTGATGGACTCTTCTGTATCTCAGGCTGCCT
TCCTGCCCCTGAGGGGTTAAAATGCCAGGGTCCTGGGGGCCCCAGGGCATTCTAAGCCAGCT
CCCACTGTCCCAGGAAAACAGCATAGGGGAGGGGAGGTGGGAGGCAAGGCCAGGGGCTGCTT
CCTCCACTCTGAGGCTCCCTTGCTCTTGAGGCAAAGGAGGGCAGTGGAGAGCAGCCAGGCTG
CAGTCAGCACAGCTAAAGTCCTGGCTCTGCTGTGGCCTTAGTGGGGGCCCAGGTCCCTCTCC
AGCCCCAGTCTCCTCCTTCTGTCCAATGAGAAAGCTGGGATCAGGGGTCCCTGAGGCCCCTG
TCCACTCTGCATGCCTCGATGGTGAAGCTCTGTTGGTATGGCAGAGGGGAGGCTGCTCAGGC
ATCTGCATTTCCCCTGCCAATCTAGAGGATGAGGAAAGCTCTCAGGAATAGTAAGCAGAATG
TTTGCCCTGGATGAATAACTGAGCTGCCAATTAACAAGGGGCAGGGAGCCTTAGACAGAAGG
TACCAAATATGCCTGATGCTCCAACATTTTATTTGTAATATCCAAGACACCCTCAAATAAAC
ATATGATTCCAATAAAAATGCACAGCCACGATGGCATCTCTTAGCCTGACATCGCCACGATG
TAGAAATTCTGCATCTTCCTCTAGTTTTGAATTATCCCCACACAATCTTTTTCGGCAGCTTG
GATGGTCAGTTTCAGCACCTTTTACAGATGATGAAGCTGAGCCTCGAGGGATGTGTGTCGTC
AAGGGGGCTCAGGGCTTCTCAGGGAGGGGACTCATGGTTTCTTTATTCTGCTACACTCTTCC
AAACCTTCACTCACCCCTGGTGATGCCCACCTTCCCCTCTCTCCAGGCAAATGGGAGAGACC
CTTTGAAGTCAAGGACACCGAGGAAGAGGACTTCCACGTGGACCAGGTGACCACCGTGAAGG
TGCCTATGATGAAGCGTTTAGGCATGTTTAACATCCAGCACTGTAAGAAGCTGTCCAGCTGG
GTGCTGCTGATGAAATACCTGGGCAATGCCACCGCCATCTTCTTCCTGCCTGATGAGGGGAA
ACTACAGCACCTGGAAAATGAACTCACCCACGATATCATCACCAAGTTCCTGGAAAATGAAG
ACAGAAGGTGATTCCCCAACCTGAGGGTGACCAAGAAGCTGCCCACACCTCTTAGCCATGTT
GGGACTGAGGCCCATCAGGACTGGCCAGAGGGCTGAGGAGGGTGAACCCCACATCCCTGGGT
CACTGCTACTCTGTATAAACTTGGCTTCCAGAATGAGGCCACCACTGAGTTCAGGCAGCGCC
ATCCATGCTCCATGAGGAGGACAGTACCCAGGGGTGAGGAGGTAAAGGTCTCGTCCCTGGGG
ACTTCCCACTCCAGTGTGGACACTGTCCCTTCCCAATATCCAGTGCCCAGGGCAGGGACAGC
AGCACCACCACACGTTCTGGCAGAACCAAAAAGGAACAGATGGGCTTCCTGGCAAAGGCAGC
AGTGGAGTGTGGAGTTCAAGGGTAGAATGTCCCTGGGGGGACGGGGGAAGAGCCTGTGTGGC
AAGGCCCAGAAAAGCAAGGTTCGGAATTGGAACAGCCAGGCCATGTTCGCAGAAGGCTTGCG
TTTCTCTGTCACTTTATCGGTGCTGTTAGATTGGGTGTCCTGTAGTAAGTGATACTTAAACA
TGAGCCACACATTAGTGTATGTGTGTGCATTCGTGATTATGCCCATGCCCTGCTGATCTAGT
TCGTTTTGTACACTGTAAAACCAAGATGAAAATACAAAAGGTGTCGGGTTCATAATAGGAAT
CGAGGCTGGAATTTCTCTGTTCCATGCCAGCACCTCCTGAGGTCTCTGCTCCAGGGGTTGAG
AAAGAACAAAGAGGCTGAGAGGGTAACGGATCAGAGAGCCCAGAGCCAAGCTGCCCGCTCAC
ACCAGACCCTGCTCAGGGTGGCATTGTCTCCCCATGGAAAACCAGAGAGGAGCACTCAGCCT
GGTGTGGTCACTCTTCTCTTATCCACTAAACGGTTGTCACTGGGCACTGCCACCAGCCCCGT
GTTTCTCTGGGTGTAGGGCCCTGGGGATGTTACAGGCTGGGGGCCAGGTGACCCAACACTAC
AGGGCAAGATGAGACAGGCTTCCAGGACACCTAGAATATCAGAGGAGGTGGCATTTCAAGCT
TTTGTGATTCATTCGATGTTAACATTCTTTGACTCAATGTAGAAGAGCTAAAAGTAGAACAA
ACCAAAGCCGAGTTCCCATCTTAGTGTGGGTGGAGGACACAGGAGTAAGTGGCAGAAATAAT
CAGAAAAGAAAACACTTGCACTGTGGTGGGTCCCAGAAGAACAAGAGGAATGCTGTGCCATG
CCTTGAATTTCTTTTCTGCACGACAGGTCTGCCAGCTTACATTTACCCAAACTGTCCATTAC
TGGAACCTATGATCTGAAGAGCGTCCTGGGTCAACTGGGCATCACTAAGGTCTTCAGCAATG
GGGCTGACCTCTCCGGGGTCACAGAGGAGGCACCCCTGAAGCTCTCCAAGGTGAGATCACCC
TGACGACCTTGTTGCACCCTGGTATCTGTAGGGAAGAATGTGTGGGGGCTGCAGCTCTGTCC
TGAGGCTGAGGAAGGGGCCGAGGGAAACAAATGAAGACCCAGGCTGAGCTCCTGAAGATGCC
CGTGATTCACTGACACGGGACGTGGTCAAACAGCAAAGCCAGGCAGGGGACTGCTGTGCAGC
TGGCACTTTCGGGGCCTCCCTTGAGGTTGTGTCACTGACCCTGAATTTCAACTTTGCCCAAG
ACCTTCTAGACATTGGGCCTTGATTTATCCATACTGACACAGAAAGGTTTGGGCTAAGTTGT
TTCAAAGGAATTTCTGACTCCTTCGATCTGTGAGATTTGGTGTCTGAATTAATGAATGATTT
CAGCTAAAGATGACACTTATTTTGGAAAACTAAAGGCGACCAATGAACAACTGCAGTTCCAT
GAATGGCTGCATTATCTTGGGGTCTGGGCACTGTGAAGGTCACTGCCAGGGTCCGTGTCCTC
AAGGAGCTTCAAGCCGTGTACTAGAAAGGAGAGAGCCCTGGAGGCAGACGTGGAGTGACGAT
GCTCTTCCCTGTTCTGAGTTGTGGGTGCACCTGAGCAGGGGGAGAGGCGCTTGTCAGGAAGA
TGGACAGAGGGGAGCCAGCCCCATCAGCCAAAGCCTTGAGGAGGAGCAAGGCCTATGTGACA
GGGAGGGAGAGGATGTGCAGGGCCAGGGCCGTCCAGGGGGAGTGAGCGCTTCCTGGGAGGTG
TCCACGTGAGCCTTGCTCGAGGCCTGGGATCAGCCTTACAACGTGTCTCTGCTTCTCTCCCC
TCCAGGCCGTGCATAAGGCTGTGCTGACCATCGACGAGAAAGGGACTGAAGCTGCTGGGGCC
ATGTTTTTAGAGGCCATACCCATGTCTATCCCCCCCGAGGTCAAGTTCAACAAACCCTTTGT
CTTCTTAATGATTGAACAAAATACCAAGTCTCCCCTCTTCATGGGAAAAGTGGTGAATCCCA
CCCAAAAATAACTGCCTCTCGCTCCTCAACCCCTCCCCTCCATCCCTGGCCCCCTCCCTGGA
TGACATTAAAGAAGGGTTGAGCTGGTCCCTGCCTGCATGTGACTGTAAATCCCTCCCATGTT
TTCTCTGAGTCTCCCTTTGCCTGCTGAGGCTGTATGTGGGCTCCAGGTAACAGTGCTGTCTT
CGGGCCCCCTGAACTGTGTTCATGGAGCATCTGGCTGGGTAGGCACATGCTGGGCTTGAATC
CAGGGGGGACTGAATCCTCAGCTTACGGACCTGGGCCCATCTGTTTCTGGAGGGCTCCAGTC
TTCCTTGTCCTGTCTTGGAGTCCCCAAGAAGGAATCACAGGGGAGGAACCAGATACCAGCCA
TGACCCCAGGCTCCACCAAGCATCTTCATGTCCCCCTGCTCATCCCCCACTCCCCCCCACCC
AGAGTTGCTCATCCTGCCAGGGCTGGCTGTGCCCACCCCAAGGCTGCCCTCCTGGGGGCCCC
AGAACTGCCTGATCGTGCCGTGGCCCAGTTTTGTGGCATCTGCAGCAACACAAGAGAGAGGA
CAATGTCCTCCTCTTGACCCGCTGTCACCTAACCAGACTCGGGCCCTGCACCTCTCAGGCAC
TTCTGGAAAATGACTGAGGCAGATTCTTCCTGAAGCCCATTCTCCATGGGGCAACAAGGACA
CCTATTCTGTCCTTGTCCTTCCATCGCTGCCCCAGAAAGCCTCACATATCTCCGTTTAGAAT
CAGGTCCCTTCTCCCCAGATGAAGAGGAGGGTCTCTGCTTTGTTTTCTCTATCTCCTCCTCA
GACTTGACCAGGCCCAGCAGGCCCCAGAAGACCATTACCCTATATCCCTTCTCCTCCCTAGT
CACATGGCCATAGGCCTGCTGATGGCTCAGGAAGGCCATTGCAAGGACTCCTCAGCTATGGG
AGAGGAAGCACATCACCCATTGACCCCCGCAACCCCTCCCTTTCCTCCTCTGAGTCCCGACT
GGGGCCACATGCAGCCTGACTTCTTTGTGCCTGTTGCTGTCCCTGCAGTCTTCAGAGGGCCA
CCGCAGCTCCAGTGCCACGGCAGGAGGCTGTTCCTGAATAGCCCCTGTGGTAAGGGCCAGGA
GAGTCCTTCCATCCTCCAAGGCCCTGCTAAAGGACACAGCAGCCAGGAAGTCCCCTGGGCCC
CTAGCTGAAGGACAGCCTGCTCCCTCCGTCTCTACCAGGAATGGCCTTGTCCTATGGAAGGC
ACTGCCCCATCCCAAACTAATCTAGGAATCACTGTCTAACCACTCACTGTCATGAATGTGTA
CTTAAAGGATGAGGTTGAGTCATACCAAATAGTGATTTCGATAGTTCAAAATGGTGAAATTA
GCAATTCTACATGATTCAGTCTAATCAATGGATACCGACTGTTTCCCACACAAGTCTCCTGT
TCTCTTAAGCTTACTCACTGACAGCCTTTCACTCTCCACAAATACATTAAAGATATGGCCAT
CACCAAGCCCCCTAGGATGACACCAGACCTGAGAGTCTGAAGACCTGGATCCAAGTTCTGAC
TTTTCCCCCTGACAGCTGTGTGACCTTCGTGAAGTCGCCAAACCTCTCTGAGCCCCAGTCAT
TGCTAGTAAGACCTGCCTTTGAGTTGGTATGATGTTCAAGTTAGATAACAAAATGTTTATAC
CCATTAGAACAGAGAATAAATAGAACTACATTTCTTGCACTTATGAGCTTTCTGTGAATCAG
ACATCCCTATGAAGTACCTCCCCTGGCTGTTTCTCATTTACTCACTGTAGCAGCACTGCGAT
GTGTGAGTATATCTGCTGTGCTCTTAAACTCCAAATCTGAGGAAACTGAGGCTCAGAGAGGC
TACTGGTCTCCCACAATGTCACACAGCTCATAAGTGGCAAAGCTGGCTTGATGGGCTACTTG
TTCCTCTGAACCATACCACCTCACCACACTCTCCCCTTCGAGGGTCACGCTAAACTTCTGCA
GAGGTAATTCCTCCTTAAACCAGAAGGGTTGCTGGTGGCCCACAGCTCACGCCTAGCACACT
TCATGAGAAAAACACCCTGTGCCCAGTGTGGAGCAGGCATTGAGCTGAAGGTGGTGAGCAGA
AGCTCATCCACCAGATGTTGACACAGCCCGCAGCCTTGGGCGACCCACAGGACTCCTCTTAT
TTAACTGGCATTTGGTAGGAGAACAGGGGCAGAGTCAAAGACAAGTTGGCTTTCTGGAGAGC
CCAGGGCAGGGAAGGAGGTGGCAGCGCTGAGGGCGGTCACCTTAGACACCATCGTTTTACTT
TGAAGAATTGTCTGTCACA.

>Exemplary Serpin1 A polynucleotide sequence corresponding to NM_000295. In embodiments, the nucleobase shown by bold underlined text corresponds to a nucleobase targeted for base editing according to the methods provided herein. In some embodiments, a PAM sequence corresponding to a guide polynucleotide used to edit the target base is shown as bold double-underlined text.

```
(SEQ ID NO: 592)
      1 acaatgactc ctttcggtaa gtgcagtgga agctgtacac tgcccaggca aagcgtccgg
    61 gcagcgtagg cgggcgactc agatcccagc cagtggactt agcccctgtt tgctcctccg
  121 ataactgggg tgaccttggt taatattcac cagcagcctc ccccgttgcc cctctggatc
  181 cactgcttaa atacggacga ggacagggcc ctgtctcctc agcttcaggc accaccactg
  241 acctgggaca gtgaatcgac aatgccgtct tctgtctcgt ggggcatcct cctgctggca
  301 ggcctgtgct gcctggtccc tgtctccctg gctgaggatc cccagggaga tgctgcccag
  361 aagacagata catcccacca tgatcaggat cacccaacct tcaacaagat cacccccaac
  421 ctggctgagt tcgccttcag cctataccgc cagctggcac accagtccaa cagcaccaat
  481 atcttcttct ccccagtgag catcgctaca gcctttgcaa tgctctccct ggggaccaag
  541 gctgacactc acgatgaaat cctggagggc ctgaatttca acctcacgga gattccggag
  601 gctcagatcc atgaaggctt ccaggaactc ctccgtaccc tcaaccagcc agacagccag
  661 ctccagctga ccaccggcaa tggcctgttc ctcagcgagg gcctgaagct agtggataag
  721 tttttggagg atgttaaaaa gttgtaccac tcagaagcct tcactgtcaa cttcggggac
  781 accgaagagg ccaagaaaca gatcaacgat tacgtggaga agggtactca agggaaaatt
  841 gtggatttgg tcaaggagct tgacagagac acagtttttg ctctggtgaa ttacatcttc
  901 tttaaaggca aatgggagag accctttgaa gtcaaggaca ccgaggaaga ggacttccac
  961 gtggaccagg tgaccaccgt gaaggtgcct atgatgaagc gtttaggcat gtttaacatc
1021 cagcactgta agaagctgtc cagctgggtg ctgctgatga aatacctggg aatgccacc
1081 gccatcttct tcctgcctga tgaggggaaa ctacagcacc tggaaaatga ctcacccac
1141 gatatcatca ccaagttcct ggaaaatgaa gacagaaggt ctgccagctt catttaccc
1201 aaactgtcca ttactggaac ctatgatctg aagagcgtcc tgggtcaact ggcatcact
1261 aaggtcttca gcaatggggc tgacctctcc ggggtcacag aggaggcacc ctgaagctc
1321 tccaaggccg tgcataaggc tgtgctgacc atcgacgaga aagggactga gctgctggg
1381 gccatgtttt tagaggccat acccatgtct atcccccccg aggtcaagtt aacaaaccc
1441 tttgtcttct taatgattga acaaaatacc aagtctcccc tcttcatggg aaagtggtg
1501 aatcccaccc aaaaataact gcctctcgct cctcaacccc tcccctccat cctggcccc
1561 ctccctggat gacattaaag aagggttgag ctggtccctg cctgcatgtg ctgtaaatc
1621 cctcccatgt tttctctgag tctccctttg cctgctgagg ctgtatgtgg ctccaggta
1681 acagtgctgt cttcgggccc cctgaactgt gttcatggag catctggctg gtaggcaca
1741 tgctgggctt gaatccaggg gggactgaat cctcagctta cggacctggg ccatctgtt
1801 tctggagggc tccagtcttc cttgtcctgt cttggagtcc ccaagaagga tcacagggg
1861 aggaaccaga taccagccat gaccccaggc tccaccaagc atcttcatgt cccctgctc
1921 atcccccact cccccccacc cagagttgct catcctgcca gggctggctg gcccacccc
1981 aaggctgccc tcctgggggc cccagaactg cctgatcgtg ccgtggccca ttttgtggc
2041 atctgcagca acacaagaga gaggacaatg tcctcctctt gacccgctgt acctaacca
2101 gactcgggcc ctgcacctct caggcacttc tggaaaatga ctgaggcaga tcttcctga
2161 agcccattct ccatggggca acaaggacac ctattctgtc cttgtccttc atcgctgcc
2221 ccagaaagcc tcacatatct ccgtttagaa tcaggtccct tctccccaga gaagaggag
2281 ggtctctgct ttgttttctc tatctcctcc tcagacttga ccaggcccag aggccccag
2341 aagaccatta ccctatatcc cttctcctcc ctagtcacat ggccataggc tgctgatgg
2401 ctcaggaagg ccattgcaag gactcctcag ctatgggaga ggaagcacat acccattga
2461 cccccgcaac ccctcccttt cctcctctga gtcccgactg gggccacatg agcctgact
2521 tctttgtgcc tgttgctgtc cctgcagtct tcagagggcc accgcagctc agtgccacg
2581 gcaggaggct gttcctgaat agcccctgtg gtaagggcca ggagagtcct ccatcctcc
2641 aaggccctgc taaaggacac agcagccagg aagtcccctg ggcccctagc gaaggacag
2701 cctgctccct ccgtctctac caggaatggc cttgtcctat ggaaggcact ccccatccc
2761 aaactaatct aggaatcact gtctaaccac tcactgtcat gaatgtgtac taaaggatg
2821 aggttgagtc ataccaaata gtgatttcga tagttcaaaa tggtgaaatt gcaattcta
2881 catgattcag tctaatcaat ggataccgac tgtttcccac acaagtctcc gttctctta
2941 agcttactca ctgacagcct ttcactctcc acaaatacat taaagatatg ccatcacca
3001 agccccctag gatgacacca gacctgagag tctgaagacc tggatccaag tctgacttt
3061 tccccctgac agctgtgtga ccttcgtgaa gtcgccaaac ctctctgagc ccagtcatt
3121 gctagtaaga cctgcctttg agttggtatg atgttcaagt tagataacaa atgtttata
3181 cccattagaa cagagaataa atagaactac atttcttgca
```

By “alteration” is meant a change in the level, structure, or activity of an analyte, gene or polypeptide as detected by standard art known methods such as those described herein. As used herein, an alteration includes a change (e.g., increase or reduce) in expression levels. In embodiments, the increase or reduction in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “base editing activity” is meant acting to chemically alter a base within a polynucleotide. In one embodiment, a first base is converted to a second base. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is an adenine or adenosine base editor (ABE). In some embodiments, the base editor is an adenine or adenosine base editor (ABE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and x-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include Alpha-1 antitrypsin deficiency, chronic obstructive pulmonary disease (COPD), liver disease, skin problems (e.g., panniculitis), and inflammation of the blood vessels (e.g., vasculitis). In some embodiments such diseases are amenable to treatment with base editors, for example, correcting a mutation in a gene encoding an A1AT protein.

The term “effective amount,” as used herein, refers to an amount of a biologically active agent that is sufficient to elicit a desired biological response. In particular embodiments, an effective amount is the amount of a base editor system (e.g., a fusion protein comprising a programable DNA binding protein, a nucleobase editor and gRNA) that is sufficient to alter a A1AT mutation in a cell to achieve a therapeutic effect. Such therapeutic effect need not be sufficient to alter a A1AD in all cells of a tissue or organ, but only in about 1%, 5%, 10%, 25%, 50%, 75% or more of the cells present in a subject, tissue, cell, or organ. In one embodiment, an effective amount is sufficient to ameliorate one or more symptoms of A1AD. The effective amount of an active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the invention (e.g., a fusion protein comprising a programable DNA binding protein, a nucleobase editor and gRNA) sufficient to introduce an alteration in a gene of interest in a cell (e.g., a cell in vitro or in vivo). In one embodiment, an effective amount is the amount of a base editor required to achieve a therapeutic effect (e.g., to reduce or control a disease or a symptom or condition thereof). By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

By “heterologous,” or “exogenous” is meant a polynucleotide or polypeptide that 1) has been experimentally incorporated into a polynucleotide or polypeptide sequence to which the polynucleotide or polypeptide is not normally found in nature; and/or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker. In some embodiments the linker comprises one or more amino acids that are covalently linked.

By “marker” is meant any protein or polynucleotide having an alteration in expression, level, structure, or activity that is associated with a disease or disorder, such as alpha-1 antitrypsin deficiency (A1AD).

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi: 10.1038/nbt.4172. In some embodiments, an NLS comprises the amino acid sequence KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRK (SEQ ID NO: 194), PKKKRKV (SEQ ID NO: 195), MDSLLMNRRKFLYQFKNVRWAKGRRETYLC (SEQ ID NO: 196), PKKKRKVEGADKRTADGSEFESPKKKRKV (SEQ ID NO: 328), or RKSGKIAAIVVKRPRKPKKKRKV (SEQ ID NO: 329).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363 (6422): 88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-231, 232-245, 254-257, 260, and 378. In some embodiments, the napDNAbp is a (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9). Further non-limiting examples of nucleic acid programmable DNA binding proteins include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. In some embodiments, the napDNAbp is OpenCRISPR-1, or a variant thereof (e.g., a variant comprising a D10A amino acid alteration and/or lacking an N-terminal methionine).

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest. In embodiments, a reference is a cell that has not been treated according to the methods provided herein, a cell associated with a disease or disorder, or a healthy cell. In embodiments, a reference is a subject diagnosed with an alpha-1 antitrypsin deficiency and not treated, not recently treated (i.e., within 1 month, 6 months, 1 year, 5 years, or 10 years), or prior to treatment, according to the methods provided herein and/or using a composition provided herein. In some embodiments, a reference is a healthy subject. In some embodiments, the reference is at an earlier time point in treatment.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The terms “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA).

The term “single nucleotide polymorphism (SNP)” refers to a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g. a Cas9) or a base editor.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 depicts a SERPINA1 correction editing strategy with an adenosine base editor. The nucleotide sequence shown in FIG. 1 corresponds to SEQ ID NO: 430 (ATC GAC AAG AAA GGG ACT GAA GC). The amino acid sequence shown in FIG. 1 corresponds to SEQ ID NO: 431 (IDKKGTEA).

FIG. 2 are schematics depicting yeast-based screens and structure-based design to optimize editors to improve selectivity for NGC PAM and the A1AT target site.

FIG. 3 is a schematic representation of a screening and rational design plan to develop improved adenosine base editors (ABEs) for targeting SERPINA1.

FIG. 4 is a graph showing editing efficiency in patient derived fibroblast cells (PiZZ cells), which were transfected with mRNA encoding the indicated variants and synthetic guides by electroporation.

FIGS. 5A-5D show editing efficiency of ABEs using different linker sequences. Wild-type HEK293T cells were transfected in 96-well format and each well contains plasmids expressing base editor variants and plasmids expressing guides. To evaluate on-target editing efficiency, each editor was co-transfected with plasmids expressing guides targeting A1 target site without E342K mutation. FIG. 5A shows % A to G editing at position A5. FIG. 5B shows linker EGGSEEEEESGS (pYY-1359/1379) (SEQ ID NO: 432). FIG. 5C shows the ratio of A5/A8 editing. FIG. 5D shows the % max A to G editing of ABE variants. In FIG. 5D, each set of three bars corresponds from left-to-right to “OT1,” “OT2,” and “OT4-A7G,” respectively.

FIGS. 6A-6B are graphs showing % A to G editing. FIG. 6A shows positions A7 and A5. FIG. 6B shows OT1-A7, OT2-A5, and OT4-A5. Patient derived fibroblast cells (PiZZ cells) were transfected with mRNA encoding for indicated variants (by plasmid number) and synthetic guides using lipofectamine messengerMax. Editing was shown for indicated As in the editing window. 50 ng mRNA and 25 ng guide RNA were used for each well containing 18K cells, and data is collected 24 h after transfection. In FIG. 6B, each set of three bars corresponds from left-to-right to “OT1-A7,” “OT2-A5,” and “OT4-A5,” respectively.

FIGS. 7A-7B are graphs showing % A to G editing. FIG. 7A shows positions A7 and A5. FIG. 7B shows OT1-A7, OT2-A5, and OT4-A5. Patient derived fibroblast cells (PiZZ cells) were transfected with mRNA encoding for indicated variants (by plasmid number) and synthetic guides using lipofectamine messengerMax. Editing was shown for indicated As in the editing window. 25 ng mRNA and 25 ng guide RNA were used for each well containing 18K cells, and data is collected 72 h after transfection. In FIG. 7B, each set of three bars corresponds from left-to-right to “OT1-A7,” “OT2-A5,” and “OT4-A5,” respectively.

FIG. 8 is a graph showing % A to G editing in patient derived fibroblast cells (PiZZ cells), which were transfected with mRNA encoding for indicated variants derived from var 12 (mutations in TadA) and synthetic guides using lipofectamine messengerMax.

FIG. 9 is a graph showing % A to G editing in Patient derived fibroblast cells (PiZZ cells), which were transfected with mRNA encoding for indicated variants derived from var 12 (mutations in TadA) and synthetic guides using lipofectamine messengerMax.

FIG. 10 is a graph showing % A to G editing in patient derived fibroblast cells (PiZZ cells), which were transfected with mRNA encoding for indicated variants derived from var 12 (mutations in NGC Cas9) and synthetic guides using lipofectamine messengerMax.

FIG. 11 is a graph showing % A to G editing in patient derived fibroblast cells (PiZZ cells), which were transfected with mRNA encoding for indicated variants derived from var 12 (mutations in NGC Cas9) and synthetic guides using lipofectamine messengerMax.

FIG. 12 is a graph showing % A to G editing in patient derived fibroblast cells (PiZZ cells), which were transfected with mRNA encoding for indicated variants derived from var 12 with TadA5 (mutations in TadA) and synthetic guides using lipofectamine messengerMax.

FIG. 13 is a graph showing % A to G editing in patient derived fibroblast cells (PiZZ cells), which were transfected with mRNA encoding for indicated variants derived from var 12 (mutations in NGC Cas9) and synthetic guides using lipofectamine messengerMax.

FIG. 14 is a graph showing % A to G editing in patient derived fibroblast cells (PiZZ cells), which were transfected with mRNA encoding for indicated variants derived from var 12 (mutations in NGC Cas9) and synthetic guides using lipofectamine messengerMax.

FIG. 15 is a graph showing % A to G editing in patient derived fibroblast cells (PiZZ cells), which were transfected with mRNA encoding for indicated variants derived from var 12 (mutations in NGC Cas9) and synthetic guides using lipofectamine messengerMax.

FIG. 16 is a graph showing % A to G editing in patient derived fibroblast cells (PiZZ cells), which were transfected with mRNA encoding for indicated variants derived from var 12 (mutations in NGC Cas9) and synthetic guides using lipofectamine messengerMax.

FIG. 17 is a graph showing % base editing efficiency using patient fibroblasts that carry the PiZ mutation, which were electroporated with mRNA encoding editor variants and gRNA856 to assess on-target correction editing and off-target editing at a candidate site (OT454) using amplicon sequencing.

FIG. 18 is a graph showing average % max A to G editing in patient fibroblasts that carry the PiZ mutation, which were electroporated with mRNAs encoding 11 editor variants and gRNA856 to assess guide-dependent off-target editing at 6 candidate OT sites using amplicon sequencing. All editor variants tested had average OT editing lower than that observed for var12 codon optimized.

FIG. 19 is a graph showing % editing efficiency of 11 editor variants and guide RNA 856, which were tested alongside var12 codon optimized in vivo in NSG-PiZ mice at a sub-saturating dose of 0.25 mpk. Liver editing was assessed using NGS.

FIGS. 20A-20B show in vivo assessments of ABE variants on serum A1AT levels. FIG. 20A shows serum A1AT levels in mice treated with ABE variants. FIG. 20B shows fold-change A1AT levels in mice treated with ABE variants.

FIG. 21 shows data for ABE editor Variants A, B, E, F, G, H, I, J, and K, which show a decrease in number of off target (OT) sites relative to codon optimized variant 12. Variant G shows improvement across most criteria including improved in vivo on-target editing, similar bystander editing and lower number of guide-dependent off target (OT) sites.

FIGS. 22A-22C show improved efficacy of ABE Variant G in vivo. FIG. 22A shows editing efficiency of ABE Variant G in mice. FIG. 22B shows serum A1AT levels of mice treated with ABE Variant G. FIG. 22C shows fold increase in functional A1AT of mice treated with ABE Variant G. In FIG. 22A, each three sets of datapoints correspond, respectively, to “Beneficial Edits,” “Bystander Edits,” and “Indels.”

FIG. 23 provides a schematic diagram providing a description of alpha-1 antitrypsin deficiency (AATD). Direct correction of the PiZ mutation through base editing may: 1) reduce liver toxicity caused by the mutant Z-AAT protein (i.e., an AAT protein encoded by a SERPINA1 gene having the PiZ mutation) aggregates (referred to as polymers); 2) restore circulating functional AAT and decrease circulating Z-AAT polymers to protect lungs.

FIG. 24 provides a schematic diagram providing an overview of a strategy for correcting the PiZ mutation using base editing. A base editor system is delivered to a subject by administering a lipid nanoparticle (LNP) comprising mRNA encoding a base editor polypeptide and a guide RNA. The lipid nanoparticle may be referred to as a “base editor system LNP.” Correction of the PiZ mutation results in the M (medium mobility) allele (PiM) of the A1AT gene.

FIGS. 25A and 25B provide a schematic diagram and stacked bar graph relating to the in vivo correction of the PiZ mutation in an AATD mouse model (also referred to as NSG-PiZ mice). FIG. 25A provides a schematic diagram presenting the method used to correct the PiZ mutation in the AATD mouse model. FIG. 25B provides a stacked bar graph showing maximum percent A to G editing measured in mice administered the indicated mg/kg (mpk) of total RNA (1:1 mass ratio of the guide polynucleotide gRNA856 and mRNA encoding Variant G). The mice were administered lipid nanoparticles containing a base editor system containing an mRNA encoding a base editor (ABE Variant G) and the guide RNA gRNA856. Beneath the stacked bar graph of FIG. 25B is provided the sequence targeted for base editing (i.e., ATCGACAAGAAAGGGACTGAAGCTGCTG, (SEQ ID NO: 593), where the nucleotide in bold is the target nucleotide, the underlined nucleotides are additional nucleotides that were within the editing window of the base editor (i.e., “additional base-edited alleles”), and the double-underlined nucleotides correspond to a PAM sequence) and the amino acid sequence encoded thereby (i.e., IDKKGTEAA (SEQ ID NO: 594)). In each of the stacked bars of FIG. 25B, the bars within each stacked bar are as follows from bottom-to-top: additional base-edited alleles; corrected alleles; and indels.

FIGS. 26A and 26B provide a plot and a stacked bar graph demonstrating that base editing in vivo to correct the PiZ mutation in an AATD mouse model resulted in increased serum total and corrected AAT and decreased serum PiZ AAT levels. The data of FIGS. 26A and 26B was collected using mice treated as described for FIGS. 25A and 25B. FIG. 26A provides a plot showing levels of total AAT (left axis and upper curve) and total PiZ AAT (right axis and lower curve) in the AATD mice FIG. 26B provides a stacked bar graph showing the percent of AAT in the serum of AATD mice corresponding to corrected AAT and PiZ AAT. The AATD mice were administered the indicated mg/kg (mpk) of total RNA (1:1 mass ratio of the guide polynucleotide gRNA856 and mRNA encoding Variant G). In each of the stacked bars of FIG. 25B, the bars within each stacked bar are as follows from bottom-to-top: corrected AAT and PiZ AAT. In FIGS. 26A and 26B measurements were taken one week after administering the base editor system to the mice.

FIGS. 27A and 27B provide a schematic diagram and a plot demonstrating that base editing in vivo to correct the PiZ mutation in an AATD mouse model resulted in increased functional serum AAT. The mice were treated as described for FIGS. 25A and 25B. FIG. 27A provides a schematic diagram showing how levels of functional AAT in the serum of mice was measured. FIG. 27B provides a bar graph showing levels of functional AAT in mice prior to administration of the base editor system (pre-dose) and after administration of base editor systems containing the indicated mg/kg (mpk) doses of total RNA (1:1 mass ratio of the guide polynucleotide gRNA856 and mRNA encoding Variant G). For each pair of bars in FIG. 27B, the left bar represents a pre-dose measurement, and the right bar represents a post-dose measurement.

FIG. 28 provides a stacked bar graph demonstrating that correction of the PiZ mutation in an AATD mouse model using base editing was durable. The AATD mice were treated as described in FIGS. 25A and 25B and maximum percent A to G base editing was measured at 1 week and 2 weeks following administration of the base editor system. In each of the stacked bars of FIG. 28, the bars within each stacked bar are as follows from bottom-to-top: additional base-edited alleles; corrected alleles; and indels.

FIGS. 29A and 29B provide a schematic diagram and a bar graph presenting a new knock-out/knock-in humanized PiZ rat. The rat SerpinA1 gene was knocked out in the rats and the human SERPINA1 gene with the c. 1096G>A (PiZ) mutation was knocked in at the rat SerpinA1 locus. FIG. 29A provides a schematic diagram showing how the genomes of rats were edited to create a humanized PiZ rat with expression of the rat Serpin1 gene knocked out. A human SERPINA1 polynucleotide encoding an AAT polynucleotide containing the c. 1096G>A PiZ mutation (i.e., the human PiZ allele) and also encoding a Simian virus 40 Poly A tail (SV40-pA) was inserted into the genome of the rats between the rat Serpin1 gene 5′ untranslated region (UTR) and Exon 1 of the rat Serpin gene. The rats had a Sprague-Dawley genomic background, were immunocompetent, did not express rat AAT, contained a 1-to-1 knock-in of huSERPINA1 c.1096G>A (PiZ) within each rat Serpin1 allele, and expressed about 3.6 μM of the human PiZ polypeptide at 18 weeks following genome editing. The rats were referred to as “hSERPINA1 PiZ rats.” FIG. 29B provides a bar graph demonstrating that rats homozygous for the human PiZ allele did not express rat AAT. In FIG. 29B, “MS” indicates mass spectrometry, “WT” indicates wild-type, “HET” indicates heterozygous for the human PiZ allele and the rat SERPINA1 allele, and “HOM” indicates homozygous for the human PiZ allele.

FIGS. 30A and 30B provide a schematic diagram and a stacked bar graph demonstrating that correction of the PiZ mutation in hSERPINA1 PiZ rats (see FIGS. 29A and 29B) using base editor systems was dose-dependent. FIG. 29A provides a schematic diagram presenting the method used to correct the PiZ mutation in the hSERPINA1 PiZ rats. The rats were administered lipid nanoparticles containing a base editor system containing the guide polynucleotide gRNA856 and an mRNA molecule encoding a base editor (ABE Variant G). FIG. 30B provides a stacked bar graph showing maximum percent A to G editing measured in rats administered the indicated mg/kg (mpk) total RNA (1:1 mass ratio of the guide polynucleotide gRNA856 and mRNA encoding Variant G). Beneath the stacked bar graph of FIG. 30B is provided the sequence targeted for base editing (i.e., ATCGACAAGAAAGGGACTGAAGCTGCTG, (SEQ ID NO: 593), where the nucleotide in bold is the target nucleotide, the underlined nucleotides are additional nucleotides that were within the editing window of the base editor (i.e., “additional base-edited alleles”), and the double-underlined nucleotides correspond to a PAM sequence) and the amino acid sequence encoded thereby (i.e., IDKKGTEAA (SEQ ID NO: 594)). In each of the stacked bars of FIG. 30, the bars within each stacked bar are as follows from bottom-to-top: additional base-edited alleles; corrected alleles; and indels.

FIGS. 31A and 31B provide a plot and a stacked bar graph demonstrating that base editing in vivo to correct the PiZ mutation in hSERPINA1 PiZ rats resulted in increased serum total and corrected AAT and decreased serum PiZ AAT levels. The data of FIGS. 31A and 31B was collected using rats treated as described for FIGS. 30A and 30B. FIG. 30A provides a plot showing levels of total AAT (left axis and upper curve) and total PiZ AAT (right axis and lower curve) in the hSERPINA1 PiZ rats. FIG. 31B provides a stacked bar graph showing the percent of AAT in the serum of hSERPINA1 PiZ rats corresponding to corrected AAT and PiZ AAT. The hSERPINA1 PiZ rats were administered the indicated mg/kg (mpk) of total RNA (1:1 mass ratio of the guide polynucleotide gRNA856 and mRNA encoding Variant G). In each of the stacked bars of FIG. 30B, the bars within each stacked bar are as follows from bottom-to-top: corrected AAT and PiZ AAT. In FIGS. 26A and 26B measurements were taken one week after administering the base editor system to the mice.

FIG. 32 provides a schematic diagram summarizing the effects of using base editing to correct a PiZ mutation to restore SERPINA1 gene function.

FIGS. 33A to 33C provide a schematic diagram, tissue images, and a bar graph showing that correction of the PiZ mutation in mice treated using base editing as described for FIGS. 25A and 25B led to decreased liver Z-AAT aggregates (i.e., aggregates of an AAT protein encoded by a SERPINA1 gene having the PiZ mutation) in mice. The mice were administered a base editor system containing 0.25 mpk of total RNA (1:1 mass ratio of the guide polynucleotide gRNA856 and mRNA encoding Variant G). FIG. 33A provides a schematic diagram showing how mice were treated and how AAT aggregates were measured. FIG. 33B shows PAS-D stained liver tissue from mice administered lipid nanoparticles containing the base editor system (lower panel) and in mice not administered the base editor system (i.e., “vehicle”; upper panel). FIG. 33C provides a bar graph showing levels of PAS-D staining in liver tissue from mice administered lipid nanoparticles containing the base editor system and in mice not administered the base editor system. The scale bar in FIG. 33B represent 200 μm.

FIGS. 34A and 34B provide a schematic diagram and a bar graph demonstrating that the 5G+7G allele resulting from base editing of the PiZ allele of SERPINA1 yields a D365G AAT protein that is secreted by the liver at levels comparable to the PiM allele (the wild-type allele) of SERPINA1. FIG. 34A provides a schematic diagram providing a summary of nucleotide edits and corresponding A1AT mutants prepared by treating mice according to the method described for FIGS. 25A and 25B. The nucleotide sequence shown in FIG. 34A is ATCGACAAGAAAGGGACTGAAGCTGCTG (SEQ ID NO: 593), where the nucleotide in bold is the A7 target nucleotide, the underlined nucleotide is the A5 bystander nucleotide, and the double-underlined nucleotides correspond to a PAM sequence. The amino acid sequence encoded by the nucleotide sequence of FIG. 34A is shown beneath the nucleotide sequence and is IDKKGTEAA (SEQ ID NO: 594). FIG. 34B provides a bar graph demonstrating that levels of AAT protein secreted from cells containing the 5G+7G allele of SERPINA1 were comparable to those secreted from cells containing the PiM allele of SERPINA1. In FIG. 34B, the bars each represent, from left-to-right, the following: Unedited; A7; A5; and A5+A7.

FIG. 35 provides a plot demonstrating that the 5G+7G allele resulting from base editing of the PiZ allele of SERPINA1 (see FIG. 34A) yielded a D365G AAT protein that functioned comparably to an AAT protein encoded by the PiM allele of SERPINA1. The D365G AAT protein inhibited neutrophil elastase comparably to wild-type AAT encoded by the PiM allele of SERPINA1.

FIG. 36 provides a plot demonstrating that levels of AAT encoded by a PiZ allele altered through base editing to encode the amino acid D365G (“Corrected AAT”), as shown in FIG. 34A, correlated with increased in functional AAT in mice treated as described for FIGS. 25A and 25B. Functional AAT levels were measured as described for FIGS. 27A and 27B.

FIGS. 37A and 37B provide a schematic diagram and a stacked bar graph demonstrating increased editing in AATD mice (also referred to as NSG-PiZ mice) following a second administration of a base editor system. FIG. 37A provides a schematic diagram presenting the method used to correct the PiZ mutation in the AATD mouse model. The mice were administered lipid nanoparticles containing a base editor system containing an mRNA encoding a base editor (ABE Variant G) and the guide RNA gRNA856 twice with the second administration taking place about two weeks after the first. FIG. 37B provides a stacked bar graph showing maximum percent A to G editing measured in the mice at two weeks following the second administration of the base editor system. The mice were administered the indicated mg/kg (mpk) of total RNA (1:1 mass ratio of the guide polynucleotide gRNA856 and mRNA encoding Variant G). In each of the stacked bars of FIG. 37B, the bars within each stacked bar are as follows from bottom-to-top: additional base-edited alleles; corrected alleles; and indels.

FIG. 38 provides images showing that correction of the PiZ mutation in mice according to the method described for FIGS. 25A and 25B led to decreased liver Z-AAT aggregates (i.e., aggregates of an AAT protein encoded by a SERPINA1 gene having the PiZ mutation) in AATD mice at one-week following administration of the base editor system. The scale bars in FIG. 38 represent 350 μm.

FIG. 39 provides a plot demonstrating that wild type alpha-1 antitrypsin (AAT) protein (PiM) was secreted comparably to an AAT protein with a D365G alteration (PiM+bystander) in vivo. PiM AAT and PiM+bystander AAT levels resulting from editing of PiZ mouse hepatocytes using a base editor system containing the guide gRNA856 and mRNA encoding the base editor ABE Variant G correlated similarly to serum AAT levels of the corresponding proteins in vivo, indicating similar secretion of both proteins from the liver. In FIG. 39, “Allelic Editing” indicates the percent of hepatocytes in the mice containing the indicated allele (i.e., PiM or PiM+Bystander).

FIGS. 40A to 40C provide plots and a stacked bar graph demonstrating that a human alpha-1 antitrypsin (hAAT or AAT) protein with a D365G alteration (PiM+bystander) was functionally active and indistinguishable from a wild type hAAT protein (piM) in vitro and ex vivo. FIG. 40A provides a plot presenting data from a neutrophil elastase (NE) binding assay. FIG. 40B provides a plot presenting data from a NE inhibition assay. FIG. 40C provides a plot showing serum AAT levels and functional AAT levels measured as NE inhibition in mice administered the indicated doses of lipid nanoparticles containing a base editor system containing gRNA856 and mRNA encoding the base editor ABE variant G (the “Variant G Formulation”). In the stacked bar graphs of FIG. 40C each of the bars from left-to-right represent, from bottom-to-top the following: 1) PiZ; 2) PiM, PiM+Bystander, PiZ, and 3) PiZ+Bystander; and PiM, PiM+Bystander, PiZ, and PiZ+Bystander. Functional activity of recombinant PiM+bystander was comparable to PiM (wildtype) protein based on both human neutrophil elastase (NE) binding in vitro (FIG. 40A) and enzymatic inhibition assays (FIG. 40B). Corrected AAT (i.e., a combination of PiM+bystander and PiM AAT polypeptides) in serum was mostly PiM+bystander AAT in mice administered the dose of 2 mpk and correlated with functional AAT, e.g., the capacity to inhibit human NE (dots) ex vivo (FIG. 40C). In FIGS. 40A to 40C doses represent amounts of total RNA.

FIGS. 41A and 41B provide bar graphs demonstrating that hepatocytes that expressed PiM+Bystander AAT proteins demonstrated a survival advantage in NSG-PiZ mice. FIG. 41A provides a bar graph showing editing efficiency for the indicated edits (i.e., PiZ+Bystander; PiM+Bystander (D365G); and PiM (WT)) in NSG-PiZ mice administered the Variant G Formulation. FIG. 41B provides a bar graph showing serum levels of the indicated proteins (i.e., PiM (WT); PiM+Bystander (D365G); PiZ; and PiZ+Bystander) in NSG-PiZ mice administered the Variant G Formulation. Each set of bars in FIG. 41A correspond, from left-to-right, to PiZ+Bystander, PiM+Bystander (D365G), and PiM (WT), respectively. Each set of three bars in FIG. 41B correspond, from left-to-right to PiM (WT), PiM+Bystander (D365G), PiZ, and PiZ+Bystander, respectively. PiM indicates a wild-type alpha-1 antitrypsin (AAT) protein, PiM+Bystander indicates a wild-type AAT protein altered to include a D365G alteration, PiZ+Bystander indicates a PiZ AAT protein altered to include a D365G alteration, and PiZ indicates a PiZ AAT protein.

FIG. 42 provides a stacked bar graph demonstrating increased levels of hepatocyte base editing in PiZ Rats administered a second dose (“double”) rather than a single dose (“single”) of the Variant G Formulation. In FIG. 42, “Corrected Alleles” indicates total PiM alleles and PiM+Bystander alleles, “Additional base-edited alleles” indicates alleles other than PiM alleles and PiM+Bystander alleles, excluding insertion/deletion variants of AAT, and “Indels” indicates insertion/deletion variants of AAT. For each stacked bar of FIG. 42, the following are represented from bottom-to-top: “Additional base-edited alleles”, “Corrected alleles”, and “Indels”. In FIG. 42, doses represent amounts of total RNA.

FIGS. 43A and 43B provide stacked bar graphs demonstrating durable editing of liver cells in PiZ-Rats using the Variant G Formulation. FIG. 43A provides a stacked bar graph showing levels of each of the indicated alleles over time in PiZ rats administered the Variant G Formulation. FIG. 43B provides a stacked bar graph showing levels of each of the indicated proteins (“proteoforms”) in the blood serum of PiZ rats administered the Variant G formulation. In FIGS. 43A and 43B, “Corrected Alleles” indicates total PiM alleles and PiM+Bystander alleles, “Additional base-edited alleles” indicates alleles other than PiM alleles and PiM+Bystander alleles, Z-AAT indicates an AAT protein encoded by a SERPINA1 gene having the PiZ mutation, and “Vehicle” indicates PiZ rats that were not administered the Variant G Formulation.

FIGS. 44A and 44B provide bar graphs demonstrating a durable decrease in liver PiZ AAT in PiZ rats after dosing with the Variant G Formulation (i.e., lipid nanoparticles containing a base editor system containing gRNA856 and mRNA encoding the base editor ABE variant G). FIG. 44A provides a bar graph showing liver PiZ AAT levels in PiZ rats one week after administration of the Variant G Formulation. FIG. 44B provides a bar graph showing liver PiZ AAT levels in PiZ rats at 1 week and 14 weeks after administration of the Variant G Formulation at a dose of 0.5 mpk. Protein levels were measured using liquid chromatography-mass spectrometry (LC-MS). In each of FIGS. 44A and 44B, the first set of four bars from the left correspond to male rats and the set of four bars closest to the right correspond to female rats. The indicated doses represent amounts of total RNA.

FIG. 45 provides a bar graph showing improved hepatocyte base editing in PiZ rats administered the Variant G Formulation as opposed to a formulation containing an alternative lipid nanoparticle. The first three bars from the left correspond to PiZ rats administered the Variant G Formulation and the three bars closest to the right correspond to PiZ rats administered a formulation containing a lipid nanoparticle different from that used in the Variant G Formulation containing guide gRNA856 and mRNA encoding the base editor ABE Variant G. Each stacked bar of FIG. 45 represents from bottom-to-top “Additional base-edited alleles”, “Corrected alleles”, and “Indels”. In FIG. 45, “Corrected Alleles” indicates total PiM alleles and PiM+Bystander alleles, “Additional base-edited alleles” indicates alleles other than PiM alleles and PiM+Bystander alleles, and excluding insertion/deletion variants, “Indels” indicates insertion/deletion variants of AAT, and “Saline” indicates PiZ rats that were not administered the Variant G Formulation.

FIG. 46 provides a plot showing relative frequencies of on-target base-edited, corrected SERPINA1 alleles (i.e., PiM and PiM+Bystander alleles) in the liver of male and female NSG-PiZ mice one week after administration of the indicated doses of the Variant G Formulation. Doses of Variant G Formulation are expressed as total RNA. Horizontal lines represent group means, error bars represent SD, and circles represent values for individual mice. All male mice administered 0.05 mg/kg of Variant G Formulation and all female mice administered 2.0 mg/kg of Variant G Formulation were excluded from the data analysis at Day 7 due to discrepancy in sample ID assignment during collection.

FIG. 47 provides plots showing a correlation between relative frequency of on-target base-edited, corrected SERPINA1 alleles (i.e., PiM and PiM+Bystander alleles) in liver and levels of total human AAT and PiZ AAT variants in the serum of male and female NSG-PiZ mice one week after administration of the Variant G Formulation. Data points represent levels of total human AAT (triangles) and of PiZ AAT variants (squares) in the serum of individual mice. Nonlinear regression fit curves and R² values are indicated.

FIG. 48 provides a plot showing relative levels (vs Day 2) of functional AAT in the serum of male and female NSG-PiZ mice one week after administration of the Variant G Formulation. Doses of Variant G Formulation are expressed as total RNA. Horizontal lines represent group means, error bars represent SD, and circles represent values for individual mice. The horizontal dashed line represents the 1-fold relative level.

FIG. 49 provides a plot showing relative frequencies of on-target base-edited, corrected SERPINA1 alleles in the liver of male and female NSG-PiZ mice one and 13 weeks after administration of the Variant G Formulation at a total RNA dose of 0.25 mg/kg. Doses of Variant G Formulation are expressed as total RNA. Horizontal lines represent group means, error bars represent SD, and circles represent values for individual mice. One male mouse administered vehicle was euthanized before the end of the study due to fight wounds; no samples were collected at Week 13.

FIG. 50 provides a plot showing relative levels (vs Day 3) of total human AAT in the serum of male and female NSG-PiZ mice 1, 4, and 13 weeks after administration of a total RNA dose of 0.25 mg/kg of the Variant G Formulation. Doses of Variant G Formulation are expressed as total RNA. Horizontal lines represent group means, error bars represent SD, and symbols represent values for individual mice. The horizontal dashed line represents the 1-fold relative level. One male mouse administered vehicle was euthanized before the end of the study due to fight wounds; no samples were collected at Week 13.

FIG. 51 provides a plot showing relative levels (vs Day 3) of functional AAT in the serum of male and female NSG-PiZ mice 1 and 13 weeks after administration of the Variant G formulation at a dose of 0.25 mg/kg total RNA. Doses of the Variant G Formulation are expressed as total RNA. Horizontal lines represent group means, error bars represent SD, and circles represent values for individual mice. The horizontal dashed line represents the 1-fold relative level.

FIG. 52 provides a plot showing levels of human PiZ AAT protein in the serum of male and female PiZ rats at week 26 (6 month study) following administration of the Variant G Formulation. Circles represent individual male (M) and female (F) rats, and horizontal lines represent group median.

FIG. 53 presents plots showing longitudinal changes in the levels of human PiZ AAT protein in the serum of male and female PiZ rats (1-year study). Circles represent individual measurements and lines connect longitudinal measurements for each rat.

FIG. 54 provides a plot showing relative frequencies of on-target base-edited, corrected SERPINA1 alleles in the liver of male and female PiZ rats one week after administration of the Variant G Formulation. Doses of Variant G Formulation are expressed as total RNA. Horizontal lines represent group means, error bars represent SD, and circles represent values for individual rats. One of 5 male rats administered 1.0 mg/kg of Variant G Formulation was excluded from the analysis because the full Variant G Formulation dose was not injected.

FIG. 55 provides a plot showing relative abundance of PiZ AAT variants in the liver of male and female PiZ rats one week after administration of the Variant G Formulation (vs abundance in the liver of vehicle control rats). Doses of Variant G Formulation are expressed as total RNA. Symbols represent the mean abundances of PiZ AAT variants observed in each study group, normalized to the mean values observed in the vehicle control group.

FIG. 56 provides plots showing a correlation between the relative frequency of on-target base-edited, corrected SERPINA1 alleles in the liver and the levels of total human AAT and PiZ AAT variants in the serum of male and female PiZ rats one week after administration of the Variant G Formulation. Data points represent levels of total human AAT (triangles) and of PiZ AAT variants (squares) in the serum of individual rats. Nonlinear regression fit curves and R2 values are indicated.

FIG. 57 provides a plot showing relative frequencies of on-target base-edited, corrected SERPINA1 alleles in the liver of PiZ rats one and 14 weeks after administration of a total RNA dose of 0.50 mg/kg of the Variant G Formulation. Doses of Variant G Formulation are expressed as total RNA. Horizontal lines represent group means, error bars represent SD, and circles represent values for individual rats.

FIG. 58 provides a plot showing relative levels (vs Day 2) of total human AAT in the serum of male and female PiZ rats 1, 4, and 14 weeks after administration of a total RNA dose of 0.50 mg/kg of the Variant G Formulation. Data shown correspond to Group 1 (vehicle control; evaluated at Week 1), Group 2 (Variant G Formulation; evaluated at Week 1), and Group 3 (Variant G Formulation; evaluated at Weeks 4 and 5). Horizontal lines represent group means, error bars represent SD, and circles represent values for individual rats. The horizontal dashed line represents the mean total AAT value observed in the serum of control rats at Day-2.

FIG. 59 provides plots showing mean relative abundance of corrected AAT and PiZ AAT variants in the serum of male and female PiZ rats 1, 4, and 14 weeks after administration of a total RNA dose of 0.50 mg/kg of the Variant G Formulation. Data shown correspond to Group 2 (Variant G Formulation; evaluated at Week 1) and Group 3 (Variant G Formulation; evaluated at Day-2, Week 4, and Week 14). Data points represent mean relative abundance values of corrected AAT (circles) and PiZ AAT variants (triangles) for individual rats. Horizontal ticks represent group means and error bars represent SD. “D-2” represents the Day-2 baseline time point.

### DETAILED DESCRIPTION

As described below, the present invention features compositions and methods for altering mutations associated with alpha-1 antitrypsin deficiency (A1AD). In some embodiments, adenosine base editors, provided herein, correct a deleterious mutation, such that the edited polynucleotide is indistinguishable from a wild-type reference polynucleotide sequence. In some embodiments, such adenosine base editors, adenosine base editor systems, and methods of using the same have improved properties including e.g., increased on-target editing and decreased off-target editing. In another embodiment, the editing alters the deleterious mutation, such that the edited polynucleotide comprises a benign mutation.

The invention is based, at least in part, on the discovery that adenosine base editor variants can more efficiently and precisely edit a deleterious mutation associated with A1AD. Accordingly, the disclosure provides novel proteins and base editor variants capable of conferring improved base editor properties.

### Alpha-1 Antitrypsin Deficiency (A1AD)

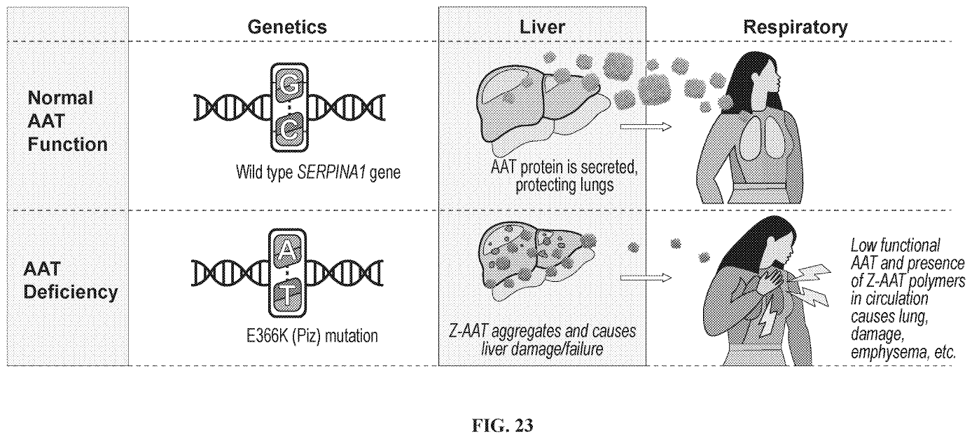

Alpha-1 antitrypsin (A1A) is a protease inhibitor encoded by the SERPINA1 gene on chromosome 14 (FIG. 23). This glycoprotein is synthesized mainly in the liver and is secreted into the blood, with serum concentrations of 1.5-3.0 g/L (20-52 μmol/L) in healthy adults. It diffuses into the lung interstitium and alveolar lining fluid, where it inactivates neutrophil elastase, thereby protecting the lung tissue from protease-mediated damage. Alpha-1 antitrypsin deficiency (A1AD) is inherited in an autosomal codominant fashion. Over 100 genetic variants of the SERPINA1 gene have been described, but not all are associated with disease. The alphabetic designation of these variants is based on their speed of migration on gel electrophoresis. The most common variant is the M (medium mobility) allele (PiM), and the two most frequent deficiency alleles are PiS and PiZ (the latter having the slowest rate of migration). Several mutations have been described that produce no measurable serum protein; these are referred to as “null” alleles. The most common genotype is MM, which produces normal serum levels of alpha-1 antitrypsin. Most people with severe deficiency are homozygous for the Z allele (ZZ). More than 60,000 patients with A1AD in the United States have the severe ZZ phenotype. The Z protein misfolds and polymerizes during its production in the endoplasmic reticulum of hepatocytes; these abnormal polymers are trapped in the liver, greatly reducing the serum levels of alpha-1 antitrypsin. Deficient or unstable A1AT production causes liver and/or lung pathologies in patients afflicted with A1AD. The liver disease seen in patients with alpha-1 antitrypsin deficiency is caused by the accumulation of abnormal alpha-1 antitrypsin protein in hepatocytes and the consequent cellular responses, including autophagy, the endoplasmic reticulum stress response and apoptosis. Reduced circulating levels of alpha-1 antitrypsin lead to increased neutrophil elastase activity in the lungs; this imbalance of protease and antiprotease results in the lung disease associated with this condition.

Alpha-1 antitrypsin deficiency (“A1AD”) is most common in Caucasians, and it most frequently affects the lungs and liver. In the lungs, the most common manifestation is early-onset (patients in their 30s and 40s) panacinar emphysema most pronounced in the lung bases. However, diffuse or upper lobe emphysema can occur, as can bronchiectasis. The most frequently described symptoms include dyspnea, wheezing and cough. Pulmonary function testing of affected individuals shows findings consistent with COPD; however, bronchodilator responsiveness may be observed and may be misdiagnosed as asthma. Liver disease caused by the ZZ genotype manifests in various ways. Affected infants can present in the newborn period with cholestatic jaundice, sometimes with acholic stools (pale or clay-colored) and hepatomegaly. Conjugated bilirubin, transaminases and gamma-glutamyl transferase levels in blood are elevated. Liver disease in older children and adults can present with an incidental finding of elevated transaminases or with signs of established cirrhosis, including variceal hemorrhage or ascites. Alpha-1 antitrypsin deficiency also predisposes patients to hepatocellular carcinoma. Although the homozygous ZZ genotype is necessary for liver disease to develop, a heterozygous Z mutation can act as a genetic modifier for other diseases by conferring a greater risk of more severe liver disease, such as in hepatitis C infection and cystic fibrosis liver disease.

The two most common clinical variants of A1AD are E264V (PiS) and E342K (PiZ) alleles. The clinical single nucleotide variant E342K (PiZ) leads to unstable and/or inactive A1AT protein and, as a consequence, causes liver and lung toxicities. Inheritance is autosomal codominant. More than a half of A1AD patients harbor at least one copy of the mutation E342K.

In some embodiments, the disease or disorder is alpha-1 antitrypsin deficiency (A1AD). In some embodiments, the pathogenic mutation is in gene SERPINA1. In some embodiments, the mutation of SERPINA1 is E342K (PiZ allele). In some embodiments, A at position 7 is edited to G to restore PiZ allele to a wild type allele.

### Editing of Target Genes

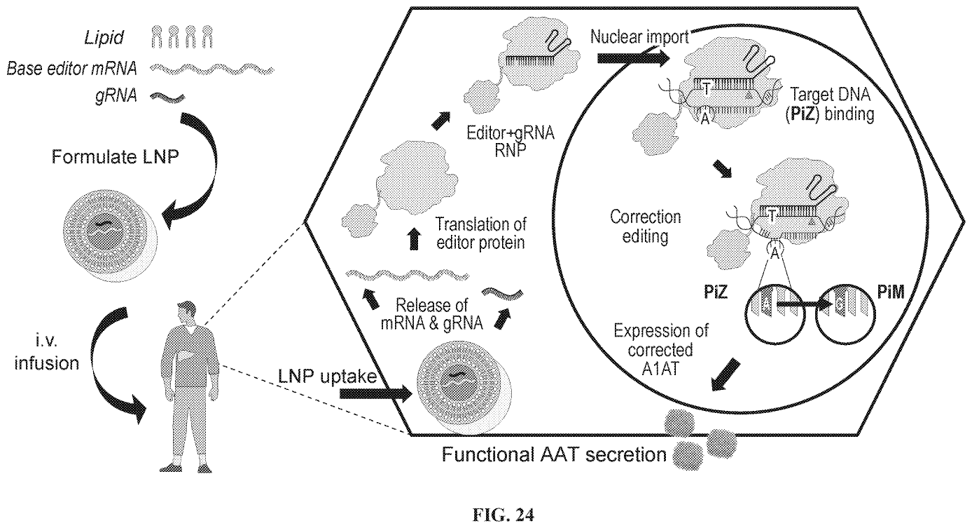

To produce the gene edits described above, cells within or collected from a subject are contacted with one or more guide RNAs and a nucleobase editor polypeptide comprising a nucleic acid programmable DNA binding protein (napDNAbp) and a deaminase (see, e.g., FIG. 24). In some embodiments, cells to be edited are contacted with at least one nucleic acid, wherein the at least one nucleic acid encodes one or more guide RNAs and/or a nucleobase editor polypeptide comprising a nucleic acid programmable DNA binding protein (napDNAbp) and a deaminase. In some embodiments, the gRNA comprises nucleotide analogs. In some instances, the gRNA is added directly to a cell. These nucleotide analogs can inhibit degradation of the gRNA from cellular processes. Table 1 provides representative guide polynucleotide sequences suitable for use in methods, base editor systems, and/or compositions of the disclosure. Any guide polynucleotide sequence provided in the disclosure may be suitable for use in methods, base editor systems, and/or compositions of the disclosure.

Variants of the spacer sequences listed in Table 1 comprising 1, 2, 3, 4, or 5 nucleobase alterations are contemplated. For example, variation of a target polynucleotide sequence within a population (e.g., single nucleotide polymorphisms) may require said alterations to a spacer sequence to allow the spacer to better bind a variant of a target sequence in a subject.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science. 1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

TABLE 1
Guide polynucleotide sequences for correcting
pathogenic mutations.

| | | Pathogenic | Base | gRNA Targetin | |
|---|---|---|---|---|---|
| | Gene | Mutation | Editor | Sequence | PAM |
| 1. | SERPINA1 | E342K | ABE | GACAAGAAAGGGA | NGC |
| | | | | CUGAAGC (SEQ | |
| | | | | ID NO: 433) | |
| 2. | SERPINA1 | E342K | ABE | AUCGACAAGAAAG | NGC |
| | | | | GGACUGA (SEQ | |
| | | | | ID NO: 434) | |
| 3. | SERPINCI | R48C | ABE | ACACACCGGUUGG | NGG |
| | | (R79C) | | UGGCCUC (SEQ | |
| | | | | ID NO: 435) | |

### Nucleobase Editors

Useful in the methods and compositions described herein are nucleobase editors (alternatively, base editors) that edit, modify or alter a target nucleotide sequence of a polynucleotide. Nucleobase editors described herein typically include a polynucleotide programmable nucleotide binding domain and a nucleobase editing domain (e.g., an adenosine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

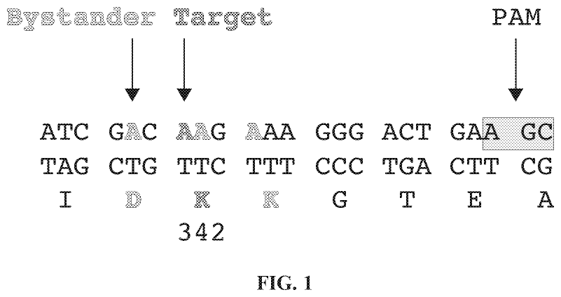

Some aspects of the disclosure provide variants of adenosine base editors (ABEs). In some embodiments, the ABE variants provided herein are useful for targeting a mutation in a SERPINA1 gene, for example a E342K (PiZ allele) mutation in SERPINA1. In some embodiments, the base editors are useful for treating a disease or disorder, such as alpha-1 antitrypsin deficiency (A1AD). In some embodiments, an A at position 7 is edited to G to restore PiZ allele to a wild type allele. A schematic representation of an exemplary target A at position 7 in SERPINA1 is shown in FIG. 1, which is indicated as “Target” in FIG. 1.

In some embodiments, the disclosure provides variants of ABE Variant 12. An amino acid sequence of ABE Variant 12 is provided herein. In the below sequence of ABE Variant 12, the amino acid sequence of TadA*7.10 (L36H, I76Y, V82T, Y147T, Q154S, and N157K) is indicated with bold; the amino acid sequence of the linker is indicated with underlining, the amino acid sequence of SpCas9-MQKFRAER nickase is indicated with italics, and the amino acid sequence of the NLS is indicated with bold and underlining.

(SEQ ID NO: 436)
SEVEFSHEYWMRHALTLAKRARDEREVPVGAVLVHNNRVIGEGWNRAIGLHD
PTAHAEIMALRQGGLVMQNYRLYDATLYTTFEPCVMCAGAMIHSRIGRVVFGV
RNAKTGAAGSLMDVLHYPGMNHRVEITEGILADECAALLCTFFRMPRSVFKAQ
KKAQSSTDSGGSSGGSSGSETPGTSESATPESSGGSSGGSDKKYSIGLAIGTNSVGWAVI
TDEYKVPSKKFKVLGNTDRHSIKKNLIGALLFDSGETAEATRLKRTARRRYTRRKNRICYLQ
EIFSNEMAKVDDSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQLFEENPINASG
VDAKAILSARLSKSRRLENLIAOLPGEKKNGLFGNLIALSLGLTPNFKSNFDLAEDAKLQLS
KDTYDDDLDNLLAQIGDQYADLFLAAKNLSDAILLSDILRVNTEITKAPLSASMVKRYDEH
HODLTLLKALVROQLPEKYKEIFFDQSKNGYAGYIDGGASQEEFYKFIKPILEKMDGTEEL
LVKLNREDLLRKORTFDNGIIPHQIHLGELHAILRRQGDFYPFLKDNREKIEKILTFRIPYY
VGPLARGNSRFAWMTRKSEETITPWNFEEVVDKGASAOSFIERMTNFDKNLPNEKVLPKH
SLLYEYFTVYNELTKVKYVTEGMRKPAFLSGEQKKAIVDLLFKTNRKVTVKOLKEDYFKKI
ECFDSVEISGVEDRFNASLGTYHDLLKIIKDKDFLDNEENEDILEDIVLTLTLFEDREMIEE
RLKTYAHLFDDKVMKOLKRLRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGGH
KPENIVIEMARENOTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRGKSDNVPSEEV
VKKMKNYWROLLNAKLITORKFDNLTKAERGGLSELDKAGFIKROLVETROITKHVAQIL
DSRMNTKYDENDKLIREVKVITLKSKLVSDFRKDFOFYKVREINNYHHAHDAYLNAVVGT
ALIKKYPKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESILPKGNSDK
LIARKKDWDPKKYGGFMOPTVAYSVLVVAKVEKGKSKKLKSVKELLGITIMERSSFEKNPI
DFLEAKGYKEVKKDLIIKLPKYSLFELENGRKRMLASAKFLQKGNELALPSKYVNFLYLAS
HYEKLKGSPEDNEOKOLFVEOHKHYLDEIIEOISEFSKRVILADANLDKVLSAYNKHRDKP
IREQAENIIHLFTLTNLGAPRAFKYFDTTIARKEYRSTKEVLDATLIHQSITGLYETRIDLS
QLGGDEGADKRTADGSEFESPKKKRKV (ABE Variant 12)

An exemplary codon-optimized nucleotide sequence encoding ABE Variant 12 is provided below:

(SEQ ID NO: 437)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAGCGGCGGGAGCTCTGGGGGCTCCTCCGGGAGCGAGACCCCCGGGACCAGCGAGTCCGCC
ACCCCCGAGAGCAGCGGCGGCAGCTCCGGGGGGAGCGACAAGAAGTACTCCATCGGCCTGGC
CATCGGCACCAACTCCGTGGGTTGGGCCGTGATCACCGATGAGTACAAGGTGCCCAGCAAGA
AGTTCAAGGTGCTGGGCAACACCGACAGGCACTCTATCAAGAAGAACCTGATCGGCGCCCTG
CTGTTCGACAGCGGGGAGACCGCTGAGGCCACTCGGCTGAAGAGAACCGCCAGGCGCAGATA
CACCCGGAGAAAGAACCGGATCTGCTACCTGCAGGAGATCTTCAGCAACGAGATGGCCAAGG
TGGACGACAGCTTCTTCCACAGGCTGGAGGAGAGCTTCCTGGTGGAGGAGGACAAGAAGCAC
GAGCGCCACCCCATCTTCGGCAACATCGTGGATGAGGTGGCCTACCACGAGAAGTACCCCAC
CATCTACCACCTGCGGAAGAAGCTGGTGGACAGCACCGATAAGGCCGATCTGCGGCTGATCT
ACCTGGCCCTGGCCCACATGATCAAGTTCAGGGGGCACTTCCTGATCGAGGGCGACCTGAAC
CCCGACAACTCCGATGTGGATAAACTGTTCATCCAGCTGGTGCAGACCTACAACCAGCTGTT
TGAGGAGAATCCCATCAACGCCTCCGGCGTGGACGCCAAGGCCATCCTGAGCGCCCGGCTGT
CCAAGAGCCGGAGGCTGGAGAATCTGATCGCCCAGCTGCCTGGCGAGAAGAAGAACGGCCTG
TTCGGCAACCTGATCGCCCTGAGCCTGGGCCTGACCCCCAATTTCAAGAGCAACTTCGACCT
GGCCGAGGATGCTAAGCTGCAGCTGTCCAAGGACACCTACGACGATGACCTGGACAACCTGC
TGGCTCAGATCGGCGATCAGTACGCCGACCTGTTCCTGGCCGCCAAGAACCTGTCAGACGCC
ATCCTGCTGTCTGACATTCTGCGGGTGAACACCGAGATCACCAAGGCCCCACTGTCCGCCTC
TATGGTGAAGAGGTACGATGAGCACCACCAGGACCTGACCCTGCTGAAGGCTCTGGTGCGGC
AGCAGCTGCCGGAGAAGTACAAGGAGATCTTCTTCGACCAGAGCAAGAACGGCTACGCCGGC
TACATCGACGGCGGGGCCAGCCAGGAGGAGTTCTACAAGTTCATCAAGCCTATCCTGGAGAA
GATGGACGGCACCGAGGAGCTGCTGGTGAAGCTGAACCGGGAGGACCTGCTGCGGAAGCAGA
GGACCTTCGACAACGGCATCATTCCCCACCAGATCCACCTGGGCGAGCTGCACGCCATCCTG
CGGAGGCAGGGCGACTTCTACCCTTTCCTGAAGGACAACAGGGAGAAGATCGAGAAGATCCT
GACCTTCAGGATCCCCTACTACGTGGGCCCCCTGGCCCGCGGCAACTCCCGCTTTGCCTGGA
TGACCAGAAAGAGCGAGGAGACCATCACCCCTTGGAACTTCGAGGAGGTGGTGGACAAGGGC
GCCAGCGCCCAGAGCTTCATCGAGCGCATGACCAACTTCGACAAGAATCTGCCCAACGAGAA
GGTGCTGCCCAAGCACTCCCTGCTGTACGAGTACTTCACCGTGTACAACGAGCTGACAAAGG
TGAAGTACGTGACAGAGGGCATGCGCAAGCCCGCCTTCCTGTCTGGCGAGCAGAAGAAGGCC
ATCGTGGACCTGCTGTTCAAGACCAACAGGAAGGTGACCGTGAAGCAGCTGAAGGAGGACTA
CTTCAAGAAGATCGAGTGCTTCGACAGCGTGGAGATCAGCGGCGTGGAGGACAGGTTCAACG
CCAGCCTGGGCACCTACCACGACCTGCTGAAGATCATCAAGGACAAGGACTTCCTGGATAAC
GAGGAGAACGAGGATATCCTGGAGGACATTGTCCTGACCCTGACCCTGTTCGAGGATCGGGA
GATGATCGAGGAGCGCCTCAAGACCTACGCCCACCTGTTCGACGATAAGGTGATGAAGCAGC
TGAAGCGGCTGCGCTACACCGGCTGGGGCCGCCTGTCCCGGAAGCTGATCAACGGCATCCGG
GACAAGCAGTCCGGCAAGACCATCCTGGACTTCCTGAAGTCCGATGGTTTTGCCAACAGAAA
CTTCATGCAGCTGATCCACGACGACAGCCTGACCTTCAAGGAGGACATCCAGAAGGCTCAGG
TGTCCGGACAGGGGGACTCCCTGCACGAGCACATCGCCAACCTGGCCGGCTCTCCCGCCATC
AAGAAGGGCATCCTGCAGACCGTGAAGGTGGTGGACGAGCTGGTGAAGGTGATGGGCGGGCA
CAAGCCCGAGAACATCGTGATCGAGATGGCCCGGGAGAACCAGACCACCCAGAAGGGCCAGA
AGAACAGCAGGGAGCGCATGAAGCGCATCGAGGAGGGCATCAAGGAGCTGGGTAGCCAGATC
CTGAAGGAGCACCCTGTGGAGAATACCCAGCTGCAGAACGAGAAGCTGTATCTGTACTACCT
GCAGAACGGCAGGGACATGTACGTGGATCAGGAGCTGGACATCAACCGGCTGTCTGACTACG
ACGTGGACCACATCGTGCCCCAGTCTTTCCTGAAGGACGACAGCATCGACAACAAGGTGCTG
ACCCGCAGCGACAAGAACAGGGGCAAGAGCGATAACGTGCCCTCCGAGGAGGTGGTCAAGAA
GATGAAGAACTACTGGCGGCAGCTGCTGAACGCCAAGCTGATCACCCAGCGGAAGTTCGACA
ACCTGACCAAGGCCGAGAGAGGCGGCCTGTCCGAGCTGGACAAGGCCGGCTTCATCAAGCGG
CAGCTGGTGGAGACCCGGCAGATCACCAAGCACGTCGCCCAGATCCTGGACTCCAGGATGAA
CACCAAGTACGACGAGAACGACAAGCTGATCAGAGAGGTGAAGGTGATCACCCTGAAGTCCA
AGCTGGTGAGCGACTTCAGGAAGGATTTCCAGTTCTACAAGGTGCGGGAGATCAACAACTAT
CACCACGCCCACGACGCCTACCTGAACGCCGTCGTGGGCACCGCTCTGATCAAGAAGTATCC
TAAGCTGGAGAGCGAGTTCGTGTACGGCGACTACAAGGTGTACGACGTGCGGAAGATGATCG
CCAAGAGCGAGCAGGAGATCGGCAAGGCCACCGCCAAGTACTTCTTCTACTCCAATATCATG
AACTTCTTCAAGACCGAGATCACCCTGGCCAACGGGGAGATCCGGAAGCGCCCACTGATCGA
GACCAACGGCGAGACCGGCGAGATCGTGTGGGACAAGGGCAGGGACTTCGCCACCGTGAGGA
AGGTGCTGTCCATGCCCCAGGTGAACATCGTGAAAAAGACCGAGGTGCAGACCGGGGGGTTC
TCCAAGGAGAGCATCCTGCCCAAGGGCAACAGCGACAAGCTGATCGCCCGGAAGAAGGACTG
GGACCCCAAGAAGTACGGAGGCTTCATGCAGCCTACCGTGGCCTACTCCGTGCTGGTGGTGG
CCAAGGTGGAGAAGGGCAAGTCCAAGAAGCTGAAGTCAGTGAAGGAGCTGCTGGGCATCACC
ATCATGGAGCGGTCTAGCTTCGAGAAGAATCCTATTGACTTCCTGGAGGCCAAGGGCTACAA
GGAGGTCAAGAAGGATCTGATCATCAAGCTGCCCAAGTACAGCCTGTTCGAGCTGGAGAATG
GCCGGAAGAGGATGCTGGCCAGCGCCAAGTTCCTGCAGAAGGGCAACGAGCTGGCCCTGCCC
AGCAAGTACGTGAACTTCCTCTATCTGGCCAGCCACTACGAGAAGCTGAAGGGCTCTCCAGA
GGACAACGAGCAGAAGCAGCTGTTCGTGGAGCAGCACAAGCACTACCTGGACGAGATCATCG
AGCAGATCTCCGAGTTCAGCAAGCGCGTGATCCTGGCCGACGCCAACCTGGACAAGGTGCTG
TCCGCTTACAACAAGCACAGGGACAAGCCCATCAGGGAGCAGGCCGAGAACATCATCCACCT
GTTCACCCTGACCAACCTGGGCGCCCCCAGGGCCTTCAAGTACTTCGATACCACCATCGCCA
GGAAGGAGTACAGAAGTACTAAGGAGGTCCTGGATGCCACTCTGATCCACCAGAGCATCACC
GGGCTGTACGAGACCCGGATCGACCTGTCCCAGCTGGGCGGCGACGAGGGCGCCGACAAGCG
GACAGCCGACGGCAGCGAGTTCGAGAGCCCCAAGAAGAAGCGGAAGGTG.

In some embodiments, the ABE variant comprises the adenosine deaminase (TadA*7.10 L36H, I76Y, V82T, Y147T, Q154S, and N157K). In some embodiments, ABE Variants A-K, provided herein, comprise the adenosine deaminase TadA*7.10 (L36H, I76Y, V82T, Y147T, Q154S, and N157K). Table 2 shows the configuration of ABE variants A-K, indicating the mutations in SpCas9-MQKFRAER nickase and the linker sequences. Each ABE variant listed in the table comprises the adenosine deaminase TadA*7.10 (L36H, 176Y, V82T, Y147T, Q154S, and N157K) and the NLS amino acid sequence

(SEQ ID NO: 438)
EGADKRTADGSEFESPKKKRKV.

TABLE 2
A summary of ABE Variants A-K.

| | ABE | SpCas9-MQKFRAER | | SEQ |
|---|---|---|---|---|
| Plasmid | Variant | nickcase mutant | Linker | ID NO |
| pMSP1115 | Variant A | R1337K | SGGSSGGSSGSE | 357 |
| | | | TPGTSESATPESS | |
| | | | GGSSGGS | |
| pMSP1116 | Variant B | Q1136Y, and R1337K | SGGSSGGSSGSE | 357 |
| | | | TPGTSESATPESS | |
| | | | GGSSGGS | |
| pMSP1130 | Variant C | M1135L, Q1136Y, | EGGSEEEEESGS | 432 |
| | | and R1337K | | |
| pMSP1131 | Variant D | Q1136Y, and R1337K | EGGSEEEEESGS | 432 |
| pMSP1132 | Variant E | M1135L, Q1136Y, | KGPKPKKEESEK | 439 |
| | | and R1337K | | |
| pMSP1160 | Variant F | M1135L, A1283D, | SGGSSGGSSGSE | 357 |
| | | Q1136Y, and R1337K | TPGTSESATPESS | |
| | | | GGSSGGS | |
| pMSP1167 | Variant G | M1135L, A1283D, | SGGSSGGSSGSE | 357 |
| | | E1250K, and R1337K | TPGTSESATPESS | |
| | | | GGSSGGS | |
| pMSP1168 | Variant H | A1283D, E1250K, | SGGSSGGSSGSE | 357 |
| | | and Q1136Y | TPGTSESATPESS | |
| | | | GGSSGGS | |
| pMSP1177 | Variant I | M1135L, A1283D, | KGPKPKKEESEK | 439 |
| | | Q1136Y, and R1337K | | |
| pMSP1179 | Variant J | M1135L, A1283D, | SGGSSGGSSGSE | 357 |
| | | Q1136Y, R1337K, | TPGTSESATPESS | |
| | | R765A, and Q768A | GGSSGGS | |
| pMSP1188 | Variant K | A1283D, E1250K, | KGPKPKKEESEK | 439 |
| | | and Q1136Y | | |

Exemplary polypeptide sequences for Variants A-K are provided below. In the below sequences of Variants A-K, the amino acid sequence of the TadA is indicated with bold; the amino acid sequence of the linker is indicated with underlining, the amino acid sequence of the SpCas9-MQKFRAER nickase mutant is indicated with italics, and the amino acid sequence of the NLS is indicated with bold and underlining.

Variant A amino acid sequence:
(SEQ ID NO: 440)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDSGGSSGGSSGSETPGTSESA
TPESSGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDA
ILLSDILRVNTEITKAPLSASMVKRYDEHHQDLTLLKALVROQLPEKYKEIFFDQSKNGYAG
YIDGGASQEEFYKFIKPILEKMDGTEELLVKLNREDLLRKORTFDNGIIPHQIHLGELHAIL
RRQGDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKOLKRLRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGGHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGF
SKESILPKGNSDKLIARKKDWDPKKYGGFMQPTVAYSVLVVAKVEKGKSKKLKSVKELLGIT
IMERSSFEKNPIDFLEAKGYKEVKKDLIIKLPKYSLFELENGRKRMLASAKFLQKGNELALP
SKYVNFLYLASHYEKLKGSPEDNEQKOLFVEQHKHYLDEIIEQISEFSKRVILADANLDKVL
SAYNKHRDKPIREQAENIIHLFTLTNLGAPRAFKYFDTTIARKEYKSTKEVLDATLIHQSIT
GLYETRIDLSQLGGDEGADKRTADGSEFESPKKKRKV.
Variant B amino acid sequence:
(SEQ ID NO: 441)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDSGGSSGGSSGSETPGTSESA
TPESSGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDA
ILLSDILRVNTEITKAPLSASMVKRYDEHHQDLTLLKALVRQQLPEKYKEIFFDQSKNGYAG
YIDGGASQEEFYKFIKPILEKMDGTEELLVKLNREDLLRKORTFDNGIIPHQIHLGELHAIL
RRQGDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKOLKRLRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGGHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSIDNKSE
VEFSHEYWMRHALTLAKRARDEREVPVGAVLVHNNRVIGEGWNRAIGLHDPTAHAEIMALRQ
GGLVMQNYRLYDATLYTTFEPCVMCAGAMIHSRIGRVVFGVRNAKTGAAGSLMDVLHYPGMN
HRVEITEGILADECAALLCTFFRMPRSVFKAQKKAQSSTDVLTRSDKNRGKSDNVPSEEVVK
KMKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKGNSDKLIARKKD
WDPKKYGGFMYPTVAYSVLVVAKVEKGKSKKLKSVKELLGITIMERSSFEKNPIDFLEAKGY
KEVKKDLIIKLPKYSLFELENGRKRMLASAKFLQKGNELALPSKYVNFLYLASHYEKLKGSP
EDNEQKQLFVEQHKHYLDEIIEQISEFSKRVILADANLDKVLSAYNKHRDKPIREQAENIIH
LFTLTNLGAPRAFKYFDTTIARKEYKSTKEVLDATLIHQSITGLYETRIDLSQLGGDEGADK
RTADGSEFESPKKKRKV.
Variant C amino acid sequence:
(SEQ ID NO: 442)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDEGGSEEEEESGSDKKYSIGL
AIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSA
SMVKRYDEHHQDLTLLKALVRQQLPEKYKEIFFDQSKNGYAGYIDGGASQEEFYKFIKPILE
KMDGTEELLVKLNREDLLRKORTFDNGIIPHQIHLGELHAILRRQGDFYPFLKDNREKIEKI
LTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDR
EMIEERLKTYAHLFDDKVMKQLKRLRYTGWGRLSRKLINGIRDKQSGKTILDFLKSDGFANR
NFMQLIHDDSLTFKEDIQKAQVSGQGDSLHEHIANLAGSPAIKKGILQTVKVVDELVKVMGG
HKPENIVIEMARENQTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETROITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKGNSDKLIARKKD
WDPKKYGGFLYPTVAYSVLVVAKVEKGKSKKLKSVKELLGITIMERSSFEKNPIDFLEAKGY
KEVKKDLIIKLPKYSLFELENGRKRMLASAKFLQKGNELALPSKYVNFLYLASHYEKLKGSP
EDNEQKQLFVEQHKHYLDEIIEQISEFSKRVILADANLDKVLSAYNKHRDKPIREQAENIIH
LFTLTNLGAPRAFKYFDTTIARKEYKSTKEVLDATLIHQSITGLYETRIDLSQLGGDEGADK
RTADGSEFESPKKKRKV.
Variant D amino acid sequence:
(SEQ ID NO: 443)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDEGGSEEEEESGSDKKYSIGL
AIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSA
SMVKRYDEHHQDLTLLKALVRQQLPEKYKEIFFDQSKNGYAGYIDGGASQEEFYKFIKPILE
KMDGTEELLVKLNREDLLRKORTFDNGIIPHQIHLGELHAILRRQGDFYPFLKDNREKIEKI
LTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDR
EMIEERLKTYAHLFDDKVMKQLKRLRYTGWGRLSRKLINGIRDKQSGKTILDFLKSDGFANR
NFMQLIHDDSLTFKEDIQKAQVSGQGDSLHEHIANLAGSPAIKKGILQTVKVVDELVKVMGG
HKPENIVIEMARENQTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKGNSDKLIARKKD
WDPKKYGGFMYPTVAYSVLVVAKVEKGKSKKLKSVKELLGITIMERSSFEKNPIDFLEAKGY
KEVKKDLIIKLPKYSLFELENGRKRMLASAKFLQKGNELALPSKYVNFLYLASHYEKLKGSP
EDNEQKQLFVEQHKHYLDEIIEQISEFSKRVILADANLDKVLSAYNKHRDKPIREQAENIIH
LFTLTNLGAPRAFKYFDTTIARKEYKSTKEVLDATLIHQSITGLYETRIDLSQLGGDEGADK
RTADGSEFESPKKKRKV.
Variant E amino acid sequence:
(SEQ ID NO: 444)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDKGPKPKKEESEKDKKYSIGL
AIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSA
SMVKRYDEHHQDLTLLKALVRQQLPEKYKEIFFDQSKNGYAGYIDGGASQEEFYKFIKPILE
KMDGTEELLVKLNREDLLRKORTFDNGIIPHQIHLGELHAILRRQGDFYPFLKDNREKIEKI
LTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDR
EMIEERLKTYAHLFDDKVMKQLKRLRYTGWGRLSRKLINGIRDKQSGKTILDFLKSDGFANR
NFMQLIHDDSLTFKEDIQKAQVSGQGDSLHEHIANLAGSPAIKKGILQTVKVVDELVKVMGG
HKPENIVIEMARENQTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKGNSDKLIARKKD
WDPKKYGGFLYPTVAYSVLVVAKVEKGKSKKLKSVKELLGITIMERSSFEKNPIDFLEAKGY
KEVKKDLIIKLPKYSLFELENGRKRMLASAKFLQKGNELALPSKYVNFLYLASHYEKLKGSP
EDNEQKQLFVEQHKHYLDEIIEQISEFSKRVILADANLDKVLSAYNKHRDKPIREQAENIIH
LFTLTNLGAPRAFKYFDTTIARKEYKSTKEVLDATLIHQSITGLYETRIDLSQLGGDEGADK
RTADGSEFESPKKKRKV.
Variant F amino acid sequence:
(SEQ ID NO: 445)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDSGGSSGGSSGSETPGTSESA
TPESSGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDA
ILLSDILRVNTEITKAPLSASMVKRYDEHHQDLTLLKALVRQQLPEKYKEIFFDQSKNGYAG
YIDGGASQEEFYKFIKPILEKMDGTEELLVKLNREDLLRKORTFDNGIIPHQIHLGELHAIL
RRQGDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRLRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGGHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGF
SKESILPKGNSDKLIARKKDWDPKKYGGFLYPTVAYSVLVVAKVEKGKSKKLKSVKELLGIT
IMERSSFEKNPIDFLEAKGYKEVKKDLIIKLPKYSLFELENGRKRMLASAKFLQKGNELALP
SKYVNFLYLASHYEKLKGSPEDNEQKOLFVEQHKHYLDEIIEQISEFSKRVILDDANLDKVL
SAYNKHRDKPIREQAENIIHLFTLTNLGAPRAFKYFDTTIARKEYKSTKEVLDATLIHQSIT
GLYETRIDLSQLGGDEGADKRTADGSEFESPKKKRKV.
Variant G amino acid sequence:
(SEQ ID NO: 446)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDSGGSSGGSSGSETPGTSESA
TPESSGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDA
ILLSDILRVNTEITKAPLSASMVKRYDEHHQDLTLLKALVRQQLPEKYKEIFFDQSKNGYAG
YIDGGASQEEFYKFIKPILEKMDGTEELLVKLNREDLLRKORTFDNGIIPHQIHLGELHAIL
RRQGDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRLRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGGHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGF
SKESILPKGNSDKLIARKKDWDPKKYGGFLQPTVAYSVLVVAKVEKGKSKKLKSVKELLGIT
IMERSSFEKNPIDFLEAKGYKEVKKDLIIKLPKYSLFELENGRKRMLASAKFLQKGNELALP
SKYVNFLYLASHYEKLKGSPKDNEQKQLFVEQHKHYLDEIIEQISEFSKRVILDDANLDKVL
SAYNKHRDKPIREQAENIIHLFTLTNLGAPRAFKYFDTTIARKEYKSTKEVLDATLIHQSIT
GLYETRIDLSQLGGDEGADKRTADGSEFESPKKKRKV.
Variant H amino acid sequence:
(SEQ ID NO: 447)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDSGGSSGGSSGSETPGTSESA
TPESSGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDA
ILLSDILRVNTEITKAPLSASMVKRYDEHHQDLTLLKALVROQLPEKYKEIFFDQSKNGYAG
YIDGGASQEEFYKFIKPILEKMDGTEELLVKLNREDLLRKORTFDNGIIPHQIHLGELHAIL
RRQGDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRLRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGGHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSIDNKVL
TRSDKNRGKSDNVPSEEVVKKMKNYWRQLLNAKLITORKFDNLTKAERGGLSELDKAGFIKR
QLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGF
SKESILPKGNSDKLIARKKDWDPKKYGGFMYPTVAYSVLVVAKVEKGKSKKLKSVKELLGIT
IMERSSFEKNPIDFLEAKGYKEVKKDLIIKLPKYSLFELENGRKRMLASAKFLQKGNELALP
SKYVNFLYLASHYEKLKGSPKDNEQKOLFVEQHKHYLDEIIEQISEFSKRVILDDANLDKVL
SAYNKHRDKPIREQAENIIHLFTLTNLGAPRAFKYFDTTIARKEYRSTKEVLDATLIHQSIT
GLYETRIDLSQLGGDEGADKRTADGSEFESPKKKRKV.
Variant I amino acid sequence:
(SEQ ID NO: 448)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDKGPKPKKEESEKDKKYSIGL
AIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSA
SMVKRYDEHHQDLTLLKALVRQQLPEKYKEIFFDQSKNGYAGYIDGGASQEEFYKFIKPILE
KMDGTEELLVKLNREDLLRKORTFDNGIIPHQIHLGELHAILRRQGDFYPFLKDNREKIEKI
LTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDR
EMIEERLKTYAHLFDDKVMKQLKRLRYTGWGRLSRKLINGIRDKQSGKTILDFLKSDGFANR
NFMQLIHDDSLTFKEDIQKAQVSGQGDSLHEHIANLAGSPAIKKGILQTVKVVDELVKVMGG
HKPENIVIEMARENQTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKGNSDKLIARKKD
WDPKKYGGFLYPTVAYSVLVVAKVEKGKSKKLKSVKELLGITIMERSSFEKNPIDFLEAKGY
KEVKKDLIIKLPKYSLFELENGRKRMLASAKFLQKGNELALPSKYVNFLYLASHYEKLKGSP
EDNEQKQLFVEQHKHYLDEIIEQISEFSKRVILDDANLDKVLSAYNKHRDKPIREQAENIIH
LFTLTNLGAPRAFKYFDTTIARKEYKSTKEVLDATLIHQSITGLYETRIDLSQLGGDEGADK
RTADGSEFESPKKKRKV.
Variant J amino acid sequence:
(SEQ ID NO: 449)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDSGGSSGGSSGSETPGTSESA
TPESSGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDA
ILLSDILRVNTEITKAPLSASMVKRYDEHHQDLTLLKALVRQQLPEKYKEIFFDQSKNGYAG
YIDGGASQEEFYKFIKPILEKMDGTEELLVKLNREDLLRKORTFDNGIIPHQIHLGELHAIL
RRQGDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRLRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGGHKPENIVIEMAAENATTQKGQKNSRERMKRIEEGIKELGSQI
LKEHPVENTQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGF
SKESILPKGNSDKLIARKKDWDPKKYGGFLYPTVAYSVLVVAKVEKGKSKKLKSVKELLGIT
IMERSSFEKNPIDFLEAKGYKEVKKDLIIKLPKYSLFELENGRKRMLASAKFLQKGNELALP
SKYVNFLYLASHYEKLKGSPEDNEQKQLFVEQHKHYLDEIIEQISEFSKRVILDDANLDKVL
SAYNKHRDKPIREQAENIIHLFTLTNLGAPRAFKYFDTTIARKEYKSTKEVLDATLIHQSIT
GLYETRIDLSQLGGDEGADKRTADGSEFESPKKKRKV.
Variant K amino acid sequence:
(SEQ ID NO: 450)
SEVEFSHEYWMRHALTLAKRARDEREVPVGAVLVHNNRVIGEGWNRAIGLHDPTAHAEIMAL
RQGGLVMQNYRLYDATLYTTFEPCVMCAGAMIHSRIGRVVFGVRNAKTGAAGSLMDVLHYPG
MNHRVEITEGILADECAALLCTFFRMPRSVFKAQKKAQSSTDKGPKPKKEESEKDKKYSIGL
AIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSA
SMVKRYDEHHQDLTLLKALVRQQLPEKYKEIFFDQSKNGYAGYIDGGASQEEFYKFIKPILE
KMDGTEELLVKLNREDLLRKORTFDNGIIPHQIHLGELHAILRRQGDFYPFLKDNREKIEKI
LTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDR
EMIEERLKTYAHLFDDKVMKQLKRLRYTGWGRLSRKLINGIRDKQSGKTILDFLKSDGFANR
NFMQLIHDDSLTFKEDIQKAQVSGQGDSLHEHIANLAGSPAIKKGILQTVKVVDELVKVMGG
HKPENIVIEMARENQTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKGNSDKLIARKKD
WDPKKYGGFMYPTVAYSVLVVAKVEKGKSKKLKSVKELLGITIMERSSFEKNPIDFLEAKGY
KEVKKDLIIKLPKYSLFELENGRKRMLASAKFLQKGNELALPSKYVNFLYLASHYEKLKGSP
KDNEQKQLFVEQHKHYLDEIIEQISEFSKRVILDDANLDKVLSAYNKHRDKPIREQAENIIH
LFTLTNLGAPRAFKYFDTTIARKEYRSTKEVLDATLIHQSITGLYETRIDLSQLGGDEGADK
RTADGSEFESPKKKRKV.

Exemplary polynucleotide sequences for Variants A-K are provided below.

Variant A polynucleotide sequence:
(SEQ ID NO: 451)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAGCGGCGGGAGCTCTGGGGGCTCCTCCGGGAGCGAGACCCCCGGGACCAGCGAGTCCGCC
ACCCCCGAGAGCAGCGGCGGCAGCTCCGGGGGGAGCGACAAGAAGTACTCCATCGGCCTGGC
CATCGGCACCAACTCCGTGGGTTGGGCCGTGATCACCGATGAGTACAAGGTGCCCAGCAAGA
AGTTCAAGGTGCTGGGCAACACCGACAGGCACTCTATCAAGAAGAACCTGATCGGCGCCCTG
CTGTTCGACAGCGGGGAGACCGCTGAGGCCACTCGGCTGAAGAGAACCGCCAGGCGCAGATA
CACCCGGAGAAAGAACCGGATCTGCTACCTGCAGGAGATCTTCAGCAACGAGATGGCCAAGG
TGGACGACAGCTTCTTCCACAGGCTGGAGGAGAGCTTCCTGGTGGAGGAGGACAAGAAGCAC
GAGCGCCACCCCATCTTCGGCAACATCGTGGATGAGGTGGCCTACCACGAGAAGTACCCCAC
CATCTACCACCTGCGGAAGAAGCTGGTGGACAGCACCGATAAGGCCGATCTGCGGCTGATCT
ACCTGGCCCTGGCCCACATGATCAAGTTCAGGGGGCACTTCCTGATCGAGGGCGACCTGAAC
CCCGACAACTCCGATGTGGATAAACTGTTCATCCAGCTGGTGCAGACCTACAACCAGCTGTT
TGAGGAGAATCCCATCAACGCCTCCGGCGTGGACGCCAAGGCCATCCTGAGCGCCCGGCTGT
CCAAGAGCCGGAGGCTGGAGAATCTGATCGCCCAGCTGCCTGGCGAGAAGAAGAACGGCCTG
TTCGGCAACCTGATCGCCCTGAGCCTGGGCCTGACCCCCAATTTCAAGAGCAACTTCGACCT
GGCCGAGGATGCTAAGCTGCAGCTGTCCAAGGACACCTACGACGATGACCTGGACAACCTGC
TGGCTCAGATCGGCGATCAGTACGCCGACCTGTTCCTGGCCGCCAAGAACCTGTCAGACGCC
ATCCTGCTGTCTGACATTCTGCGGGTGAACACCGAGATCACCAAGGCCCCACTGTCCGCCTC
TATGGTGAAGAGGTACGATGAGCACCACCAGGACCTGACCCTGCTGAAGGCTCTGGTGCGGC
AGCAGCTGCCGGAGAAGTACAAGGAGATCTTCTTCGACCAGAGCAAGAACGGCTACGCCGGC
TACATCGACGGCGGGGCCAGCCAGGAGGAGTTCTACAAGTTCATCAAGCCTATCCTGGAGAA
GATGGACGGCACCGAGGAGCTGCTGGTGAAGCTGAACCGGGAGGACCTGCTGCGGAAGCAGA
GGACCTTCGACAACGGCATCATTCCCCACCAGATCCACCTGGGCGAGCTGCACGCCATCCTG
CGGAGGCAGGGCGACTTCTACCCTTTCCTGAAGGACAACAGGGAGAAGATCGAGAAGATCCT
GACCTTCAGGATCCCCTACTACGTGGGCCCCCTGGCCCGCGGCAACTCCCGCTTTGCCTGGA
TGACCAGAAAGAGCGAGGAGACCATCACCCCTTGGAACTTCGAGGAGGTGGTGGACAAGGGC
GCCAGCGCCCAGAGCTTCATCGAGCGCATGACCAACTTCGACAAGAATCTGCCCAACGAGAA
GGTGCTGCCCAAGCACTCCCTGCTGTACGAGTACTTCACCGTGTACAACGAGCTGACAAAGG
TGAAGTACGTGACAGAGGGCATGCGCAAGCCCGCCTTCCTGTCTGGCGAGCAGAAGAAGGCC
ATCGTGGACCTGCTGTTCAAGACCAACAGGAAGGTGACCGTGAAGCAGCTGAAGGAGGACTA
CTTCAAGAAGATCGAGTGCTTCGACAGCGTGGAGATCAGCGGCGTGGAGGACAGGTTCAACG
CCAGCCTGGGCACCTACCACGACCTGCTGAAGATCATCAAGGACAAGGACTTCCTGGATAAC
GAGGAGAACGAGGATATCCTGGAGGACATTGTCCTGACCCTGACCCTGTTCGAGGATCGGGA
GATGATCGAGGAGCGCCTCAAGACCTACGCCCACCTGTTCGACGATAAGGTGATGAAGCAGC
TGAAGCGGCTGCGCTACACCGGCTGGGGCCGCCTGTCCCGGAAGCTGATCAACGGCATCCGG
GACAAGCAGTCCGGCAAGACCATCCTGGACTTCCTGAAGTCCGATGGTTTTGCCAACAGAAA
CTTCATGCAGCTGATCCACGACGACAGCCTGACCTTCAAGGAGGACATCCAGAAGGCTCAGG
TGTCCGGACAGGGGGACTCCCTGCACGAGCACATCGCCAACCTGGCCGGCTCTCCCGCCATC
AAGAAGGGCATCCTGCAGACCGTGAAGGTGGTGGACGAGCTGGTGAAGGTGATGGGCGGGCA
CAAGCCCGAGAACATCGTGATCGAGATGGCCCGGGAGAACCAGACCACCCAGAAGGGCCAGA
AGAACAGCAGGGAGCGCATGAAGCGCATCGAGGAGGGCATCAAGGAGCTGGGTAGCCAGATC
CTGAAGGAGCACCCTGTGGAGAATACCCAGCTGCAGAACGAGAAGCTGTATCTGTACTACCT
GCAGAACGGCAGGGACATGTACGTGGATCAGGAGCTGGACATCAACCGGCTGTCTGACTACG
ACGTGGACCACATCGTGCCCCAGTCTTTCCTGAAGGACGACAGCATCGACAACAAGGTGCTG
ACCCGCAGCGACAAGAACAGGGGCAAGAGCGATAACGTGCCCTCCGAGGAGGTGGTCAAGAA
GATGAAGAACTACTGGCGGCAGCTGCTGAACGCCAAGCTGATCACCCAGCGGAAGTTCGACA
ACCTGACCAAGGCCGAGAGAGGCGGCCTGTCCGAGCTGGACAAGGCCGGCTTCATCAAGCGG
CAGCTGGTGGAGACCCGGCAGATCACCAAGCACGTCGCCCAGATCCTGGACTCCAGGATGAA
CACCAAGTACGACGAGAACGACAAGCTGATCAGAGAGGTGAAGGTGATCACCCTGAAGTCCA
AGCTGGTGAGCGACTTCAGGAAGGATTTCCAGTTCTACAAGGTGCGGGAGATCAACAACTAT
CACCACGCCCACGACGCCTACCTGAACGCCGTCGTGGGCACCGCTCTGATCAAGAAGTATCC
TAAGCTGGAGAGCGAGTTCGTGTACGGCGACTACAAGGTGTACGACGTGCGGAAGATGATCG
CCAAGAGCGAGCAGGAGATCGGCAAGGCCACCGCCAAGTACTTCTTCTACTCCAATATCATG
AACTTCTTCAAGACCGAGATCACCCTGGCCAACGGGGAGATCCGGAAGCGCCCACTGATCGA
GACCAACGGCGAGACCGGCGAGATCGTGTGGGACAAGGGCAGGGACTTCGCCACCGTGAGGA
AGGTGCTGTCCATGCCCCAGGTGAACATCGTGAAAAAGACCGAGGTGCAGACCGGGGGGTTC
TCCAAGGAGAGCATCCTGCCCAAGGGCAACAGCGACAAGCTGATCGCCCGGAAGAAGGACTG
GGACCCCAAGAAGTACGGAGGCTTCATGCAGCCTACCGTGGCCTACTCCGTGCTGGTGGTGG
CCAAGGTGGAGAAGGGCAAGTCCAAGAAGCTGAAGTCAGTGAAGGAGCTGCTGGGCATCACC
ATCATGGAGCGGTCTAGCTTCGAGAAGAATCCTATTGACTTCCTGGAGGCCAAGGGCTACAA
GGAGGTCAAGAAGGATCTGATCATCAAGCTGCCCAAGTACAGCCTGTTCGAGCTGGAGAATG
GCCGGAAGAGGATGCTGGCCAGCGCCAAGTTCCTGCAGAAGGGCAACGAGCTGGCCCTGCCC
AGCAAGTACGTGAACTTCCTCTATCTGGCCAGCCACTACGAGAAGCTGAAGGGCTCTCCAGA
GGACAACGAGCAGAAGCAGCTGTTCGTGGAGCAGCACAAGCACTACCTGGACGAGATCATCG
AGCAGATCTCCGAGTTCAGCAAGCGCGTGATCCTGGCCGACGCCAACCTGGACAAGGTGCTG
TCCGCTTACAACAAGCACAGGGACAAGCCCATCAGGGAGCAGGCCGAGAACATCATCCACCT
GTTCACCCTGACCAACCTGGGCGCCCCCAGGGCCTTCAAGTACTTCGATACCACCATCGCCA
GGAAGGAGTACAaAAGTACTAAGGAGGTCCTGGATGCCACTCTGATCCACCAGAGCATCACC
GGGCTGTACGAGACCCGGATCGACCTGTCCCAGCTGGGCGGCGACGAGGGCGCCGACAAGCG
GACAGCCGACGGCAGCGAGTTCGAGAGCCCCAAGAAGAAGCGGAAGGTG
Variant B polynucleotide sequence:
(SEQ ID NO: 452)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAGCGGCGGGAGCTCTGGGGGCTCCTCCGGGAGCGAGACCCCCGGGACCAGCGAGTCCGCC
ACCCCCGAGAGCAGCGGCGGCAGCTCCGGGGGGAGCGACAAGAAGTACTCCATCGGCCTGGC
CATCGGCACCAACTCCGTGGGTTGGGCCGTGATCACCGATGAGTACAAGGTGCCCAGCAAGA
AGTTCAAGGTGCTGGGCAACACCGACAGGCACTCTATCAAGAAGAACCTGATCGGCGCCCTG
CTGTTCGACAGCGGGGAGACCGCTGAGGCCACTCGGCTGAAGAGAACCGCCAGGCGCAGATA
CACCCGGAGAAAGAACCGGATCTGCTACCTGCAGGAGATCTTCAGCAACGAGATGGCCAAGG
TGGACGACAGCTTCTTCCACAGGCTGGAGGAGAGCTTCCTGGTGGAGGAGGACAAGAAGCAC
GAGCGCCACCCCATCTTCGGCAACATCGTGGATGAGGTGGCCTACCACGAGAAGTACCCCAC
CATCTACCACCTGCGGAAGAAGCTGGTGGACAGCACCGATAAGGCCGATCTGCGGCTGATCT
ACCTGGCCCTGGCCCACATGATCAAGTTCAGGGGGCACTTCCTGATCGAGGGCGACCTGAAC
CCCGACAACTCCGATGTGGATAAACTGTTCATCCAGCTGGTGCAGACCTACAACCAGCTGTT
TGAGGAGAATCCCATCAACGCCTCCGGCGTGGACGCCAAGGCCATCCTGAGCGCCCGGCTGT
CCAAGAGCCGGAGGCTGGAGAATCTGATCGCCCAGCTGCCTGGCGAGAAGAAGAACGGCCTG
TTCGGCAACCTGATCGCCCTGAGCCTGGGCCTGACCCCCAATTTCAAGAGCAACTTCGACCT
GGCCGAGGATGCTAAGCTGCAGCTGTCCAAGGACACCTACGACGATGACCTGGACAACCTGC
TGGCTCAGATCGGCGATCAGTACGCCGACCTGTTCCTGGCCGCCAAGAACCTGTCAGACGCC
ATCCTGCTGTCTGACATTCTGCGGGTGAACACCGAGATCACCAAGGCCCCACTGTCCGCCTC
TATGGTGAAGAGGTACGATGAGCACCACCAGGACCTGACCCTGCTGAAGGCTCTGGTGCGGC
AGCAGCTGCCGGAGAAGTACAAGGAGATCTTCTTCGACCAGAGCAAGAACGGCTACGCCGGC
TACATCGACGGCGGGGCCAGCCAGGAGGAGTTCTACAAGTTCATCAAGCCTATCCTGGAGAA
GATGGACGGCACCGAGGAGCTGCTGGTGAAGCTGAACCGGGAGGACCTGCTGCGGAAGCAGA
GGACCTTCGACAACGGCATCATTCCCCACCAGATCCACCTGGGCGAGCTGCACGCCATCCTG
CGGAGGCAGGGCGACTTCTACCCTTTCCTGAAGGACAACAGGGAGAAGATCGAGAAGATCCT
GACCTTCAGGATCCCCTACTACGTGGGCCCCCTGGCCCGCGGCAACTCCCGCTTTGCCTGGA
TGACCAGAAAGAGCGAGGAGACCATCACCCCTTGGAACTTCGAGGAGGTGGTGGACAAGGGC
GCCAGCGCCCAGAGCTTCATCGAGCGCATGACCAACTTCGACAAGAATCTGCCCAACGAGAA
GGTGCTGCCCAAGCACTCCCTGCTGTACGAGTACTTCACCGTGTACAACGAGCTGACAAAGG
TGAAGTACGTGACAGAGGGCATGCGCAAGCCCGCCTTCCTGTCTGGCGAGCAGAAGAAGGCC
ATCGTGGACCTGCTGTTCAAGACCAACAGGAAGGTGACCGTGAAGCAGCTGAAGGAGGACTA
CTTCAAGAAGATCGAGTGCTTCGACAGCGTGGAGATCAGCGGCGTGGAGGACAGGTTCAACG
CCAGCCTGGGCACCTACCACGACCTGCTGAAGATCATCAAGGACAAGGACTTCCTGGATAAC
GAGGAGAACGAGGATATCCTGGAGGACATTGTCCTGACCCTGACCCTGTTCGAGGATCGGGA
GATGATCGAGGAGCGCCTCAAGACCTACGCCCACCTGTTCGACGATAAGGTGATGAAGCAGC
TGAAGCGGCTGCGCTACACCGGCTGGGGCCGCCTGTCCCGGAAGCTGATCAACGGCATCCGG
GACAAGCAGTCCGGCAAGACCATCCTGGACTTCCTGAAGTCCGATGGTTTTGCCAACAGAAA
CTTCATGCAGCTGATCCACGACGACAGCCTGACCTTCAAGGAGGACATCCAGAAGGCTCAGG
TGTCCGGACAGGGGGACTCCCTGCACGAGCACATCGCCAACCTGGCCGGCTCTCCCGCCATC
AAGAAGGGCATCCTGCAGACCGTGAAGGTGGTGGACGAGCTGGTGAAGGTGATGGGCGGGCA
CAAGCCCGAGAACATCGTGATCGAGATGGCCCGGGAGAACCAGACCACCCAGAAGGGCCAGA
AGAACAGCAGGGAGCGCATGAAGCGCATCGAGGAGGGCATCAAGGAGCTGGGTAGCCAGATC
CTGAAGGAGCACCCTGTGGAGAATACCCAGCTGCAGAACGAGAAGCTGTATCTGTACTACCT
GCAGAACGGCAGGGACATGTACGTGGATCAGGAGCTGGACATCAACCGGCTGTCTGACTACG
ACGTGGACCACATCGTGCCCCAGTCTTTCCTGAAGGACGACAGCATCGACAACAAGGTGCTG
ACCCGCAGCGACAAGAACAGGGGCAAGAGCGATAACGTGCCCTCCGAGGAGGTGGTCAAGAA
GATGAAGAACTACTGGCGGCAGCTGCTGAACGCCAAGCTGATCACCCAGCGGAAGTTCGACA
ACCTGACCAAGGCCGAGAGAGGCGGCCTGTCCGAGCTGGACAAGGCCGGCTTCATCAAGCGG
CAGCTGGTGGAGACCCGGCAGATCACCAAGCACGTCGCCCAGATCCTGGACTCCAGGATGAA
CACCAAGTACGACGAGAACGACAAGCTGATCAGAGAGGTGAAGGTGATCACCCTGAAGTCCA
AGCTGGTGAGCGACTTCAGGAAGGATTTCCAGTTCTACAAGGTGCGGGAGATCAACAACTAT
CACCACGCCCACGACGCCTACCTGAACGCCGTCGTGGGCACCGCTCTGATCAAGAAGTATCC
TAAGCTGGAGAGCGAGTTCGTGTACGGCGACTACAAGGTGTACGACGTGCGGAAGATGATCG
CCAAGAGCGAGCAGGAGATCGGCAAGGCCACCGCCAAGTACTTCTTCTACTCCAATATCATG
AACTTCTTCAAGACCGAGATCACCCTGGCCAACGGGGAGATCCGGAAGCGCCCACTGATCGA
GACCAACGGCGAGACCGGCGAGATCGTGTGGGACAAGGGCAGGGACTTCGCCACCGTGAGGA
AGGTGCTGTCCATGCCCCAGGTGAACATCGTGAAAAAGACCGAGGTGCAGACCGGGGGGTTC
TCCAAGGAGAGCATCCTGCCCAAGGGCAACAGCGACAAGCTGATCGCCCGGAAGAAGGACTG
GGACCCCAAGAAGTACGGAGGCTTCATGtAtCCTACCGTGGCCTACTCCGTGCTGGTGGTGG
CCAAGGTGGAGAAGGGCAAGTCCAAGAAGCTGAAGTCAGTGAAGGAGCTGCTGGGCATCACC
ATCATGGAGCGGTCTAGCTTCGAGAAGAATCCTATTGACTTCCTGGAGGCCAAGGGCTACAA
GGAGGTCAAGAAGGATCTGATCATCAAGCTGCCCAAGTACAGCCTGTTCGAGCTGGAGAATG
GCCGGAAGAGGATGCTGGCCAGCGCCAAGTTCCTGCAGAAGGGCAACGAGCTGGCCCTGCCC
AGCAAGTACGTGAACTTCCTCTATCTGGCCAGCCACTACGAGAAGCTGAAGGGCTCTCCAGA
GGACAACGAGCAGAAGCAGCTGTTCGTGGAGCAGCACAAGCACTACCTGGACGAGATCATCG
AGCAGATCTCCGAGTTCAGCAAGCGCGTGATCCTGGCCGACGCCAACCTGGACAAGGTGCTG
TCCGCTTACAACAAGCACAGGGACAAGCCCATCAGGGAGCAGGCCGAGAACATCATCCACCT
GTTCACCCTGACCAACCTGGGCGCCCCCAGGGCCTTCAAGTACTTCGATACCACCATCGCCA
GGAAGGAGTACAaAAGTACTAAGGAGGTCCTGGATGCCACTCTGATCCACCAGAGCATCACC
GGGCTGTACGAGACCCGGATCGACCTGTCCCAGCTGGGCGGCGACGAGGGCGCCGACAAGCG
GACAGCCGACGGCAGCGAGTTCGAGAGCCCCAAGAAGAAGCGGAAGGTG
Variant C polynucleotide sequence:
(SEQ ID NO: 453)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACGAGGGCGGATCTGAGGAAGAGGAAGAGAGCGGCAGCGACAAGAAGTACTCCATCGGCCTG
GCCATCGGCACCAACTCCGTGGGTTGGGCCGTGATCACCGATGAGTACAAGGTGCCCAGCAA
GAAGTTCAAGGTGCTGGGCAACACCGACAGGCACTCTATCAAGAAGAACCTGATCGGCGCCC
TGCTGTTCGACAGCGGGGAGACCGCTGAGGCCACTCGGCTGAAGAGAACCGCCAGGCGCAGA
TACACCCGGAGAAAGAACCGGATCTGCTACCTGCAGGAGATCTTCAGCAACGAGATGGCCAA
GGTGGACGACAGCTTCTTCCACAGGCTGGAGGAGAGCTTCCTGGTGGAGGAGGACAAGAAGC
ACGAGCGCCACCCCATCTTCGGCAACATCGTGGATGAGGTGGCCTACCACGAGAAGTACCCC
ACCATCTACCACCTGCGGAAGAAGCTGGTGGACAGCACCGATAAGGCCGATCTGCGGCTGAT
CTACCTGGCCCTGGCCCACATGATCAAGTTCAGGGGGCACTTCCTGATCGAGGGCGACCTGA
ACCCCGACAACTCCGATGTGGATAAACTGTTCATCCAGCTGGTGCAGACCTACAACCAGCTG
TTTGAGGAGAATCCCATCAACGCCTCCGGCGTGGACGCCAAGGCCATCCTGAGCGCCCGGCT
GTCCAAGAGCCGGAGGCTGGAGAATCTGATCGCCCAGCTGCCTGGCGAGAAGAAGAACGGCC
TGTTCGGCAACCTGATCGCCCTGAGCCTGGGCCTGACCCCCAATTTCAAGAGCAACTTCGAC
CTGGCCGAGGATGCTAAGCTGCAGCTGTCCAAGGACACCTACGACGATGACCTGGACAACCT
GCTGGCTCAGATCGGCGATCAGTACGCCGACCTGTTCCTGGCCGCCAAGAACCTGTCAGACG
CCATCCTGCTGTCTGACATTCTGCGGGTGAACACCGAGATCACCAAGGCCCCACTGTCCGCC
TCTATGGTGAAGAGGTACGATGAGCACCACCAGGACCTGACCCTGCTGAAGGCTCTGGTGCG
GCAGCAGCTGCCGGAGAAGTACAAGGAGATCTTCTTCGACCAGAGCAAGAACGGCTACGCCG
GCTACATCGACGGCGGGGCCAGCCAGGAGGAGTTCTACAAGTTCATCAAGCCTATCCTGGAG
AAGATGGACGGCACCGAGGAGCTGCTGGTGAAGCTGAACCGGGAGGACCTGCTGCGGAAGCA
GAGGACCTTCGACAACGGCATCATTCCCCACCAGATCCACCTGGGCGAGCTGCACGCCATCC
TGCGGAGGCAGGGCGACTTCTACCCTTTCCTGAAGGACAACAGGGAGAAGATCGAGAAGATC
CTGACCTTCAGGATCCCCTACTACGTGGGCCCCCTGGCCCGCGGCAACTCCCGCTTTGCCTG
GATGACCAGAAAGAGCGAGGAGACCATCACCCCTTGGAACTTCGAGGAGGTGGTGGACAAGG
GCGCCAGCGCCCAGAGCTTCATCGAGCGCATGACCAACTTCGACAAGAATCTGCCCAACGAG
AAGGTGCTGCCCAAGCACTCCCTGCTGTACGAGTACTTCACCGTGTACAACGAGCTGACAAA
GGTGAAGTACGTGACAGAGGGCATGCGCAAGCCCGCCTTCCTGTCTGGCGAGCAGAAGAAGG
CCATCGTGGACCTGCTGTTCAAGACCAACAGGAAGGTGACCGTGAAGCAGCTGAAGGAGGAC
TACTTCAAGAAGATCGAGTGCTTCGACAGCGTGGAGATCAGCGGCGTGGAGGACAGGTTCAA
CGCCAGCCTGGGCACCTACCACGACCTGCTGAAGATCATCAAGGACAAGGACTTCCTGGATA
ACGAGGAGAACGAGGATATCCTGGAGGACATTGTCCTGACCCTGACCCTGTTCGAGGATCGG
GAGATGATCGAGGAGCGCCTCAAGACCTACGCCCACCTGTTCGACGATAAGGTGATGAAGCA
GCTGAAGCGGCTGCGCTACACCGGCTGGGGCCGCCTGTCCCGGAAGCTGATCAACGGCATCC
GGGACAAGCAGTCCGGCAAGACCATCCTGGACTTCCTGAAGTCCGATGGTTTTGCCAACAGA
AACTTCATGCAGCTGATCCACGACGACAGCCTGACCTTCAAGGAGGACATCCAGAAGGCTCA
GGTGTCCGGACAGGGGGACTCCCTGCACGAGCACATCGCCAACCTGGCCGGCTCTCCCGCCA
TCAAGAAGGGCATCCTGCAGACCGTGAAGGTGGTGGACGAGCTGGTGAAGGTGATGGGCGGG
CACAAGCCCGAGAACATCGTGATCGAGATGGCCCGGGAGAACCAGACCACCCAGAAGGGCCA
GAAGAACAGCAGGGAGCGCATGAAGCGCATCGAGGAGGGCATCAAGGAGCTGGGTAGCCAGA
TCCTGAAGGAGCACCCTGTGGAGAATACCCAGCTGCAGAACGAGAAGCTGTATCTGTACTAC
CTGCAGAACGGCAGGGACATGTACGTGGATCAGGAGCTGGACATCAACCGGCTGTCTGACTA
CGACGTGGACCACATCGTGCCCCAGTCTTTCCTGAAGGACGACAGCATCGACAACAAGGTGC
TGACCCGCAGCGACAAGAACAGGGGCAAGAGCGATAACGTGCCCTCCGAGGAGGTGGTCAAG
AAGATGAAGAACTACTGGCGGCAGCTGCTGAACGCCAAGCTGATCACCCAGCGGAAGTTCGA
CAACCTGACCAAGGCCGAGAGAGGCGGCCTGTCCGAGCTGGACAAGGCCGGCTTCATCAAGC
GGCAGCTGGTGGAGACCCGGCAGATCACCAAGCACGTCGCCCAGATCCTGGACTCCAGGATG
AACACCAAGTACGACGAGAACGACAAGCTGATCAGAGAGGTGAAGGTGATCACCCTGAAGTC
CAAGCTGGTGAGCGACTTCAGGAAGGATTTCCAGTTCTACAAGGTGCGGGAGATCAACAACT
ATCACCACGCCCACGACGCCTACCTGAACGCCGTCGTGGGCACCGCTCTGATCAAGAAGTAT
CCTAAGCTGGAGAGCGAGTTCGTGTACGGCGACTACAAGGTGTACGACGTGCGGAAGATGAT
CGCCAAGAGCGAGCAGGAGATCGGCAAGGCCACCGCCAAGTACTTCTTCTACTCCAATATCA
TGAACTTCTTCAAGACCGAGATCACCCTGGCCAACGGGGAGATCCGGAAGCGCCCACTGATC
GAGACCAACGGCGAGACCGGCGAGATCGTGTGGGACAAGGGCAGGGACTTCGCCACCGTGAG
GAAGGTGCTGTCCATGCCCCAGGTGAACATCGTGAAAAAGACCGAGGTGCAGACCGGGGGGT
TCTCCAAGGAGAGCATCCTGCCCAAGGGCAACAGCGACAAGCTGATCGCCCGGAAGAAGGAC
TGGGACCCCAAGAAGTACGGAGGCTTCtTGtAtCCTACCGTGGCCTACTCCGTGCTGGTGGT
GGCCAAGGTGGAGAAGGGCAAGTCCAAGAAGCTGAAGTCAGTGAAGGAGCTGCTGGGCATCA
CCATCATGGAGCGGTCTAGCTTCGAGAAGAATCCTATTGACTTCCTGGAGGCCAAGGGCTAC
AAGGAGGTCAAGAAGGATCTGATCATCAAGCTGCCCAAGTACAGCCTGTTCGAGCTGGAGAA
TGGCCGGAAGAGGATGCTGGCCAGCGCCAAGTTCCTGCAGAAGGGCAACGAGCTGGCCCTGC
CCAGCAAGTACGTGAACTTCCTCTATCTGGCCAGCCACTACGAGAAGCTGAAGGGCTCTCCA
GAGGACAACGAGCAGAAGCAGCTGTTCGTGGAGCAGCACAAGCACTACCTGGACGAGATCAT
CGAGCAGATCTCCGAGTTCAGCAAGCGCGTGATCCTGGCCGACGCCAACCTGGACAAGGTGC
TGTCCGCTTACAACAAGCACAGGGACAAGCCCATCAGGGAGCAGGCCGAGAACATCATCCAC
CTGTTCACCCTGACCAACCTGGGCGCCCCCAGGGCCTTCAAGTACTTCGATACCACCATCGC
CAGGAAGGAGTACAaAAGTACTAAGGAGGTCCTGGATGCCACTCTGATCCACCAGAGCATCA
CCGGGCTGTACGAGACCCGGATCGACCTGTCCCAGCTGGGCGGCGACGAGGGCGCCGACAAG
CGGACAGCCGACGGCAGCGAGTTCGAGAGCCCCAAGAAGAAGCGGAAGGTG
Variant D polynucleotide sequence:
(SEQ ID NO: 454)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACGAGGGCGGATCTGAGGAAGAGGAAGAGAGCGGCAGCGACAAGAAGTACTCCATCGGCCTG
GCCATCGGCACCAACTCCGTGGGTTGGGCCGTGATCACCGATGAGTACAAGGTGCCCAGCAA
GAAGTTCAAGGTGCTGGGCAACACCGACAGGCACTCTATCAAGAAGAACCTGATCGGCGCCC
TGCTGTTCGACAGCGGGGAGACCGCTGAGGCCACTCGGCTGAAGAGAACCGCCAGGCGCAGA
TACACCCGGAGAAAGAACCGGATCTGCTACCTGCAGGAGATCTTCAGCAACGAGATGGCCAA
GGTGGACGACAGCTTCTTCCACAGGCTGGAGGAGAGCTTCCTGGTGGAGGAGGACAAGAAGC
ACGAGCGCCACCCCATCTTCGGCAACATCGTGGATGAGGTGGCCTACCACGAGAAGTACCCC
ACCATCTACCACCTGCGGAAGAAGCTGGTGGACAGCACCGATAAGGCCGATCTGCGGCTGAT
CTACCTGGCCCTGGCCCACATGATCAAGTTCAGGGGGCACTTCCTGATCGAGGGCGACCTGA
ACCCCGACAACTCCGATGTGGATAAACTGTTCATCCAGCTGGTGCAGACCTACAACCAGCTG
TTTGAGGAGAATCCCATCAACGCCTCCGGCGTGGACGCCAAGGCCATCCTGAGCGCCCGGCT
GTCCAAGAGCCGGAGGCTGGAGAATCTGATCGCCCAGCTGCCTGGCGAGAAGAAGAACGGCC
TGTTCGGCAACCTGATCGCCCTGAGCCTGGGCCTGACCCCCAATTTCAAGAGCAACTTCGAC
CTGGCCGAGGATGCTAAGCTGCAGCTGTCCAAGGACACCTACGACGATGACCTGGACAACCT
GCTGGCTCAGATCGGCGATCAGTACGCCGACCTGTTCCTGGCCGCCAAGAACCTGTCAGACG
CCATCCTGCTGTCTGACATTCTGCGGGTGAACACCGAGATCACCAAGGCCCCACTGTCCGCC
TCTATGGTGAAGAGGTACGATGAGCACCACCAGGACCTGACCCTGCTGAAGGCTCTGGTGCG
GCAGCAGCTGCCGGAGAAGTACAAGGAGATCTTCTTCGACCAGAGCAAGAACGGCTACGCCG
GCTACATCGACGGCGGGGCCAGCCAGGAGGAGTTCTACAAGTTCATCAAGCCTATCCTGGAG
AAGATGGACGGCACCGAGGAGCTGCTGGTGAAGCTGAACCGGGAGGACCTGCTGCGGAAGCA
GAGGACCTTCGACAACGGCATCATTCCCCACCAGATCCACCTGGGCGAGCTGCACGCCATCC
TGCGGAGGCAGGGCGACTTCTACCCTTTCCTGAAGGACAACAGGGAGAAGATCGAGAAGATC
CTGACCTTCAGGATCCCCTACTACGTGGGCCCCCTGGCCCGCGGCAACTCCCGCTTTGCCTG
GATGACCAGAAAGAGCGAGGAGACCATCACCCCTTGGAACTTCGAGGAGGTGGTGGACAAGG
GCGCCAGCGCCCAGAGCTTCATCGAGCGCATGACCAACTTCGACAAGAATCTGCCCAACGAG
AAGGTGCTGCCCAAGCACTCCCTGCTGTACGAGTACTTCACCGTGTACAACGAGCTGACAAA
GGTGAAGTACGTGACAGAGGGCATGCGCAAGCCCGCCTTCCTGTCTGGCGAGCAGAAGAAGG
CCATCGTGGACCTGCTGTTCAAGACCAACAGGAAGGTGACCGTGAAGCAGCTGAAGGAGGAC
TACTTCAAGAAGATCGAGTGCTTCGACAGCGTGGAGATCAGCGGCGTGGAGGACAGGTTCAA
CGCCAGCCTGGGCACCTACCACGACCTGCTGAAGATCATCAAGGACAAGGACTTCCTGGATA
ACGAGGAGAACGAGGATATCCTGGAGGACATTGTCCTGACCCTGACCCTGTTCGAGGATCGG
GAGATGATCGAGGAGCGCCTCAAGACCTACGCCCACCTGTTCGACGATAAGGTGATGAAGCA
GCTGAAGCGGCTGCGCTACACCGGCTGGGGCCGCCTGTCCCGGAAGCTGATCAACGGCATCC
GGGACAAGCAGTCCGGCAAGACCATCCTGGACTTCCTGAAGTCCGATGGTTTTGCCAACAGA
AACTTCATGCAGCTGATCCACGACGACAGCCTGACCTTCAAGGAGGACATCCAGAAGGCTCA
GGTGTCCGGACAGGGGGACTCCCTGCACGAGCACATCGCCAACCTGGCCGGCTCTCCCGCCA
TCAAGAAGGGCATCCTGCAGACCGTGAAGGTGGTGGACGAGCTGGTGAAGGTGATGGGCGGG
CACAAGCCCGAGAACATCGTGATCGAGATGGCCCGGGAGAACCAGACCACCCAGAAGGGCCA
GAAGAACAGCAGGGAGCGCATGAAGCGCATCGAGGAGGGCATCAAGGAGCTGGGTAGCCAGA
TCCTGAAGGAGCACCCTGTGGAGAATACCCAGCTGCAGAACGAGAAGCTGTATCTGTACTAC
CTGCAGAACGGCAGGGACATGTACGTGGATCAGGAGCTGGACATCAACCGGCTGTCTGACTA
CGACGTGGACCACATCGTGCCCCAGTCTTTCCTGAAGGACGACAGCATCGACAACAAGGTGC
TGACCCGCAGCGACAAGAACAGGGGCAAGAGCGATAACGTGCCCTCCGAGGAGGTGGTCAAG
AAGATGAAGAACTACTGGCGGCAGCTGCTGAACGCCAAGCTGATCACCCAGCGGAAGTTCGA
CAACCTGACCAAGGCCGAGAGAGGCGGCCTGTCCGAGCTGGACAAGGCCGGCTTCATCAAGC
GGCAGCTGGTGGAGACCCGGCAGATCACCAAGCACGTCGCCCAGATCCTGGACTCCAGGATG
AACACCAAGTACGACGAGAACGACAAGCTGATCAGAGAGGTGAAGGTGATCACCCTGAAGTC
CAAGCTGGTGAGCGACTTCAGGAAGGATTTCCAGTTCTACAAGGTGCGGGAGATCAACAACT
ATCACCACGCCCACGACGCCTACCTGAACGCCGTCGTGGGCACCGCTCTGATCAAGAAGTAT
CCTAAGCTGGAGAGCGAGTTCGTGTACGGCGACTACAAGGTGTACGACGTGCGGAAGATGAT
CGCCAAGAGCGAGCAGGAGATCGGCAAGGCCACCGCCAAGTACTTCTTCTACTCCAATATCA
TGAACTTCTTCAAGACCGAGATCACCCTGGCCAACGGGGAGATCCGGAAGCGCCCACTGATC
GAGACCAACGGCGAGACCGGCGAGATCGTGTGGGACAAGGGCAGGGACTTCGCCACCGTGAG
GAAGGTGCTGTCCATGCCCCAGGTGAACATCGTGAAAAAGACCGAGGTGCAGACCGGGGGGT
TCTCCAAGGAGAGCATCCTGCCCAAGGGCAACAGCGACAAGCTGATCGCCCGGAAGAAGGAC
TGGGACCCCAAGAAGTACGGAGGCTTCATGtAtCCTACCGTGGCCTACTCCGTGCTGGTGGT
GGCCAAGGTGGAGAAGGGCAAGTCCAAGAAGCTGAAGTCAGTGAAGGAGCTGCTGGGCATCA
CCATCATGGAGCGGTCTAGCTTCGAGAAGAATCCTATTGACTTCCTGGAGGCCAAGGGCTAC
AAGGAGGTCAAGAAGGATCTGATCATCAAGCTGCCCAAGTACAGCCTGTTCGAGCTGGAGAA
TGGCCGGAAGAGGATGCTGGCCAGCGCCAAGTTCCTGCAGAAGGGCAACGAGCTGGCCCTGC
CCAGCAAGTACGTGAACTTCCTCTATCTGGCCAGCCACTACGAGAAGCTGAAGGGCTCTCCA
GAGGACAACGAGCAGAAGCAGCTGTTCGTGGAGCAGCACAAGCACTACCTGGACGAGATCAT
CGAGCAGATCTCCGAGTTCAGCAAGCGCGTGATCCTGGCCGACGCCAACCTGGACAAGGTGC
TGTCCGCTTACAACAAGCACAGGGACAAGCCCATCAGGGAGCAGGCCGAGAACATCATCCAC
CTGTTCACCCTGACCAACCTGGGCGCCCCCAGGGCCTTCAAGTACTTCGATACCACCATCGC
CAGGAAGGAGTACAaAAGTACTAAGGAGGTCCTGGATGCCACTCTGATCCACCAGAGCATCA
CCGGGCTGTACGAGACCCGGATCGACCTGTCCCAGCTGGGCGGCGACGAGGGCGCCGACAAG
CGGACAGCCGACGGCAGCGAGTTCGAGAGCCCCAAGAAGAAGCGGAAGGTG
Variant E polynucleotide sequence:
(SEQ ID NO: 455)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAAAGGCCCTAAGCCCAAGAAGGAAGAGAGCGAGAAGGACAAGAAGTACTCCATCGGCCTG
GCCATCGGCACCAACTCCGTGGGTTGGGCCGTGATCACCGATGAGTACAAGGTGCCCAGCAA
GAAGTTCAAGGTGCTGGGCAACACCGACAGGCACTCTATCAAGAAGAACCTGATCGGCGCCC
TGCTGTTCGACAGCGGGGAGACCGCTGAGGCCACTCGGCTGAAGAGAACCGCCAGGCGCAGA
TACACCCGGAGAAAGAACCGGATCTGCTACCTaCAGGAGATCTTCAGCAACGAGATGGCCAA
GGTGGACGACAGCTTCTTCCACAGGCTGGAGGAGAGCTTCCTGGTGGAGGAGGACAAGAAGC
ACGAGCGCCACCCCATCTTCGGCAACATCGTGGATGAGGTGGCCTACCACGAGAAGTACCCC
ACCATCTACCACCTGCGGAAGAAGCTGGTGGACAGCACCGATAAGGCCGATCTGCGGCTGAT
CTACCTGGCCCTGGCCCACATGATCAAGTTCAGGGGGCACTTCCTGATCGAGGGCGACCTGA
ACCCCGACAACTCCGATGTGGATAAACTGTTCATCCAGCTGGTGCAGACCTACAACCAGCTG
TTTGAGGAGAATCCCATCAACGCCTCCGGCGTGGACGCCAAGGCCATCCTGAGCGCCCGGCT
GTCCAAGAGCCGGAGGCTGGAGAATCTGATCGCCCAGCTGCCTGGCGAGAAGAAGAACGGCC
TGTTCGGCAACCTGATCGCCCTGAGCCTGGGCCTGACCCCCAATTTCAAGAGCAACTTCGAC
CTGGCCGAGGATGCTAAGCTGCAGCTGTCCAAGGACACCTACGACGATGACCTGGACAACCT
GCTGGCTCAGATCGGCGATCAGTACGCCGACCTGTTCCTGGCCGCCAAGAACCTGTCAGACG
CCATCCTGCTGTCTGACATTCTGCGGGTGAACACCGAGATCACCAAGGCCCCACTGTCCGCC
TCTATGGTGAAGAGGTACGATGAGCACCACCAGGACCTGACCCTGCTGAAGGCTCTGGTGCG
GCAGCAGCTGCCGGAGAAGTACAAGGAGATCTTCTTCGACCAGAGCAAGAACGGCTACGCCG
GCTACATCGACGGCGGGGCCAGCCAGGAGGAGTTCTACAAGTTCATCAAGCCTATCCTGGAG
AAGATGGACGGCACCGAGGAGCTGCTGGTGAAGCTGAACCGGGAGGACCTGCTGCGGAAGCA
GAGGACCTTCGACAACGGCATCATTCCCCACCAGATCCACCTGGGCGAGCTGCACGCCATCC
TGCGGAGGCAGGGCGACTTCTACCCTTTCCTGAAGGACAACAGGGAGAAGATCGAGAAGATC
CTGACCTTCAGGATCCCCTACTACGTGGGCCCCCTGGCCCGCGGCAACTCCCGCTTTGCCTG
GATGACCAGAAAGAGCGAGGAGACCATCACCCCTTGGAACTTCGAGGAGGTGGTGGACAAGG
GCGCCAGCGCCCAGAGCTTCATCGAGCGCATGACCAACTTCGACAAGAATCTGCCCAACGAG
AAGGTGCTGCCCAAGCACTCCCTGCTGTACGAGTACTTCACCGTGTACAACGAGCTGACAAA
GGTGAAGTACGTGACAGAGGGCATGCGCAAGCCCGCCTTCCTGTCTGGCGAGCAGAAGAAGG
CCATCGTGGACCTGCTGTTCAAGACCAACAGGAAGGTGACCGTGAAGCAGCTGAAGGAGGAC
TACTTCAAGAAGATCGAGTGCTTCGACAGCGTGGAGATCAGCGGCGTGGAGGACAGGTTCAA
CGCCAGCCTGGGCACCTACCACGACCTGCTGAAGATCATCAAGGACAAGGACTTCCTGGATA
ACGAGGAGAACGAGGATATCCTGGAGGACATTGTCCTGACCCTGACCCTGTTCGAGGATCGG
GAGATGATCGAGGAGCGCCTCAAGACCTACGCCCACCTGTTCGACGATAAGGTGATGAAGCA
GCTGAAGCGGCTGCGCTACACCGGCTGGGGCCGCCTGTCCCGGAAGCTGATCAACGGCATCC
GGGACAAGCAGTCCGGCAAGACCATCCTGGACTTCCTGAAGTCCGATGGTTTTGCCAACAGA
AACTTCATGCAGCTGATCCACGACGACAGCCTGACCTTCAAGGAGGACATCCAGAAGGCTCA
GGTGTCCGGACAGGGGGACTCCCTGCACGAGCACATCGCCAACCTGGCCGGCTCTCCCGCCA
TCAAGAAGGGCATCCTGCAGACCGTGAAGGTGGTGGACGAGCTGGTGAAGGTGATGGGCGGG
CACAAGCCCGAGAACATCGTGATCGAGATGGCCCGGGAGAACCAGACCACCCAGAAGGGCCA
GAAGAACAGCAGGGAGCGCATGAAGCGCATCGAGGAGGGCATCAAGGAGCTGGGTAGCCAGA
TCCTGAAGGAGCACCCTGTGGAGAATACCCAGCTGCAGAACGAGAAGCTGTATCTGTACTAC
CTGCAGAACGGCAGGGACATGTACGTGGATCAGGAGCTGGACATCAACCGGCTGTCTGACTA
CGACGTGGACCACATCGTGCCCCAGTCTTTCCTGAAGGACGACAGCATCGACAACAAGGTGC
TGACCCGCAGCGACAAGAACAGGGGCAAGAGCGATAACGTGCCCTCCGAGGAGGTGGTCAAG
AAGATGAAGAACTACTGGCGGCAGCTGCTGAACGCCAAGCTGATCACCCAGCGGAAGTTCGA
CAACCTGACCAAGGCCGAGAGAGGCGGCCTGTCCGAGCTGGACAAGGCCGGCTTCATCAAGC
GGCAGCTGGTGGAGACCCGGCAGATCACCAAGCACGTCGCCCAGATCCTGGACTCCAGGATG
AACACCAAGTACGACGAGAACGACAAGCTGATCAGAGAGGTGAAGGTGATCACCCTGAAGTC
CAAGCTGGTGAGCGACTTCAGGAAGGATTTCCAGTTCTACAAGGTGCGGGAGATCAACAACT
ATCACCACGCCCACGACGCCTACCTGAACGCCGTCGTGGGCACCGCTCTGATCAAGAAGTAT
CCTAAGCTGGAGAGCGAGTTCGTGTACGGCGACTACAAGGTGTACGACGTGCGGAAGATGAT
CGCCAAGAGCGAGCAGGAGATCGGCAAGGCCACCGCCAAGTACTTCTTCTACTCCAATATCA
TGAACTTCTTCAAGACCGAGATCACCCTGGCCAACGGGGAGATCCGGAAGCGCCCACTGATC
GAGACCAACGGCGAGACCGGCGAGATCGTGTGGGACAAGGGCAGGGACTTCGCCACCGTGAG
GAAGGTGCTGTCCATGCCCCAGGTGAACATCGTGAAAAAGACCGAGGTGCAGACCGGGGGGT
TCTCCAAGGAGAGCATCCTGCCCAAGGGCAACAGCGACAAGCTGATCGCCCGGAAGAAGGAC
TGGGACCCCAAGAAGTACGGAGGCTTCtTGtAtCCTACCGTGGCCTACTCCGTGCTGGTGGT
GGCCAAGGTGGAGAAGGGCAAGTCCAAGAAGCTGAAGTCAGTGAAGGAGCTGCTGGGCATCA
CCATCATGGAGCGGTCTAGCTTCGAGAAGAATCCTATTGACTTCCTGGAGGCCAAGGGCTAC
AAGGAGGTCAAGAAGGATCTGATCATCAAGCTGCCCAAGTACAGCCTGTTCGAGCTGGAGAA
TGGCCGGAAGAGGATGCTGGCCAGCGCCAAGTTCCTGCAGAAGGGCAACGAGCTGGCCCTGC
CCAGCAAGTACGTGAACTTCCTCTATCTGGCCAGCCACTACGAGAAGCTGAAGGGCTCTCCA
GAGGACAACGAGCAGAAGCAGCTGTTCGTGGAGCAGCACAAGCACTACCTGGACGAGATCAT
CGAGCAGATCTCCGAGTTCAGCAAGCGCGTGATCCTGGCCGACGCCAACCTGGACAAGGTGC
TGTCCGCTTACAACAAGCACAGGGACAAGCCCATCAGGGAGCAGGCCGAGAACATCATCCAC
CTGTTCACCCTGACCAACCTGGGCGCCCCCAGGGCCTTCAAGTACTTCGATACCACCATCGC
CAGGAAGGAGTACAaAAGTACTAAGGAGGTCCTGGATGCCACTCTGATCCACCAGAGCATCA
CCGGGCTGTACGAGACCCGGATCGACCTGTCCCAGCTGGGCGGCGACGAGGGCGCCGACAAG
CGGACAGCCGACGGCAGCGAGTTCGAGAGCCCCAAGAAGAAGCGGAAGGTG
Variant F polynucleotide sequence:
(SEQ ID NO: 456)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAGCGGCGGGAGCTCTGGGGGCTCCTCCGGGAGCGAGACCCCCGGGACCAGCGAGTCCGCC
ACCCCCGAGAGCAGCGGCGGCAGCTCCGGGGGGAGCGACAAGAAGTACTCCATCGGCCTGGC
CATCGGCACCAACTCCGTGGGTTGGGCCGTGATCACCGATGAGTACAAGGTGCCCAGCAAGA
AGTTCAAGGTGCTGGGCAACACCGACAGGCACTCTATCAAGAAGAACCTGATCGGCGCCCTG
CTGTTCGACAGCGGGGAGACCGCTGAGGCCACTCGGCTGAAGAGAACCGCCAGGCGCAGATA
CACCCGGAGAAAGAACCGGATCTGCTACCTGCAGGAGATCTTCAGCAACGAGATGGCCAAGG
TGGACGACAGCTTCTTCCACAGGCTGGAGGAGAGCTTCCTGGTGGAGGAGGACAAGAAGCAC
GAGCGCCACCCCATCTTCGGCAACATCGTGGATGAGGTGGCCTACCACGAGAAGTACCCCAC
CATCTACCACCTGCGGAAGAAGCTGGTGGACAGCACCGATAAGGCCGATCTGCGGCTGATCT
ACCTGGCCCTGGCCCACATGATCAAGTTCAGGGGGCACTTCCTGATCGAGGGCGACCTGAAC
CCCGACAACTCCGATGTGGATAAACTGTTCATCCAGCTGGTGCAGACCTACAACCAGCTGTT
TGAGGAGAATCCCATCAACGCCTCCGGCGTGGACGCCAAGGCCATCCTGAGCGCCCGGCTGT
CCAAGAGCCGGAGGCTGGAGAATCTGATCGCCCAGCTGCCTGGCGAGAAGAAGAACGGCCTG
TTCGGCAACCTGATCGCCCTGAGCCTGGGCCTGACCCCCAATTTCAAGAGCAACTTCGACCT
GGCCGAGGATGCTAAGCTGCAGCTGTCCAAGGACACCTACGACGATGACCTGGACAACCTGC
TGGCTCAGATCGGCGATCAGTACGCCGACCTGTTCCTGGCCGCCAAGAACCTGTCAGACGCC
ATCCTGCTGTCTGACATTCTGCGGGTGAACACCGAGATCACCAAGGCCCCACTGTCCGCCTC
TATGGTGAAGAGGTACGATGAGCACCACCAGGACCTGACCCTGCTGAAGGCTCTGGTGCGGC
AGCAGCTGCCGGAGAAGTACAAGGAGATCTTCTTCGACCAGAGCAAGAACGGCTACGCCGGC
TACATCGACGGCGGGGCCAGCCAGGAGGAGTTCTACAAGTTCATCAAGCCTATCCTGGAGAA
GATGGACGGCACCGAGGAGCTGCTGGTGAAGCTGAACCGGGAGGACCTGCTGCGGAAGCAGA
GGACCTTCGACAACGGCATCATTCCCCACCAGATCCACCTGGGCGAGCTGCACGCCATCCTG
CGGAGGCAGGGCGACTTCTACCCTTTCCTGAAGGACAACAGGGAGAAGATCGAGAAGATCCT
GACCTTCAGGATCCCCTACTACGTGGGCCCCCTGGCCCGCGGCAACTCCCGCTTTGCCTGGA
TGACCAGAAAGAGCGAGGAGACCATCACCCCTTGGAACTTCGAGGAGGTGGTGGACAAGGGC
GCCAGCGCCCAGAGCTTCATCGAGCGCATGACCAACTTCGACAAGAATCTGCCCAACGAGAA
GGTGCTGCCCAAGCACTCCCTGCTGTACGAGTACTTCACCGTGTACAACGAGCTGACAAAGG
TGAAGTACGTGACAGAGGGCATGCGCAAGCCCGCCTTCCTGTCTGGCGAGCAGAAGAAGGCC
ATCGTGGACCTGCTGTTCAAGACCAACAGGAAGGTGACCGTGAAGCAGCTGAAGGAGGACTA
CTTCAAGAAGATCGAGTGCTTCGACAGCGTGGAGATCAGCGGCGTGGAGGACAGGTTCAACG
CCAGCCTGGGCACCTACCACGACCTGCTGAAGATCATCAAGGACAAGGACTTCCTGGATAAC
GAGGAGAACGAGGATATCCTGGAGGACATTGTCCTGACCCTGACCCTGTTCGAGGATCGGGA
GATGATCGAGGAGCGCCTCAAGACCTACGCCCACCTGTTCGACGATAAGGTGATGAAGCAGC
TGAAGCGGCTGCGCTACACCGGCTGGGGCCGCCTGTCCCGGAAGCTGATCAACGGCATCCGG
GACAAGCAGTCCGGCAAGACCATCCTGGACTTCCTGAAGTCCGATGGTTTTGCCAACAGAAA
CTTCATGCAGCTGATCCACGACGACAGCCTGACCTTCAAGGAGGACATCCAGAAGGCTCAGG
TGTCCGGACAGGGGGACTCCCTGCACGAGCACATCGCCAACCTGGCCGGCTCTCCCGCCATC
AAGAAGGGCATCCTGCAGACCGTGAAGGTGGTGGACGAGCTGGTGAAGGTGATGGGCGGGCA
CAAGCCCGAGAACATCGTGATCGAGATGGCCCGGGAGAACCAGACCACCCAGAAGGGCCAGA
AGAACAGCAGGGAGCGCATGAAGCGCATCGAGGAGGGCATCAAGGAGCTGGGTAGCCAGATC
CTGAAGGAGCACCCTGTGGAGAATACCCAGCTGCAGAACGAGAAGCTGTATCTGTACTACCT
GCAGAACGGCAGGGACATGTACGTGGATCAGGAGCTGGACATCAACCGGCTGTCTGACTACG
ACGTGGACCACATCGTGCCCCAGTCTTTCCTGAAGGACGACAGCATCGACAACAAGGTGCTG
ACCCGCAGCGACAAGAACAGGGGCAAGAGCGATAACGTGCCCTCCGAGGAGGTGGTCAAGAA
GATGAAGAACTACTGGCGGCAGCTGCTGAACGCCAAGCTGATCACCCAGCGGAAGTTCGACA
ACCTGACCAAGGCCGAGAGAGGCGGCCTGTCCGAGCTGGACAAGGCCGGCTTCATCAAGCGG
CAGCTGGTGGAGACCCGGCAGATCACCAAGCACGTCGCCCAGATCCTGGACTCCAGGATGAA
CACCAAGTACGACGAGAACGACAAGCTGATCAGAGAGGTGAAGGTGATCACCCTGAAGTCCA
AGCTGGTGAGCGACTTCAGGAAGGATTTCCAGTTCTACAAGGTGCGGGAGATCAACAACTAT
CACCACGCCCACGACGCCTACCTGAACGCCGTCGTGGGCACCGCTCTGATCAAGAAGTATCC
TAAGCTGGAGAGCGAGTTCGTGTACGGCGACTACAAGGTGTACGACGTGCGGAAGATGATCG
CCAAGAGCGAGCAGGAGATCGGCAAGGCCACCGCCAAGTACTTCTTCTACTCCAATATCATG
AACTTCTTCAAGACCGAGATCACCCTGGCCAACGGGGAGATCCGGAAGCGCCCACTGATCGA
GACCAACGGCGAGACCGGCGAGATCGTGTGGGACAAGGGCAGGGACTTCGCCACCGTGAGGA
AGGTGCTGTCCATGCCCCAGGTGAACATCGTGAAAAAGACCGAGGTGCAGACCGGGGGGTTC
TCCAAGGAGAGCATCCTGCCCAAGGGCAACAGCGACAAGCTGATCGCCCGGAAGAAGGACTG
GGACCCCAAGAAGTACGGAGGCTTCCTGTATCCTACCGTGGCCTACTCCGTGCTGGTGGTGG
CCAAGGTGGAGAAGGGCAAGTCCAAGAAGCTGAAGTCAGTGAAGGAGCTGCTGGGCATCACC
ATCATGGAGCGGTCTAGCTTCGAGAAGAATCCTATTGACTTCCTGGAGGCCAAGGGCTACAA
GGAGGTCAAGAAGGATCTGATCATCAAGCTGCCCAAGTACAGCCTGTTCGAGCTGGAGAATG
GCCGGAAGAGGATGCTGGCCAGCGCCAAGTTCCTGCAGAAGGGCAACGAGCTGGCCCTGCCC
AGCAAGTACGTGAACTTCCTCTATCTGGCCAGCCACTACGAGAAGCTGAAGGGCTCTCCAGA
GGACAACGAGCAGAAGCAGCTGTTCGTGGAGCAGCACAAGCACTACCTGGACGAGATCATCG
AGCAGATCTCCGAGTTCAGCAAGCGCGTGATCCTGGACGACGCCAACCTGGACAAGGTGCTG
TCCGCTTACAACAAGCACAGGGACAAGCCCATCAGGGAGCAGGCCGAGAACATCATCCACCT
GTTCACCCTGACCAACCTGGGCGCCCCCAGGGCCTTCAAGTACTTCGATACCACCATCGCCA
GGAAGGAGTACAAAAGTACTAAGGAGGTCCTGGATGCCACTCTGATCCACCAGAGCATCACC
GGGCTGTACGAGACCCGGATCGACCTGTCCCAGCTGGGCGGCGACGAGGGCGCCGACAAGCG
GACAGCCGACGGCAGCGAGTTCGAGAGCCCCAAGAAGAAGCGGAAGGTG
Variant G nucleotide sequence:
(SEQ ID NO: 457)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAGCGGCGGGAGCTCTGGGGGCTCCTCCGGGAGCGAGACCCCCGGGACCAGCGAGTCCGCC
ACCCCCGAGAGCAGCGGCGGCAGCTCCGGGGGGAGCGACAAGAAGTACTCCATCGGCCTGGC
CATCGGCACCAACTCCGTGGGTTGGGCCGTGATCACCGATGAGTACAAGGTGCCCAGCAAGA
AGTTCAAGGTGCTGGGCAACACCGACAGGCACTCTATCAAGAAGAACCTGATCGGCGCCCTG
CTGTTCGACAGCGGGGAGACCGCTGAGGCCACTCGGCTGAAGAGAACCGCCAGGCGCAGATA
CACCCGGAGAAAGAACCGGATCTGCTACCTGCAGGAGATCTTCAGCAACGAGATGGCCAAGG
TGGACGACAGCTTCTTCCACAGGCTGGAGGAGAGCTTCCTGGTGGAGGAGGACAAGAAGCAC
GAGCGCCACCCCATCTTCGGCAACATCGTGGATGAGGTGGCCTACCACGAGAAGTACCCCAC
CATCTACCACCTGCGGAAGAAGCTGGTGGACAGCACCGATAAGGCCGATCTGCGGCTGATCT
ACCTGGCCCTGGCCCACATGATCAAGTTCAGGGGGCACTTCCTGATCGAGGGCGACCTGAAC
CCCGACAACTCCGATGTGGATAAACTGTTCATCCAGCTGGTGCAGACCTACAACCAGCTGTT
TGAGGAGAATCCCATCAACGCCTCCGGCGTGGACGCCAAGGCCATCCTGAGCGCCCGGCTGT
CCAAGAGCCGGAGGCTGGAGAATCTGATCGCCCAGCTGCCTGGCGAGAAGAAGAACGGCCTG
TTCGGCAACCTGATCGCCCTGAGCCTGGGCCTGACCCCCAATTTCAAGAGCAACTTCGACCT
GGCCGAGGATGCTAAGCTGCAGCTGTCCAAGGACACCTACGACGATGACCTGGACAACCTGC
TGGCTCAGATCGGCGATCAGTACGCCGACCTGTTCCTGGCCGCCAAGAACCTGTCAGACGCC
ATCCTGCTGTCTGACATTCTGCGGGTGAACACCGAGATCACCAAGGCCCCACTGTCCGCCTC
TATGGTGAAGAGGTACGATGAGCACCACCAGGACCTGACCCTGCTGAAGGCTCTGGTGCGGC
AGCAGCTGCCGGAGAAGTACAAGGAGATCTTCTTCGACCAGAGCAAGAACGGCTACGCCGGC
TACATCGACGGCGGGGCCAGCCAGGAGGAGTTCTACAAGTTCATCAAGCCTATCCTGGAGAA
GATGGACGGCACCGAGGAGCTGCTGGTGAAGCTGAACCGGGAGGACCTGCTGCGGAAGCAGA
GGACCTTCGACAACGGCATCATTCCCCACCAGATCCACCTGGGCGAGCTGCACGCCATCCTG
CGGAGGCAGGGCGACTTCTACCCTTTCCTGAAGGACAACAGGGAGAAGATCGAGAAGATCCT
GACCTTCAGGATCCCCTACTACGTGGGCCCCCTGGCCCGCGGCAACTCCCGCTTTGCCTGGA
TGACCAGAAAGAGCGAGGAGACCATCACCCCTTGGAACTTCGAGGAGGTGGTGGACAAGGGC
GCCAGCGCCCAGAGCTTCATCGAGCGCATGACCAACTTCGACAAGAATCTGCCCAACGAGAA
GGTGCTGCCCAAGCACTCCCTGCTGTACGAGTACTTCACCGTGTACAACGAGCTGACAAAGG
TGAAGTACGTGACAGAGGGCATGCGCAAGCCCGCCTTCCTGTCTGGCGAGCAGAAGAAGGCC
ATCGTGGACCTGCTGTTCAAGACCAACAGGAAGGTGACCGTGAAGCAGCTGAAGGAGGACTA
CTTCAAGAAGATCGAGTGCTTCGACAGCGTGGAGATCAGCGGCGTGGAGGACAGGTTCAACG
CCAGCCTGGGCACCTACCACGACCTGCTGAAGATCATCAAGGACAAGGACTTCCTGGATAAC
GAGGAGAACGAGGATATCCTGGAGGACATTGTCCTGACCCTGACCCTGTTCGAGGATCGGGA
GATGATCGAGGAGCGCCTCAAGACCTACGCCCACCTGTTCGACGATAAGGTGATGAAGCAGC
TGAAGCGGCTGCGCTACACCGGCTGGGGCCGCCTGTCCCGGAAGCTGATCAACGGCATCCGG
GACAAGCAGTCCGGCAAGACCATCCTGGACTTCCTGAAGTCCGATGGTTTTGCCAACAGAAA
CTTCATGCAGCTGATCCACGACGACAGCCTGACCTTCAAGGAGGACATCCAGAAGGCTCAGG
TGTCCGGACAGGGGGACTCCCTGCACGAGCACATCGCCAACCTGGCCGGCTCTCCCGCCATC
AAGAAGGGCATCCTGCAGACCGTGAAGGTGGTGGACGAGCTGGTGAAGGTGATGGGCGGGCA
CAAGCCCGAGAACATCGTGATCGAGATGGCCCGGGAGAACCAGACCACCCAGAAGGGCCAGA
AGAACAGCAGGGAGCGCATGAAGCGCATCGAGGAGGGCATCAAGGAGCTGGGTAGCCAGATC
CTGAAGGAGCACCCTGTGGAGAATACCCAGCTGCAGAACGAGAAGCTGTATCTGTACTACCT
GCAGAACGGCAGGGACATGTACGTGGATCAGGAGCTGGACATCAACCGGCTGTCTGACTACG
ACGTGGACCACATCGTGCCCCAGTCTTTCCTGAAGGACGACAGCATCGACAACAAGGTGCTG
ACCCGCAGCGACAAGAACAGGGGCAAGAGCGATAACGTGCCCTCCGAGGAGGTGGTCAAGAA
GATGAAGAACTACTGGCGGCAGCTGCTGAACGCCAAGCTGATCACCCAGCGGAAGTTCGACA
ACCTGACCAAGGCCGAGAGAGGCGGCCTGTCCGAGCTGGACAAGGCCGGCTTCATCAAGCGG
CAGCTGGTGGAGACCCGGCAGATCACCAAGCACGTCGCCCAGATCCTGGACTCCAGGATGAA
CACCAAGTACGACGAGAACGACAAGCTGATCAGAGAGGTGAAGGTGATCACCCTGAAGTCCA
AGCTGGTGAGCGACTTCAGGAAGGATTTCCAGTTCTACAAGGTGCGGGAGATCAACAACTAT
CACCACGCCCACGACGCCTACCTGAACGCCGTCGTGGGCACCGCTCTGATCAAGAAGTATCC
TAAGCTGGAGAGCGAGTTCGTGTACGGCGACTACAAGGTGTACGACGTGCGGAAGATGATCG
CCAAGAGCGAGCAGGAGATCGGCAAGGCCACCGCCAAGTACTTCTTCTACTCCAATATCATG
AACTTCTTCAAGACCGAGATCACCCTGGCCAACGGGGAGATCCGGAAGCGCCCACTGATCGA
GACCAACGGCGAGACCGGCGAGATCGTGTGGGACAAGGGCAGGGACTTCGCCACCGTGAGGA
AGGTGCTGTCCATGCCCCAGGTGAACATCGTGAAAAAGACCGAGGTGCAGACCGGGGGGTTC
TCCAAGGAGAGCATCCTGCCCAAGGGCAACAGCGACAAGCTGATCGCCCGGAAGAAGGACTG
GGACCCCAAGAAGTACGGAGGCTTCCTGCAGCCTACCGTGGCCTACTCCGTGCTGGTGGTGG
CCAAGGTGGAGAAGGGCAAGTCCAAGAAGCTGAAGTCAGTGAAGGAGCTGCTGGGCATCACC
ATCATGGAGCGGTCTAGCTTCGAGAAGAATCCTATTGACTTCCTGGAGGCCAAGGGCTACAA
GGAGGTCAAGAAGGATCTGATCATCAAGCTGCCCAAGTACAGCCTGTTCGAGCTGGAGAATG
GCCGGAAGAGGATGCTGGCCAGCGCCAAGTTCCTGCAGAAGGGCAACGAGCTGGCCCTGCCC
AGCAAGTACGTGAACTTCCTCTATCTGGCCAGCCACTACGAGAAGCTGAAGGGCTCTCCAAA
GGACAACGAGCAGAAGCAGCTGTTCGTGGAGCAGCACAAGCACTACCTGGACGAGATCATCG
AGCAGATCTCCGAGTTCAGCAAGCGCGTGATCCTGGACGACGCCAACCTGGACAAGGTGCTG
TCCGCTTACAACAAGCACAGGGACAAGCCCATCAGGGAGCAGGCCGAGAACATCATCCACCT
GTTCACCCTGACCAACCTGGGCGCCCCCAGGGCCTTCAAGTACTTCGATACCACCATCGCCA
GGAAGGAGTACAAAAGTACTAAGGAGGTCCTGGATGCCACTCTGATCCACCAGAGCATCACC
GGGCTGTACGAGACCCGGATCGACCTGTCCCAGCTGGGCGGCGACGAGGGCGCCGACAAGCG
GACAGCCGACGGCAGCGAGTTCGAGAGCCCCAAGAAGAAGCGGAAGGTG
Variant H polynucleotide sequence:
(SEQ ID NO: 458)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAGCGGCGGGAGCTCTGGGGGCTCCTCCGGGAGCGAGACCCCCGGGACCAGCGAGTCCGCC
ACCCCCGAGAGCAGCGGCGGCAGCTCCGGGGGGAGCGACAAGAAGTACTCCATCGGCCTGGC
CATCGGCACCAACTCCGTGGGTTGGGCCGTGATCACCGATGAGTACAAGGTGCCCAGCAAGA
AGTTCAAGGTGCTGGGCAACACCGACAGGCACTCTATCAAGAAGAACCTGATCGGCGCCCTG
CTGTTCGACAGCGGGGAGACCGCTGAGGCCACTCGGCTGAAGAGAACCGCCAGGCGCAGATA
CACCCGGAGAAAGAACCGGATCTGCTACCTGCAGGAGATCTTCAGCAACGAGATGGCCAAGG
TGGACGACAGCTTCTTCCACAGGCTGGAGGAGAGCTTCCTGGTGGAGGAGGACAAGAAGCAC
GAGCGCCACCCCATCTTCGGCAACATCGTGGATGAGGTGGCCTACCACGAGAAGTACCCCAC
CATCTACCACCTGCGGAAGAAGCTGGTGGACAGCACCGATAAGGCCGATCTGCGGCTGATCT
ACCTGGCCCTGGCCCACATGATCAAGTTCAGGGGGCACTTCCTGATCGAGGGCGACCTGAAC
CCCGACAACTCCGATGTGGATAAACTGTTCATCCAGCTGGTGCAGACCTACAACCAGCTGTT
TGAGGAGAATCCCATCAACGCCTCCGGCGTGGACGCCAAGGCCATCCTGAGCGCCCGGCTGT
CCAAGAGCCGGAGGCTGGAGAATCTGATCGCCCAGCTGCCTGGCGAGAAGAAGAACGGCCTG
TTCGGCAACCTGATCGCCCTGAGCCTGGGCCTGACCCCCAATTTCAAGAGCAACTTCGACCT
GGCCGAGGATGCTAAGCTGCAGCTGTCCAAGGACACCTACGACGATGACCTGGACAACCTGC
TGGCTCAGATCGGCGATCAGTACGCCGACCTGTTCCTGGCCGCCAAGAACCTGTCAGACGCC
ATCCTGCTGTCTGACATTCTGCGGGTGAACACCGAGATCACCAAGGCCCCACTGTCCGCCTC
TATGGTGAAGAGGTACGATGAGCACCACCAGGACCTGACCCTGCTGAAGGCTCTGGTGCGGC
AGCAGCTGCCGGAGAAGTACAAGGAGATCTTCTTCGACCAGAGCAAGAACGGCTACGCCGGC
TACATCGACGGCGGGGCCAGCCAGGAGGAGTTCTACAAGTTCATCAAGCCTATCCTGGAGAA
GATGGACGGCACCGAGGAGCTGCTGGTGAAGCTGAACCGGGAGGACCTGCTGCGGAAGCAGA
GGACCTTCGACAACGGCATCATTCCCCACCAGATCCACCTGGGCGAGCTGCACGCCATCCTG
CGGAGGCAGGGCGACTTCTACCCTTTCCTGAAGGACAACAGGGAGAAGATCGAGAAGATCCT
GACCTTCAGGATCCCCTACTACGTGGGCCCCCTGGCCCGCGGCAACTCCCGCTTTGCCTGGA
TGACCAGAAAGAGCGAGGAGACCATCACCCCTTGGAACTTCGAGGAGGTGGTGGACAAGGGC
GCCAGCGCCCAGAGCTTCATCGAGCGCATGACCAACTTCGACAAGAATCTGCCCAACGAGAA
GGTGCTGCCCAAGCACTCCCTGCTGTACGAGTACTTCACCGTGTACAACGAGCTGACAAAGG
TGAAGTACGTGACAGAGGGCATGCGCAAGCCCGCCTTCCTGTCTGGCGAGCAGAAGAAGGCC
ATCGTGGACCTGCTGTTCAAGACCAACAGGAAGGTGACCGTGAAGCAGCTGAAGGAGGACTA
CTTCAAGAAGATCGAGTGCTTCGACAGCGTGGAGATCAGCGGCGTGGAGGACAGGTTCAACG
CCAGCCTGGGCACCTACCACGACCTGCTGAAGATCATCAAGGACAAGGACTTCCTGGATAAC
GAGGAGAACGAGGATATCCTGGAGGACATTGTCCTGACCCTGACCCTGTTCGAGGATCGGGA
GATGATCGAGGAGCGCCTCAAGACCTACGCCCACCTGTTCGACGATAAGGTGATGAAGCAGC
TGAAGCGGCTGCGCTACACCGGCTGGGGCCGCCTGTCCCGGAAGCTGATCAACGGCATCCGG
GACAAGCAGTCCGGCAAGACCATCCTGGACTTCCTGAAGTCCGATGGTTTTGCCAACAGAAA
CTTCATGCAGCTGATCCACGACGACAGCCTGACCTTCAAGGAGGACATCCAGAAGGCTCAGG
TGTCCGGACAGGGGGACTCCCTGCACGAGCACATCGCCAACCTGGCCGGCTCTCCCGCCATC
AAGAAGGGCATCCTGCAGACCGTGAAGGTGGTGGACGAGCTGGTGAAGGTGATGGGCGGGCA
CAAGCCCGAGAACATCGTGATCGAGATGGCCCGGGAGAACCAGACCACCCAGAAGGGCCAGA
AGAACAGCAGGGAGCGCATGAAGCGCATCGAGGAGGGCATCAAGGAGCTGGGTAGCCAGATC
CTGAAGGAGCACCCTGTGGAGAATACCCAGCTGCAGAACGAGAAGCTGTATCTGTACTACCT
GCAGAACGGCAGGGACATGTACGTGGATCAGGAGCTGGACATCAACCGGCTGTCTGACTACG
ACGTGGACCACATCGTGCCCCAGTCTTTCCTGAAGGACGACAGCATCGACAACAAGGTGCTG
ACCCGCAGCGACAAGAACAGGGGCAAGAGCGATAACGTGCCCTCCGAGGAGGTGGTCAAGAA
GATGAAGAACTACTGGCGGCAGCTGCTGAACGCCAAGCTGATCACCCAGCGGAAGTTCGACA
ACCTGACCAAGGCCGAGAGAGGCGGCCTGTCCGAGCTGGACAAGGCCGGCTTCATCAAGCGG
CAGCTGGTGGAGACCCGGCAGATCACCAAGCACGTCGCCCAGATCCTGGACTCCAGGATGAA
CACCAAGTACGACGAGAACGACAAGCTGATCAGAGAGGTGAAGGTGATCACCCTGAAGTCCA
AGCTGGTGAGCGACTTCAGGAAGGATTTCCAGTTCTACAAGGTGCGGGAGATCAACAACTAT
CACCACGCCCACGACGCCTACCTGAACGCCGTCGTGGGCACCGCTCTGATCAAGAAGTATCC
TAAGCTGGAGAGCGAGTTCGTGTACGGCGACTACAAGGTGTACGACGTGCGGAAGATGATCG
CCAAGAGCGAGCAGGAGATCGGCAAGGCCACCGCCAAGTACTTCTTCTACTCCAATATCATG
AACTTCTTCAAGACCGAGATCACCCTGGCCAACGGGGAGATCCGGAAGCGCCCACTGATCGA
GACCAACGGCGAGACCGGCGAGATCGTGTGGGACAAGGGCAGGGACTTCGCCACCGTGAGGA
AGGTGCTGTCCATGCCCCAGGTGAACATCGTGAAAAAGACCGAGGTGCAGACCGGGGGGTTC
TCCAAGGAGAGCATCCTGCCCAAGGGCAACAGCGACAAGCTGATCGCCCGGAAGAAGGACTG
GGACCCCAAGAAGTACGGAGGCTTCATGTATCCTACCGTGGCCTACTCCGTGCTGGTGGTGG
CCAAGGTGGAGAAGGGCAAGTCCAAGAAGCTGAAGTCAGTGAAGGAGCTGCTGGGCATCACC
ATCATGGAGCGGTCTAGCTTCGAGAAGAATCCTATTGACTTCCTGGAGGCCAAGGGCTACAA
GGAGGTCAAGAAGGATCTGATCATCAAGCTGCCCAAGTACAGCCTGTTCGAGCTGGAGAATG
GCCGGAAGAGGATGCTGGCCAGCGCCAAGTTCCTGCAGAAGGGCAACGAGCTGGCCCTGCCC
AGCAAGTACGTGAACTTCCTCTATCTGGCCAGCCACTACGAGAAGCTGAAGGGCTCTCCAAA
GGACAACGAGCAGAAGCAGCTGTTCGTGGAGCAGCACAAGCACTACCTGGACGAGATCATCG
AGCAGATCTCCGAGTTCAGCAAGCGCGTGATCCTGGACGACGCCAACCTGGACAAGGTGCTG
TCCGCTTACAACAAGCACAGGGACAAGCCCATCAGGGAGCAGGCCGAGAACATCATCCACCT
GTTCACCCTGACCAACCTGGGCGCCCCCAGGGCCTTCAAGTACTTCGATACCACCATCGCCA
GGAAGGAGTACAGGAGTACTAAGGAGGTCCTGGATGCCACTCTGATCCACCAGAGCATCACC
GGGCTGTACGAGACCCGGATCGACCTGTCCCAGCTGGGCGGCGACGAGGGCGCCGACAAGCG
GACAGCCGACGGCAGCGAGTTCGAGAGCCCCAAGAAGAAGCGGAAGGTG
Variant I polynucleotide sequence:
(SEQ ID NO: 459)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAAAGGCCCTAAGCCCAAGAAGGAAGAGAGCGAGAAGGACAAGAAGTACTCCATCGGCCTG
GCCATCGGCACCAACTCCGTGGGTTGGGCCGTGATCACCGATGAGTACAAGGTGCCCAGCAA
GAAGTTCAAGGTGCTGGGCAACACCGACAGGCACTCTATCAAGAAGAACCTGATCGGCGCCC
TGCTGTTCGACAGCGGGGAGACCGCTGAGGCCACTCGGCTGAAGAGAACCGCCAGGCGCAGA
TACACCCGGAGAAAGAACCGGATCTGCTACCTGCAGGAGATCTTCAGCAACGAGATGGCCAA
GGTGGACGACAGCTTCTTCCACAGGCTGGAGGAGAGCTTCCTGGTGGAGGAGGACAAGAAGC
ACGAGCGCCACCCCATCTTCGGCAACATCGTGGATGAGGTGGCCTACCACGAGAAGTACCCC
ACCATCTACCACCTGCGGAAGAAGCTGGTGGACAGCACCGATAAGGCCGATCTGCGGCTGAT
CTACCTGGCCCTGGCCCACATGATCAAGTTCAGGGGGCACTTCCTGATCGAGGGCGACCTGA
ACCCCGACAACTCCGATGTGGATAAACTGTTCATCCAGCTGGTGCAGACCTACAACCAGCTG
TTTGAGGAGAATCCCATCAACGCCTCCGGCGTGGACGCCAAGGCCATCCTGAGCGCCCGGCT
GTCCAAGAGCCGGAGGCTGGAGAATCTGATCGCCCAGCTGCCTGGCGAGAAGAAGAACGGCC
TGTTCGGCAACCTGATCGCCCTGAGCCTGGGCCTGACCCCCAATTTCAAGAGCAACTTCGAC
CTGGCCGAGGATGCTAAGCTGCAGCTGTCCAAGGACACCTACGACGATGACCTGGACAACCT
GCTGGCTCAGATCGGCGATCAGTACGCCGACCTGTTCCTGGCCGCCAAGAACCTGTCAGACG
CCATCCTGCTGTCTGACATTCTGCGGGTGAACACCGAGATCACCAAGGCCCCACTGTCCGCC
TCTATGGTGAAGAGGTACGATGAGCACCACCAGGACCTGACCCTGCTGAAGGCTCTGGTGCG
GCAGCAGCTGCCGGAGAAGTACAAGGAGATCTTCTTCGACCAGAGCAAGAACGGCTACGCCG
GCTACATCGACGGCGGGGCCAGCCAGGAGGAGTTCTACAAGTTCATCAAGCCTATCCTGGAG
AAGATGGACGGCACCGAGGAGCTGCTGGTGAAGCTGAACCGGGAGGACCTGCTGCGGAAGCA
GAGGACCTTCGACAACGGCATCATTCCCCACCAGATCCACCTGGGCGAGCTGCACGCCATCC
TGCGGAGGCAGGGCGACTTCTACCCTTTCCTGAAGGACAACAGGGAGAAGATCGAGAAGATC
CTGACCTTCAGGATCCCCTACTACGTGGGCCCCCTGGCCCGCGGCAACTCCCGCTTTGCCTG
GATGACCAGAAAGAGCGAGGAGACCATCACCCCTTGGAACTTCGAGGAGGTGGTGGACAAGG
GCGCCAGCGCCCAGAGCTTCATCGAGCGCATGACCAACTTCGACAAGAATCTGCCCAACGAG
AAGGTGCTGCCCAAGCACTCCCTGCTGTACGAGTACTTCACCGTGTACAACGAGCTGACAAA
GGTGAAGTACGTGACAGAGGGCATGCGCAAGCCCGCCTTCCTGTCTGGCGAGCAGAAGAAGG
CCATCGTGGACCTGCTGTTCAAGACCAACAGGAAGGTGACCGTGAAGCAGCTGAAGGAGGAC
TACTTCAAGAAGATCGAGTGCTTCGACAGCGTGGAGATCAGCGGCGTGGAGGACAGGTTCAA
CGCCAGCCTGGGCACCTACCACGACCTGCTGAAGATCATCAAGGACAAGGACTTCCTGGATA
ACGAGGAGAACGAGGATATCCTGGAGGACATTGTCCTGACCCTGACCCTGTTCGAGGATCGG
GAGATGATCGAGGAGCGCCTCAAGACCTACGCCCACCTGTTCGACGATAAGGTGATGAAGCA
GCTGAAGCGGCTGCGCTACACCGGCTGGGGCCGCCTGTCCCGGAAGCTGATCAACGGCATCC
GGGACAAGCAGTCCGGCAAGACCATCCTGGACTTCCTGAAGTCCGATGGTTTTGCCAACAGA
AACTTCATGCAGCTGATCCACGACGACAGCCTGACCTTCAAGGAGGACATCCAGAAGGCTCA
GGTGTCCGGACAGGGGGACTCCCTGCACGAGCACATCGCCAACCTGGCCGGCTCTCCCGCCA
TCAAGAAGGGCATCCTGCAGACCGTGAAGGTGGTGGACGAGCTGGTGAAGGTGATGGGCGGG
CACAAGCCCGAGAACATCGTGATCGAGATGGCCCGGGAGAACCAGACCACCCAGAAGGGCCA
GAAGAACAGCAGGGAGCGCATGAAGCGCATCGAGGAGGGCATCAAGGAGCTGGGTAGCCAGA
TCCTGAAGGAGCACCCTGTGGAGAATACCCAGCTGCAGAACGAGAAGCTGTATCTGTACTAC
CTGCAGAACGGCAGGGACATGTACGTGGATCAGGAGCTGGACATCAACCGGCTGTCTGACTA
CGACGTGGACCACATCGTGCCCCAGTCTTTCCTGAAGGACGACAGCATCGACAACAAGGTGC
TGACCCGCAGCGACAAGAACAGGGGCAAGAGCGATAACGTGCCCTCCGAGGAGGTGGTCAAG
AAGATGAAGAACTACTGGCGGCAGCTGCTGAACGCCAAGCTGATCACCCAGCGGAAGTTCGT
CAGAAGTCGAGTTTAGCCACGAATATTGGATGCGCCACGCCCTCACCCTGGCCAAGAGAGCC
CGGGACGAGCGCGAGGTGCCCGTGGGCGCCGTCCTGGTGCACAACAACAGGGTGATCGGCGA
GGGCTGGAACCGGGCCATCGGCCTGCACGACCCCACAGCCCACGCCGAGATCATGGCCCTGC
GGCAGGGTGGCCTGGTCATGCAGAACTACAGGCTGTATGACGCCACCCTGTACACCACATTC
GAGCCCTGCGTGATGTGCGCCGGGGCCATGATCCACAGCCGGATCGGCCGCGTGGTGTTCGG
CGTGCGGAACGCCAAGACCGGCGCCGCCGGCAGCCTGATGGACGTTCTGCACTACCCCGGGA
TGAACCACAGGGTGGAGATCACAGAGGGCATCCTGGCAGACGAGTGCGCCGCCCTGCTGTGC
ACTTTCTTCAGGATGCCCAGATCTGTGTTCAAGGCCCAGAAGAAGGCCCAGAGCTCCACCGA
CACAACCTGACCAAGGCCGAGAGAGGCGGCCTGTCCGAGCTGGACAAGGCCGGCTTCATCAA
GCGGCAGCTGGTGGAGACCCGGCAGATCACCAAGCACGTCGCCCAGATCCTGGACTCCAGGA
TGAACACCAAGTACGACGAGAACGACAAGCTGATCAGAGAGGTGAAGGTGATCACCCTGAAG
TCCAAGCTGGTGAGCGACTTCAGGAAGGATTTCCAGTTCTACAAGGTGCGGGAGATCAACAA
CTATCACCACGCCCACGACGCCTACCTGAACGCCGTCGTGGGCACCGCTCTGATCAAGAAGT
ATCCTAAGCTGGAGAGCGAGTTCGTGTACGGCGACTACAAGGTGTACGACGTGCGGAAGATG
ATCGCCAAGAGCGAGCAGGAGATCGGCAAGGCCACCGCCAAGTACTTCTTCTACTCCAATAT
CATGAACTTCTTCAAGACCGAGATCACCCTGGCCAACGGGGAGATCCGGAAGCGCCCACTGA
TCGAGACCAACGGCGAGACCGGCGAGATCGTGTGGGACAAGGGCAGGGACTTCGCCACCGTG
AGGAAGGTGCTGTCCATGCCCCAGGTGAACATCGTGAAAAAGACCGAGGTGCAGACCGGGGG
GTTCTCCAAGGAGAGCATCCTGCCCAAGGGCAACAGCGACAAGCTGATCGCCCGGAAGAAGG
ACTGGGACCCCAAGAAGTACGGAGGCTTCCTGTATCCTACCGTGGCCTACTCCGTGCTGGTG
GTGGCCAAGGTGGAGAAGGGCAAGTCCAAGAAGCTGAAGTCAGTGAAGGAGCTGCTGGGCAT
CACCATCATGGAGCGGTCTAGCTTCGAGAAGAATCCTATTGACTTCCTGGAGGCCAAGGGCT
ACAAGGAGGTCAAGAAGGATCTGATCATCAAGCTGCCCAAGTACAGCCTGTTCGAGCTGGAG
AATGGCCGGAAGAGGATGCTGGCCAGCGCCAAGTTCCTGCAGAAGGGCAACGAGCTGGCCCT
GCCCAGCAAGTACGTGAACTTCCTCTATCTGGCCAGCCACTACGAGAAGCTGAAGGGCTCTC
CAGAGGACAACGAGCAGAAGCAGCTGTTCGTGGAGCAGCACAAGCACTACCTGGACGAGATC
ATCGAGCAGATCTCCGAGTTCAGCAAGCGCGTGATCCTGGACGACGCCAACCTGGACAAGGT
GCTGTCCGCTTACAACAAGCACAGGGACAAGCCCATCAGGGAGCAGGCCGAGAACATCATCC
ACCTGTTCACCCTGACCAACCTGGGCGCCCCCAGGGCCTTCAAGTACTTCGATACCACCATC
GCCAGGAAGGAGTACAAAAGTACTAAGGAGGTCCTGGATGCCACTCTGATCCACCAGAGCAT
CACCGGGCTGTACGAGACCCGGATCGACCTGTCCCAGCTGGGCGGCGACGAGGGCGCCGACA
AGCGGACAGCCGACGGCAGCGAGTTCGAGAGCCCCAAGAAGAAGCGGAAGGTG
Variant J polynucleotide sequence:
(SEQ ID NO: 460)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAGCGGCGGGAGCTCTGGGGGCTCCTCCGGGAGCGAGACCCCCGGGACCAGCGAGTCCGCC
ACCCCCGAGAGCAGCGGCGGCAGCTCCGGGGGGAGCGACAAGAAGTACTCCATCGGCCTGGC
CATCGGCACCAACTCCGTGGGTTGGGCCGTGATCACCGATGAGTACAAGGTGCCCAGCAAGA
AGTTCAAGGTGCTGGGCAACACCGACAGGCACTCTATCAAGAAGAACCTGATCGGCGCCCTG
CTGTTCGACAGCGGGGAGACCGCTGAGGCCACTCGGCTGAAGAGAACCGCCAGGCGCAGATA
CACCCGGAGAAAGAACCGGATCTGCTACCTGCAGGAGATCTTCAGCAACGAGATGGCCAAGG
TGGACGACAGCTTCTTCCACAGGCTGGAGGAGAGCTTCCTGGTGGAGGAGGACAAGAAGCAC
GAGCGCCACCCCATCTTCGGCAACATCGTGGATGAGGTGGCCTACCACGAGAAGTACCCCAC
CATCTACCACCTGCGGAAGAAGCTGGTGGACAGCACCGATAAGGCCGATCTGCGGCTGATCT
ACCTGGCCCTGGCCCACATGATCAAGTTCAGGGGGCACTTCCTGATCGAGGGCGACCTGAAC
CCCGACAACTCCGATGTGGATAAACTGTTCATCCAGCTGGTGCAGACCTACAACCAGCTGTT
TGAGGAGAATCCCATCAACGCCTCCGGCGTGGACGCCAAGGCCATCCTGAGCGCCCGGCTGT
CCAAGAGCCGGAGGCTGGAGAATCTGATCGCCCAGCTGCCTGGCGAGAAGAAGAACGGCCTG
TTCGGCAACCTGATCGCCCTGAGCCTGGGCCTGACCCCCAATTTCAAGAGCAACTTCGACCT
GGCCGAGGATGCTAAGCTGCAGCTGTCCAAGGACACCTACGACGATGACCTGGACAACCTGC
TGGCTCAGATCGGCGATCAGTACGCCGACCTGTTCCTGGCCGCCAAGAACCTGTCAGACGCC
ATCCTGCTGTCTGACATTCTGCGGGTGAACACCGAGATCACCAAGGCCCCACTGTCCGCCTC
TATGGTGAAGAGGTACGATGAGCACCACCAGGACCTGACCCTGCTGAAGGCTCTGGTGCGGC
AGCAGCTGCCGGAGAAGTACAAGGAGATCTTCTTCGACCAGAGCAAGAACGGCTACGCCGGC
TACATCGACGGCGGGGCCAGCCAGGAGGAGTTCTACAAGTTCATCAAGCCTATCCTGGAGAA
GATGGACGGCACCGAGGAGCTGCTGGTGAAGCTGAACCGGGAGGACCTGCTGCGGAAGCAGA
GGACCTTCGACAACGGCATCATTCCCCACCAGATCCACCTGGGCGAGCTGCACGCCATCCTG
CGGAGGCAGGGCGACTTCTACCCTTTCCTGAAGGACAACAGGGAGAAGATCGAGAAGATCCT
GACCTTCAGGATCCCCTACTACGTGGGCCCCCTGGCCCGCGGCAACTCCCGCTTTGCCTGGA
TGACCAGAAAGAGCGAGGAGACCATCACCCCTTGGAACTTCGAGGAGGTGGTGGACAAGGGC
GCCAGCGCCCAGAGCTTCATCGAGCGCATGACCAACTTCGACAAGAATCTGCCCAACGAGAA
GGTGCTGCCCAAGCACTCCCTGCTGTACGAGTACTTCACCGTGTACAACGAGCTGACAAAGG
TGAAGTACGTGACAGAGGGCATGCGCAAGCCCGCCTTCCTGTCTGGCGAGCAGAAGAAGGCC
ATCGTGGACCTGCTGTTCAAGACCAACAGGAAGGTGACCGTGAAGCAGCTGAAGGAGGACTA
CTTCAAGAAGATCGAGTGCTTCGACAGCGTGGAGATCAGCGGCGTGGAGGACAGGTTCAACG
CCAGCCTGGGCACCTACCACGACCTGCTGAAGATCATCAAGGACAAGGACTTCCTGGATAAC
GAGGAGAACGAGGATATCCTGGAGGACATTGTCCTGACCCTGACCCTGTTCGAGGATCGGGA
GATGATCGAGGAGCGCCTCAAGACCTACGCCCACCTGTTCGACGATAAGGTGATGAAGCAGC
TGAAGCGGCTGCGCTACACCGGCTGGGGCCGCCTGTCCCGGAAGCTGATCAACGGCATCCGG
GACAAGCAGTCCGGCAAGACCATCCTGGACTTCCTGAAGTCCGATGGTTTTGCCAACAGAAA
CTTCATGCAGCTGATCCACGACGACAGCCTGACCTTCAAGGAGGACATCCAGAAGGCTCAGG
TGTCCGGACAGGGGGACTCCCTGCACGAGCACATCGCCAACCTGGCCGGCTCTCCCGCCATC
AAGAAGGGCATCCTGCAGACCGTGAAGGTGGTGGACGAGCTGGTGAAGGTGATGGGCGGGCA
CAAGCCCGAGAACATCGTGATCGAGATGGCCGCTGAGAACGCCACCACCCAGAAGGGCCAGA
AGAACAGCAGGGAGCGCATGAAGCGCATCGAGGAGGGCATCAAGGAGCTGGGTAGCCAGATC
CTGAAGGAGCACCCTGTGGAGAATACCCAGCTGCAGAACGAGAAGCTGTATCTGTACTACCT
GCAGAACGGCAGGGACATGTACGTGGATCAGGAGCTGGACATCAACCGGCTGTCTGACTACG
ACGTGGACCACATCGTGCCCCAGTCTTTCCTGAAGGACGACAGCATCGACAACAAGGTGCTG
ACCCGCAGCGACAAGAACAGGGGCAAGAGCGATAACGTGCCCTCCGAGGAGGTGGTCAAGAA
GATGAAGAACTACTGGCGGCAGCTGCTGAACGCCAAGCTGATCACCCAGCGGAAGTTCGACA
ACCTGACCAAGGCCGAGAGAGGCGGCCTGTCCGAGCTGGACAAGGCCGGCTTCATCAAGCGG
CAGCTGGTGGAGACCCGGCAGATCACCAAGCACGTCGCCCAGATCCTGGACTCCAGGATGAA
CACCAAGTACGACGAGAACGACAAGCTGATCAGAGAGGTGAAGGTGATCACCCTGAAGTCCA
AGCTGGTGAGCGACTTCAGGAAGGATTTCCAGTTCTACAAGGTGCGGGAGATCAACAACTAT
CACCACGCCCACGACGCCTACCTGAACGCCGTCGTGGGCACCGCTCTGATCAAGAAGTATCC
TAAGCTGGAGAGCGAGTTCGTGTACGGCGACTACAAGGTGTACGACGTGCGGAAGATGATCG
CCAAGAGCGAGCAGGAGATCGGCAAGGCCACCGCCAAGTACTTCTTCTACTCCAATATCATG
AACTTCTTCAAGACCGAGATCACCCTGGCCAACGGGGAGATCCGGAAGCGCCCACTGATCGA
GACCAACGGCGAGACCGGCGAGATCGTGTGGGACAAGGGCAGGGACTTCGCCACCGTGAGGA
AGGTGCTGTCCATGCCCCAGGTGAACATCGTGAAAAAGACCGAGGTGCAGACCGGGGGGTTC
TCCAAGGAGAGCATCCTGCCCAAGGGCAACAGCGACAAGCTGATCGCCCGGAAGAAGGACTG
GGACCCCAAGAAGTACGGAGGCTTCCTGTATCCTACCGTGGCCTACTCCGTGCTGGTGGTGG
CCAAGGTGGAGAAGGGCAAGTCCAAGAAGCTGAAGTCAGTGAAGGAGCTGCTGGGCATCACC
ATCATGGAGCGGTCTAGCTTCGAGAAGAATCCTATTGACTTCCTGGAGGCCAAGGGCTACAA
GGAGGTCAAGAAGGATCTGATCATCAAGCTGCCCAAGTACAGCCTGTTCGAGCTGGAGAATG
GCCGGAAGAGGATGCTGGCCAGCGCCAAGTTCCTGCAGAAGGGCAACGAGCTGGCCCTGCCC
AGCAAGTACGTGAACTTCCTCTATCTGGCCAGCCACTACGAGAAGCTGAAGGGCTCTCCAGA
GGACAACGAGCAGAAGCAGCTGTTCGTGGAGCAGCACAAGCACTACCTGGACGAGATCATCG
AGCAGATCTCCGAGTTCAGCAAGCGCGTGATCCTGGACGACGCCAACCTGGACAAGGTGCTG
TCCGCTTACAACAAGCACAGGGACAAGCCCATCAGGGAGCAGGCCGAGAACATCATCCACCT
GTTCACCCTGACCAACCTGGGCGCCCCCAGGGCCTTCAAGTACTTCGATACCACCATCGCCA
GGAAGGAGTACAAAAGTACTAAGGAGGTCCTGGATGCCACTCTGATCCACCAGAGCATCACC
GGGCTGTACGAGACCCGGATCGACCTGTCCCAGCTGGGCGGCGACGAGGGCGCCGACAAGCG
GACAGCCGACGGCAGCGAGTTCGAGAGCCCCAAGAAGAAGCGGAAGGTG
Variant K polynucleotide sequence:
(SEQ ID NO: 461)
TCAGAAGTCGAGTTTAGCCACGAATATTGGATGCGCCACGCCCTCACCCTGGCCAAGAGAGC
CCGGGACGAGCGCGAGGTGCCCGTGGGCGCCGTCCTGGTGCACAACAACAGGGTGATCGGCG
AGGGCTGGAACCGGGCCATCGGCCTGCACGACCCCACAGCCCACGCCGAGATCATGGCCCTG
CGGCAGGGTGGCCTGGTCATGCAGAACTACAGGCTGTATGACGCCACCCTGTACACCACATT
CGAGCCCTGCGTGATGTGCGCCGGGGCCATGATCCACAGCCGGATCGGCCGCGTGGTGTTCG
GCGTGCGGAACGCCAAGACCGGCGCCGCCGGCAGCCTGATGGACGTTCTGCACTACCCCGGG
ATGAACCACAGGGTGGAGATCACAGAGGGCATCCTGGCAGACGAGTGCGCCGCCCTGCTGTG
CACTTTCTTCAGGATGCCCAGATCTGTGTTCAAGGCCCAGAAGAAGGCCCAGAGCTCCACCG
ACAAAGGCCCTAAGCCCAAGAAGGAAGAGAGCGAGAAGGACAAGAAGTACTCCATCGGCCTG
GCCATCGGCACCAACTCCGTGGGTTGGGCCGTGATCACCGATGAGTACAAGGTGCCCAGCAA
GAAGTTCAAGGTGCTGGGCAACACCGACAGGCACTCTATCAAGAAGAACCTGATCGGCGCCC
TGCTGTTCGACAGCGGGGAGACCGCTGAGGCCACTCGGCTGAAGAGAACCGCCAGGCGCAGA
TACACCCGGAGAAAGAACCGGATCTGCTACCTGCAGGAGATCTTCAGCAACGAGATGGCCAA
GGTGGACGACAGCTTCTTCCACAGGCTGGAGGAGAGCTTCCTGGTGGAGGAGGACAAGAAGC
ACGAGCGCCACCCCATCTTCGGCAACATCGTGGATGAGGTGGCCTACCACGAGAAGTACCCC
ACCATCTACCACCTGCGGAAGAAGCTGGTGGACAGCACCGATAAGGCCGATCTGCGGCTGAT
CTACCTGGCCCTGGCCCACATGATCAAGTTCAGGGGGCACTTCCTGATCGAGGGCGACCTGA
ACCCCGACAACTCCGATGTGGATAAACTGTTCATCCAGCTGGTGCAGACCTACAACCAGCTG
TTTGAGGAGAATCCCATCAACGCCTCCGGCGTGGACGCCAAGGCCATCCTGAGCGCCCGGCT
GTCCAAGAGCCGGAGGCTGGAGAATCTGATCGCCCAGCTGCCTGGCGAGAAGAAGAACGGCC
TGTTCGGCAACCTGATCGCCCTGAGCCTGGGCCTGACCCCCAATTTCAAGAGCAACTTCGAC
CTGGCCGAGGATGCTAAGCTGCAGCTGTCCAAGGACACCTACGACGATGACCTGGACAACCT
GCTGGCTCAGATCGGCGATCAGTACGCCGACCTGTTCCTGGCCGCCAAGAACCTGTCAGACG
CCATCCTGCTGTCTGACATTCTGCGGGTGAACACCGAGATCACCAAGGCCCCACTGTCCGCC
TCTATGGTGAAGAGGTACGATGAGCACCACCAGGACCTGACCCTGCTGAAGGCTCTGGTGCG
GCAGCAGCTGCCGGAGAAGTACAAGGAGATCTTCTTCGACCAGAGCAAGAACGGCTACGCCG
GCTACATCGACGGCGGGGCCAGCCAGGAGGAGTTCTACAAGTTCATCAAGCCTATCCTGGAG
AAGATGGACGGCACCGAGGAGCTGCTGGTGAAGCTGAACCGGGAGGACCTGCTGCGGAAGCA
GAGGACCTTCGACAACGGCATCATTCCCCACCAGATCCACCTGGGCGAGCTGCACGCCATCC
TGCGGAGGCAGGGCGACTTCTACCCTTTCCTGAAGGACAACAGGGAGAAGATCGAGAAGATC
CTGACCTTCAGGATCCCCTACTACGTGGGCCCCCTGGCCCGCGGCAACTCCCGCTTTGCCTG
GATGACCAGAAAGAGCGAGGAGACCATCACCCCTTGGAACTTCGAGGAGGTGGTGGACAAGG
GCGCCAGCGCCCAGAGCTTCATCGAGCGCATGACCAACTTCGACAAGAATCTGCCCAACGAG
AAGGTGCTGCCCAAGCACTCCCTGCTGTACGAGTACTTCACCGTGTACAACGAGCTGACAAA
GGTGAAGTACGTGACAGAGGGCATGCGCAAGCCCGCCTTCCTGTCTGGCGAGCAGAAGAAGG
CCATCGTGGACCTGCTGTTCAAGACCAACAGGAAGGTGACCGTGAAGCAGCTGAAGGAGGAC
TACTTCAAGAAGATCGAGTGCTTCGACAGCGTGGAGATCAGCGGCGTGGAGGACAGGTTCAA
CGCCAGCCTGGGCACCTACCACGACCTGCTGAAGATCATCAAGGACAAGGACTTCCTGGATA
ACGAGGAGAACGAGGATATCCTGGAGGACATTGTCCTGACCCTGACCCTGTTCGAGGATCGG
GAGATGATCGAGGAGCGCCTCAAGACCTACGCCCACCTGTTCGACGATAAGGTGATGAAGCA
GCTGAAGCGGCTGCGCTACACCGGCTGGGGCCGCCTGTCCCGGAAGCTGATCAACGGCATCC
GGGACAAGCAGTCCGGCAAGACCATCCTGGACTTCCTGAAGTCCGATGGTTTTGCCAACAGA
AACTTCATGCAGCTGATCCACGACGACAGCCTGACCTTCAAGGAGGACATCCAGAAGGCTCA
GGTGTCCGGACAGGGGGACTCCCTGCACGAGCACATCGCCAACCTGGCCGGCTCTCCCGCCA
TCAAGAAGGGCATCCTGCAGACCGTGAAGGTGGTGGACGAGCTGGTGAAGGTGATGGGCGGG
CACAAGCCCGAGAACATCGTGATCGAGATGGCCCGGGAGAACCAGACCACCCAGAAGGGCCA
GAAGAACAGCAGGGAGCGCATGAAGCGCATCGAGGAGGGCATCAAGGAGCTGGGTAGCCAGA
TCCTGAAGGAGCACCCTGTGGAGAATACCCAGCTGCAGAACGAGAAGCTGTATCTGTACTAC
CTGCAGAACGGCAGGGACATGTACGTGGATCAGGAGCTGGACATCAACCGGCTGTCTGACTA
CGACGTGGACCACATCGTGCCCCAGTCTTTCCTGAAGGACGACAGCATCGACAACAAGGTGC
TGACCCGCAGCGACAAGAACAGGGGCAAGAGCGATAACGTGCCCTCCGAGGAGGTGGTCAAG
AAGATGAAGAACTACTGGCGGCAGCTGCTGAACGCCAAGCTGATCACCCAGCGGAAGTTCGA
CAACCTGACCAAGGCCGAGAGAGGCGGCCTGTCCGAGCTGGACAAGGCCGGCTTCATCAAGC
GGCAGCTGGTGGAGACCCGGCAGATCACCAAGCACGTCGCCCAGATCCTGGACTCCAGGATG
AACACCAAGTACGACGAGAACGACAAGCTGATCAGAGAGGTGAAGGTGATCACCCTGAAGTC
CAAGCTGGTGAGCGACTTCAGGAAGGATTTCCAGTTCTACAAGGTGCGGGAGATCAACAACT
ATCACCACGCCCACGACGCCTACCTGAACGCCGTCGTGGGCACCGCTCTGATCAAGAAGTAT
CCTAAGCTGGAGAGCGAGTTCGTGTACGGCGACTACAAGGTGTACGACGTGCGGAAGATGAT
CGCCAAGAGCGAGCAGGAGATCGGCAAGGCCACCGCCAAGTACTTCTTCTACTCCAATATCA
TGAACTTCTTCAAGACCGAGATCACCCTGGCCAACGGGGAGATCCGGAAGCGCCCACTGATC
GAGACCAACGGCGAGACCGGCGAGATCGTGTGGGACAAGGGCAGGGACTTCGCCACCGTGAG
GAAGGTGCTGTCCATGCCCCAGGTGAACATCGTGAAAAAGACCGAGGTGCAGACCGGGGGGT
TCTCCAAGGAGAGCATCCTGCCCAAGGGCAACAGCGACAAGCTGATCGCCCGGAAGAAGGAC
TGGGACCCCAAGAAGTACGGAGGCTTCATGTATCCTACCGTGGCCTACTCCGTGCTGGTGGT
GGCCAAGGTGGAGAAGGGCAAGTCCAAGAAGCTGAAGTCAGTGAAGGAGCTGCTGGGCATCA
CCATCATGGAGCGGTCTAGCTTCGAGAAGAATCCTATTGACTTCCTGGAGGCCAAGGGCTAC
AAGGAGGTCAAGAAGGATCTGATCATCAAGCTGCCCAAGTACAGCCTGTTCGAGCTGGAGAA
TGGCCGGAAGAGGATGCTGGCCAGCGCCAAGTTCCTGCAGAAGGGCAACGAGCTGGCCCTGC
CCAGCAAGTACGTGAACTTCCTCTATCTGGCCAGCCACTACGAGAAGCTGAAGGGCTCTCCA
AAGGACAACGAGCAGAAGCAGCTGTTCGTGGAGCAGCACAAGCACTACCTGGACGAGATCAT
CGAGCAGATCTCCGAGTTCAGCAAGCGCGTGATCCTGGACGACGCCAACCTGGACAAGGTGC
TGTCCGCTTACAACAAGCACAGGGACAAGCCCATCAGGGAGCAGGCCGAGAACATCATCCAC
CTGTTCACCCTGACCAACCTGGGCGCCCCCAGGGCCTTCAAGTACTTCGATACCACCATCGC
CAGGAAGGAGTACAGGAGTACTAAGGAGGTCCTGGATGCCACTCTGATCCACCAGAGCATCA
CCGGGCTGTACGAGACCCGGATCGACCTGTCCCAGCTGGGCGGCGACGAGGGCGCCGACAAG
CGGACAGCCGACGGCAGCGAGTTCGAGAGCCCCAAGAAGAAGCGGAAGGTG.

In some embodiments, the ABE variants provided herein comprise the following arrangement of proteins (From N-terminus to C-terminus):

[adenosine deaminase]-[optional linker]-[Cas9 domain]-[optional NLS].

In some embodiments, the arrangement of proteins of an ABE variant is [TadA*7.10 (L36H, I76Y, V82T, Y147T, Q154S, and N157K)]-[optional linker]-[SpCas9-MQKFRAER nickase]-[optional NLS]. In some embodiments the ABE variant is any one of the following ABE variants (i.e., ABE Variants A-K):

Variant A:
[TadA*7.10 (L36H, 176Y, V82T, Y147T, Q154S, and N157K)]-
[SGGSSGGSSGSETPGTSESATPESSGGSSGGS (SEQ ID NO: 357)]-[SpCas9-MQKFRAER
nickase R1337K]- [EGADKRTADGSEFESPKKKRKV (SEQ ID NO: 438)]
Variant B:
[TadA*7.10 (L36H, I76Y, V82T, Y147T, Q154S, and N157K)]-
[SGGSSGGSSGSETPGTSESATPESSGGSSGGS (SEQ ID NO: 357)]-[SpCas9-MQKFRAER
nickase Q1136Y, and R1337K]- [EGADKRTADGSEFESPKKKRKV (SEQ ID NO: 438)]
Variant C:
[TadA*7.10 (L36H, 176Y, V82T, Y147T, Q154S, and N157K)]-[EGGSEEEEESGS (SEQ ID
NO: 432)]-[SpCas9-MQKFRAER nickase M1135L, Q1136Y, and R1337K]-
[EGADKRTADGSEFESPKKKRKV (SEQ ID NO: 438)]
Variant D:
[TadA*7.10 (L36H, I76Y, V82T, Y147T, Q154S, and N157K)]-[EGGSEEEEESGS (SEQ ID
NO: 432)]-[SpCas9-MQKFRAER nickase Q1136Y, and R1337K]-
[EGADKRTADGSEFESPKKKRKV (SEQ ID NO: 438)]
Variant E:
[TadA*7.10 (L36H, I76Y, V82T, Y147T, Q154S, and N157K)]-[KGPKPKKEESEK (SEQ ID
NO: 439)]-[SpCas9-MQKFRAER nickase M1135L, Q1136Y, and R1337K]-
[EGADKRTADGSEFESPKKKRKV (SEQ ID NO: 438)]
Variant F:
[TadA*7.10 (L36H, I76Y, V82T, Y147T, Q154S, and N157K)]-
[SGGSSGGSSGSETPGTSESATPESSGGSSGGS (SEQ ID NO: 357)]-[SpCas9-MQKFRAER
nickase M1135L, A1283D, Q1136Y, and R1337K]- [EGADKRTADGSEFESPKKKRKV (SEQ
ID NO: 438)]
Variant G:
[TadA*7.10 (L36H, I76Y, V82T, Y147T, Q154S, and N157K)]-
[SGGSSGGSSGSETPGTSESATPESSGGSSGGS (SEQ ID NO: 357)]-[SpCas9-MQKFRAER
nickase M1135L, A1283D, E1250K, and R1337K]- [EGADKRTADGSEFESPKKKRKV (SEQ
ID NO: 438)]
Variant H:
[TadA*7.10 (L36H, I76Y, V82T, Y147T, Q154S, and N157K)]-
[SGGSSGGSSGSETPGTSESATPESSGGSSGGS (SEQ ID NO: 357)]-[SpCas9-MQKFRAER
nickase A1283D, E1250K, and Q1136Y]- [EGADKRTADGSEFESPKKKRKV (SEQ ID NO:
438)]
Variant I:
[TadA*7.10 (L36H, I76Y, V82T, Y147T, Q154S, and N157K)]- [KGPKPKKEESEK (SEQ ID
NO: 439)]-[SpCas9-MQKFRAER nickase M1135L, A1283D, Q1136Y, and R1337K]-
[EGADKRTADGSEFESPKKKRKV (SEQ ID NO: 438)]
Variant J:
[TadA*7.10 (L36H, I76Y, V82T, Y147T, Q154S, and N157K)]-
[SGGSSGGSSGSETPGTSESATPESSGGSSGGS (SEQ ID NO: 357)]-[SpCas9-MQKFRAER
nickase M1135L, A1283D, Q1136Y, R1337K, R765A, and Q768A]-
[EGADKRTADGSEFESPKKKRKV (SEQ ID NO: 438)]
Variant K:
[TadA*7.10 (L36H, I76Y, V82T, Y147T, Q154S, and N157K)]- [KGPKPKKEESEK (SEQ ID
NO: 439)]-[SpCas9-MQKFRAER nickase A1283D, E1250K, and Q1136Y]-
[EGADKRTADGSEFESPKKKRKV (SEQ ID NO: 438)]

In some embodiments the ABE Variant is ABE Variant G. In some embodiments, the ABE Variant is ABE Variant A, ABE Variant B, ABE Variant C, ABE Variant D, ABE Variant E, ABE Variant F, ABE Variant G, ABE Variant H, ABE Variant I, ABE Variant J, or ABE Variant K. In embodiments, an ABE variant comprises an amino acid sequence with at least about 85% sequence identity to an amino acid sequence of any one of ABE Variants A-K or comprises an amino acid sequence with at least 85% sequence identity to a deaminase, linker, and/or napDNAbp amino acid sequence of any one of ABE Variants A-K.

### Polynucleotide Programmable Nucleotide Binding Domain

Some aspects of the disclosure provide polynucleotide programmable nucleotide binding domains, and novel variants thereof. Such polynucleotide programmable nucleotide binding proteins, such as Cas9 proteins or variants thereof, may be part of a base editor or base editor system. In some embodiments, the polynucleotide programmable nucleotide binding protein is a Cas9 protein, or variant thereof. Non-limiting, exemplary Cas9 domains are provided herein. The Cas9 domain may be a nuclease active Cas9 domain, a nuclease inactive Cas9 domain (dCas9), or a Cas9 nickase (nCas9). In some embodiments, the Cas9 domain is a nuclease active domain. For example, the Cas9 domain may be a Cas9 domain that cuts both strands of a duplexed nucleic acid (e.g., both strands of a duplexed DNA molecule). In some embodiments, the Cas9 domain comprises any one of the amino acid sequences as set forth herein. In some embodiments the Cas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth herein. In some embodiments, the Cas9 domain comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the amino acid sequences set forth herein. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth herein.

In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild-type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to wild-type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild-type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild-type Cas9. In some embodiments, the fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, the disclosure provides a modified SpCas9, including amino acid substitutions D1135M, S1136Q, G1218K, E1219F, A1322R, D1332A, R1335E, and T1337R (SpCas9-MQKFRAER), which are indicated in bold-underlined text in the following SpCas9-MQKFRAER amino acid sequence. In some embodiments, the SpCas9-MQKFRAER is a nickase (e.g., the SpCas9-MQKFRAER polynucleotide may contain a G10A alteration, where a G10A alteration is indicated by underlined text in the below SpCas9-MQKFRAER amino acid sequence). In some embodiments, the modified SpCas9 has specificity for the altered PAM 5′-NGC-3′. In embodiments, an SpCas9 polypeptide of the disclosure has at least 85% sequence identity to the following amino acid sequence and is capable of functioning as a nucleic acid programmable DNA binding protein (napDNAbp).

SpCas9-MQKFRAER amino acid sequence:
(SpCas9-MQKFRAER nickase, SEQ ID NO: 462)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMVKRYDEHHQDLTLLKALVRQQLPEKYKEIFFDQSKNGYAGYIDGGASQEEF
YKFIKPILEKMDGTEELLVKLNREDLLRKQRTFDNGIIPHQIHLGELHAILRRQGDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRLRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGGHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKGNS
DKLIARKKDWDPKKYGGFMQPTVAYSVLVVAKVEKGKSKKLKSVKELLGITIMERSSFEKNP
IDFLEAKGYKEVKKDLIIKLPKYSLFELENGRKRMLASAKFLQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPRAFKYFDTTIARKEYRSTKEVLDATLIHQSITGLYETRIDLSQ
LGGD.

In the interest of clarity, amino acid substitutions D1135M, S1136Q, G1218K, E1219F, A1322R, D1332A, R1335E, and T1337R as compared to wild-type SpCas9 are indicated in the above sequence using bold and underlining. Amino acid substitution D10A, as compared to wild-type SpCas9 is indicated using underlining. In some embodiments, the SpCas9-MQKFRAER nickase does not comprise an N-terminal methionine.

In some embodiments, the disclosure provides novel Cas9 protein variants. In some embodiments, the disclosure provides novel SpCas9 variants. It should be appreciated that any of the Cas9 amino acid mutations disclosed herein may be made in any of the Cas9 protein provided herein. In some embodiments, any of the Cas9 proteins provided herein, including an SpCas9, a Hi-Fi Cas9, an SpCas9-MQKFRAER, or anSpCas9-MQKFRAER nickase comprises any one, two, three, four, five, six, seven, eight, nine, or ten of the following amino acid substitutions in a corresponding residue: W1126R, R1359W, E1250K, A1239T, A1239V, E1335D, M1135L, M1135R, M1135W, Q1136H, Q1136Y, K1218D, K1218R, K1218E, K1218L, F1219K, F1219N, R1322A, R1322K, R1337K, R1337T, and M1135V. In some embodiments an SpCas9-MQKFRAER nickase comprises any one of the following amino acid substitutions: W1126R, R1359W, E1250K, A1239T, A1239V, E1335D, M1135L, M1135R, M1135W, Q1136H, Q1136Y, K1218D, K1218R, K1218E, K1218L, F1219K, F1219N, R1322A, R1322K, R1337K, R1337T, and M1135V.

In some embodiments, any of the Cas9 proteins provided herein, including an SpCas9, a Hi-Fi Cas9, an SpCas9-MQKFRAER, or anSpCas9-MQKFRAER nickase comprises any two of the following amino acid substitutions in a corresponding residue: W1126R, R1359W, E1250K, A1239T, A1239V, E1335D, M1135L, M1135R, M1135W, Q1136H, Q1136Y, K1218D, K1218R, K1218E, K1218L, F1219K, F1219N, R1322A, R1322K, R1337K, R1337T, and M1135V. In some embodiments, the two amino acid substitutions are M1135L and Q1136H; M1135R and Q1136H; M1135L and Q1136Y; M1135R and Q1136Y; K1218D and F1219K; K1218R and F1219K; or E1335D and R1337K. In some embodiments an SpCas9-MQKFRAER nickase comprises any two of the following amino acid substitutions: W1126R, R1359W, E1250K, A1239T, A1239V, E1335D, M1135L, M1135R, M1135W, Q1136H, Q1136Y, K1218D, K1218R, K1218E, K1218L, F1219K, F1219N, R1322A, R1322K, R1337K, R1337T, and M1135V. In some embodiments, the two amino acid substitutions are M1135L and Q1136H; M1135R and Q1136H; M1135L and Q1136Y; M1135R and Q1136Y; K1218D and F1219K; K1218R and F1219K; Q1136Y and R1337K, or E1335D and R1337K.

In some embodiments, any of the Cas9 proteins provided herein, including an SpCas9, a Hi-Fi Cas9, an SpCas9-MQKFRAER, or anSpCas9-MQKFRAER nickase comprises any six of the following amino acid substitutions in a corresponding residue: W1126R, R1359W, E1250K, A1239T, A1239V, E1335D, M1135L, M1135R, M1135W, Q1136H, Q1136Y, K1218D, K1218R, K1218E, K1218L, F1219K, F1219N, R1322A, R1322K, R1337K, R1337T, and M1135V. In some embodiments, the six amino acid substitutions are M1135R, Q1136H, K1218D, R1322K, E1335D, and R1337K; or M1135V, Q1136H, K1218D, R1322K, E1335D, and R1337K. In some embodiments an SpCas9-MQKFRAER nickase comprises any six of the following amino acid substitutions: W1126R, R1359W, E1250K, A1239T, A1239V, E1335D, M1135L, M1135R, M1135W, Q1136H, Q1136Y, K1218D, K1218R, K1218E, K1218L, F1219K, F1219N, R1322A, R1322K, R1337K, R1337T, and M1135V. In some embodiments, the six amino acid substitutions are M1135R, Q1136H, K1218D, R1322K, E1335D, and R1337K; or M1135V, Q1136H, K1218D, R1322K, E1335D, and R1337K.

In some embodiments, the disclosure provides novel Cas9 protein variants. In some embodiments, the disclosure provides novel SpCas9 variants. It should be appreciated that any of the Cas9 amino acid mutations disclosed herein may be made in any of the Cas9 protein provided herein. In some embodiments, any of the Cas9 proteins provided herein, including an SpCas9, a Hi-Fi Cas9, an SpCas9-MQKFRAER, or an SpCas9-MQKFRAER nickase comprises any one, two, three, four, five, six, seven, eight, nine, or ten of the following amino acid substitutions in a corresponding residue: S217A, K218A, S219A, R220A, R221A, D699K, D700K, R765A, Q768A, K772A, K775A, K913A, K918A, R919A, K877A, K878A, K880A, R884A, K890A, R895A, and K896A. In some embodiments a Hi-Fi Cas9 or an SpCas9-MQKFRAER nickase comprises any one of the following amino acid substitutions: S217A, K218A, S219A, R220A, R221A, D699K, D700K, R765A, Q768A, K772A, K775A, K913A, K918A, R919A, K877A, K878A, K880A, R884A, K890A, R895A, and K896A.

In some embodiments, any of the Cas9 proteins provided herein, including an SpCas9, a Hi-Fi Cas9, an SpCas9-MQKFRAER, or anSpCas9-MQKFRAER nickase comprises any two, three, four, five, or six of the following amino acid substitutions in a corresponding residue: S217A, K218A, S219A, R220A, R221A, D699K, D700K, R765A, Q768A, K772A, K775A, K913A, K918A, R919A, K877A, K878A, K880A, R884A, K890A, R895A, and K896A. In some embodiments, the two amino acid substitutions are S217A and K218A; R220A and R221A; D699K and D700K; R765A and Q768A; K772A and K775A; or R895A and K896A. In some embodiments, a Hi-Fi Cas9 or an SpCas9-MQKFRAER nickase comprises the two amino acid substitutions S217A and K218A; R220A and R221A; D699K and D700K; R765A and Q768A; K772A and K775A; or R895A and K896A. In some embodiments, the three amino acid substitutions are S219A, R220A and R221A; or K913A, K918A and R919A. In some embodiments, a Hi-Fi Cas9 or an SpCas9-MQKFRAER nickase comprises the three amino acid substitutions S219A, R220A and R221A; or K913A, K918A and R919A. In some embodiments, the four amino acid substitutions are R765A, Q768A, K772A and K775A. In some embodiments, a Hi-Fi Cas9 or an SpCas9-MQKFRAER nickase comprises the four amino acid substitutions R765A, Q768A, K772A and K775A. In some embodiments, the five amino acid substitutions are S217A, K218A, S219A, R220A and R221A; or K877A, K878A, K880A, R884A and K890A. In some embodiments, a Hi-Fi Cas9 or an SpCas9-MQKFRAER nickase comprises the five amino acid substitutions S217A, K218A, S219A, R220A and R221A; or K877A, K878A, K880A, R884A and K890A. In some embodiments, the seven amino acid substitutions are K877A, K878A, K880A, R884A, K890A, R895A and K896A. In some embodiments, a Hi-Fi Cas9 or an SpCas9-MQKFRAER nickase comprises the seven amino acid substitutions K877A, K878A, K880A, R884A, K890A, R895A and K896A.

In some embodiments, the disclosure provides novel Cas9 protein variants. In some embodiments, the disclosure provides novel SpCas9 variants. It should be appreciated that any of the Cas9 amino acid mutations disclosed herein may be made in any of the Cas9 protein provided herein. In some embodiments, any of the Cas9 proteins provided herein, including an SpCas9, a Hi-Fi Cas9, an SpCas9-MQKFRAER, or anSpCas9-MQKFRAER nickase comprises any one, two, three, four, five, six, seven, eight, nine, or ten of the following amino acid substitutions in a corresponding residue: W18R, R40W, 148V, T58A, K65R, K76R, I85T, V93A, D94G, S96G, F119I, H129L, T134A, Y136H, L138Q, A159V, K163E, D173G, I170T, D173G, Q187R, S204G, S204I, D207G, I211N, P230S, K233M, K234E, N240S, K263I, T270I, L275Q, L291P, L301Q, L302Q, V322A, and H328L. In some embodiments a SpCas9-MQKFRAER nickase comprises any one of the following amino acid substitutions: W18R, R40W, 148V, T58A, K65R, K76R, 185T, V93A, D94G, S96G, F119I, H129L, T134A, Y136H, L138Q, A159V, K163E, D173G, 1170T, D173G, Q187R, S204G, S204I, D207G, I211N, P230S, K233M, K234E, N240S, K263I, T270I, L275Q, L291P, L301Q, L302Q, V322A, and H328L.

In some embodiments, any of the Cas9 proteins provided herein, including an SpCas9, a Hi-Fi Cas9, an SpCas9-MQKFRAER, or anSpCas9-MQKFRAER nickase comprises any two, three, four, five, or six of the following amino acid substitutions in a corresponding residue: W18R, R40W, 148V, T58A, K65R, K76R, 185T, V93A, D94G, S96G, F119I, H129L, T134A, Y136H, L138Q, A159V, K163E, D173G, 1170T, D173G, Q187R, S204G, S204I, D207G, I211N, P230S, K233M, K234E, N240S, K263I, T270I, L275Q, L291P, L301Q, L302Q, V322A, and H328L. In some embodiments, the two amino acid substitutions are V93A, and F119I; D94G, and A159V; S96G, and Y136H; T134A, and K163E; 1170T, and I211N; D173G, and D207G; D173G, and P230S; D173G, and K234E; S204G, and N240S; K263I, and T270I; L301Q, and V322A; or L302Q, and H328L. In some embodiments, a SpCas9-MQKFRAER nickase comprises the two amino acid substitutions V93A, and F119I; D94G, and A159V; S96G, and Y136H; T134A, and K163E; I170T, and I211N; D173G, and D207G; D173G, and P230S; D173G, and K234E; S204G, and N240S; K263I, and T270I; L301Q, and V322A; or L302Q, and H328L.

In some embodiments, the disclosure provides novel Cas9 protein variants. In some embodiments, the disclosure provides novel SpCas9 variants. It should be appreciated that any of the Cas9 amino acid mutations disclosed herein may be made in any of the Cas9 protein provided herein. In some embodiments, any of the Cas9 proteins provided herein, including an SpCas9, a Hi-Fi Cas9, an SpCas9-MQKFRAER, or anSpCas9-MQKFRAER nickase comprises any one, two, three, four, five, six, seven, eight, nine, or ten of the following amino acid substitutions in a corresponding residue: Q190L, N202S, K209M, K263I, T270I, V322A, G1104D, S1106N, D1117N, A1121T, D1127N, K1129E, T1138I, V1139M, A1147T, V1160M, L1164Q, A1184T, E1205V, A1217T, H1241L, K1246E, D1251G, H1264L, Y1265C, L1266Q, R1279G, A1283D, K1325G, I1331T, S1338G, L1343Q, H1349Y, R1359W, 11360S, G1367C, G1367Y, and G1378D. In some embodiments a SpCas9-MQKFRAER nickase comprises any one of the following amino acid substitutions: Q190L, N202S, K209M, K263I, T270I, V322A, G1104D, S1106N, D1117N, A1121T, D1127N, K1129E, T1138I, V1139M, A1147T, V1160M, L1164Q, A1184T, E1205V, A1217T, H1241L, K1246E, D1251G, H1264L, Y1265C, L1266Q, R1279G, A1283D, K1325G, I1331T, S1338G, L1343Q, H1349Y, R1359W, 11360S, G1367C, G1367Y, and G1378D.

In some embodiments, any of the Cas9 proteins provided herein, including an SpCas9, a Hi-Fi Cas9, an SpCas9-MQKFRAER, or anSpCas9-MQKFRAER nickase comprises any two, three, four, five, or six of the following amino acid substitutions in a corresponding residue: Q190L, N202S, K209M, K263I, T270I, V322A, G1104D, S1106N, D1117N, A1121T, D1127N, K1129E, T1138I, V1139M, A1147T, V1160M, L1164Q, A1184T, E1205V, A1217T, H1241L, K1246E, D1251G, H1264L, Y1265C, L1266Q, R1279G, A1283D, K1325G, 11331T, S1338G, L1343Q, H1349Y, R1359W, I1360S, G1367C, G1367Y, and G1378D. In some embodiments, the two amino acid substitutions are H1241L, and H1264L; or K1246E, and L1266Q; D1251G, and Y1265C; or Y1265C, and A1283D. In some embodiments, a SpCas9-MQKFRAER nickase comprises the two amino acid substitutions H1241L, and H1264L; or K1246E, and L1266Q; D1251G, and Y1265C; or Y1265C, and A1283D. In some embodiments, the three amino acid substitutions are Q190L, N202S, and K209M; K263I, T270I, and V322A; or D1251G, Y1265C, and A1283D. In some embodiments, a SpCas9-MQKFRAER nickase comprises the three amino acid substitutions Q190L, N202S, and K209M; K263I, T270I, and V322A; or D1251G, Y1265C, and A1283D.

Some aspects of the disclosure provide Cas9 variants, e.g., for use in base editors or base editor systems. In some embodiments the Cas9 variants are used in adenosine base editors or adenosine base editor systems. In some embodiments, the Cas9 variant is a SpCas9-MQKFRAER nickase variant. In some embodiments, the Cas9 Variant comprises an amino acid sequence that is at least 70%, 75%, 80%, 85%, 90%, 95%, 96%, 97%, 98%, 99%, or 100% identical to the amino acid sequence of the SpCas9-MQKFRAER nickase or the SpCas9-MQKFRAER nickase without the N-terminal methionine. In some embodiments, the Cas9 variant comprises an R1337K mutation, which is the Cas9 variant used in ABE Variant A. In some embodiments, the Cas9 variant comprises an R1337K, and a Q1136Y mutation, which is the Cas9 variant used in ABE Variant B. In some embodiments, the Cas9 variant comprises an M1135L, Q1136Y, and R1337K mutation, which is the Cas9 variant used in ABE Variant C. In some embodiments, the Cas9 variant comprises an Q1136Y, and R1337K mutation, which is the Cas9 variant used in ABE Variant D. In some embodiments, the Cas9 variant comprises an M1135L, R1337K, and Q1136Y mutation, which is the Cas9 variant used in ABE Variant E. In some embodiments, the Cas9 variant comprises an M1135L, A1283D, Q1136Y, and R1337K mutation, which is the Cas9 variant used in ABE Variant F. In some embodiments, the Cas9 variant comprises an M1135L, A1283D, E1250K, and R1337K mutation, which is the Cas9 variant used in ABE Variant G. In some embodiments, the Cas9 variant comprises an A1283D, E1250K, and Q1136Y mutation, which is the Cas9 variant used in ABE Variant H. In some embodiments, the Cas9 variant comprises an M1135L, A1283D, Q1136Y, and R1337K mutation, which is the Cas9 variant used in ABE Variant I. In some embodiments, the Cas9 variant comprises an M1135L, A1283D, Q1136Y, R1337K, R765A, and Q768A mutation, which is the Cas9 variant used in ABE Variant J. In some embodiments, the Cas9 variant comprises an A1283D, E1250K, and Q1136Y mutation, which is the Cas9 variant used in ABE Variant K. In some embodiments, the Cas9 variant comprises the amino acid sequence

(SEQ ID NO: 463)
DKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDAILLSDILRVNTE
ITKAPLSASMVKRYDEHHQDLTLLKALVRQQLPEKYKEIFFDQSKNGYAGYIDGGASQEEFY
KFIKPILEKMDGTEELLVKLNREDLLRKQRTFDNGIIPHQIHLGELHAILRRQGDFYPFLKD
NREKIEKILTFRIPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKAIVDLLFKTNRKV
TVKQLKEDYFKKIECFDSVEISGVEDRFNASLGTYHDLLKIIKDKDFLDNEENEDILEDIVL
TLTLFEDREMIEERLKTYAHLFDDKVMKQLKRLRYTGWGRLSRKLINGIRDKQSGKTILDFL
KSDGFANRNFMQLIHDDSLTFKEDIQKAQVSGQGDSLHEHIANLAGSPAIKKGILQTVKVVD
ELVKVMGGHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKGNSD
KLIARKKDWDPKKYGGFLQPTVAYSVLVVAKVEKGKSKKLKSVKELLGITIMERSSFEKNPI
DFLEAKGYKEVKKDLIIKLPKYSLFELENGRKRMLASAKFLQKGNELALPSKYVNFLYLASH
YEKLKGSPKDNEQKQLFVEQHKHYLDEIIEQISEFSKRVILDDANLDKVLSAYNKHRDKPIR
EQAENIIHLFTLTNLGAPRAFKYFDTTIARKEYKSTKEVLDATLIHQSITGLYETRIDLSQL
GGD (Variant G).

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, Turbo Cas9 (i.e., an SpCas9 with the amino acid alterations Q844R, V842L, F846Y, L847M, and I852F), homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152 (5): 1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR (N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

N is A, C, T, or G; and Vis A, C, or G.

In some embodiments, the PAM is NGC. In some embodiments, the NGC PAM is recognized by a Cas9 variant. In some embodiments, the NGC PAM Cas9 variant includes one or more amino acid substitutions selected from D1135M, S1136Q, G1218K, E1219F, A1322R, D1332A, R1335E, and T1337R (collectively termed “MQKFRAER”) of spCas9 (SEQ ID No: 197), or a corresponding mutation in another Cas9. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218).

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556 (7699), 57-63; Miller et al., “Continuous evolution of SpCas9 variants compatible with non-G PAMs” Nat. Biotechnol., 2020 April; 38 (4): 471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in SEQ ID NO: 197. Cas9 polypeptide regions comprising a higher than average B-factor can include, for example, residues 792-872, 792-906, and 2-791 as numbered in SEQ ID NO: 197.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in SEQ ID NO: 197, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in SEQ ID NO: 197, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in SEQ ID NO: 197, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)n (SEQ ID NO: 248), (GGS) n, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

(SEQ ID NO: 262)
ATGGCCCCAAAGAAGAAGCGGAAGGTCGGTATCCACGGAGTCCCAGCAG
CC.

In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### Adenosine Deaminases and A to G Editing

Some aspects of the disclosure provide adenosine deaminases, and novel variants thereof, which may be useful for base editor proteins and base editor systems.

In some embodiments, the adenosine deaminase comprises at least one alteration in the following sequence:

(SEQ ID NO: 1)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD (also termed TadA*7.10).

In the interest of clarity, residues L36, I76, V82, Y147, Q154, and N157 are indicated by bold and underlined text. In some embodiments any of the TadA*7.10 polypeptides, or variants thereof, provided herein do not comprise a methionine (M) residue at the beginning of the sequence. For example, TadA*7.10 without the methionine at the beginning of the sequence corresponds to the following amino acid sequence:

(SEQ ID NO: 464)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMPRQVFNAQKKAQSSTD.

It should be appreciated, however, that the amino acid numbering scheme of the TadA*7.10 without the methionine may correspond to the same amino acid numbering scheme of the TadA*7.10 having the initiating methionine present.

In some embodiments, TadA*7.10 comprises at least one amino acid alteration. In some embodiments, TadA*7.10 comprises an alteration in any one of amino acid residues L36, I76, V82, Y147, Q154, and N157 of TadA*7.10. In some embodiments, TadA*7.10 comprises any one of the amino acid alterations L36H, I76Y, V82T, Y147T, Q154S, and N157K of TadA*7.10.

In some embodiments, TadA*7.10 comprises the amino acid alterations L36H, I76Y, V82T, Y147T, Q154S, and N157K of TadA*7.10. For example, the TadA may have a sequence of

(SEQ ID NO: 426, TadA*7.10 L36H, I76Y,
V82T, Y147T, Q154S, and N157K)
SEVEFSHEYWMRHALTLAKRARDEREVPVGAVLVHNNRVIGEGWNRAIGL
HDPTAHAEIMALRQGGLVMQNYRLYDATLYTTFEPCVMCAGAMIHSRIGR
VVFGVRNAKTGAAGSLMDVLHYPGMNHRVEITEGILADECAALLCTFFRM
PRSVFKAQKKAQSSTD

In some embodiments, the disclosure provides TadA variants comprising a V82T, Y147T, and/or a Q154S mutation. In some embodiments, the disclosure provides TadA*8.8 further comprising a V82T mutation. In some embodiments, the disclosure provides TadA*8.8 further comprising a V82T, a Y147T, and a Q154S mutation. In some embodiments, the disclosure provides TadA*8.17 further comprising a V82T mutation. In some embodiments, the disclosure provides TadA*8.17 further comprising a V82T, a Y147T, and a Q154S mutation. In some embodiments, the disclosure provides TadA*8.20 further comprising a V82T mutation. In some embodiments, the disclosure provides TadA*8.20 further comprising a V82T, a Y147T, and a Q154S mutation. In some embodiments, the disclosure provides any of the TadA proteins, or variants thereof, that have at least 70%, 75%, 80%, 85%, 90%, 95%, 96%, 97%, 98%, or 99% identity to any TadA protein, or variant thereof, provided herein.

Some aspects of the disclosure provide novel adenosine deaminase variants. In some embodiments, such variants have improved trinucleotide specificity of the target A and adjacent nucleotides (i.e., NAN). In some embodiments, the disclosure provides novel TadA variants. It should be appreciated that any of the TadA amino acid mutations disclosed herein may be made in any of the TadA proteins or variants provided herein. In some embodiments, any of the TadA proteins provided herein, including TadA*7.10, comprises any one, two, three, four, five, six, seven, eight, nine, or ten of the following amino acid substitutions in a corresponding residue: F84Y, A109L, A109V, A109I, A109F, A109S, A109T, A109N, V155S, V155T, V155N, F156Y, F156W, F156R, F156N, and F156Q.

In some embodiments, any of the TadA proteins provided herein, including TadA*7.10 comprises any two of the following amino acid substitutions in a corresponding residue: F84Y, A109L, A109V, A109I, A109F, A109S, A109T, A109N, V155S, V155T, V155N, F156Y, F156W, F156R, F156N, and F156Q. In some embodiments, the two amino acid substitutions are F84Y and A109L; F84Y and A109S; F84Y and V155S; F84Y and V155N; F84Y and F156R; F84Y and F156N; F84Y and F156Q; A109L and V155S; A109L and V155N; A109L and F156R; A109L and F156N; A109L and F156Q; A109S and V155S; A109S and V155N; A109S and F156R; A109S and F156N; A109S and F156Q; V155S and F156R; V155S and F156N; V155S and F156Q; V155N and F156R; V155N and F156N; or V155N and F156Q.

In some embodiments, any of the TadA proteins provided herein, including TadA*7.10 comprises any three of the following amino acid substitutions in a corresponding residue: F84Y, A109L, A109V, A109I, A109F, A109S, A109T, A109N, V155S, V155T, V155N, F156Y, F156W, F156R, F156N, and F156Q. In some embodiments, the three amino acid substitutions are F84Y, A109L, and V155S; F84Y, A109L, and V155N; F84Y, A109S, and V155S; F84Y, A109S, and V155N; A109L, V155S, and F156N; A109L, V155N, and F156N; A109S, V155S, and F156N; or A109S, V155N, and F156N.

In some embodiments, any of the TadA proteins provided herein, including TadA*7.10 comprises any four of the following amino acid substitutions in a corresponding residue: F84Y, A109L, A109V, A109I, A109F, A109S, A109T, A109N, V155S, V155T, V155N, F156Y, F156W, F156R, F156N, and F156Q. In some embodiments, the four amino acid substitutions are F84Y, A109L, V155S, and F156N.

In some embodiments, any of the TadA proteins provided herein, including a TadA*5, comprises any one, two, three, four, five, six, seven, eight, nine, or ten of the following amino acid substitutions in a corresponding residue: E3N, E3K, E3G, F6A, H14D, L18A, W23I, W23R, P29T, P29Y, P29Q, V35Q, L36S, N38D, G42M, N46Y, P48A, G50A, H52L, A62V, L63R, L63F, Q65R, G67N, L68V, M70I, N72Y, T79H, Y81V, V82S, M94R, G100V, V102E, V102S, R107A, A114C, G115E, M118L, D119L, H122T, P124H, P124K, P124Q, H128R, V130F, 1132K, 1132T, E140L, A142N, A142S, L144Q, L145R, L145N, Y147A, F149A, R152P, F156N, and K160E. In some embodiments, any of the TadA proteins provided herein, including a TadA*5, comprises two of the amino acid substitutions. In some embodiments, the two amino acid substitutions are V102S, and G115E; G100V, and P29T; L145R, and G42M; R107A, and L63F; V82S, and E3K; V82S, and I132K; V82S, and V102E; V82S, and D119L; V82S, and L144Q; V82S, and Y147A; V82S, and M118L; V82S, and A62V; Y81V, and L18A; L145R, and G42M; K160E, and H14D; V82S, and G67N; V82S, and P124K; V35Q, and V130F; T79H, and L145N; V82S, and E3N; V82S, and I132T; V82S, and L63R; V82S, and N46Y; V82S, and F149A; V82S, and G67N; V82S, and Q65R; V82S, and M70I; V82S, and P124H; or V82S, and H52L. In some embodiments, any of the TadA proteins provided herein, including a TadA*5, comprises three of the amino acid substitutions. In some embodiments, the three amino acid substitutions are A114C, E140L, and W23I; E3G, 1132T, and F6A; W23R, P48A, and R152P; V82S, G100V, and P29T; V82S, L145R, and G42M; V82S, V102S, and G115E; V82S, R107A, and L63F; N72Y, F156N, and H128R; N38D, P124Q, and L68V; P29Y, M94R, and A142N; V82S, Y81V, and L18A; V82S, L145R, and G42M; V82S, K160E, and H14D; V82S, V35Q, and V130F; or V82S, T79H, and L145N. In some embodiments, any of the TadA proteins provided herein, including a TadA*5, comprises four of the amino acid substitutions. In some embodiments, the four amino acid substitutions are V82S, A114C, E140L, and W23I; V82S, E3G, I132T, and F6A; V82S, N72Y, F156N, and H128R; V82S, N38D, P124Q, and L68V; V82S, P29Y, M94R, and A142N. In some embodiments, any of the TadA proteins provided herein, including a TadA*5, comprises five of the amino acid substitutions. In some embodiments, the five amino acid substitutions are G50A, H122T, A142S, P29Q, and L36S. In some embodiments, any of the TadA proteins provided herein, including a TadA*5, comprises six of the amino acid substitutions. In some embodiments, the six amino acid substitutions are V82S, G50A, H122T, A142S, P29Q, and L36S.

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, 1156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced
to TadA*7.10. Additional details of TadA*9 adenosine
deaminases are described in International PCT Application
No. PCT/US2020/049975, which is incorporated herein by
reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F, I76Y, V82S, Y123H, Y147R, Q154R |
| TadA*9.30 | I76Y, V82T, Y123H, Y147R, Q154R |
| TadA*9.31 | N38G, I76Y, V82S, Y123H, Y147R, Q154R |
| TadA*9.32 | N38G, I76Y, V82T, Y123H, Y147R, Q154R |
| TadA*9.33 | R23H, I76Y, V82S, Y123H, Y147R, Q154R |
| TadA*9.34 | P54C, I76Y, V82S, Y123H, Y147R, Q154R |
| TadA*9.35 | R21N, I76Y, V82S, Y123H, Y147R, Q154R |
| TadA*9.36 | I76Y, V82S, Y123H, D138M, Y147R, Q154R |
| TadA*9.37 | Y72S, I76Y, V82S, Y123H, Y147R, Q154R |
| TadA*9.38 | E25F, I76Y, V82S, Y123H, Y147R, Q154R |
| TadA*9.39 | I76Y, V82T, Y123H, Y147R, Q154R |
| TadA*9.40 | N38G, I76Y, V82S, Y123H, Y147R, Q154R |
| TadA*9.41 | N38G, I76Y, V82T, Y123H, Y147R, Q154R |
| TadA*9.42 | R23H, I76Y, V82S, Y123H, Y147R, Q154R |
| TadA*9.43 | P54C, I76Y, V82S, Y123H, Y147R, Q154R |
| TadA*9.44 | R21N, I76Y, V82S, Y123H, Y147R, Q154R |
| TadA*9.45 | I76Y, V82S, Y123H, D138M, Y147R, Q154R |
| TadA*9.46 | Y72S, I76Y, V82S, Y123H, Y147R, Q154R |
| TadA*9.47 | N72K, V82S, Y123H, Y147R, Q154R |
| TadA*9.48 | Q71M, V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K, V82S, Y123H, Y147R, Q154R |
| TadA*9.54 | Q71M, V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises one or more of M1I, S2A, S2E, V4D, V4E, V4M, F6S, H8E, H8Y, E9Y, M12S, R13H, R13I, R13Y, T17L, T17S, L18A, L18E, A19N, R21N, K20K, K20R, R21A, G22P, W23D, R23H, W23G, W23Q, W23L, W23R, D24E, D24G, E25F, E25M, E25D, E25A, E25G, E25R, E25V, E25S, E25Y, R26D, R26E, R26G, R26N, R26Q, R26C, R26L, R26K, R26W, E27V, E27D, P29V, V30G, L34S, L34V, L36H, H36L, H36N, N37N, N37T, N37S, N38G, N38R, W45A, W45L, W45N, N46N, R46W, R46F, R46Q, R46M, R47A, R47Q, R47F, R47K, R47P, R47W, R47M, P48T, P48L, P48A, P48I, P48S, 149G, 149H, 149V, 149F, 149H, G50L, R51H, R51L, R51N, L51W, R51Y, H52D, H52Y, D53P, P54C, P54T, A55H, T55A, A56E, A56S, E59A, E59G, E591, E59Q, E59W, M61A, M61I, M61L, M61V, L63S, L63V, Q65V, G66C, G67D, G67L, G67V, L68Q, M70H, M70Q, L84F, M70V, M70L, E70A, M70V, Q71M, Q71N, Q71L, Q71R, N72A, N72K, N72S, N72D, N72Y, Y73G, Y73I, Y73K, Y73R, Y73S, R74A, R74Q, R74G, R74K, R74L, R74N, I76D, I76F, 1761, I76N, 176T, 176Y, D77G, A78I, T79M, L80M, L80Y, V82A, V82S, V82G, V82T, L84E, L84F, L84Y, E85K, E85G, E85P, E85S, S87C, S87L, S87V, V88A, V88M, C90S, A91A, A91G, A91S, A91V, A91T, G92T, A93I, M94A, M94V, M94L, M94I, M94H, 195S, 195G, 195L, 195H, 195V, H96A, H96L, H96R, H96S, S97C, S97G, S971, S97M, S97R, S97S, R98K, R981, R98N, R98Q, G100R, G100V, R101V, R101R, V102A, V102F, V102I, V102V, D103A, F104G, D104N, F104V, F104I, F104L, A106T, V106Q, V106F, V106W, V106M, A106A, A106Q, A106F, A106G, A106W, A106M, A106V, A106R, R107C, R107G, R107P, R107K, R107A, R107N, R107W, R107H, R107S, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, A109H, A109K, A109R, A109S, A109T, A109V, K110G, K110H, K110I, K110R, K110T, T111A, T111G, T111H, T111R, G112A, A114G, A114H, A114V, G115S, L117M, L117N, L117V, M118D, M118G, M118K, M118N, M118V, D119L, D119N, D119S, D119V, V120H, V120L, H122H, H122N, H122P, H122R, H122S, H122Y, H123C, H123G, H123P, H123V, H123Y, Y123H, P124G, P124I, P124L, P124W, G125H, G125I, G125A, G125M, G125K, M126D, M126H, M126K, M126I, M126N, M1260, M126S, M126Y, N127H, N127S, N127D, N127K, N127R, H128R, R129H, R129Q, R129V, R129I, R129E, R129V, 11321, 1132F, T133V, T133E, T133G, T133K, E134A, E134E, E134G, E134I, G135G, G135V, 1136G, 1136L, 1136T, 1137A, 1137D, 1137E, L137M, 1137S, A138D, A138E, A138G, S138A, A138N, A138S, A138T, A138V, A138Y, D139E, D139I, D139C, D139L, D139M, E140A, E140C, E140L, E140R, A142N, A142D, A142G, A142A, A142L, A142S, A142T, A142N, A142S, A142V, A143D, A143E, A143G, A143D, A143G, A143E, A143L, A143W, A143M, A143S, A143Q, A143R, C146R, S146A, S146C, S146D, S146F, S146R, S146T, D147D, D147L, D147F, D147G, D147Y, Y147T, Y147R, Y147D, D147R, F148L, F148F, F148R, F148Y, F149C, F149M, F149R, F149Y, M151F, M151P, M151R, M151V, R152C, R152F, R152H, R152P, R152R, R153C, R153Q, R153R, R153V, Q154E, Q154H, Q154M, Q154R, Q154L, Q154S, Q154V, E155F, E155G, E155I, E155K, E155P, E155V, E155D, 1156A, 1156F, 1156D, 1156K, 1156N, 1156R, 1156Y, E157A, E157F, E157I, E157P, E157T, E157V, N157K, K157N, K157R, A158Q, A158K, A158V, Q159F, Q159K, Q159L, Q159N, K160A, K160S, K160E, K160K, K160N, K161I, K161A, K161N, K161Q, K161S, K161T, A162D, A162Q, R162H, R162P, A162S, Q163G, Q163H, Q163N, Q163R, S164I, S164R, S164Y, S165A, S165D, S165I, S165T, S165Y, T166D, T166K, T166I, T166N, T166P, T166R, D167S and/or D167N mutation in a TadA reference sequence (e.g., TadA*7.10, ecTadA, or TadA8e), and any alternative mutation at the corresponding position, or any substitution from R26, W23, E27, H36, R47, P48, R51, H52, R74, 176, V82, V88, M94, 195, H96, A106, D108, A109, K110, T111, A114, D119, H122, H123, M126, N127, A142, S146, D147, F149, R152, Q154, E155, 1156, E157, K161, T166, and/or D167, with respect to a TadA reference sequence, or a substitution of 2-50 amino acids in a TadA reference sequence, which may be selected from W23R, E27D, H36L, R47K, P48A, R51H, R51L, I76F, 176Y, V82S, A106V, D108G, A109S, K110R, T111H, A114V, D119N, H122R, H122N, H123Y, M126I, N127K, S146C, D147R, R152P, Q154R, E155V, 1156F, K157N, K161N, T166I, and D167N, or one or more corresponding mutations in another adenosine deaminase. Additional mutations are described in U.S. Patent Application Publication No. 2022/0307003 A1 and International Patent Application Publications No. WO 2023/288304 A2 and WO 2023/034959 A2, the disclosures of which are incorporated herein by reference in their entirety for all purposes.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA- | R | V | A | T | D | H | Y | F | T | D |
| | 7.10 | | | | | | | | | | |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA (wt)) linked to a TadA*. Throughout the present disclosure, an adenosine deaminase base editor that comprises a single TadA* domain and a TadA (wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA (wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ~20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

Some aspects of the disclosure provide guide RNAs (e.g., single guide RNAs) comprising a sequence (e.g., a targeting sequence) that is complementary to an alpha-1 antitrypsin nucleic acid sequence comprising a SNP associated with alpha-1 antytrypsin deficiency. In some embodiments, the gRNA comprises the nucleic acid sequence (e.g., targeting sequence): ACCAUCGACAAGAAAGGGACUGA (SEQ ID NO: 466), CCAUCGACAAGAAAGGGACUGA (SEQ ID NO: 467), CAUCGACAAGAAAGGGACUGA (SEQ ID NO: 469), AUCGACAAGAAAGGGACUGA (SEQ ID NO: 468), CAUCGACAAGAAAGGGACUGA (SEQ ID NO: 469), AUCGACAAGAAAGGGACUGA (SEQ ID NO: 434) or a variant thereof comprising one or more modifications. In some embodiments, the gRNA comprises the nucleic acid sequence (e.g., targeting sequence): AUCGACAAGAAAGGGACUGA (SEQ ID NO: 434), CAUCGACAAGAAAGGGACUGA (SEQ ID NO: 469), or a variant thereof comprising one or more modifications.

In some embodiments, the gRNA further comprises the nucleic acid sequence (e.g., a scaffold sequence):

(SEQ ID NO: 317)
5'- GUUUUAGAGC UAGAAAUAGC AAGUUAAAAU AAGGCUAGUC
CGUUAUCAAC UUGAAAAAGU GGCACCGAGU CGGUGCUUUU-3',

or any variant thereof (e.g., as provided herein).

In some embodiments any of the gRNAs provided herein comprise one or more modifications. Exemplary modifications are provided herein (e.g., in the Modified Polynucleotides section). Exemplary gRNAs designed to target an alpha-1 antitrypsin nucleic acid sequence comprising a SNP associated with alpha-1 antytrypsin deficiency are provided below. It should be appreciated that the exemplary guides and modifications thereof are not intended to be limiting. In the following guide sequences, “N” represents any nucleotide, “mN” indicates a 2′-OMe modification of the nucleotide “N”, fN indicates a 2′-fluoro (F) modification of the nucleotide “N,” and “Ns” indicates that the nucleotide “N” is linked to the following nucleotide by a phosphorothioate (PS):

(SEQ ID NO: 569)
mAsmUsmCsGACAAGAAAGGGACUGAGUUUUAGAGCUAGAAAUAGCAAGUUAAAAUAAGGCU
AGUCCGUUAUCAACUUGAAAAAGUGGCACCGAGUCGGUGCmUsmUsmUsU (gRNA025)
(SEQ ID NOs: 570 and 543))
mAsmUsmCsGACAAGAAAGGGACUGAGUUUUAGAGCUAGAAAUAGCAAGUUAAAAUAAGGCU
AGUCCGUUAUCAACUUGAAAAAGUGGCACCGAGUCGGUGCUsmUsmUsmU-
CKRTADGSEFESPKKKRKV (sgRNA056)
(SEQ ID NO: 571)
mAsmUsmCsmGmAmCmAmAmGmAfAfAfGIGGsAsfCfUGsmAmGUsUsUsfUfAmGmAmGm
CmUmAmGmAmAmAmUmAmGmCmAmAmGUUmAAmAmAUmAmAmGmGCUmAGUCmCGUUmAmUm
CmAmAmCmUmUmGmAmAmAmAmAmGmUmGmGmCmAmCmCmGmAmGmUmCmGmGmUmGmCsmU
smUsmU (sgRNA058)
(SEQ ID NO: 572)
mAsmUsmCsGACAAGAAAGGGACUGAmGUUUUAGmAmGmCmUmAmGmAmAmAmUmAmGmCmA
mAGUUmAAmAAmUAmAmGmGmCmUmAGUmCmCGUUAmUmCAAmCmUmUmGmAmAmAmAmAmG
mUmGGmCmAmCmCmGmAmGmUmCmGmGmUmGmCmUsmUsmUsmU (sgRNA086)
(SEQ ID NO: 573)
mCsmAsmUsCGACAAGAAAGGGACUGAGUUUUAGAGCUAGAAAUAGCAAGUUAAAAUAAGGC
UAGUCCGUUAUCAACUUGAAAAAGUGGCACCGAGUCGGUGCmUsmUsmUsU (sgRNA182)
(SEQ ID NO: 574)
mAsmUsmCsmGmAmCmAmAmGmAfAfAfGfGGsAsfCUGmAmGUSUUfUfAmGmAmGmCmUm
AmGmAmAmAmUmAmGmCmAmAmGUUmAAmAmAUmAmAmGmGCUmAGUCmCGUUmAmUmCmAm
AmCmUmUmGmAmAmAmAmAmGUGmGmCmAmCmCmGmAmGmUmCmGmGmUmGmCsmUsmU
smU (gRNA855)
(SEQ ID NO: 575)
mAsmUsmCsGACAAGAAAGGGACUGAmGUUUUAGmAmGmCmUmAmGmAmAmAmUmAmGmCmA
mAGUUmAAmAAmUAmAmGmGmCmUmAGUmCmCGUUAmUmCAAmCmUmUmGmAmAmAmAmAmG
UGGmCmAmCmCmGmAmGmUmCmGmGmUmGmCmUsmUsmUsmU (gRNA856)
(SEQ ID NO: 576)
mAsmUsmCsmGmAmCmAmAmGmAAAGGGACUGmAmGUUUUAmGmAmGmCmUmAmGmAmAmAm
UmAmGmCmAmAmGUUmAAmAmAUmAmAmGmGCUmAGUCmCGUUmAmUmCmAmAmCmUmUmGm
AmAmAmAmAmGUGmGmCmAmCmCmGmAmGmUmCmGmGmUmGmCsmUsmUsmU (gRNA857)
(SEQ ID NO: 577)
mAsmUsmCsmGmAmCmAmAmGmAAAGGGACUGAmGUUUUAGmAmGmCmUmAmGmAmAmAmUm
AmGmCmAmAGUUmAAmAAmUAmAmGmGmCmUmAGUmCmCGUUAmUmCAAmCmUmUmGmAmAm
AmAmAmGUGGmCmAmCmCmGmAmGmUmCmGmGmUmGmCmUsmUsmUsmU (gRNA1447)
(SEQ ID NO: 578)
mCsmAsmUsmCmGmAmCmAmAmGmAAAGGGACUGAmGUUUUAGmAmGmCmUmAmGmAmAmAm
UmAmGmCmAmAGUUmAAmAAmUAmAmGmGmCmUmAGUmCmCGUUAmUmCAAmCmUmUmGmAm
AmAmAmAmGUGGmCmAmCmCmGmAmGmUmCmGmGmUmGmCmUsmUsmUsmU (gRNA1448)
(SEQ ID NO: 579)
mAsmUsmCsmGmAmCmAmAmGmAmAAmGmGGAmCUGmAmGUUUUAGmAmGmCmUmAmGmAmA
mAmUmAmGmCmAmAGUUmAAmAAmUAmAmGmGmCmUmAGUmCmCGUUAmUmCAAmCmUmUmG
mAmAmAmAmAmGUGGmCmAmCmCmGmAmGmUmCmGmGmUmGmCmUsmUsmUsmU (gRNA1449)
(SEQ ID NO: 580)
mCsmAsmUsmCmGmAmCmAmAmGmAmAAmGmGGAmCUGmAmGUUUUAGmAmGmCmUmAmGmA
mAmAmUmAmGmCmAmAGUUmAAmAAmUAmAmGmGmCmUmAGUmCmCGUUAmUmCAAmCmUmU
mGmAmAmAmAmAmGUGGmCmAmCmCmGmAmGmUmCmGmGmUmGmCmUsmUsmUs
mU (gRNA1450)
(SEQ ID NO: 581)
mCsmAsmUsCGACAAGAAAGGGACUGAmGUUUUAGmAmGmCmUmAmGmAmAmAmUmAmGmCm
AmAGUUmAAmAAmUAmAmGmGmCmUmAGUmCmCGUUAmUmCAAmCmUmUmGmAmAmAmAmAm
GUGGmCmAmCmCmGmAmGmUmCmGmGmUmGmCmUsmUsmUsmU (gRNA1451)
(SEQ ID NO: 582)
mAsmUsmCsGACAAGAAAGGGACUGAGUUUUAGAmGmCmCmGmGmCmGmGmAmAmAmCmGmC
mCmGmGmCAAGUUAAAAUAAGGCUAGUCCGUUAUCAmAmCmUmUmGmAmAmAmAmAmGmUmG
mGmCmAmCmCmGmAmGmUmCmGmGmUmGmCmUsmUsmUsmU (gRNA1071)
(SEQ ID NO: 583)
mAsmUsmCsmGmAmCmAmAmGmAAAGGGACUGAGUUUUAGAmGmCmCmGmGmCmGmGmAmAm
AmCmGmCmCmGmGmCAAGUUAAAAUAAGGCUAGUCCGUUAUCAmAmCmUmUmGmAmAmAmAm
AmGmUmGmGmCmAmCmCmGmAmGmUmCmGmGmUmGmCmUsmUsmUsmU (gRNA1850)
(SEQ ID NO: 584)
mAsmUsmCsGACAAGAAAGGGACUGAGUUUUAGAmGmCmUmAmGmAmAmAmUmAmGmCAAGU
UAAAAUAAGGCUAGUCCGUUAmUmCAAmCmUmUGGACUUCGGUCCmAmAmGmUmGGmCmAmC
mCmGmAmGmUmCmGmGmUmGmCmUsmUsmUsmU (gRNA1851)
(SEQ ID NO: 585)
mAsmUsmCsGACAAGAAAGGGACUGAGUUUUAGAmGmCmCmGmGmCmGmGmAmAmAmCmGmC
mCmGmGmCAAGUUAAAAUAAGGCUAGUCCGUUAmUmCAAmCmUmUGGACUUCGGUCCmAmAm
GmUmGGmCmAmCmCmGmAmGmUmCmGmGmUmGmCmUmUmUmU (gRNA1852)

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications. Such modifications may increase base editing ~2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold. In some embodiments, the disclosure provides gRNAs that comprise 2′-O-methyl or phosphorothioate modifications other than, or in addition to, the three nucleotides at the 5′ and 3′ end of the gRNA. In some embodiments at least 40%, 45%, 47%, 50%, 55%, 60%, 65%, 70%, 75%, 80%, 85%, 88%, 90%, 91%, 92%, 93%, 94%, 95%, 96%, 97%, 98%, 99%, or 100% of the nucleotides of a gRNA comprise a 2′-O-methyl or phosphorothioate modification.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

In some embodiments, a guide polynucleotide of the disclosure contains a scaffold with one of the following chemical modification schemes, where “N” represents any nucleotide, “mN” indicates a 2′-OMe modification of the nucleotide “N”, and “Ns” indicates that the nucleotide “N” is linked to the following nucleotide by a phosphorothioate (PS):

End-mod SpCas9 guide polynucleotide
(SEQ ID NO: 487)
mNsmNsmNsNNNNNNNNNNNNNNNNNGUUUUAGAGCUAGAAAUAGCAAGUUAAAAUAAGGCU
AGUCCGUUAUCAACUUGAAAAAGUGGCACCGAGUCGGUGCUsmUsmUsmU
End-mod SaCas9 guide polynucleotide
(SEQ ID NO: 488)
mNsmNsmNsNNNNNNNNNNNNNNNNNNGUUUUAGUACUCUGUAAUGAAAAUUACAGAAUCUA
CUAAAACAAGGCAAAAUGCCGUGUUUAUCUCGUCAACUUGUUGGCGAGAUsmUsmUsmU
HM01:
(SEQ ID NO: 489)
mNsmNsmNsNNNNNNNNNNNNNNNNNGUUUUAGAmGmCmUmAmGmAmAmAmUmAmGmCAAGU
UAAAAUAAGGCUAGUCCGUUAUCAmAmCmUmUmGmAmAmAmAmAmGmUmGmGmCmAmCmCmG
mAmGmUmCmGmGmUmGmCmUsmUsmUsmU
HM07:
(SEQ ID NO: 490)
mNsmNsmNsmNmNmNmNmNmNmNNNNNNNNNNNmGUUUUAGmAmGmCmUmAmGmAmAmAmUm
AmGmCmAmAGUUmAAmAAmUAmAmGmGmCmUmAGUmCmCGUUAmUmCAAmCmUmUmGmAmAm
AmAmAmGmUmGGmCmAmCmCmGmAmGmUmCmGmGmUmGmCmUsmUsmUsmU
NLS (bpsv40):
(SEQ ID NOs: 491 and 543)
mNsmNsmNsNNNNNNNNNNNNNNNNNGUUUUAGAGCUAGAAAUAGCAAGUUAAAAUAAGGCU
AGUCCGUUAUCAACUUGAAAAAGUGGCACCGAGUCGGUGCmUsmUsmUsmU-NHC6-CrossL-
ac-CKRTADGSEFESPKKKRKV
LONGEST:
(SEQ ID NO: 493)
mNsmNsmNsNNNNNNNNNNNNNNNNNGUUUUAGAmGmCmCmGmGmCmGmGmAmAmAmCmGmC
mCmGmGmCAAGUUAAAAUAAGGCUAGUCCGUUAUCAmAmCmUmUmGmAmAmAmAmAmGUGmG
mCmAmCmCmGmAmGmUmCmGmGmUmGmCmUsmUsmUsmU
NLS + LONGEST:
(SEQ ID NOs: 494 and 543)
mNsmNsmNsNNNNNNNNNNNNNNNNNGUUUUAGAmGmCmCmGmGmCmGmGmAmAmAmCmGmC
mCmGmGmCAAGUUAAAAUAAGGCUAGUCCGUUAUCAmAmCmUmUmGmAmAmAmAmAmGmUmG
mGmCmAmCmCmGmAmGmUmCmGmGmUmGmCmUsmUsmUsmU-NHC5-CrossL-
CKRTADGSEFESPKKKRKV
LONGEST + GOLD:
(SEQ ID NO: 496)
mNsmNsmNsNNNNNNNNNNNNNNNNNGUUUUAGAmGmCmCmGmGmCmGmGmAmAmAmCmGmC
mCmGmGmCAAGUUAAAAUAAGGCUAGUCCGUUAmUmCAAmCmUmUGGACUUCGGUCCmAmAm
GUGGmCmAmCmCmGmAmGmUmCmGmGmUmGmCmUsmUsmUsmU

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite-2 parts, while monopartite NLSs are not). The NLS of nucleoplasmin, KR [PAATKKAGQA] KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO: 328). In some embodiments, any of the adenosine base editors provided herein, for example ABE Variant A, ABE Variant B, ABE Variant C, ABE Variant D, ABE Variant E, ABE Variant F, ABE Variant G, ABE Variant H, ABE Variant I, ABE Variant J, ABE Variant K, or ABE Variant D comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fe domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a Spy Tag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. In some embodiments, the adenosine base editor (ABE) can deaminate adenine in DNA. In some embodiments, ABE is generated by replacing APOBECI component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 6 refers to a monomeric form of TadA*7.10 comprising the alterations described. The term “heterodimer” as used in Table 6 refers to the specified wild-type E. coli TadA adenosine deaminase fused to a TadA*7.10 comprising the alterations as described.

TABLE 6
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + 176Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS) n (SEQ ID NO: 247), and (G) n to more rigid linkers of the form (EAAAK), (SEQ ID NO: 248), (SGGS) n (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32 (6): 577-82; the entire contents are incorporated herein by reference) and (XP) n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS) n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGSP
TSTEEGTSTEPSEGSAPGTSTEPSEGSAPGTSESATPESGPGSEPATSGG
SGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEEG
TSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10 (1): 439; the entire contents are incorporated herein by reference). Such proline-rich linkers are also termed “rigid” linkers.

Some aspects of the disclosure provide novel polypeptides. In some embodiments the polypeptides, provided herein, are used as linkers and may be used to link any of the peptides or peptide domains of the disclosure. In some embodiments the linkers are used to link a TadA domain (e.g., a TadA*7.10) to a Cas9 domain (e.g., a SpCas9-MQKFRAER nickase). In some embodiments, such linkers are rationally designed to bring a deaminase domain (e.g., a TadA domain) in proximity to a target adenine (e.g., at position A7) in a target site. In some embodiments, the polypeptide comprises any one of the amino acid sequences:

(SEQ ID NO: 497)
STDTTKDTSPKPQDKI,
(SEQ ID NO: 498)
STDANAHSDISTGDKI,
(SEQ ID NO: 499)
STDEDKRSDLGKQDKI,
(SEQ ID NO: 500)
STDEKDQQPNSTSDKI,
(SEQ ID NO: 501)
STDQKGQKIPSSNDKI,
(SEQ ID NO: 502)
STDDRSQKQDQQDDKI,
(SEQ ID NO: 503)
STDSKSVPLSSDGDKI,
(SEQ ID NO: 504)
STDRQPRKDNSSGDKI,
(SEQ ID NO: 505)
STDKEGDQPPPPKDKI,
(SEQ ID NO: 506)
STDDVEYYSRPPDDKI,
(SEQ ID NO: 507)
STDDPRKREIPPPDKI,
(SEQ ID NO: 508)
STDERQHSTPTTDDKI,
(SEQ ID NO: 509)
STDSSKOPENPPQDKI,
(SEQ ID NO: 510)
STDPGGAPETNSGDKI,
(SEQ ID NO: 511)
STDNISLSYDQQDDKI,
(SEQ ID NO: 512)
STDSKSQQFVTYEDKI,
and
(SEQ ID NO: 513)
STDLTESRPPQESDKI.

In some embodiments, the polypeptide comprises any one of the amino acid sequences: EGSSSKEEEEPG (SEQ ID NO: 514), NSISSSNGQK (SEQ ID NO: 515), GEEGEGSGGGEK (SEQ ID NO: 516), EGEGGKESGSSE (SEQ ID NO: 517), GGGGSSKSPGSE (SEQ ID NO: 518), PIGSDQDD (SEQ ID NO: 519), TEKGQVPHGS (SEQ ID NO: 520), ESGEGGGGSEKK (SEQ ID NO: 521), EEGKPKEGEGSG (SEQ ID NO: 522), ASREPKDSS (SEQ ID NO: 523), KOGSEHDE (SEQ ID NO: 524), SESKSEKGSSEK (SEQ ID NO: 525), QYDSGERSDQ (SEQ ID NO: 526), PGANEEIPGQ (SEQ ID NO: 527), NSPTDEK (SEQ ID NO: 528), EGANEEIPGQ (SEQ ID NO: 529), EGEKEKKKSGES (SEQ ID NO: 530), PGRHEEVPGQ (SEQ ID NO: 531), SKHQTEQDDS (SEQ ID NO: 532), and ESEDDSSGRK (SEQ ID NO: 533). In some embodiments, the polypeptide comprises any one of the amino acid sequences: KESEKKESESKS (SEQ ID NO: 534), KGEGKSSIKD (SEQ ID NO: 535), DRSQKQDQQD (SEQ ID NO: 536), GPSSTSSS (SEQ ID NO: 537), GSSGEKEEGEPS (SEQ ID NO: 538), GEPKSKKSGSGS (SEQ ID NO: 539), SSGEGGKSESGP (SEQ ID NO: 540), SPQPTSSD (SEQ ID NO: 541), EGGSEEEEESGS (SEQ ID NO: 542), KGPKPKKEESEK (SEQ ID NO: 439), SKSQQFVTYE (SEQ ID NO: 544), TGNSKYQTGK (SEQ ID NO: 545), PQPIPHTNPT (SEQ ID NO: 546), ANAHSDISTG (SEQ ID NO: 547), KSQQTEDQSK (SEQ ID NO: 548), QSQDQKQKEH (SEQ ID NO: 549), NOQRPSSD (SEQ ID NO: 550), TTKDTSPKPQ (SEQ ID NO: 551), EGKDNOOTGE (SEQ ID NO: 552), and EPQPDSSE (SEQ ID NO: 553). It should be appreciated that the disclosure provides variants of the polypeptides (e.g., linkers) provided herein. In some embodiments, the polypeptide variant (e.g., linker) comprises at least 1, 2, 3, 4, or 5 amino acid changes from a reference sequence. In some embodiments the polypeptide variant (e.g., linker) has 1, 2, 3, 4, or 5 amino acid residues removed from the N-terminus of a reference sequence. In some embodiments the polypeptide variant (e.g., linker) has 1, 2, 3, 4, or 5 amino acid residues removed from the C-terminus of a reference sequence.

In some embodiments, the disclosure provides a polypeptide comprising the amino acid sequence EGGSEEEEESGS (SEQ ID NO: 542). In some embodiments the amino acid sequence EGGSEEEEESGS (SEQ ID NO: 542) is used to link a TadA domain (e.g., TadA*7.10) with a Cas9 domain (e.g., a SpCas9-MQKFRAER nickase), for example, in a base editor protein or system. In some embodiments, the amino acid linker EGGSEEEEESGS (SEQ ID NO: 542) is used to link a TadA*7.10 to a SpCas9-MQKFRAER nickase, for example, as in ABE Variant C and ABE Variant D.

In some embodiments, the disclosure provides a polypeptide comprising the amino acid sequence KGPKPKKEESEK (SEQ ID NO: 439). In some embodiments the amino acid sequence KGPKPKKEESEK (SEQ ID NO: 439) is used to link a TadA domain (e.g., TadA*7.10) with a Cas9 domain (e.g., a SpCas9-MQKFRAER nickase), for example, in a base editor protein or system. In some embodiments, the amino acid linker KGPKPKKEESEK (SEQ ID NO: 439) is used to link a TadA*7.10 to a SpCas9-MQKFRAER nickase, for example, as in ABE Variant E, ABE Variant I, and ABE Variant K.

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising an Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, an adenosine deaminase nucleobase editor described herein is capable of making multiple mutations within a target sequence. These mutations may affect the function of the target. For example, when an adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain) can be used to edit a nucleotide from A to G.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations: unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C: G-to-T: A base editors with higher efficiency and product purity” Science Advances 3: eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cell

Fusion proteins or complexes of the disclosure comprising a deaminase may be expressed in virtually any host cell of interest, including but not limited to bacteria, yeast, fungi, insects, plants, and animal cells using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A polynucleotide encoding a polypeptide described herein can be obtained by chemically synthesizing the polynucleotide, or by connecting synthesized partly overlapping oligo short chains by utilizing the PCR method and the Gibson Assembly method to construct a polynucleotide (e.g., DNA) encoding the full length thereof. The advantage of constructing a full-length polynucleotide by chemical synthesis or a combination of PCR method or Gibson Assembly method is that the codons to be used can be selected in according to the host into which the polynucleotide is to be introduced. In the expression from a heterologous DNA molecule, the protein expression level is expected to increase by converting the DNA sequence thereof to a codon highly frequently used in the host organism. Codon use data for a host cell (e.g., codon use data available at kazusa.or.jp/codon/index.html) can be used to guide codon optimization for a polynucleotide sequence encoding a polypeptide. Codons having low use frequency in the host may be converted to a codon coding the same amino acid and having high use frequency.

An expression vector containing a polynucleotide encoding a nucleic acid sequence-recognizing module and/or a nucleic acid base converting enzyme can be produced, for example, by linking the DNA to the downstream of a promoter in a suitable expression vector.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as .lambda phage and the like; insect virus vectors such as baculovirus and the like (e.g., BmNPV, AcNPV); animal virus vectors such as retrovirus, vaccinia virus, adenovirus and the like, and the like are used.

Regarding the promoter to be used, any promoter appropriate for a host to be used for gene expression can be used. In a conventional method using double-stranded breaks, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like may be used.

When the host is Escherichia coli, a trp promoter, lac promoter, recA promoter, .lamda.P.sub.L promoter, lpp promoter, T7 promoter, and the like can be used.

When the host is in the genus Bacillus, the SPO1 promoter, SPO2 promoter, penP promoter, and the like can be used.

When the host is a yeast, the Gal1/10 promoter, PHO5 promoter, PGK promoter, GAP promoter, ADH promoter, and the like can be used.

When the host is an insect cell, the polyhedrin promoter, P10 promoter, and the like can be used.

When the host is a plant cell, the CaMV35S promoter, CaMV19S promoter, NOS promoter, and the like can be used.

Expression vectors for use in the present disclosure, besides those mentioned above, can comprise an enhancer, a splicing signal, a terminator, a poly A addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or complexes disclosed herein.

A fusion protein or complex of the disclosure can be intracellularly expressed by introducing into the cell an expression vector comprising a nucleic acid sequence encoding the fusion protein or complex.

Host cells of interest, include but are not limited to bacteria, yeast, fungi, insects, plants, and animal cells. For example, a host cell may comprise bacteria from the genus Escherichia, such as Escherichia coli K12.cndot.DH1 [Proc. Natl. Acad. Sci. USA, 60, 160 (1968)], Escherichia coli JM103 [Nucleic Acids Research, 9, 309 (1981)], Escherichia coli JA221 [Journal of Molecular Biology, 120, 517 (1978)], Escherichia coli HB101 [Journal of Molecular Biology, 41, 459 (1969)], Escherichia coli C600 [Genetics, 39, 440 (1954)] and the like.

A host cell may comprise bacteria from the genus Bacillus, for example Bacillus subtilis M1114 [Gene, 24, 255 (1983)], Bacillus subtilis 207-21 [Journal of Biochemistry, 95, 87 (1984)] and the like.

A host cell may be a yeast cell. Examples of yeast cells include Saccharomyces cerevisiae AH22, AH22R.sup.-, NA87-11A, DKD-5D, 20B-12, Schizosaccharomyces pombe NCYC1913, NCYC2036, Pichia pastoris KM71 and the like.

When the viral delivery methods utilize the virus AcNPV, cells from a cabbage armyworm larva-derived established line (Spodoptera frugiperda cell; Sf cell), MG1 cells derived from the mid-intestine of Trichoplusia ni, High Five™ cells derived from an ovary of Trichoplusia ni, Mamestra brassicae-derived cells, Estigmena acrea-derived cells and the like can be used. When the virus is BmNPV, cells of Bombyx mori-derived established line (Bombyx mori N cell; BmN cell) and the like are used. As the Sf cell, for example, Sf9 cell (ATCC CRL1711), Sf21 cell [all above, In Vivo, 13, 213-217 (1977)] and the like are used.

An insect can be any insect, for example, larva of Bombyx mori, Drosophila, cricket, and the like [Nature, 315, 592 (1985)].

Animal cells contemplated in the present disclosure include, but are not limited to, cell lines such as monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used.

Plant cells are also contemplated in the present disclosure. Plant cells include, but are not limited to, suspended cultured cells, callus, protoplast, leaf segment, root segment and the like prepared from various plants (e.g., grain such as rice, wheat, corn, and the like; product crops such as tomato, cucumber, eggplant and the like; garden plants such as carnations, Eustoma russellianum, and the like; and other plants such as tobacco, Arabidopsis thaliana and the like) are used.

All the above-mentioned host cells may be haploid (monoploid), or polyploid (e.g., diploid, triploid, tetraploid, etc.). Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present disclosure, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

Escherichia coli can be transformed according to the methods described in, for example, Proc. Natl. Acad. Sci. USA, 69, 2110 (1972), Gene, 17, 107 (1982).

The genus Bacillus can be introduced into a vector according to the methods described in, for example, Molecular & General Genetics, 168, 111 (1979).

A yeast can be introduced into a vector according to the methods described in, for example, Methods in Enzymology, 194, 182-187 (1991), Proc. Natl. Acad. Sci. USA, 75, 1929 (1978).

An insect cell and an insect can be introduced into a vector according to the methods described in, for example, Bio/Technology, 6, 47-55 (1988).

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

A cell comprising a vector can be cultured according to a known method according to the kind of the host. For example, when Escherichia coli or genus Bacillus is cultured, a liquid medium may be used as a medium to be used for the culture. The medium may contain a carbon source, nitrogen source, inorganic substance and the like necessary for the growth of the transformant. Examples of the carbon source include glucose, dextrin, soluble starch, sucrose and the like; examples of the nitrogen source include inorganic or organic substances such as ammonium salts, nitrate salts, corn steep liquor, peptone, casein, meat extract, soybean cake, potato extract and the like; and examples of the inorganic substance include calcium chloride, sodium dihydrogen phosphate, magnesium chloride and the like. The medium may contain yeast extract, vitamins, growth promoting factor and the like. The pH of the medium is between about 5 about 8 in embodiments.

As a medium for culturing Escherichia coli, for example, M9 medium containing glucose, casamino acid [Journal of Experiments in Molecular Genetics, 431-433, Cold Spring Harbor Laboratory, New York 1972] can be used. Where necessary, for example, agents such as 3B-indolylacrylic acid may be added to the medium to ensure an efficient function of a promoter. Escherichia coli is cultured at generally about 15 to about 43° C. Where necessary, aeration and stirring may be performed.

The genus Bacillus is cultured at generally about 30 to about 40° C. Where necessary, aeration and stirring may be performed.

Examples of the medium for culturing yeast include Burkholder minimum medium [Proc. Natl. Acad. Sci. USA, 77, 4505 (1980)], SD medium containing 0.5% casamino acid [Proc. Natl. Acad. Sci. USA, 81, 5330 (1984)] and the like. The pH of the medium may be between about 5 to about 8. The culture is performed at generally about 20° C. to about 35° C. Where necessary, aeration and stirring may be performed.

As a medium for culturing an insect cell or insect, for example, Grace's Insect Medium [Nature, 195, 788 (1962)] containing an additive such as inactivated 10% bovine serum and the like as appropriate and the like are used. The pH of the medium is may be between about 6.2 to about 6.4. The culture is performed at generally about 27° C. Where necessary, aeration and stirring may be performed.

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium may be between about 6 to about 8. The culture is performed at generally about 30° C. to about 40° C. Where necessary, aeration and stirring may be performed.

As a medium for culturing a plant cell, for example, MS medium, LS medium, B5 medium and the like are used. The pH of the medium may be between about 5-about 8. The culture is performed at generally about 20° C. to about 30° C. Where necessary, aeration and stirring may be performed.

When a higher eukaryotic cell, such as animal cell, insect cell, plant cell and the like is used as a host cell, a polynucleotide encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Prokaryotic cells such as Escherichia coli and the like can utilize an inducible promoter. Examples of the inducible promoter include, but are not limited to, lac promoter (induced by IPTG), cspA promoter (induced by cold shock), araBAD promoter (induced by arabinose) and the like.

Alternatively, the above-mentioned inductive promoter can also be utilized as a vector removal mechanism when higher eukaryotic cells, such as animal cell, insect cell, plant cell and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, WO2024019936, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), rabies virus (see, e.g., U.S. Patent Application Publication No. US 2022/0290164 A1, the disclosure of which is incorporated herein by reference in its entirety for all purposes), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art (e.g., lipid nanoparticle-based delivery methods).

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient HDR with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (IssDNA) donors.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138 (7): 2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter Thy X intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publication No. WO 2013/045632, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, referenced to SEQ ID NO: 197.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Kits

The disclosure provides kits for the treatment of an alpha-1 antitrypsin deficiency in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Screening and Rational Design for ABEs

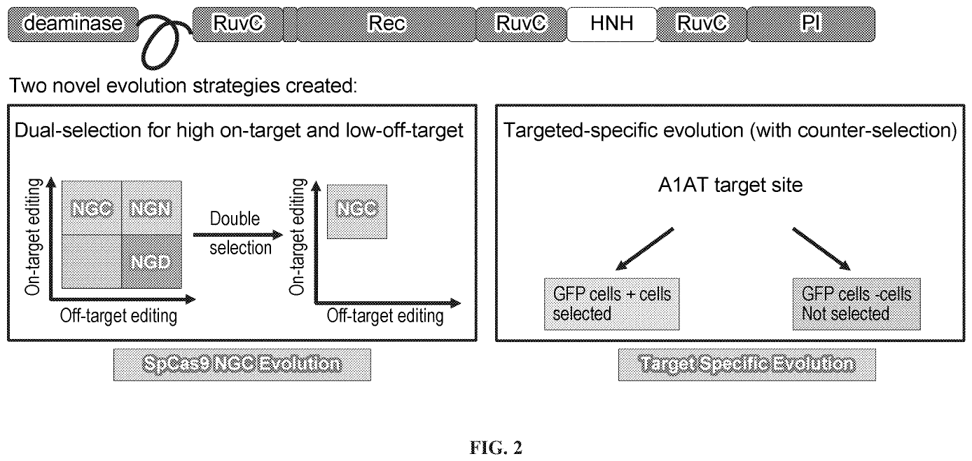

Yeast-based screens and structure-based design was performed to optimize editors to improve selectivity for NGC PAM and the Alpha-1 target site (FIG. 1). Schematics depicting yeast-based screens and structure-based design to optimize editors for improving selectivity for NGC PAM and the A1AT target site are shown in FIG. 2.

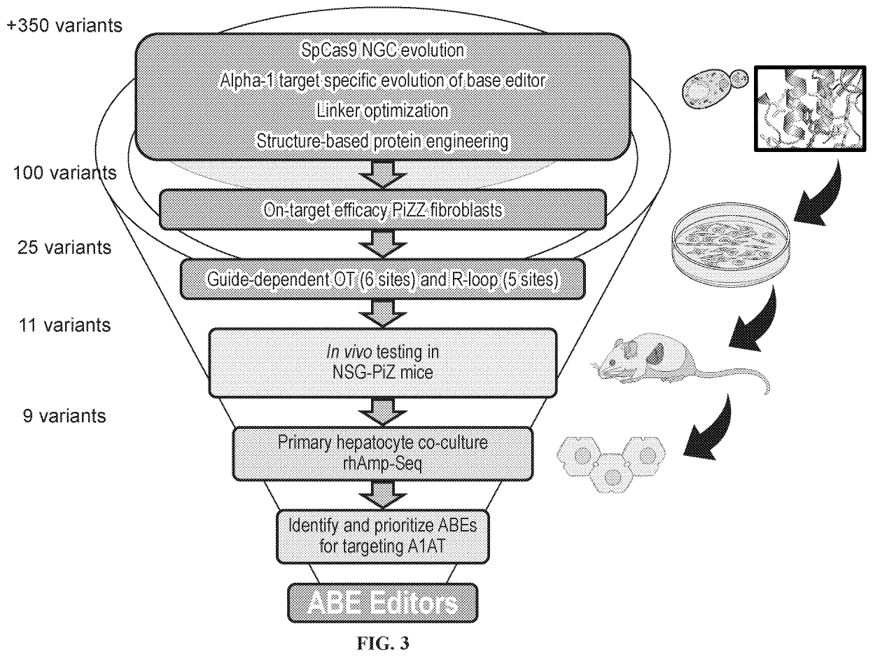

Hundreds of editor variants identified by the various screens and structure-based approaches were tested in patient fibroblasts for on-target editing. Guide-dependent off target editing at candidate OT sites and guide-independent off target editing using the sensitive R-loop assay were also performed in patient fibroblasts. High performing editors were tested at a sub-saturating dose of 0.25 mpk (mg/kg) against var12 codon optimized in vivo in two studies (SBTx760 and SBTx850) performed in NSG-PiZ mice (a widely accepted model of AATD). 9 editors were advanced into rhAmp-seq guide-dependent off-target editing in primary hepatocytes. These experiments culminated in the identification of the new high-performing development candidates FIG. 3.

### Set 1: NGC Evolution.

For this yeast evolution, a dual-selection system was used: the on-target selection was targeting an NGC target site in an inactivated blasiticin resistant (BlaR) gene, and the A6G editing resulted in restoration of the function of BlaR gene. For off-target selection, three guides were targeting the same stop codon in an inactivated mCherry gene, and their target sites were with NGA, NGG, NGT PAM, respectively. The A to G editing activities guided by any of the three guides resulted in mCherry signal. The yeast cells were transformed with a library of base editors derived from Var12. This library was designed to contain all the possible combinations of mutations that were rationally designed based on the crystal structure of Cas9, focusing on mutating all the residues important for recognizing the 3rd nucleotide in the PAM sequence. After culturing the yeast cells containing both selection plasmid and base editor plasmid, a two-step selection was performed to select for high NGC activity (Bla drug selection) and low NGD activity (flow sort for mCherry—population). Mutations identified in the screen for Cas9 (SpCas9-MQKFRAER nickase) are provided in Table 7, below.

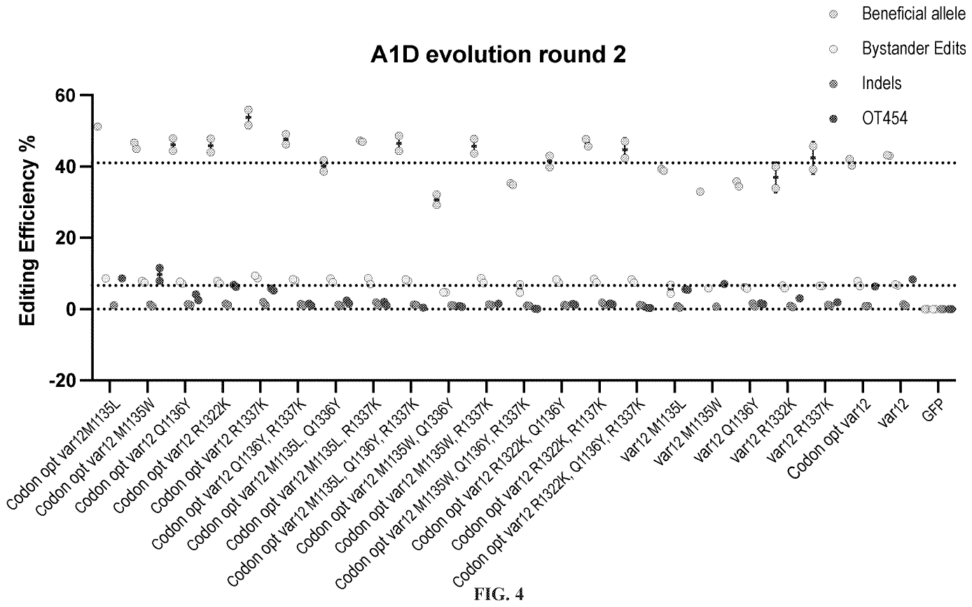

Wild-type HEK293T cells were transfected in 96-well format and each well contained plasmids expressing base editor variants and plasmids expressing guides. To calculate average on-target editing efficiency, each editor was co-transfected with plasmids expressing guides targeting A1 target site without E342K mutation, and 2 other NGC target sites, respectively. To calculate average off-target editing efficiency, each editor was co-transfected with guides targeting A1 target site, and OT editing were measured at identified OT target site OT1/2/4. Results are laid out in Table 8. Editing efficiency of variants identified in the screen were tested in in patient derived fibroblast cells (PiZZ cells), which were transfected with mRNA encoding the indicated variants and synthetic guides by electroporation (FIG. 4).

TABLE 7
List of mutations identified in SpCas9-
MQKFRAER nickase for Screen 1.

| plasmid number | Mutation in SpCas9-MQKFRAER nickase |
|---|---|
| pYY-1301 | W1126R |
| pYY-1302 | R1359W |
| pYY-1303 | E1250K |
| pYY-1304 | A1239T |
| pYY-1305 | A1239V |
| pYY-1306 | E1335D |
| pYY-1307 | M1135L |
| pYY-1308 | M1135R |
| pYY-1309 | M1135W |
| pYY-1310 | Q1136H |
| pYY-1311 | Q1136Y |
| pYY-1312 | K1218D |
| pYY-1313 | K1218R |
| pYY-1314 | K1218E |
| pYY-1315 | K1218L |
| pYY-1316 | F1219K |
| pYY-1317 | F1219N |
| pYY-1318 | R1322A |
| pYY-1319 | R1322K |
| pYY-1320 | R1337K |
| pYY-1321 | R1337T |
| pYY-1322 | M1135L Q1136H |
| pYY-1323 | M1135R Q1136H |
| pYY-1324 | M1135L Q1136Y |
| pYY-1325 | M1135R Q1136Y |
| pYY-1326 | K1218D F1219K |
| pYY-1327 | K1218R F1219K |
| pYY-1328 | E1335D R1337K |
| pYY-1329 | M1135R Q1136H K1218D R1322K E1335D R1337K |
| pYY-1330 | M1135V Q1136H K1218D R1322K E1335D R1337K |

TABLE 8
Analysis of off target and on target editing for ABE variants.
Exemplary mutants in SpCas9-MQKFRAER nickase.

| | Off | On | On/Off | |
|---|---|---|---|---|
| Plasmid | Target | Target | Ratio | Mutant Cas9 |
| pYY-1303 | 21.8 | 56.4 | 2.6 | E1250K |
| pYY-1313 | 19.1 | 54.3 | 2.8 | K1218R |
| pYY-1320 | 13.6 | 53.7 | 4 | R1337K |
| pYY-1311 | 15.1 | 53.2 | 3.5 | Q1136Y |
| pYY-1309 | 15.3 | 52.7 | 3.4 | M1135W |
| pYY-1307 | 16.5 | 52.6 | 3.2 | M1135L |
| pYY-1319 | 16.7 | 52.4 | 3.1 | R1322K |
| pYY-1306 | 12.1 | 52.2 | 4.3 | E1335D |
| pYY-1171 | 19.3 | 52.2 | 2.7 | WT |
| pYY-1318 | 13.3 | 52.2 | 3.9 | R1322A |
| pYY-1321 | 10.8 | 52.1 | 4.8 | R1337T |
| pYY-1312 | 9.5 | 51.6 | 5.4 | K1218D |
| pYY-1301 | 13.8 | 51.5 | 3.7 | W1126R |
| pYY-1310 | 12.5 | 51 | 4.1 | Q1136H |
| pYY-1324 | 12.2 | 51 | 4.2 | M1135L Q1136Y |
| pYY-1315 | 13 | 50.3 | 3.9 | K1218L |
| pYY-1302 | 12.6 | 49.7 | 4 | R1359W |
| pYY-1322 | 11.6 | 49.1 | 4.2 | M1135L Q1136H |
| pYY-1314 | 9.9 | 48.7 | 4.9 | K1218E |
| pYY-1328 | 3.7 | 47.9 | 12.8 | E1335D R1337K |
| pYY-1304 | 12.6 | 47.8 | 3.8 | A1239T |
| pYY-1308 | 12 | 47.8 | 4 | M1135R |
| pYY-1323 | 9.1 | 47.6 | 5.2 | M1135R Q1136H |
| pYY-1305 | 12.2 | 47.1 | 3.9 | A1239V |
| pYY-1325 | 6.3 | 45.8 | 7.2 | M1135R Q1136Y |
| pYY-1317 | 12 | 44.1 | 3.7 | F1219N |
| pYY-1316 | 2.8 | 41.4 | 14.9 | F1219K |
| pYY-1327 | 2.7 | 39.9 | 15 | K1218R F1219K |
| pYY-1326 | 1.4 | 37.2 | 27.4 | K1218D F1219K |
| pYY-1329 | 0.2 | 26 | 170.4 | M1135R Q1136H |
| | | | | K1218D R1322K |
| | | | | E1335D R1337K |
| pYY-1330 | 0.1 | 18.9 | 164.9 | M1135V Q1136H |
| | | | | K1218D R1322K |
| | | | | E1335D R1337K |

### Set 2: Linker Rational Design

Seventeen linkers were predicted to position deaminase near A7 in the editing window using Rosetta fold 2. A list of the linkers identified in this rational design study are provided in Table 9.

TABLE 9
List of rationally designed
linker sequences.

| Linkers | SEQ ID NO |
|---|---|
| STDTTKDTSPKPQDKI | 497 |
| STDANAHSDISTGDKI | 498 |
| STDEDKRSDLGKQDKI | 499 |
| STDEKDQQPNSTSDKI | 500 |
| STDQKGQKIPSSNDKI | 501 |
| STDDRSQKQDQQDDKI | 502 |
| STDSKSVPLSSDGDKI | 503 |
| STDRQPRKDNSSGDKI | 504 |
| STDKEGDQPPPPKDKI | 505 |
| STDDVEYYSRPPDDKI | 506 |
| STDDPRKREIPPPDKI | 507 |
| STDERQHSTPTTDDKI | 508 |
| STDSSKQPENPPQDKI | 509 |
| STDPGGAPETNSGDKI | 510 |
| STDNISLSYDQQDDKI | 511 |
| STDSKSQQFVTYEDKI | 512 |
| STDLTESRPPQESDKI | 513 |

### Set 3: Linker-Evolution

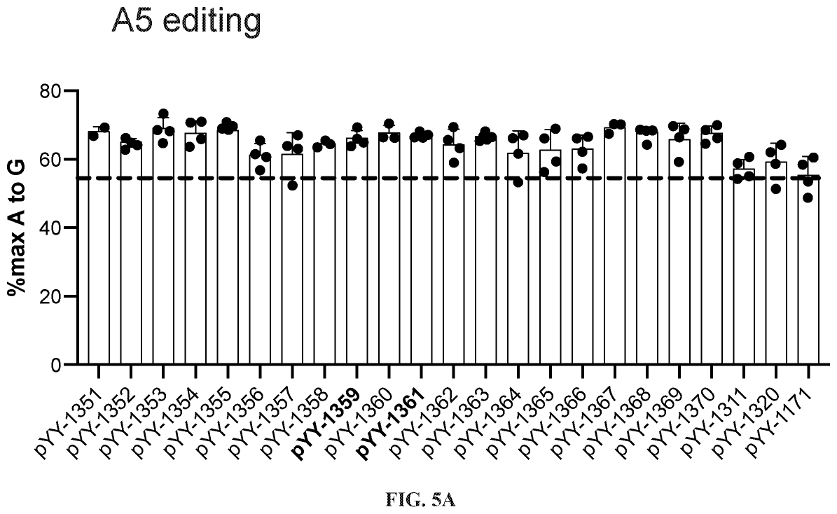

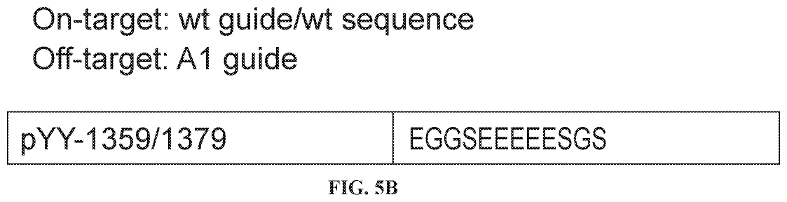

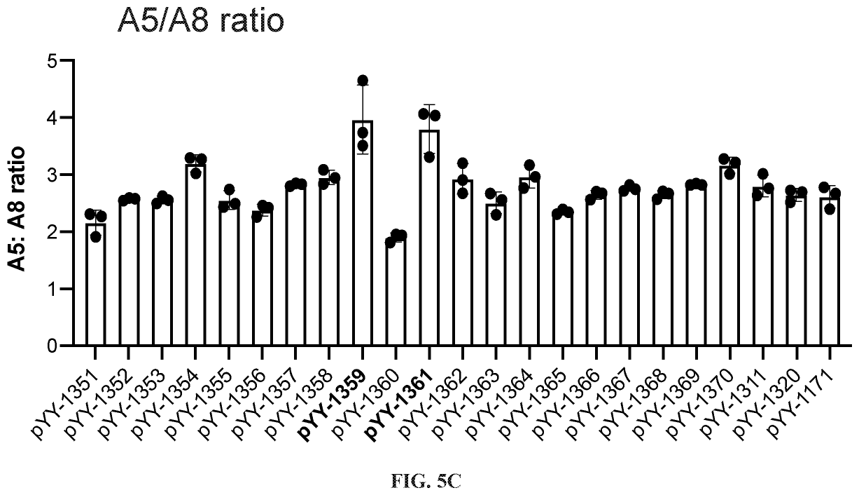

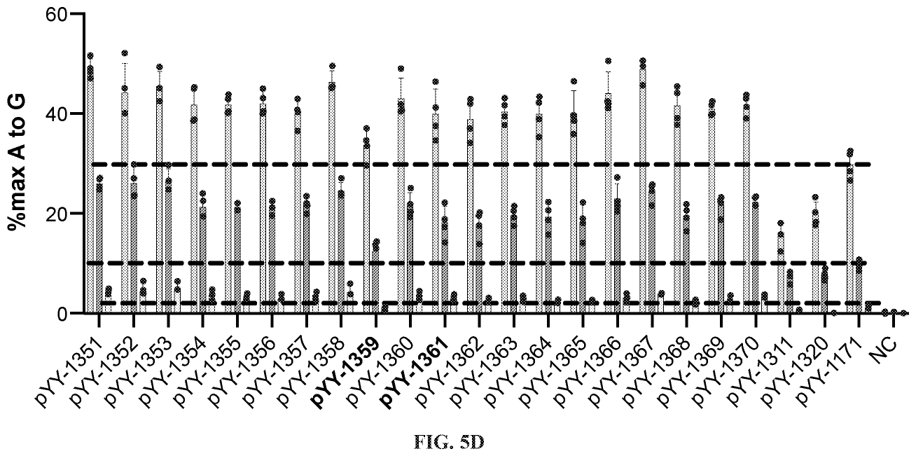

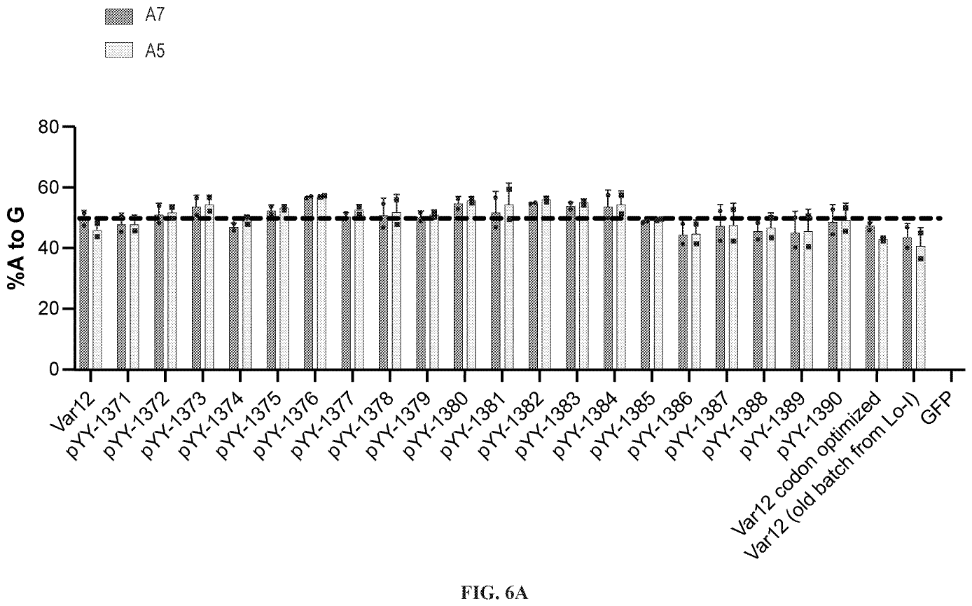

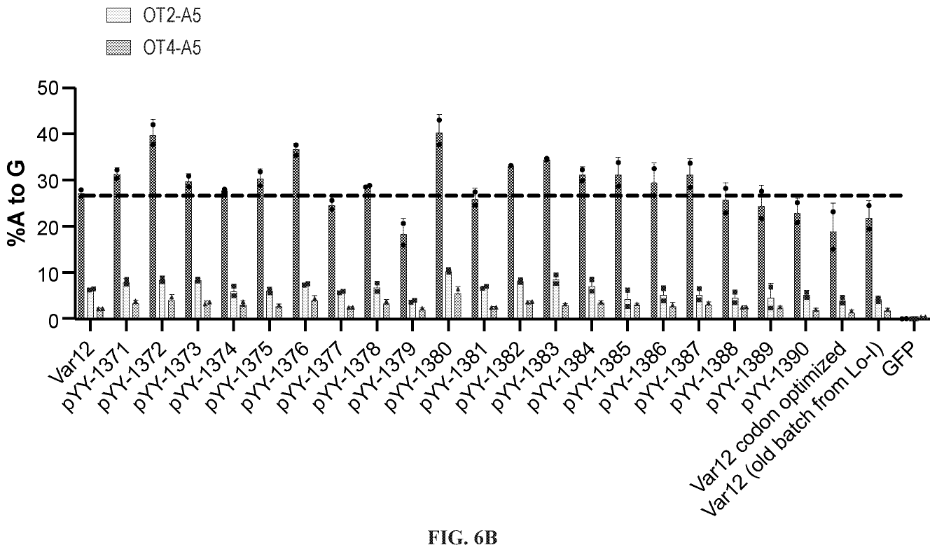

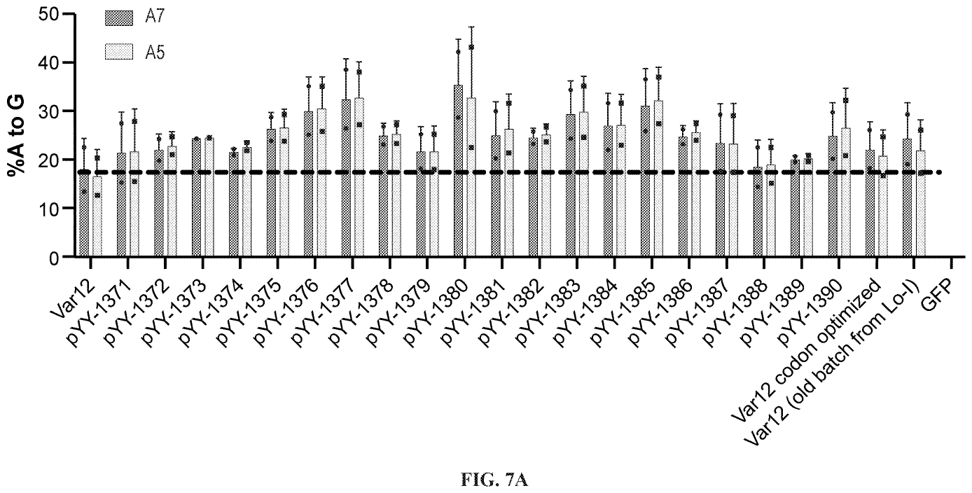

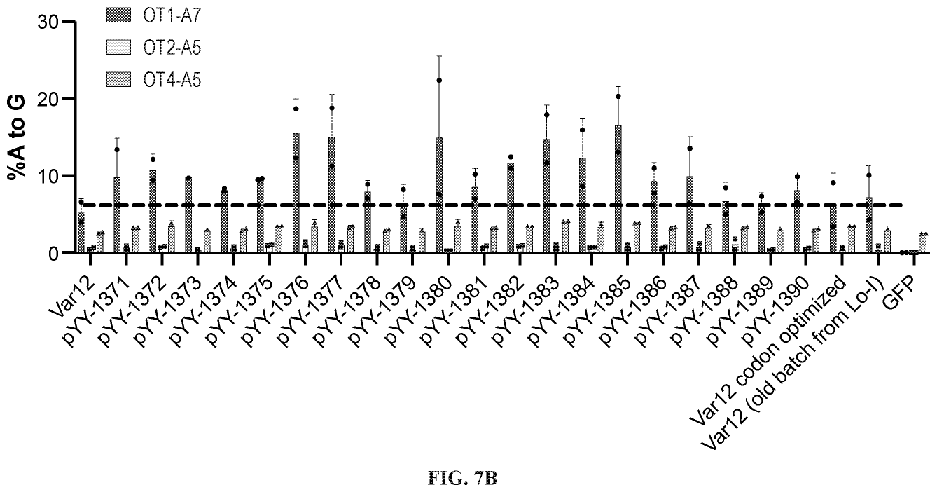

Guide-specific yeast evolution was used to select for optimal linkers from two linker libraries: one was a 12 aa random linker library composed of 5 amino acids: E, K, G, S, P; the other was computationally designed linkers for optimal A7 editing, see Table 10 and Table 11. The guide-specific yeast evolution was an evolution strategy that allowed evolving BEs/Cas enzymes towards any user-defined target site. This evolution was performed with a defined SpCas9 guide (the A1AT guide in this case), and the base editor first edited target site 1. Target site 1 was the crRNA region in a Cas12a guide, and target site 1 had the same sequence as the A1AT target site. This Cas12a guide was expressed with Cas12a protein. When target site 1 was edited by the base editor, the Cas12a complex recognized target site 2 in a disrupted EGFP gene and resulted in a DSB in the gene. This was followed by HDR repair resulting in functional EGFP gene. When the target site 1 was not edited by the base editor, the Cas12a complex was not be able to recognize target site 2, and there was no EGFP signal. This strategy allowed for selection of linkers in BEs that improved potency for A1AT target site. Editing results of variants having different linker sequences can be seen in FIGS. 5A-5D, FIGS. 6A-6B, and FIGS. 7A-7B.

TABLE 10
Linkers identified in Set 3,
linker evolution.

| Plasmid | Linker | SEQ ID NO |
|---|---|---|
| pYY-1331 | EGSSSKEEEEPG | 514 |
| pYY-1332 | NSISSSNGQK | 515 |
| pYY-1333 | GEEGEGSGGGEK | 516 |
| pYY-1334 | EGEGGKESGSSE | 517 |
| pYY-1335 | GGGGSSKSPGSE | 518 |
| pYY-1336 | PIGSDQDD | 519 |
| pYY-1337 | TEKGQVPHGS | 520 |
| pYY-1338 | ESGEGGGGSEKK | 521 |
| pYY-1339 | EEGKPKEGEGSG | 522 |
| pYY-1340 | ASREPKDSS | 523 |
| pYY-1341 | KQGSEHDE | 524 |
| pYY-1342 | SESKSEKGSSEK | 525 |
| pYY-1343 | QYDSGERSDQ | 526 |
| pYY-1344 | PGANEEIPGQ | 527 |
| pYY-1345 | NSPTDEK | 528 |
| pYY-1346 | EGANEEIPGQ | 529 |
| pYY-1347 | EGEKEKKKSGES | 530 |
| pYY-1348 | PGRHEEVPGQ | 531 |
| pYY-1349 | SKHQTEQDDS | 532 |
| pYY-1350 | ESEDDSSGRK | 533 |

TABLE 11
Linkers identified in Set 3
linker evolution.

| Plasmid | Linker | SEQ ID NO |
|---|---|---|
| pYY-1351 | KESEKKESESKS | 534 |
| pYY-1352 | KGEGKSSIKD | 535 |
| pYY-1353 | DRSQKQDQQD | 536 |
| pYY-1354 | GPSSTSSS | 537 |
| pYY-1355 | GSSGEKEEGEPS | 538 |
| pYY-1356 | GEPKSKKSGSGS | 539 |
| pYY-1357 | SSGEGGKSESGP | 540 |
| pYY-1358 | SPQPTSSD | 541 |
| pYY-1359 | EGGSEEEEESGS | 542 |
| pYY-1360 | KGPKPKKEESEK | 439 |
| pYY-1361 | SKSQQFVTYE | 544 |
| pYY-1362 | TGNSKYQTGK | 545 |
| pYY-1363 | PQPIPHTNPT | 546 |
| pYY-1364 | ANAHSDISTG | 547 |
| pYY-1365 | KSQQTEDQSK | 548 |
| pYY-1366 | QSQDQKQKEH | 549 |
| pYY-1367 | NQQRPSSD | 550 |
| pYY-1368 | TTKDTSPKPQ | 551 |
| pYY-1369 | EGKDNQQTGE | 552 |
| pYY-1370 | EPQPDSSE | 553 |
| pYY-1371 | KESEKKESESKS | 534 |
| pYY-1372 | KGEGKSSIKD | 535 |
| pYY-1373 | DRSQKQDQQD | 536 |
| pYY-1374 | GPSSTSSS | 537 |
| pYY-1375 | GSSGEKEEGEPS | 538 |
| pYY-1376 | GEPKSKKSGSGS | 539 |
| pYY-1377 | SSGEGGKSESGP | 540 |
| pYY-1378 | SPQPTSSD | 541 |
| pYY-1379 | EGGSEEEEESGS | 542 |
| pYY-1380 | KGPKPKKEESEK | 439 |
| pYY-1381 | SKSQQFVTYE | 544 |
| pYY-1382 | TGNSKYQTGK | 545 |
| pYY-1383 | PQPIPHTNPT | 546 |
| pYY-1384 | ANAHSDISTG | 547 |
| pYY-1385 | KSQQTEDQSK | 548 |
| pYY-1386 | QSQDQKQKEH | 549 |
| pYY-1387 | NQQRPSSD | 550 |
| pYY-1388 | TTKDTSPKPQ | 551 |
| pYY-1389 | EGKDNQQTGE | 552 |
| pYY-1390 | EPQPDSSE | 553 |

### Set 4: Trinucleotide Specific TadA-Rational Design

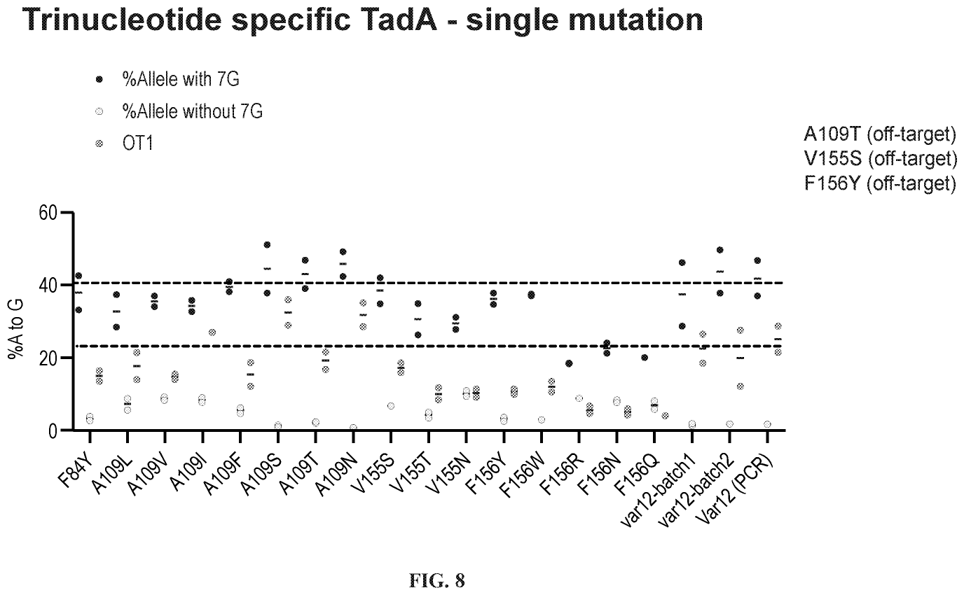

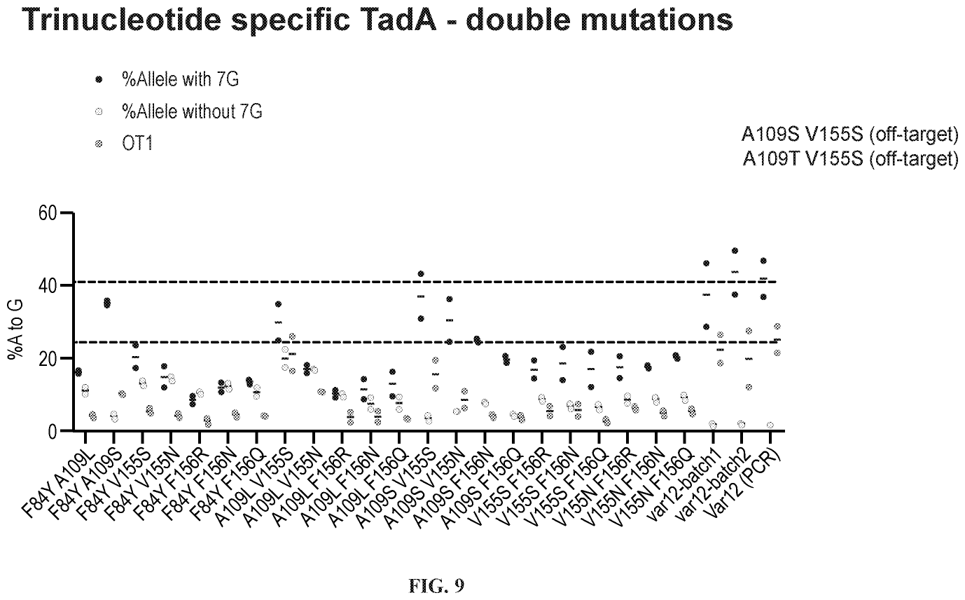

Rational design of TadA mutations that changes the trinucleotide motif preference for TadA, based on the TadA crystal structure was performed. Trinucleotide motif preference for deaminase (TadA), refers to the preference for the nucleotides flanking the target nucleotide—the “NAN” motif in the protospacer sequence where the A is the target for TadA. Mutations in TadA (e.g., TadA*7.10 of Variant 12) identified in the rational design are shown in Table 12. Results for % A to G editing of ABE variants identified in this screen for single and double TadA mutations are shown in FIG. 8 and FIG. 9, respectively.

TABLE 12
TadA mutations identified in Set 4, TadA-rational design.

| Plasmid Number | Mutation in TadA |
|---|---|
| pYY-1401 | F84Y |
| pYY-1402 | A109L |
| pYY-1403 | A109V |
| pYY-1404 | A109I |
| pYY-1405 | A109F |
| pYY-1406 | A109S |
| pYY-1407 | A109T |
| pYY-1408 | A109N |
| pYY-1409 | V155S |
| pYY-1410 | V155T |
| pYY-1411 | V155N |
| pYY-1412 | F156Y |
| pYY-1413 | F156W |
| pYY-1414 | F156R |
| pYY-1415 | F156N |
| pYY-1416 | F156Q |
| pYY-1417 | F84Y A109L |
| pYY-1418 | F84Y A109S |
| pYY-1419 | F84Y V155S |
| pYY-1420 | F84Y V155N |
| pYY-1421 | F84Y F156R |
| pYY-1422 | F84Y F156N |
| pYY-1423 | F84Y F156Q |
| pYY-1424 | A109L V155S |
| pYY-1425 | A109L V155N |
| pYY-1426 | A109L F156R |
| pYY-1427 | A109L F156N |
| pYY-1428 | A109L F156Q |
| pYY-1429 | A109S V155S |
| pYY-1430 | A109S V155N |
| pYY-1431 | A109S F156R |
| pYY-1432 | A109S F156N |
| pYY-1433 | A109S F156Q |
| pYY-1434 | V155S F156R |
| pYY-1435 | V155S F156N |
| pYY-1436 | V155S F156Q |
| pYY-1437 | V155N F156R |
| pYY-1438 | V155N F156N |
| pYY-1439 | V155N F156Q |
| pYY-1440 | F84Y A109L V155S |
| pYY-1441 | F84Y A109L V155N |
| pYY-1442 | F84Y A109S V155S |
| pYY-1443 | F84Y A109S V155N |
| pYY-1444 | A109L V155SF156N |
| pYY-1445 | A109L V155N F156N |
| pYY-1446 | A109S V155S F156N |
| pYY-1447 | A109S V155N F156N |
| pYY-1448 | F84Y A109L V155S F156N |

### Set 5: Hi-Fi Cas9-Rational Design

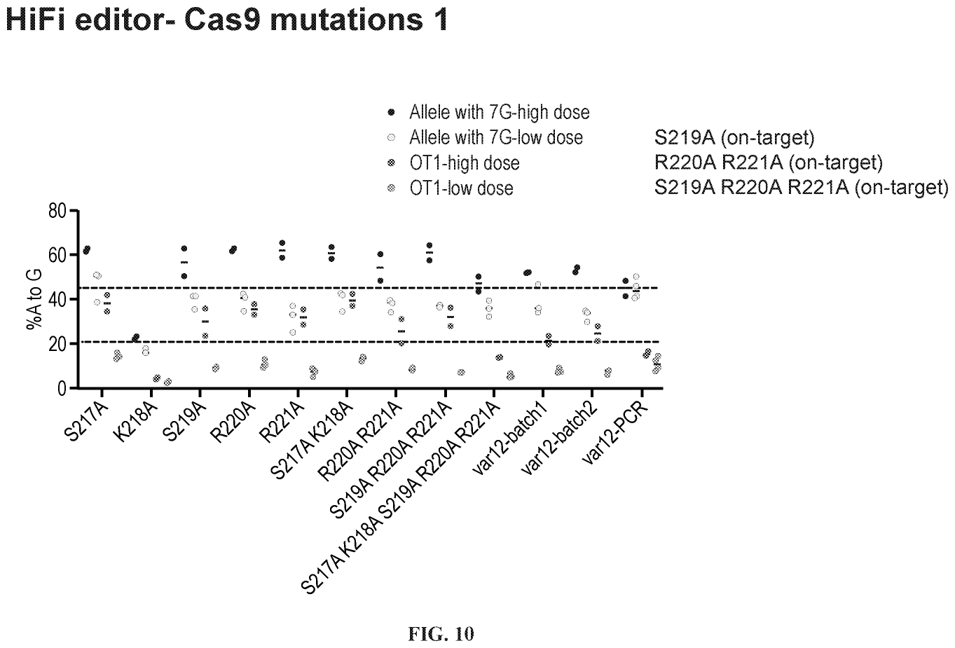

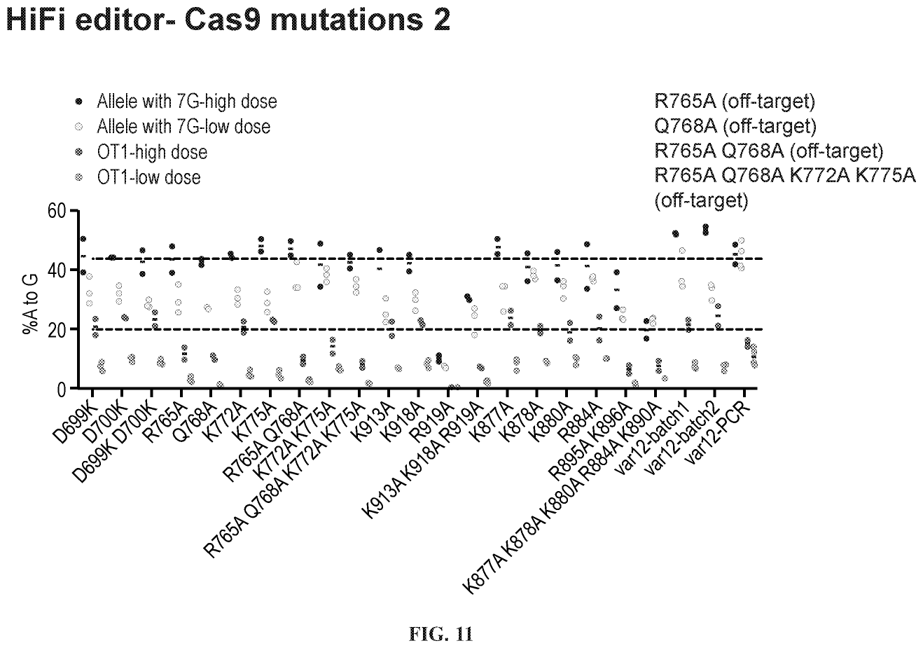

Rational design of Cas9 HiFi mutations (e.g., for mapping onto SpCas9-MQKFRAER nickase) were based on the cryo-EM structure of base editors with A1AT on-target substrate and off-target substrate. Cyro-EM structures of Variant12/A1 guide/A1 on-target sequences or one of A1 guided off-target sequences were obtained. Mutations that could reduce guided off-target (HiFi mutations) were predicted by comparing these two Cryo-EM structures. A list of Cas9 mutations identified in the rational design are shown in Table 13. Results for % A to G editing of ABE variants identified in this screen (NGC Cas9) in patient derived fibroblast cells (PiZZ cells) are shown in FIG. 10 and FIG. 11.

TABLE 13
Cas9 mutations identified in Set 5, Hi-Fi Cas9-rational design.

| Plasmid | |
|---|---|
| Number | Mutation in Mutation in SpCas9-MQKFRAER nickase |
| pYY-1451 | S217A |
| pYY-1452 | K218A |
| pYY-1453 | S219A |
| pYY-1454 | R220A |
| pYY-1455 | R221A |
| pYY-1456 | S217A/K218A |
| pYY-1457 | R220A/R221A |
| pYY-1458 | S219A/R220A/R221A |
| pYY-1459 | S217A/K218A/S219A/R220A/R221A |
| pYY-1460 | D699K |
| pYY-1461 | D700K |
| pYY-1462 | D699K/D700K |
| pYY-1463 | R765A |
| pYY-1464 | Q768A |
| pYY-1465 | K772A |
| pYY-1466 | K775A |
| pYY-1467 | R765A/Q768A |
| pYY-1468 | K772A/K775A |
| pYY-1469 | R765A/Q768A/K772A/K775A |
| pYY-1470 | K913A |
| pYY-1471 | K918A |
| pYY-1472 | R919A |
| pYY-1473 | K913A/K918A/R919A |
| pYY-1474 | K877A |
| pYY-1475 | K878A |
| pYY-1476 | K880A |
| pYY-1477 | R884A |
| pYY-1478 | R895A/K896A |
| pYY-1479 | K877A/K878A/K880A/R884A/K890A |
| pYY-1480 | K877A/K878A/K880A/R884A/K890A/R895A/K896A |

Set 6: TadA Guide-Specific Evolution with Counter Selection.

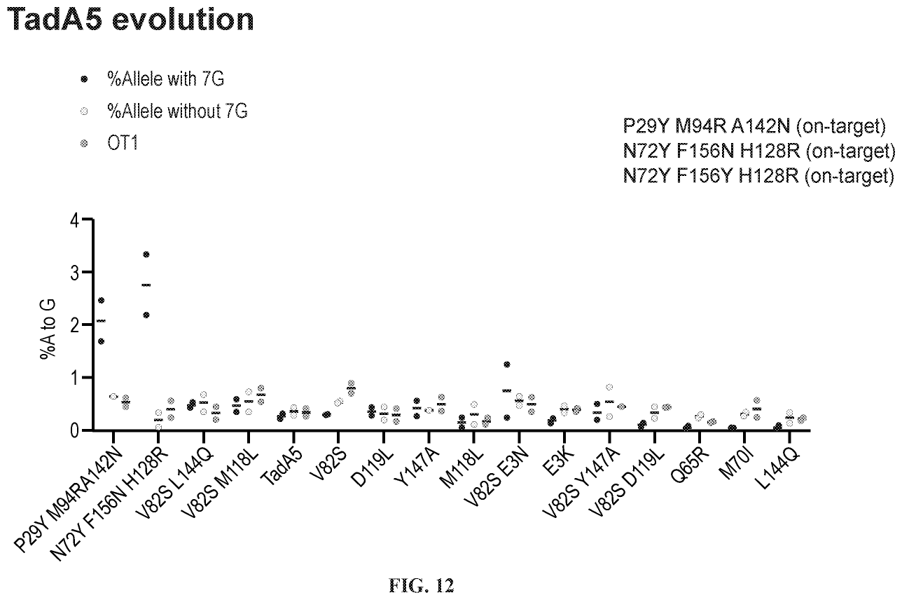

Guide specific evolution is explained above in Set 3 and off-target selection is explained above in Set 1. A TadA5 library with 2 mutations per construct was made and selected using these two selection strategies. The goal was to develop an improved TadA for use in targeting SERPINA1. TadA7.10, and variants thereof, typically show high activities across different target sites. A new evolution of TadA specific for the A1AT target site was designed by starting with an earlier version of TadA, TadA5, which only showed moderate activity on a few sites and very low activity on most sites. Both guide-specific yeast evolution and off-target selection were used in this evolution. The guide-specific yeast evolution was an evolution strategy that allowed evolving BEs/Cas enzymes toward any user-defined target site. This evolution was performed with a defined SpCas9 guide (targeting SERPINA1), and the base editor first edited target site 1. Target site 1 was the crRNA region in a Cas12a guide, and target site 1 had the same sequence as the A1AT target site. This Cas12a guide was expressed with Cas12a protein. When target site 1 was edited by the base editor, the Cas12a complex recognized target site 2 in a disrupted EGFP gene and resulted in a DSB in the gene. This was followed by HDR repair resulting in a functional EGFP gene. When the target site 1 was not edited by the base editor, the Cas12a complex was not be able to recognize target site 2, and there was no EGFP signal. This strategy enabled selection of BEs that improved potency for A1AT target site. For off-target selection, three guides were targeting the same stop codon in an inactivated mCherry gene, and their target sites were with NGA, NGG, NGT PAM, respectively. The A to G editing activities guided by any of the three guides resulted in mCherry signal. Yeast cells were transformed with a library of base editors derived from TadA5+Variant 12 Cas9 (SpCas9-MQKFRAER nickase). This TadA5 library was designed to contain 2 mutations per construct, and this library covers all the possible mutations on any 2 positions in TadA5. After culturing the yeast cells containing both selection plasmid and base editor plasmid, yeast cells with high EGFP signal and low mcherry signal were selected. Mutations in TadA5 (mappable onto TadA*7.10 of Variant 12) identified in the screen are shown in Table 14. Results for % A to G editing of ABE variants having TadA mutations identified in this screen are shown in FIG. 12.

TABLE 14
TadA mutations identified in Set 6, TadA guide-
specific evolution with counter selection.

| Plasmid Number | Mutation in TadA |
|---|---|
| pYY-1500 | TadA5 |
| pYY-1501 | E3K |
| pYY-1502 | A114C, E140L, W23I |
| pYY-1503 | G100V, P29T |
| pYY-1504 | L145R, G42M |
| pYY-1505 | I132K |
| pYY-1506 | V102S, G115E |
| pYY-1507 | V102E |
| pYY-1508 | D119L |
| pYY-1509 | L144Q |
| pYY-1510 | Y147A |
| pYY-1511 | M118L |
| pYY-1512 | R107A, L63F |
| pYY-1513 | A62V |
| pYY-1514 | E3G, I132T, F6A |
| pYY-1515 | W23R, P48A, R152P, TadA7.10 |
| pYY-1516 | V82S, TadA5 |
| pYY-1517 | V82S, E3K |
| pYY-1518 | V82S, A114C, E140L, W23I |
| pYY-1519 | V82S, G100V, P29T |
| pYY-1520 | V82S, L145R, G42M |
| pYY-1521 | V82S, I132K |
| pYY-1522 | V82S, V102S, G115E |
| pYY-1523 | V82S, V102E |
| pYY-1524 | V82S, D119L |
| pYY-1525 | V82S, L144Q |
| pYY-1526 | V82S, Y147A |
| pYY-1527 | V82S, M118L |
| pYY-1528 | V82S, R107A, L63F |
| pYY-1529 | V82S, A62V |
| pYY-1530 | V82S, E3G, I132T, F6A |
| pYY-1531 | V82S, TadA7.10 |
| pYY-1532 | Tad8.8 |
| pYY-1533 | Tad8.20 |
| pYY-1534 | Y81V, L18A |
| pYY-1535 | G50A, H122T, A142S, P29Q, L36S |
| pYY-1536 | L145R, G42M |
| pYY-1537 | G67N |
| pYY-1538 | N72Y, F156N, H128R |
| pYY-1539 | P124K |
| pYY-1540 | N38D, P124Q, L68V |
| pYY-1541 | P29Y, M94R, A142N |
| pYY-1542 | K160E, H14D |
| pYY-1543 | V82S, Y81V, L18A |
| pYY-1544 | V82S, G50A, H122T, A142S, P29Q, L36S |
| pYY-1545 | V82S, L145R, G42M |
| pYY-1546 | V82S, G67N |
| pYY-1547 | V82S, N72Y, F156N, H128R |
| pYY-1548 | V82S, P124K |
| pYY-1549 | V82S, N38D, P124Q, L68V |
| pYY-1550 | V82S, P29Y, M94R, A142N |
| pYY-1551 | V82S, K160E, H14D |
| pYY-1552 | E3N |
| pYY-1553 | I132T |
| pYY-1554 | L63R |
| pYY-1555 | N46Y |
| pYY-1556 | V35Q, V130F |
| pYY-1557 | T79H, L145N |
| pYY-1558 | F149A |
| pYY-1559 | G67N |
| pYY-1560 | Q65R |
| pYY-1561 | M70I |
| pYY-1562 | P124H |
| pYY-1563 | H52L |
| pYY-1564 | V82S, E3N |
| pYY-1565 | V82S, I132T |
| pYY-1566 | V82S, L63R |
| pYY-1567 | V82S, N46Y |
| pYY-1568 | V82S, V35Q, V130F |
| pYY-1569 | V82S, T79H, L145N |
| pYY-1570 | V82S, F149A |
| pYY-1571 | V82S, G67N |
| pYY-1572 | V82S, Q65R |
| pYY-1573 | V82S, M70I |
| pYY-1574 | V82S, P124H |
| pYY-1575 | V82S, H52L |

Sets 7 and 8: Cas9 Guide-Specific Evolution with Counter Selection.

Guide specific evolution is explained above in Set 3, and off-target selection explained above in Set 1. A Cas9 N-terminal error-prone library (Set 7) and PI domain error-prone library (Set 8) were made and selected using these two selection strategies. Both guide-specific yeast evolution and off-target selection were used in this evolution. Guide-specific yeast evolution was an evolution strategy that allowed evolving BEs/Cas enzymes towards any user-defined target site. This evolution was performed with a defined SpCas9 guide (targeting SERPINA1), and the base editor first edited target site 1. Target site 1 was the crRNA region in a Cas12a guide, and target site 1 had the same sequence as the A1AT target site. This Cas12a guide was expressed with Cas12a protein. When target site 1 was edited by the base editor, the Cas12a complex recognized target site 2 in a disrupted EGFP gene, and resulted in a DSB in the gene. This was followed by HDR repair resulting in a functional EGFP gene. When the target site 1 was not edited by the base editor, the Cas12a complex was not be able to recognize target site 2, and there was no EGFP signal. Using this strategy, enabled selection of linkers in BEs that improved potency for the A1AT target site.

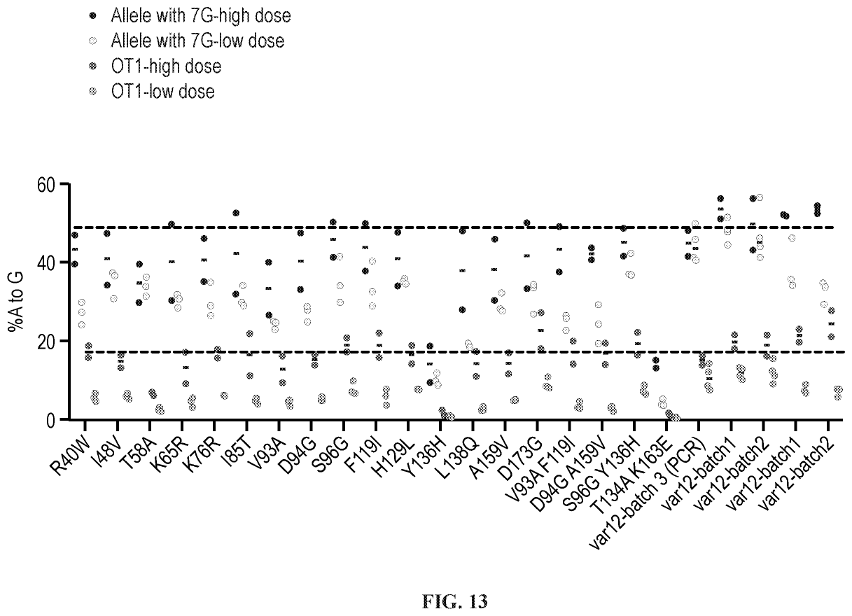

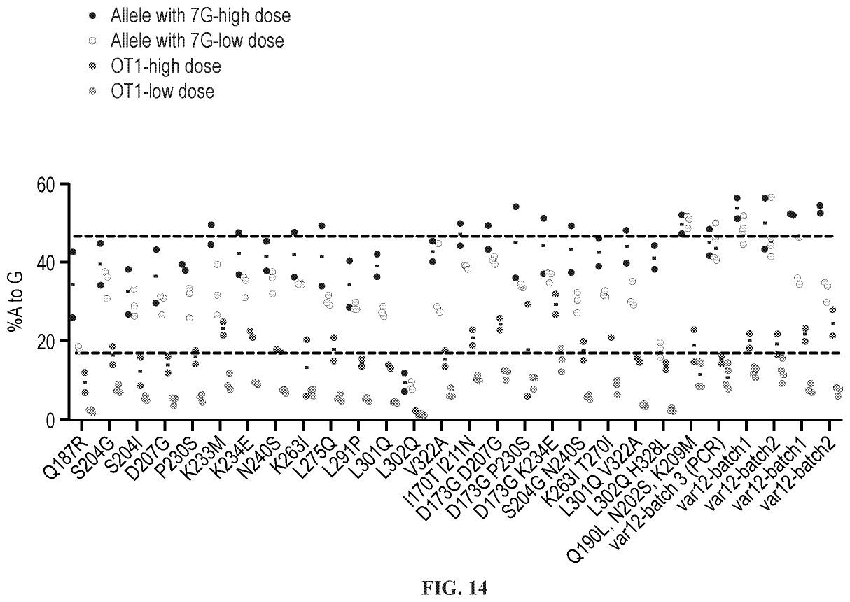

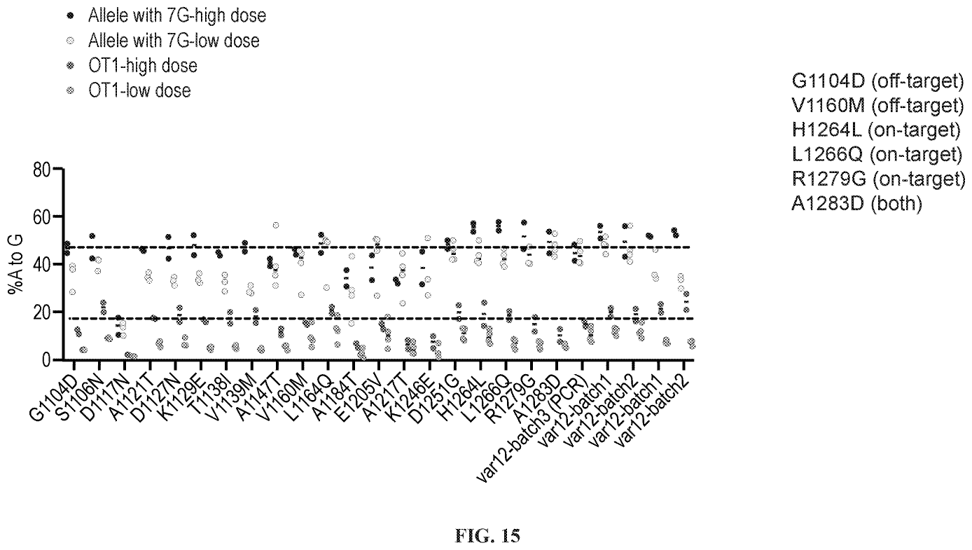

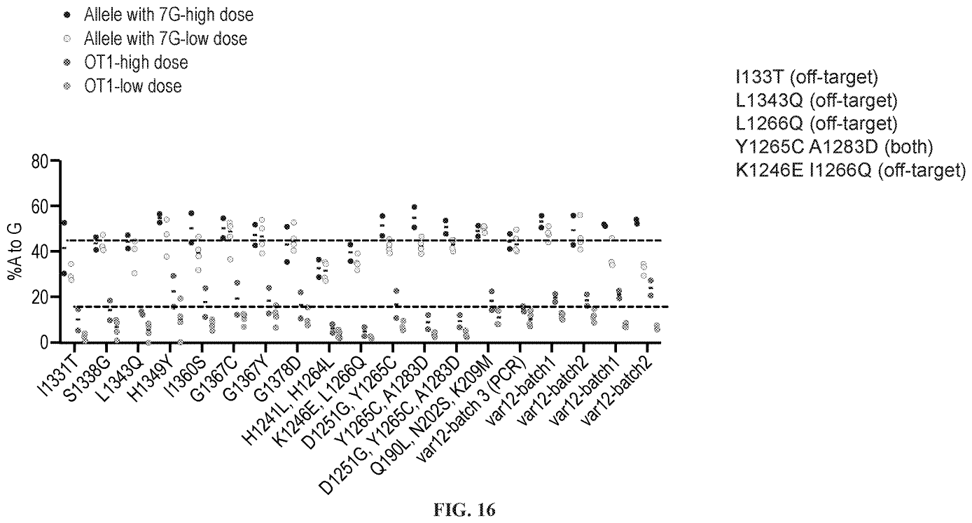

For off-target selection, three guides were targeting the same stop codon in an inactivated mCherry gene, and their target sites were with NGA, NGG, NGT PAM, respectively. The A to G editing activities guided by any of the three guides resulted in mCherry signal. The yeast cells were transformed with a library of base editors derived from Variant 12 NGC Cas9. This library contained random mutations in N-term Cas9 (aa 1-350) or Cas9 PI domain, and these random mutations were introduced by error-prone PCR. After culturing the yeast cells containing both selection plasmid and base editor plasmid, yeast cells with high EGFP signal and low mcherry signal were selected. Mutations in SpCas9-MQKFRAER nickase identified in the screen are shown in Table 15 and Table 16. Results for % A to G editing of ABE variants having SpCas9-MQKFRAER nickase mutations identified in this screen are shown in FIG. 13, FIG. 14, FIG. 15, and FIG. 16. Proposed combinations of mutations in Variant 12 TadA*7.10 and SpCas9-MQKFRAER nickase are listed in Table 17.

TABLE 15
Cas9 mutations identified in Set 7,Cas9 guide-specific evolution
with counter selection, using a Cas9 N-terminal error-prone library.

| | Mutation in SpCas9- |
|---|---|
| Plasmid Number | MQKFRAER nickase |
| pYY-1601 | W18R |
| pYY-1602 | R40W |
| pYY-1603 | I48V |
| pYY-1604 | T58A |
| pYY-1605 | K65R |
| pYY-1606 | K76R |
| pYY-1607 | I85T |
| pYY-1608 | V93A |
| pYY-1609 | D94G |
| pYY-1610 | S96G |
| pYY-1611 | F119I |
| pYY-1612 | H129L |
| pYY-1613 | Y136H |
| pYY-1614 | L138Q |
| pYY-1615 | A159V |
| pYY-1616 | D173G |
| pYY-1617 | V93A, F119I |
| pYY-1618 | D94G, A159V |
| pYY-1619 | S96G, Y136H |
| pYY-1620 | T134A, K163E |
| pYY-1621 | Q187R |
| pYY-1622 | S204G |
| pYY-1623 | S204I |
| pYY-1624 | D207G |
| pYY-1625 | P230S |
| pYY-1626 | K233M |
| pYY-1627 | K234E |
| pYY-1628 | N240S |
| pYY-1629 | K263I |
| pYY-1630 | L275Q |
| pYY-1631 | L291P |
| pYY-1632 | L301Q |
| pYY-1633 | L302Q |
| pYY-1634 | V322A |
| pYY-1635 | I170T, I211N |
| pYY-1636 | D173G, D207G |
| pYY-1637 | D173G, P230S |
| pYY-1638 | D173G, K234E |
| pYY-1639 | S204G, N240S |
| pYY-1640 | K263I, T270I |
| pYY-1641 | L301Q, V322A |
| pYY-1642 | L302Q, H328L |

TABLE 16
Cas9 mutations identified in Set 7, Cas9 guide-specific evolution
with counter selection, using a PI domain error-prone library.

| Plasmid Number | Mutation in SpCas9-MQKFRAER nickase |
|---|---|
| pYY-1643 | G1104D |
| pYY-1644 | S1106N |
| pYY-1645 | D1117N |
| pYY-1646 | A1121T |
| pYY-1647 | D1127N |
| pYY-1648 | K1129E |
| pYY-1649 | T1138I |
| pYY-1650 | V1139M |
| pYY-1651 | A1147T |
| pYY-1652 | V1160M |
| pYY-1653 | L1164Q |
| pYY-1654 | A1184T |
| pYY-1655 | E1205V |
| pYY-1656 | A1217T |
| pYY-1657 | H1241L |
| pYY-1658 | K1246E |
| pYY-1659 | D1251G |
| pYY-1660 | H1264L |
| pYY-1661 | L1266Q |
| pYY-1662 | R1279G |
| pYY-1663 | A1283D |
| pYY-1664 | K1325G |
| pYY-1665 | I1331T |
| pYY-1666 | S1338G |
| pYY-1667 | L1343Q |
| pYY-1668 | H1349Y |
| pYY-1669 | R1359W |
| pYY-1670 | I1360S |
| pYY-1671 | G1367C |
| pYY-1672 | G1367Y |
| pYY-1673 | G1378D |
| pYY-1674 | H1241L, H1264L |
| pYY-1675 | K1246E, L1266Q |
| pYY-1676 | D1251G, Y1265C |
| pYY-1677 | Y1265C, A1283D |
| pYY-1678 | D1251G, Y1265C, A1283D |
| pYY-1679 | Q190L, N202S, K209M |
| pYY-1680 | K2631, T270I, V322A |

TABLE 17
Proposed combinations of mutations in Variant
12 TadA*7.10 and SpCas9-MQKFRAER nickase.

| | | | Mutation in SpCas9-MQKFRAER |
|---|---|---|---|
| Plasmid | Derived From | Mutation in TadA | nickase |
| pYY-1800 | Var12- with | | |
| | Q1136Y + | | |
| | R1337K | | |
| pYY-1801 | Var12- with | P29Y M94R A142N | |
| | Q1136Y + | | |
| | R1337K | | |
| pYY-1802 | Var12- with | N72Y F156N H128R | |
| | Q1136Y + | | |
| | R1337K | | |
| pYY-1803 | Var12- with | N72Y F156Y H128R | |
| | Q1136Y + | | |
| | R1337K | | |
| pYY-1804 | Var12- with | P29Y M94R A142N | |
| | Q1136Y + | N72Y F156Y H128R | |
| | R1337K | | |
| pYY-1805 | Var12- with | F156Y | |
| | Q1136Y + | | |
| | R1337K | | |
| pYY-1806 | Var12- with | A109T | |
| | Q1136Y + | | |
| | R1337K | | |
| pYY-1807 | Var12- with | A109T V155S | |
| | Q1136Y + | | |
| | R1337K | | |
| pYY-1808 | Var12- with | F156Y + A109T V155S | |
| | Q1136Y + | | |
| | R1337K | | |
| pYY-1809 | Var12- with | A109T V155S + P29Y | |
| | Q1136Y + | M94R A142N | |
| | R1337K | | |
| pYY-1810 | Var12- with | A109T V155S + N72Y | |
| | Q1136Y + | F156Y H128R | |
| | R1337K | | |
| pYY-1811 | Var12- with | A109T V155S + P29Y | |
| | Q1136Y + | M94R A142N + N72Y | |
| | R1337K | F156Y H128R | |
| pYY-1812 | Var12- with | | A1283D |
| | Q1136Y + | | |
| | R1337K | | |
| pYY-1813 | Var12- with | | Y1265C A1283D |
| | Q1136Y + | | |
| | R1337K | | |
| pYY-1814 | Var12- with | | R765A |
| | Q1136Y + | | |
| | R1337K | | |
| pYY-1815 | Var12- with | | Q768A |
| | Q1136Y + | | |
| | R1337K | | |
| pYY-1816 | Var12- with | | R765A Q768A |
| | Q1136Y + | | |
| | R1337K | | |
| pYY-1817 | Var12- with | | R765A Q768A K772A K775A |
| | Q1136Y + | | |
| | R1337K | | |
| pYY-1818 | Var12- with | | H1264L |
| | Q1136Y + | | |
| | R1337K | | |
| pYY-1819 | Var12- with | | L1266Q |
| | Q1136Y + | | |
| | R1337K | | |
| pYY-1820 | Var12- with | | R1279G |
| | Q1136Y + | | |
| | R1337K | | |
| pYY-1821 | Var12- with | | S219A |
| | Q1136Y + | | |
| | R1337K | | |
| pYY-1822 | Var12- with | | R220A R221A |
| | Q1136Y + | | |
| | R1337K | | |
| pYY-1823 | Var12- with | | S219A R220A R221A |
| | Q1136Y + | | |
| | R1337K | | |
| pYY-1824 | Var12- with | | A1283D S219A |
| | Q1136Y + | | |
| | R1337K | | |
| pYY-1825 | Var12- with | | A1283D R220A R221A |
| | Q1136Y + | | |
| | R1337K | | |
| pYY-1826 | Var12- with | | A1283D S219A R220A R221A |
| | Q1136Y + | | |
| | R1337K | | |
| pYY-1827 | Var12- with | | A1283D + H1264L |
| | Q1136Y + | | |
| | R1337K | | |
| pYY-1828 | Var12- with | | A1283D + L1266Q |
| | Q1136Y + | | |
| | R1337K | | |
| pYY-1829 | Var12- with | | A1283D + R1279G |
| | Q1136Y + | | |
| | R1337K | | |
| pYY-1830 | Var12- with | | A1283D + H1264L + L1266Q |
| | Q1136Y + | | |
| | R1337K | | |
| pYY-1831 | Var12- with | | A1283D + H1264L + R1279G |
| | Q1136Y + | | |
| | R1337K | | |
| pYY-1832 | Var12- with | | A1283D + L1266Q + R1279G |
| | Q1136Y + | | |
| | R1337K | | |
| pYY-1833 | Var12- with | | A1283D + L1266Q + R1279G + |
| | Q1136Y + | | H1264L |
| | R1337K | | |
| pYY-1834 | Var12- with | | Y1265C A1283D + L1266Q + |
| | Q1136Y + | | R1279G + H1264L |
| | R1337K | | |
| pYY-1835 | Var12- with | | S219A R220A R221A + A1283D + |
| | Q1136Y + | | L1266Q + R1279G + H1264L |
| | R1337K | | |
| pYY-1836 | Var12- with | | S219A R220A R221A + Y1265C + |
| | Q1136Y + | | A1283D + L1266Q + R1279G + |
| | R1337K | | H1264L |
| pYY-1837 | Var12- with | | Y1265C A1283D + L1266Q + |
| | Q1136Y + | | R1279G + H1264L + R765A Q768A |
| | R1337K | | |
| pYY-1838 | Var12- with | | S219A R220A R221A + Y1265C + |
| | Q1136Y + | | A1283D + L1266Q + R1279G + |
| | R1337K | | H1264L + R765A Q768A |
| pYY-1839 | Var12- with | | A1283D + S219A R220A R221A |
| | Q1136Y + | | E1250K |
| | R1337K | | |
| pYY-1840 | Var12- with | | E1250K |
| | Q1136Y + | | |
| | R1337K | | |
| pYY-1841 | Var12- with | F84Y | |
| | Q1136Y + | | |
| | R1337K | | |
| pYY-1842 | Var12- with | F84Y A109T | |
| | Q1136Y + | | |
| | R1337K | | |
| pYY-1843 | Var12- with | | R765D |
| | Q1136Y + | | |
| | R1337K | | |
| pYY-1844 | Var12- with | | R765D Q768D |
| | Q1136Y + | | |
| | R1337K | | |
| pYY-1845 | Var12- with | | R765A + R220A R221A |
| | Q1136Y + | | |
| | R1337K | | |
| pYY-1846 | Var12- with | | R765A Q768A + R220A R221A |
| | Q1136Y + | | |
| | R1337K | | |
| pYY-1847 | Q1136Y | | A1283D |
| | R1337K | | |
| pYY-1848 | Q1136Y | | A1283D+ E1250K |
| | R1337K | | |
| pYY-1849 | Q1136Y | | M1135L + A1283D |
| | R1337K | | |
| pYY-1850 | Q1136Y | | M1135L + E1250K |
| | R1337K | | |
| pYY-1851 | Q1136Y | | M1135L + A1283D + E1250K |
| | R1337K | | |
| pYY-1852 | R1337K | | A1283D |
| pYY-1853 | R1337K | | A1283D + E1250K |
| pYY-1854 | R1337K | | M1135L + A1283D |
| pYY-1855 | R1337K | | M1135L + E1250K |
| pYY-1856 | R1337K | | M1135L + A1283D + E1250K |
| pYY-1857 | Q1136Y | | A1283D + E1250K |
| pYY-1858 | Q1136Y | | A1283D + H1264L + E1250K |
| | R1337K | | |
| pYY-1859 | Q1136Y | | A1283D + L1266Q + E1250K |
| | R1337K | | |
| pYY-1860 | R1337K | | A1283D + H1264L + E1250K |
| pYY-1861 | R1337K | | A1283D + L1266Q + E1250K |
| pYY-1862 | Q1136Y | | A1283D + H1264L + E1250K + |
| | R1337K | | M1135L |
| pYY-1863 | Q1136Y | | A1283D + L1266Q + E1250K + |
| | R1337K | | M1135L |
| pYY-1864 | Q1136Y | | A1283D + H1264L + L1266Q + |
| | R1337K | | E1250K |
| pYY-1865 | Q1136Y | | A1283D + H1264L + L1266Q + |
| | R1337K | | E1250K + M1135L |
| pYY-1866 | KGPKPKKEESEK | | M1135L + A1283D + Q1136Y |
| | | | R1337K |
| pYY-1867 | KGPKPKKEESEK + | | M1135L + A1283D + Q1136Y |
| | R220A R221A | | R1337K |
| pYY-1868 | R765A Q768A | | M1135L + A1283D + Q1136Y |
| | | | R1337K |
| pYY-1869 | R765AQ768A | | M1135L + A1283D + Q1136Y |
| | K772AK775A | | R1337K |
| pYY-1870 | S219A | | M1135L + A1283D + Q1136Y |
| | | | R1337K |
| pYY-1871 | R220A R221A | | M1135L + A1283D + Q1136Y |
| | | | R1337K |
| pYY-1872 | KGPKPKKEESEK | | M1135L + A1283D + E1250K + |
| | | | R1337K |
| pYY-1873 | KGPKPKKEESEK + | | M1135L + A1283D + E1250K + |
| | R220A R221A | | R1337K |
| pYY-1874 | R765A Q768A | | M1135L + A1283D + E1250K + |
| | | | R1337K |
| pYY-1875 | R765AQ768A | | M1135L + A1283D + E1250K + |
| | K772AK775A | | R1337K |
| pYY-1876 | S219A | | M1135L + A1283D + E1250K + |
| | | | R1337K |
| pYY-1877 | R220A R221A | | M1135L + A1283D + E1250K + |
| | | | R1337K |
| pYY-1878 | KGPKPKKEESEK | | A1283D + E1250K + Q1336Y |
| pYY-1879 | KGPKPKKEESEK + | | A1283D + E1250K + Q1336Y |
| | R220A R221A | | |
| pYY-1880 | R765A Q768A | | A1283D + E1250K + Q1336Y |
| pYY-1881 | R765AQ768A | | A1283D + E1250K + Q1336Y |
| | K772AK775A | | |
| pYY-1882 | S219A | | A1283D + E1250K + Q1336Y |
| pYY-1883 | R220A R221A | | A1283D + E1250K + Q1336Y |

### Example 2: Assessment of ABEs

Novel ABE Variants A-K, which are summarized in Table 18, were tested to determine whether they had improved properties, including (1) decreased guide-dependent off target sites, (2) increased on target editing, and (3) similar or decreased bystander editing. Such improved editors could be used for treating A1AT. Variants A-K were compared to ABE Variant 12 as a control. Editors having decreased guide-dependent off target sites may lower risk of biological impact from guide-dependent off target edits. Further, increased on target editing could increase the therapeutic benefit, decrease the risk of off target edits, and potentially be used at a lower dose.

TABLE 18
A summary of ABE Variants A-K.

| | | SpCas9- | | |
|---|---|---|---|---|
| | | MQKFRAER | | SEQ |
| | ABE | nickcase | | ID |
| Plasmid | Variant | mutant | Linker | NO |
| pMSP1115 | Variant | R1337K | SGGSSGGSSGSET | 357 |
| | A | | PGTSESATPESSG | |
| | | | GSSGGS | |
| pMSP1116 | Variant | Q1136Y, | SGGSSGGSSGSET | 357 |
| | B | and | PGTSESATPESSG | |
| | | R1337K | GSSGGS | |
| pMSP1130 | Variant | M1135L, | EGGSEEEEESGS | 542 |
| | C | Q1136Y, | | |
| | | and | | |
| | | R1337K | | |
| pMSP1131 | Variant | Q1136Y, | EGGSEEEEESGS | 542 |
| | D | and | | |
| | | R1337K | | |
| pMSP1132 | Variant | M1135L, | KGPKPKKEESEK | 439 |
| | E | Q1136Y, | | |
| | | and | | |
| | | R1337K | | |
| pMSP1160 | Variant | M1135L, | SGGSSGGSSGSET | 357 |
| | F | A1283D, | PGTSESATPESSG | |
| | | Q1136Y, | GSSGGS | |
| | | and | | |
| | | R1337K | | |
| pMSP1167 | Variant | M1135L, | SGGSSGGSSGSET | 357 |
| | G | A1283D, | PGTSESATPESSG | |
| | | E1250K, | GSSGGS | |
| | | and | | |
| | | R1337K | | |
| pMSP1168 | Variant | A1283D, | SGGSSGGSSGSET | 357 |
| | H | E1250K, | PGTSESATPESSG | |
| | | and | GSSGGS | |
| | | Q1136Y | | |
| pMSP1177 | Variant | M1135L, | KGPKPKKEESEK | 439 |
| | I | A1283D, | | |
| | | Q1136Y, | | |
| | | and | | |
| | | R1337K | | |
| pMSP1179 | Variant | M1135L, | SGGSSGGSSGSET | 357 |
| | J | A1283D, | PGTSESATPESSG | |
| | | Q1136Y, | GSSGGS | |
| | | R1337K, | | |
| | | R765A, | | |
| | | and | | |
| | | Q768A | | |
| pMSP1188 | Variant | A1283D, | KGPKPKKEESEK | 439 |
| | K | E1250K, | | |
| | | and | | |
| | | Q1136Y | | |

Hundreds of editor variants identified by the various screens and structure-based approaches were tested in patient fibroblasts for on-target editing. Guide-dependent off target editing at candidate OT sites and guide-independent off target editing using the sensitive R-loop assay were also performed in patient fibroblasts. Best performing editors were tested at a sub-saturating dose of 0.25 mpk against Variant 12 codon optimized in vivo in two studies (SBTx760 and SBTx850) performed in NSG-PiZ mice (a widely accepted model of AATD). Several editors were advanced into rhAmp-seq guide-dependent off-target editing in primary hepatocytes. These experiments identified new therapeutic A1AT candidates, including Variants A-K.

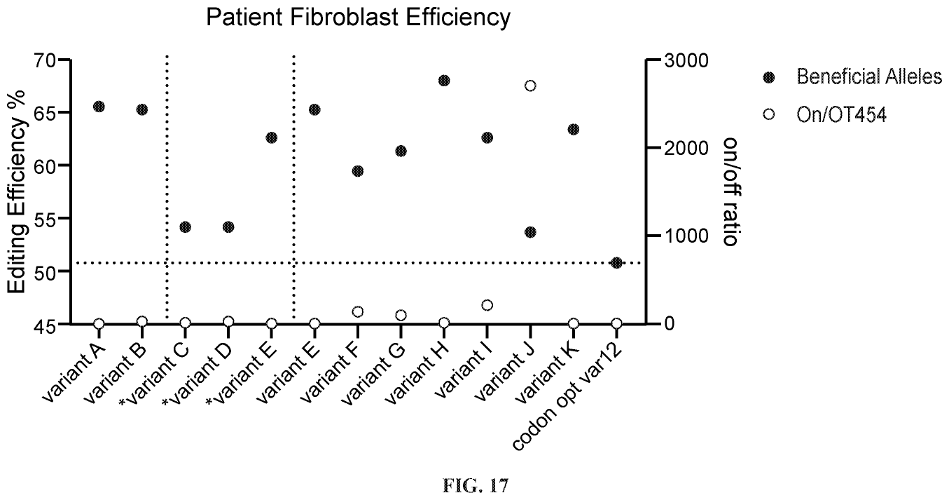

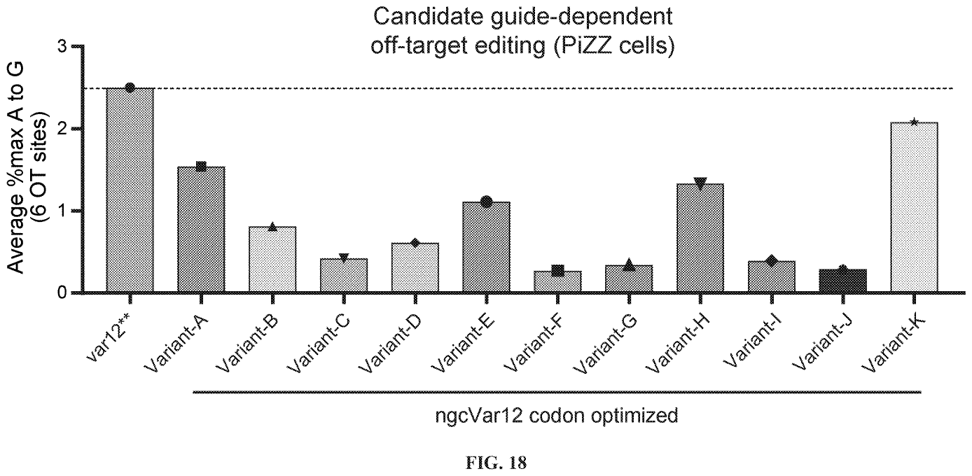

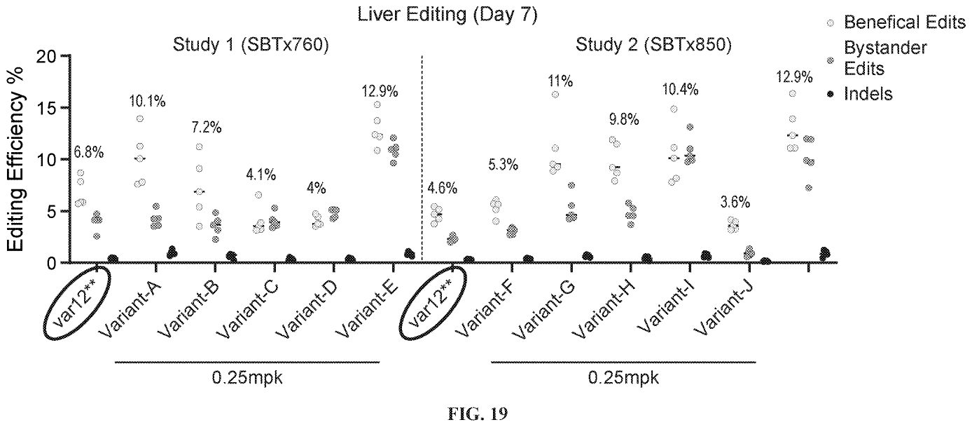

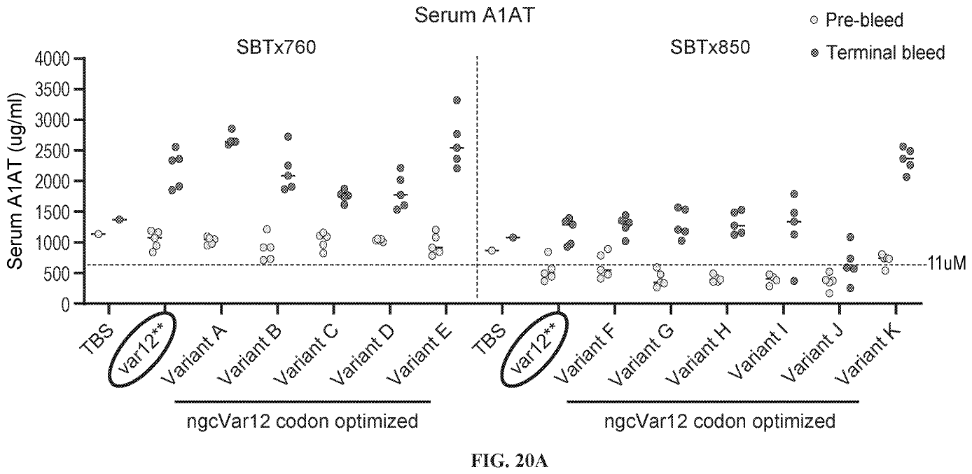

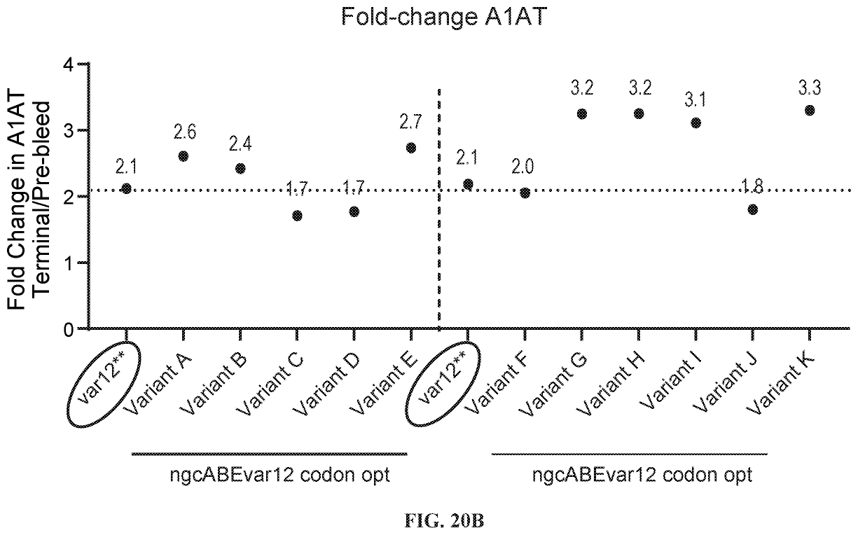

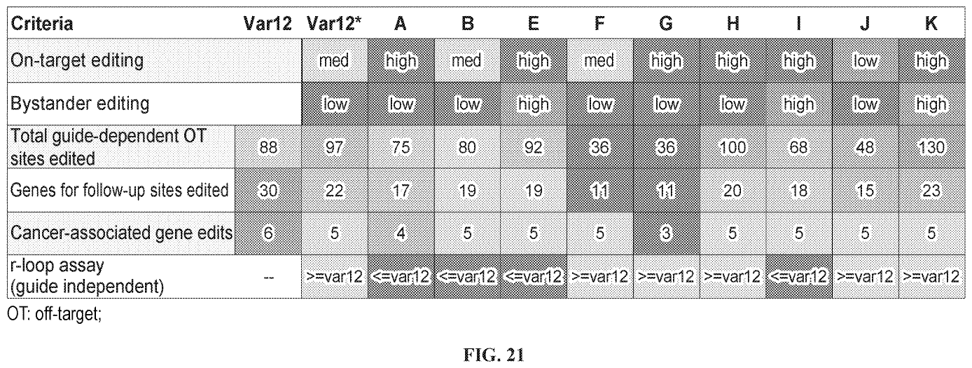

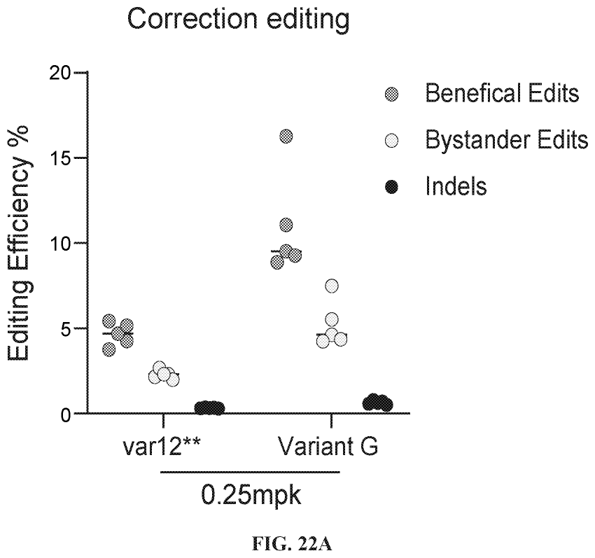

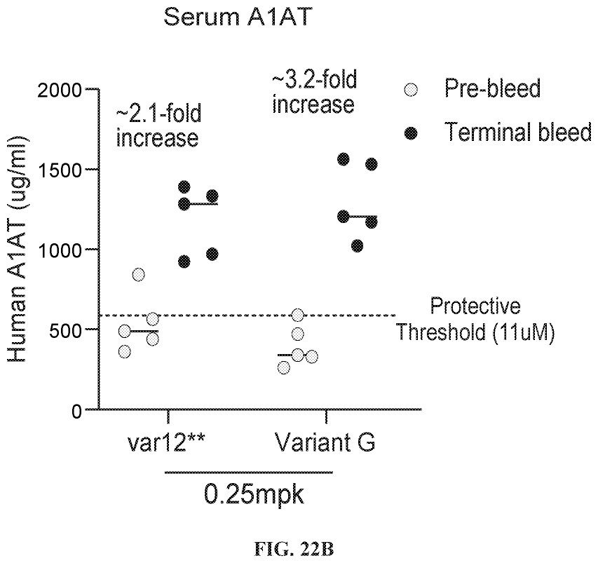

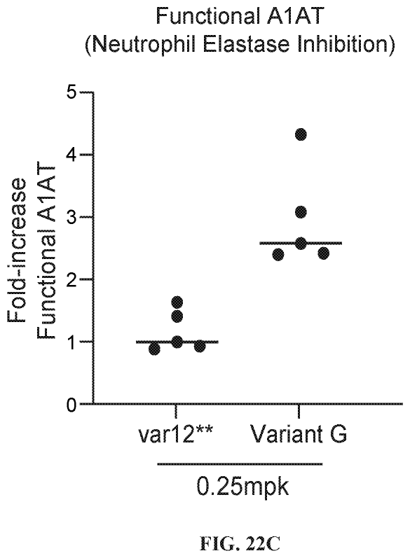

Patient fibroblasts that carry the PiZ mutation were electroporated with mRNA encoding editor variants and gRNA856 to assess on-target correction editing and off-target editing at a candidate site (OT454) using amplicon sequencing (FIG. 17). Multiple new editors demonstrated improvement in on-target editing and decreased on/off-target editing ratio. Patient fibroblasts that carry the PiZ mutation were electroporated with mRNAs encoding 11 editor variants and gRNA856 to assess guide-dependent off-target editing at 6 candidate OT sites using amplicon sequencing. All editor variants tested had average OT editing lower than that observed for codon optimized Variant (FIG. 18). Eleven editor variants and guide RNA 856 were tested alongside codon optimized Var12 in vivo in NSG-PiZ mice at a sub-saturating dose of 0.25 mpk. Liver editing was assessed using NGS. Multiple new editors demonstrate improved on-target editing relative to codon optimized Variant 12 (FIG. 19). Further, in the in vivo experiments, improved liver editing corresponded to increases in the biomarker, serum A1AT (FIG. 20A) and (FIG. 20B). A subset of new editor variants shows a decrease in number of OT sites relative to codon optimized Variant 12. Variant G shows improvement across most criteria including improved in vivo on-target editing, similar bystander editing and lower number of guide-dependent OT sites (FIG. 21). ABE Variant G exhibits improved efficacy in NSG-PiZ mice. In mice, 11% editing and 3.2-fold change in AAT at 0.25 mg/kg was achieved. It is expected to achieve ~20% editing at 0.5 mpk (FIG. 22).

Selected ABE variants were tested to compare on target and off target editing. Patient derived fibroblast cells (PiZZ cells) were transfected with mRNA encoding for indicated variants and synthetic guides using lipofectamine messengerMax. To calculate the in trans score, the editing efficiencies at 6 sites in an R-loop assay were used; to calculate the OT score, editing efficiencies at 6 A1 OT sites were used; to calculate on-target score, the on-target editing at A1 target site was used. All editing efficiencies were normalized to a parent base editor (which is Variant 12). The results of the analysis are shown in Table 19.

TABLE 19
Comparison of on-target and off-target editing for ABE Variants.

| | | | | | rank- | | rank- | Ranking | overall |
|---|---|---|---|---|---|---|---|---|---|
| | | | | on- | ing | rank- | ing | (on- | ranking |
| | Mutant/ | intrans | OT | target | (in- | ing | (on | target | (ad- |
| Plasmid | linker | score | score | score | trans) | (OT) | target) | adjusted) | justed) |
| ABE7.10 | | 10 | | | | | | | |
| MRNA | Var12 | 1 | 1 | 1 | 7 | 14 | 5 | 6 | 13 |
| 2909 | | | | | | | | | |
| MRNA | R1337K | 1.2 | 1.6 | 1.08 | 6 | 12 | 2 | 4 | 6 |
| 2118 | | | | | | | | | |
| MRNA | R1337K | 1.8 | 3.1 | 1.01 | 4 | 9 | 4 | 5 | 2 |
| 2119 | Q1136Y | | | | | | | | |
| MRNA | M1135L | 2.7 | 6 | 0.86 | 3 | 7 | 12 | 12 | 6 |
| 2898 | R1337K | | | | | | | | |
| | Q1136Y + | | | | | | | | |
| | EGGSEEEEES | | | | | | | | |
| | GGS | | | | | | | | |
| MRNA | R1337K | 3.2 | 4.1 | 0.89 | 1 | 8 | 11 | 11 | 3 |
| 2899 | Q1136Y + | | | | | | | | |
| | EGGSEEEEES | | | | | | | | |
| | GGS | | | | | | | | |
| MRNA | M1135L | 3 | 2.3 | 0.97 | 2 | 10 | 9 (?) | 3 | 1 |
| 2900 | R1337K | | | | | | | | |
| | Q1136Y + | | | | | | | | |
| | KGPKPKKEE | | | | | | | | |
| | SEK | | | | | | | | |
| MRNA | M1135L + | 0.7 | 9.3 | 1 | 10 | 3 | 6 | 7 | 3 |
| 3187 | A1283D + | | | | | | | | |
| | Q1136Y | | | | | | | | |
| | R1337K | | | | | | | | |
| MRNA | M1135L + | 0.5 | 7.3 | 1 | 12 | 5 | 7 | 8 | 10 |
| 3194 | A1283D + | | | | | | | | |
| | E1250K + | | | | | | | | |
| | R1337K | | | | | | | | |
| MRNA | A1283D + | 0.5 | 1.9 | 1.1 | 13 | 11 | 1 | 2 | 11 |
| 3195 | E1250K + | | | | | | | | |
| | Q1136Y | | | | | | | | |
| MRNA | 3187 + | 1.4 | 6.4 | 0.98 | 5 | 6 | 8 | 9 | 3 |
| 3288 | KGPKPKKEE | | | | | | | | |
| | SEK | | | | | | | | |
| MRNA | 3187 + | 0.7 | 8.7 | 0.93 | 9 | 4 | 10 | 10 | 9 |
| 3290 | R765A | | | | | | | | |
| | Q768A | | | | | | | | |
| MRNA | 3187 + | 0.6 | 17.6 | 0.83 | 11 | 1 | 14 | 14 | 12 |
| 3291 | R765AQ768A | | | | | | | | |
| | K772AK775A | | | | | | | | |
| MRNA | 3194 + | 0.3 | 11 | 0.85 | 14 | 2 | 13 | 13 | 14 |
| 3296 | R765AQ768A | | | | | | | | |
| | K772AK775A | | | | | | | | |
| MRNA | 3195 + | 0.9 | 1.2 | 1.04 | 8 | 13 | 3 (?) | 1 | 6 |
| 3299 | KGPKPKKEE | | | | | | | | |
| | SEK | | | | | | | | |

### Example 3: In Vivo Correction of the PiZ Mutation in an AATD Mouse Model

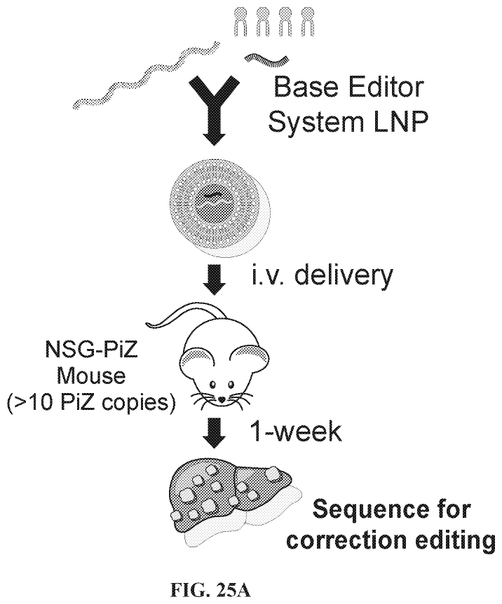

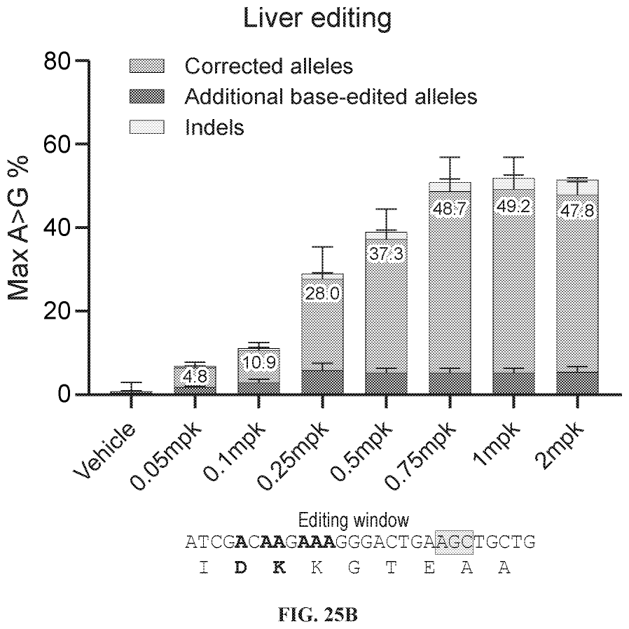

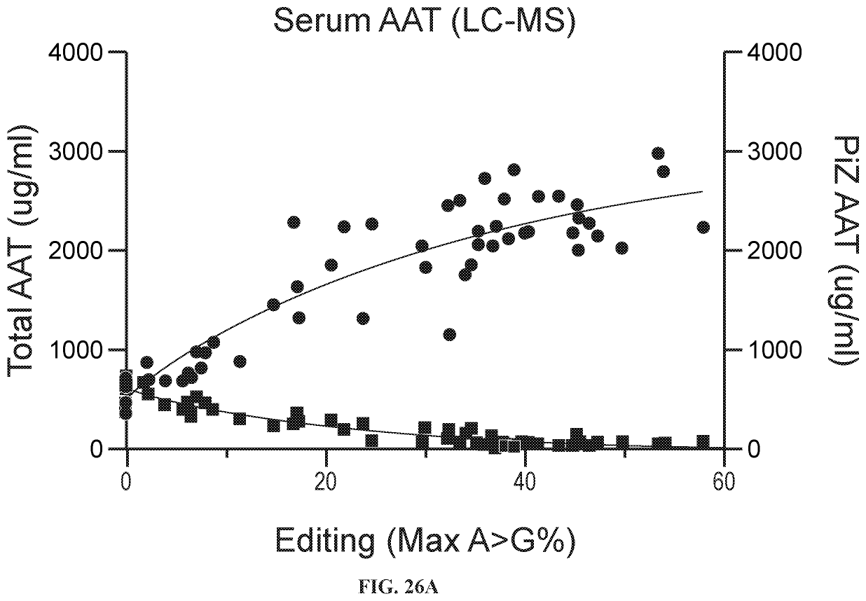

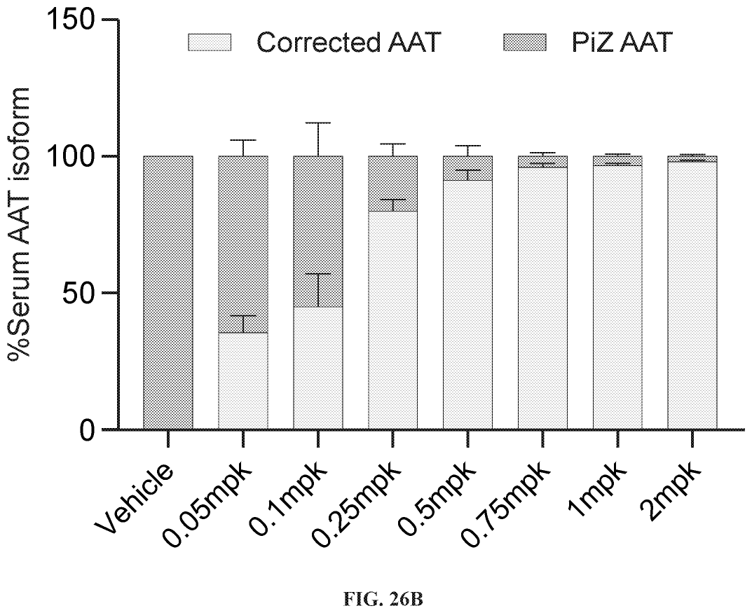

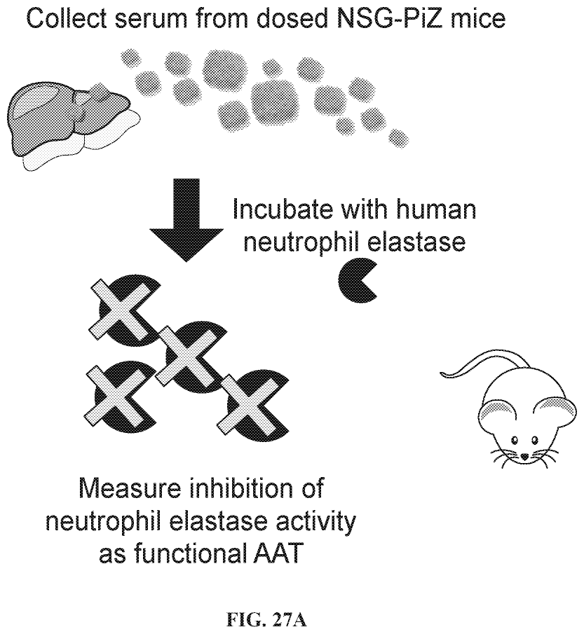

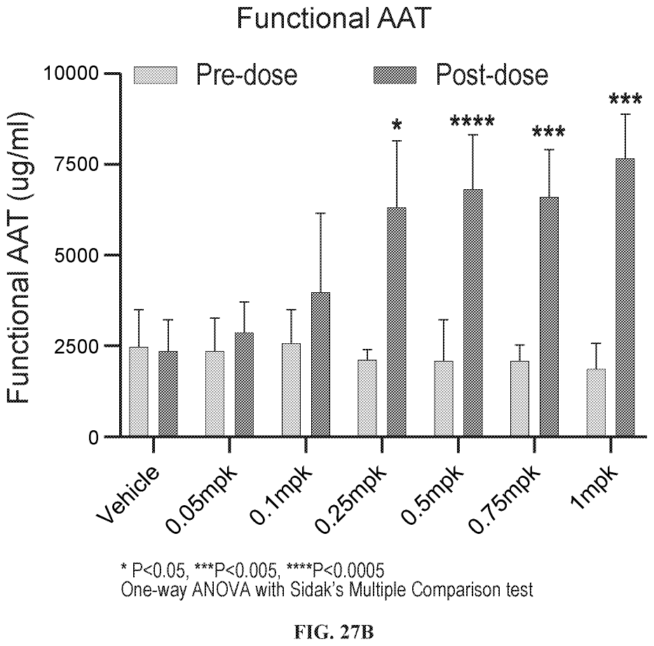

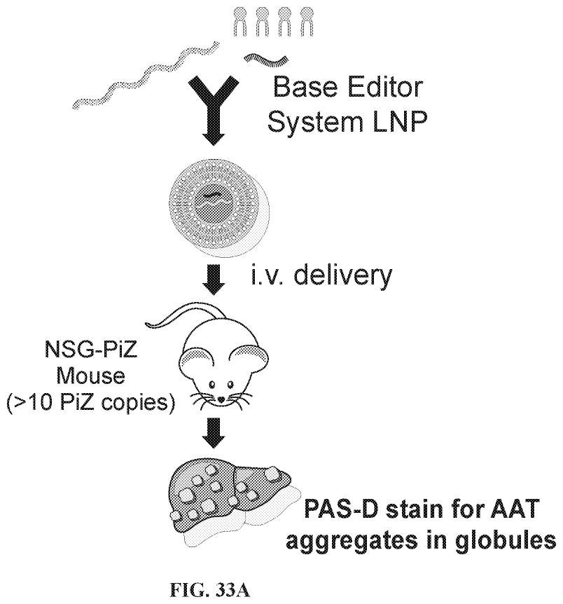

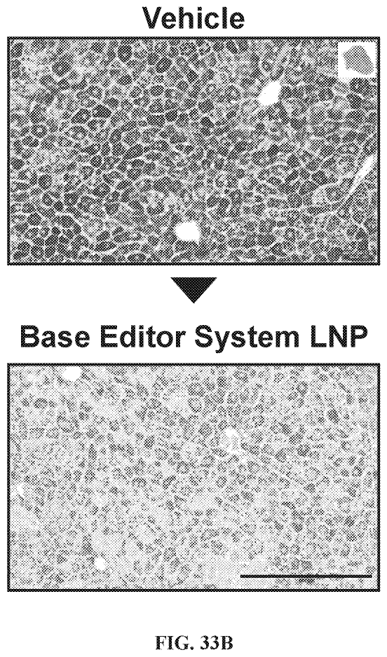

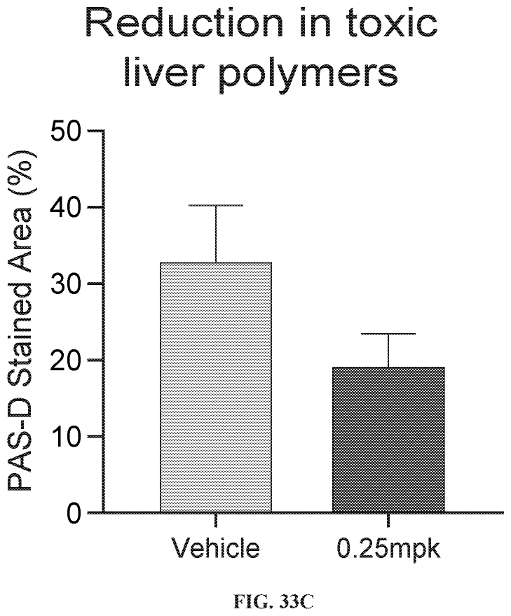

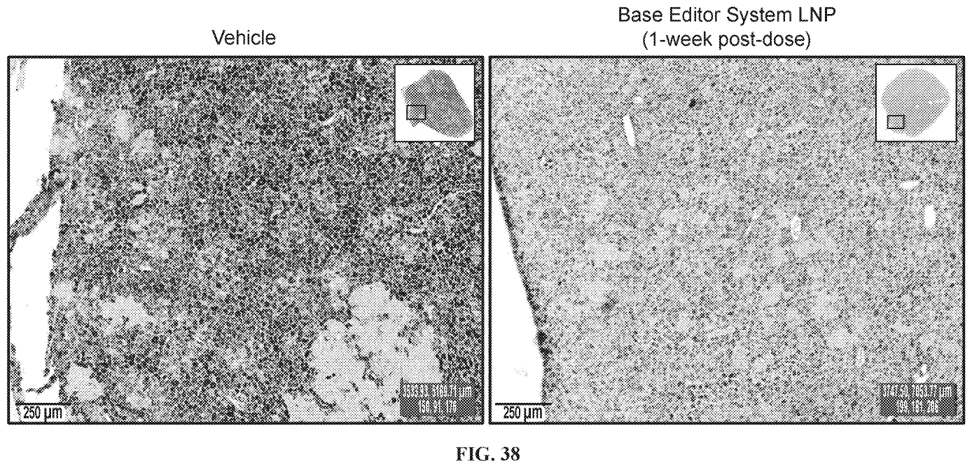

Experiments were undertaken to demonstrate that a base editor system administered to AATD mice using lipid nanoparticles containing an mRNA molecule encoding the base editor ABE Variant G and the guide polynucleotide gRNA856 can be used to correct the PiZ mutation in the mice. AATD mice are also referred to as NSG-PiZ mice, are a model of alpha-1 antitrypsin deficiency, and carry greater than 10 copies of the human SERPINA1 allele PiZ. The mice were administered the base editor system intravenously as shown in FIG. 25A. The lipid nanoparticles containing the base editor system were administered intravenously and measurements were taken one week following the administration. Saturation of corrective base editing was observed at a dose of 0.75 mg/kg total RNA (1:1 mass ratio of the guide polynucleotide gRNA856 and mRNA encoding Variant G), which corresponded to a maximum percent A to G editing of about 50% (FIG. 25B). The base editing was accompanied by a corresponding increase in circulating total corrected (i.e., no longer PiZ AAT) alpha-1 antitrypsin protein (AAT) and decreased levels of PiZ AAT (FIG. 26A), as determined using liquid chromatography-mass spectroscopy (LC-MS) of serum collected from the AATD mice. At doses of 0.25 mg/kg total RNA or higher, more than half the circulating AAT was comprised of corrected AAT rather than PiZ AAT (FIG. 26B). The increase in serum levels of corrected AAT corresponded to increased levels of functional AAT in the serum (FIGS. 27A and 27B). The corrected AAT that was secreted into serum from edited hepatocytes was able to inhibit human neutrophil elastase activity. Therefore, the corrected AAT polypeptides encoded by the base edited PiZ SERPINA1 alleles in the mice were functional. Functional AAT is an important pre-clinical biomarker. Correction of the PiZ mutation also led to decreased liver Z-AAT polymers in the mice (FIGS. 33A-33C and 38).

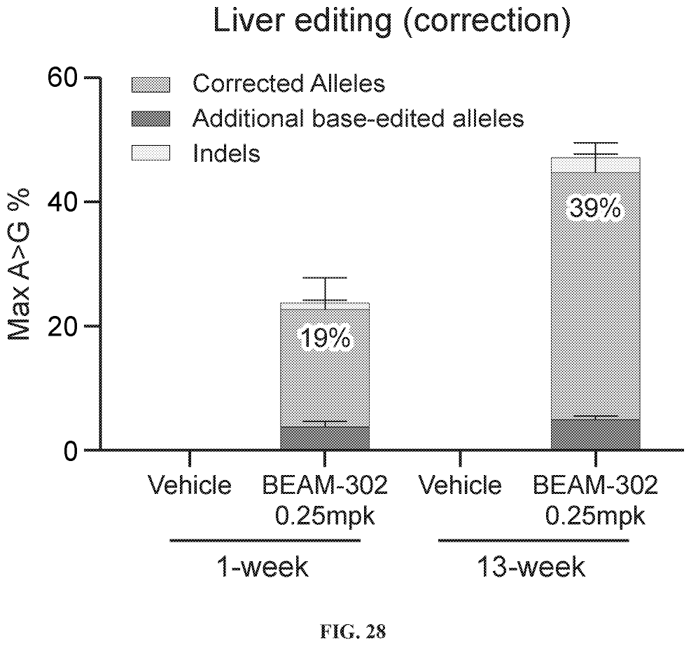

To demonstrate durability of corrective editing in the liver of the mice, parallel cohorts of AATD mice were administered lipid nanoparticles containing mRNA encoding the base editor Variant T and the guide polynucleotide gRNA856 at a dose of 0.25 mg/kg total RNA (1:1 mass ratio of the guide polynucleotide gRNA856 and mRNA encoding Variant G). Maximum percent A to G base editing of the SERPINA1 gene was then measured at one week and at three months in the mice. Livers collected from the mice were analyzed for corrective editing of the PiZ allele of the SERPINA1 gene (FIG. 28) using next-generation sequencing. Maximum percent A to G editing of about 20% was observed at one week post-administration, and this increased to about 40% at three months post-administration. Not intending to be bound by theory, this increase in correction may be the result of a survival and/or proliferative advantage that edited hepatocytes expressing a corrected AAT protein have relative to hepatocytes that still express Z-AAT from a PiZ SERPINA1 gene allele, which is hepatotoxic. A proliferative advantage of hepatocytes expressing a corrected AAT protein relative to hepatocytes expressing Z-AAT has been demonstrated previously in the AATD mouse model.

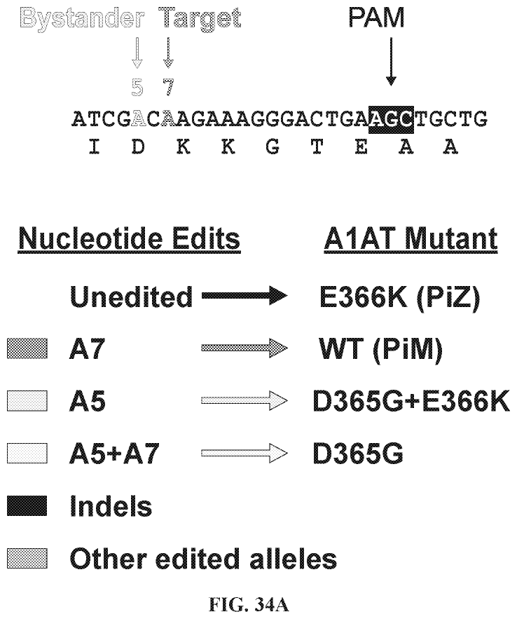

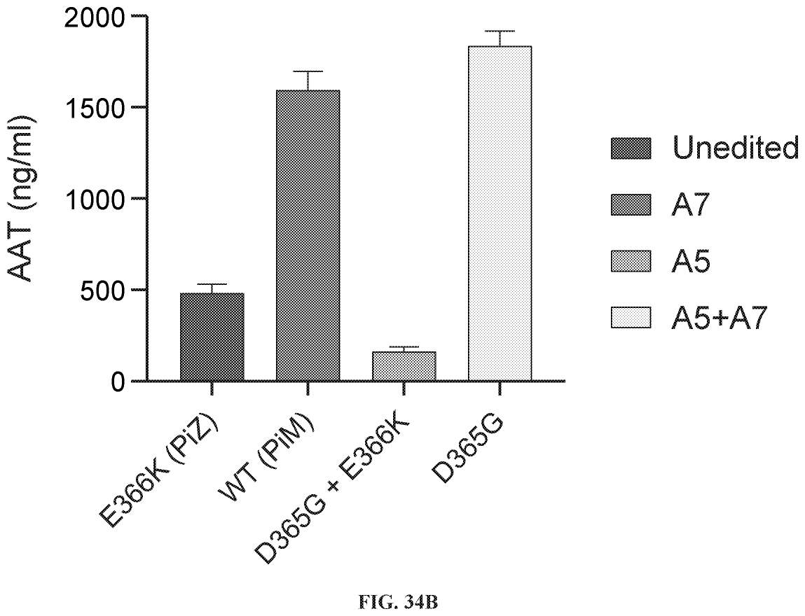

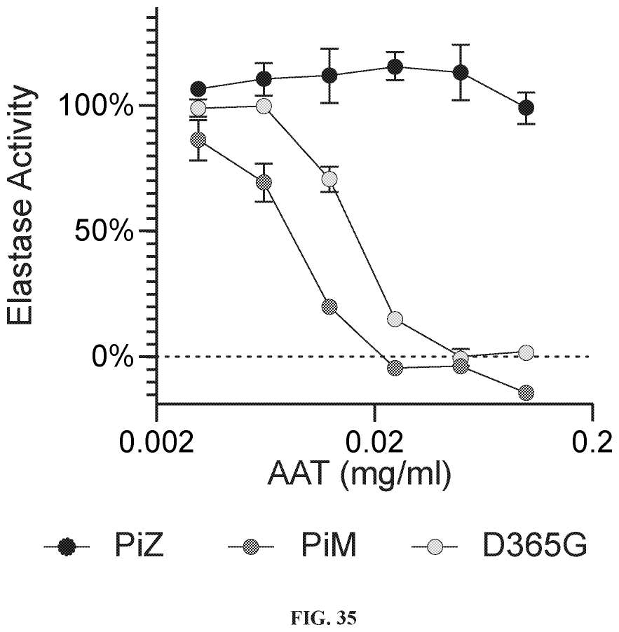

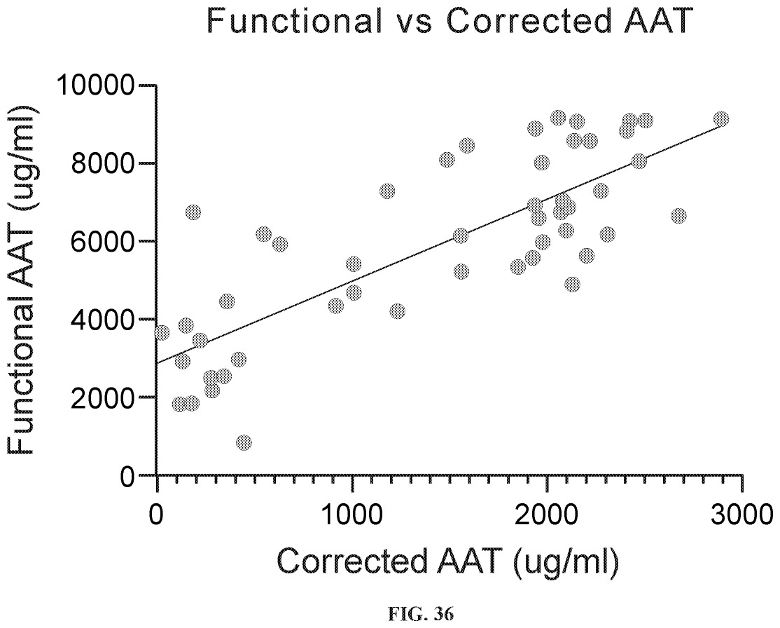

Base editing in the AAT mice, as described above, resulted in the creation of a number of SERPINA1 mutants in the mice (FIG. 34A). Measurements confirmed that the A5+A7 mutant of the SERPINA1 PiZ allele encoded a functional AAT protein having a D365G alteration (FIGS. 34B, 35, and 36). The AAT protein having the D365G alteration was secreted comparably to wild-type ATT encoded by the PiM allele of SERPINA1 (FIG. 34B), functioned in neutrophil elastase inhibition similarly to wild-type ATT (FIG. 35), and increased levels of expression of the AAT protein having the D365G alteration was directly correlated with increases in functional AAT in the mice (FIG. 36).

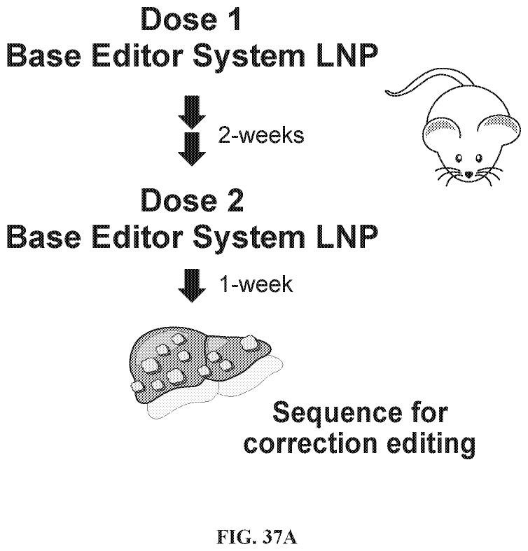

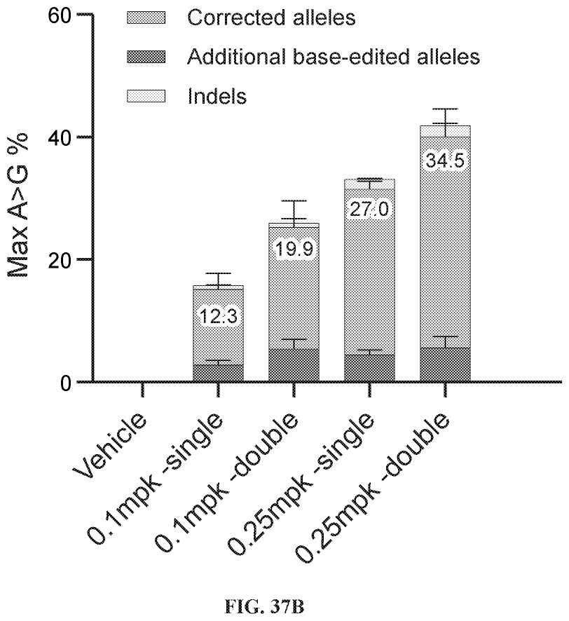

An experiment was undertaken to demonstrate that a second administration of the base editor system at two weeks following the first administration led to an increase in maximum percent A to G editing of the PiZ SERPINA1 gene allele in the mice (FIGS. 37A and 37B). As shown by comparing FIG. 37B to FIG. 25B, a second dose of the base editor system was associated with increased levels of base editing in the mice.

### Example 4: In Vivo Correction of the PiZ Mutation in a Humanized PiZ Rat Model

A humanized rat model of alpha-1 antitrypsin deficiency associated with the PiZ allele of SERPINA1 (hSERPINA1 PiZ rats) was prepared and in vivo correction of the PiZ allele using a base editor system containing the guide polynucleotide gRNA856 and mRNA encoding the base editor ABE Variant G was evaluated.

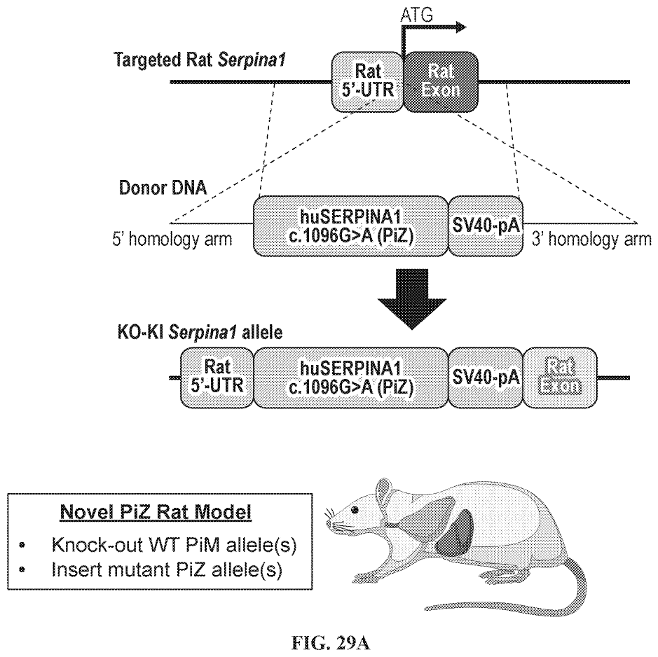

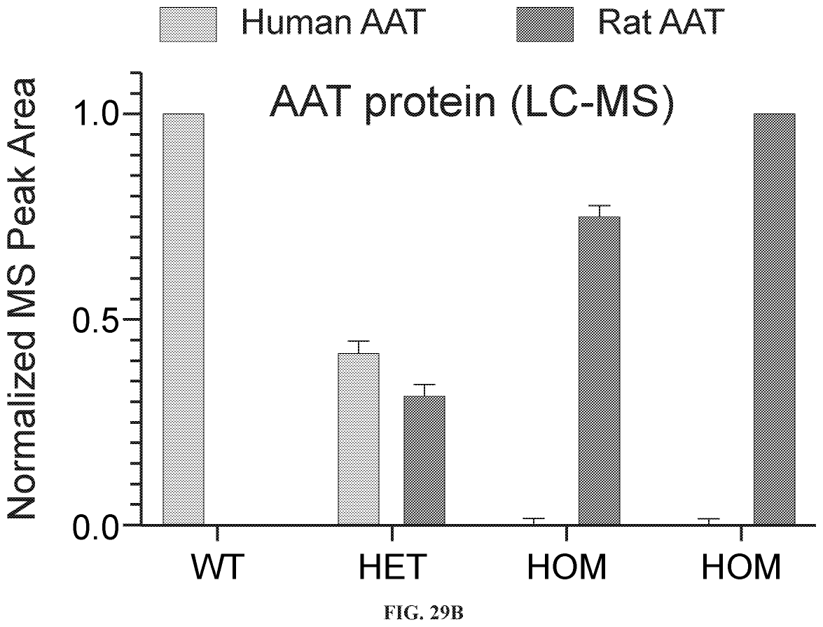

The hSERPINA1 PiZ rats were prepared as shown in FIG. 29A. The hSERPINA1 PiZ rats were prepared by inserting the human SERPINA1 PiZ allele coding sequence followed by a SV40-pA sequence (a poly(A) tail) immediately 3′ of the endogenous Serpina1 ATG codon in Sprague-Dawley rats. The insertion interrupted expression of the rat Serpina1 gene and replaced it with the SERPINA1 PiZ allele under the control of the endogenous Serpina1 gene promoter in the rats. Knock-out of the rat gene and knock-in of the human PiZ gene was confirmed by sequencing of genomic DNA collected from two founder colonies of the hSERPINA1 PiZ rats. As shown in FIG. 29B, liquid chromatography-coupled mass spectrometry (LC-MS) of serum samples from the hSERPINA1 PiZ rats confirmed that rats homozygous for the human SERPINA1 PiZ allele no longer expressed rat AAT and only expressed human Z-AAT (which is the protein expressed from the SERPINA1 PiZ alleles) (about 3.5 μM at 18 weeks). The hSERPINA1 PiZ rats may develop both liver and lung phenotypes of alpha-1 antitrypsin deficiency. The hSERPINA1 PiZ rats also represents an immunocompetent rodent model that expresses the mutant Z-AAT protein and can be used in pharmacological or toxicological studies to complement the NSG-PiZ mouse model, which has liver pathology but no immune system.

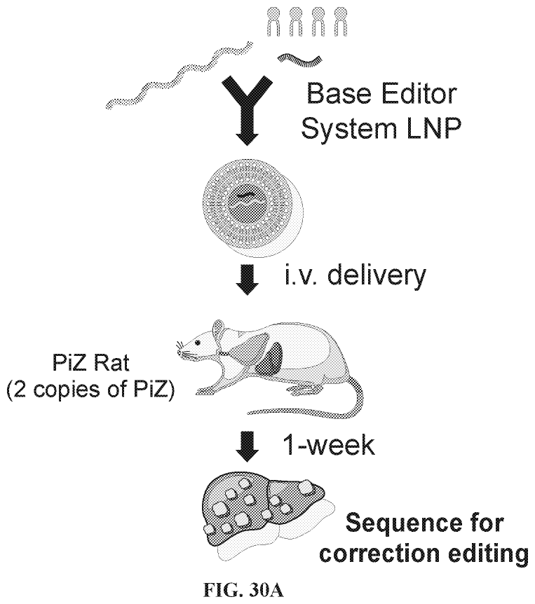

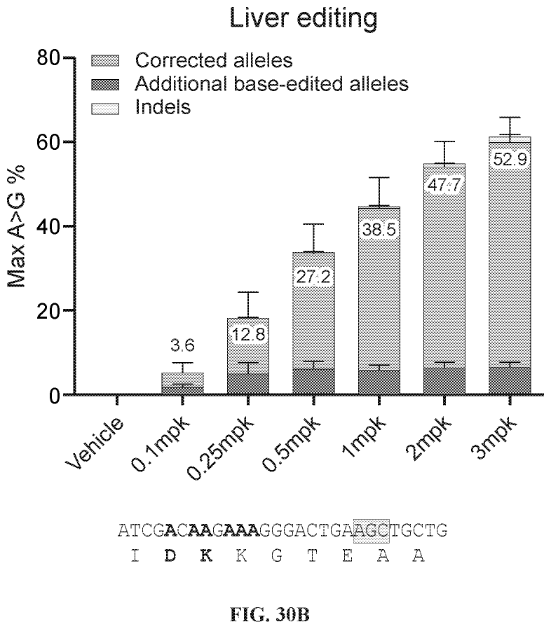

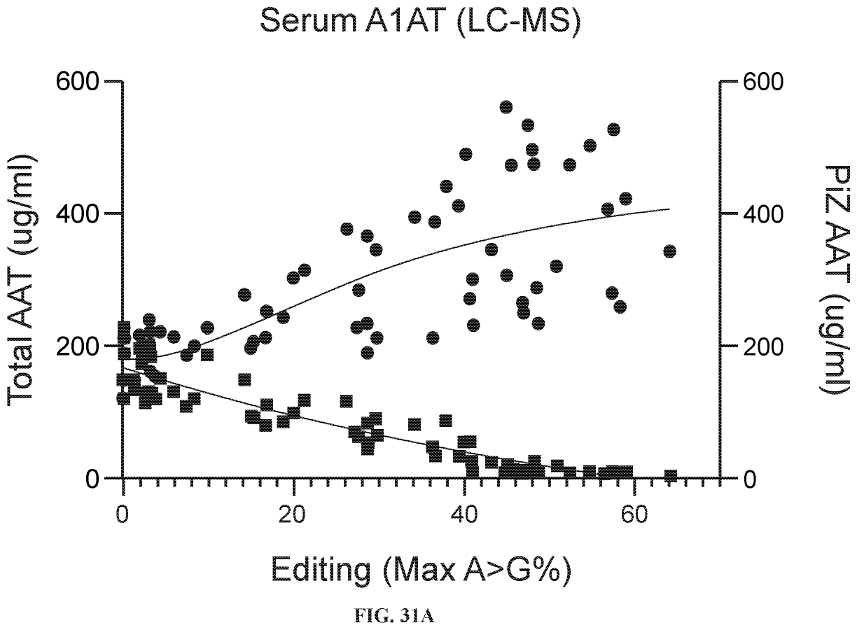

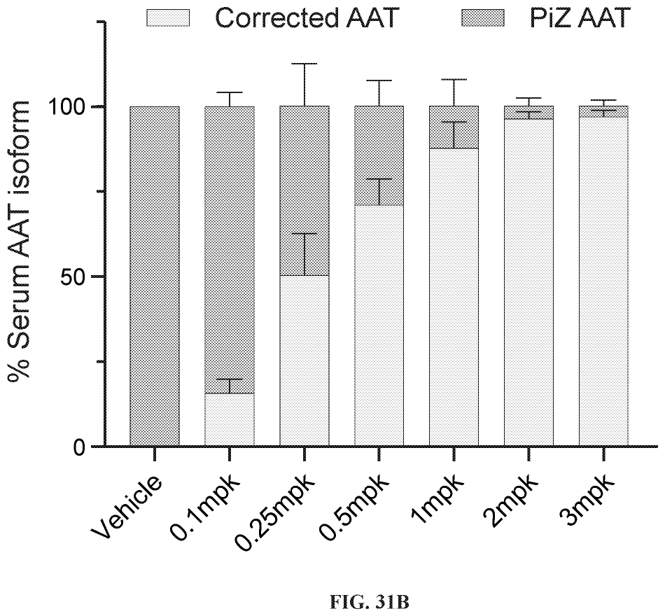

To evaluate in vivo base editing in the rats, the hSERPINA1 PiZ rats were administered the base editor system intravenously as shown in FIG. 30A. The lipid nanoparticles containing the base editor system were administered intravenously and measurements were taken one week following the administration. No saturation of corrective base editing was observed at the doses of total RNA evaluated (1:1 mass ratio of the guide polynucleotide gRNA856 and mRNA encoding Variant G) (FIG. 30B). A maximum percent A to G editing of about 50% (FIG. 30B) was observed at a total RNA dose of 3 mg/kg. The base editing was accompanied by a corresponding increase in circulating total corrected (i.e., no longer PiZ AAT) alpha-1 antitrypsin protein (AAT) and decreased levels of PiZ AAT (FIG. 31A), as determined using liquid chromatography-mass spectroscopy (LC-MS) of serum collected from the AATD mice. At doses of 0.5 mg/kg total RNA or higher, more than half the circulating AAT was comprised of corrected AAT rather than PiZ AAT (FIG. 31B).

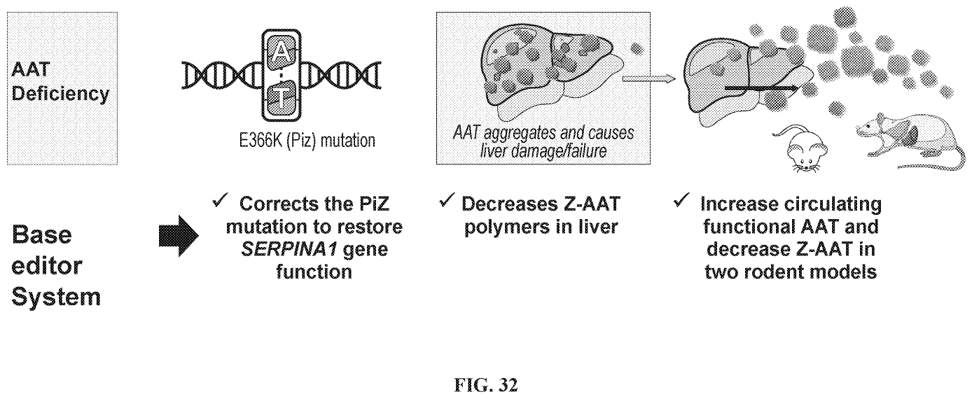

Therefore, as demonstrated above, base editing can be used to correct a PiZ mutation in vivo to restore SERPINA1 gene function, thereby decreasing Z-AAT polymers in the liver, and increasing functional AAT and decreasing Z-AAT in two rodent models (FIG. 32).

### Example 5: PiM+Bystander was Secreted Comparably to PiM Alpha-1 Antitrypsin (AAT) Protein In Vivo

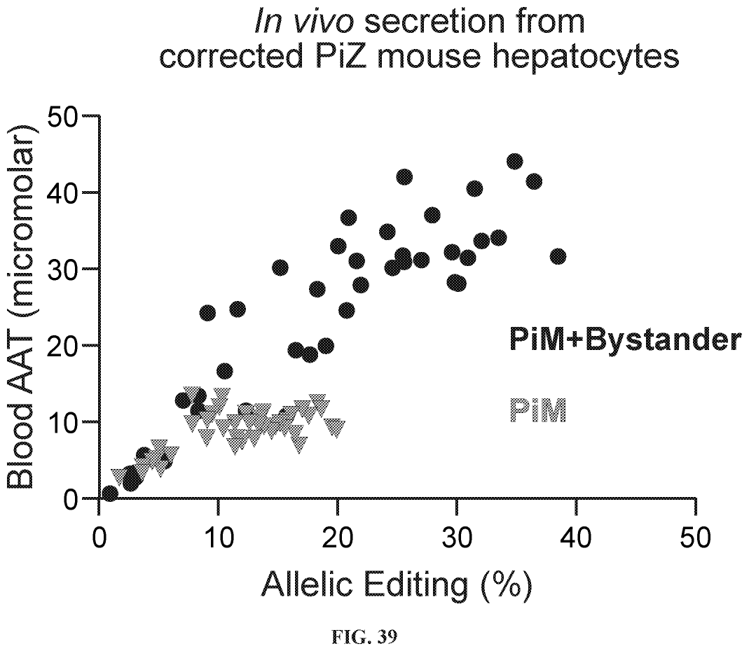

To complete a dose response study for lipid nanoparticles containing the guide RNA gRNA856 and mRNA encoding the base editor ABE Variant G (the “Variant G Formulation”), NSG-PiZ mice were administered a single dose of the Variant G Formulation at increasing dose levels. One-week after dosing, livers were collected to assess correction editing using next-generation sequencing NGS and serum to measure circulating AAT proteins by LC-MS (FIG. 39).

The PiZ mutation in AAT results in aggregation of Z-AAT in hepatocytes, which in turn leads to decreased levels of circulating AAT in serum. Correction of the PiZ mutation using the Variant G Formulations results in increased expression of corrected AAT with a corresponding decrease in Z-AAT. The two proteoforms that constitute corrected AAT are PiM (wildtype) AAT and PiM+Bystander (D365G) AAT, which correspond to precise correction (7G target nucleotide only) and concurrent editing of the target nucleotide and a neighboring bystander adenine (7G+5G), where the 5G position corresponds to that of the A underlined in the following sequence and the 7G position corresponds to that of the boldface A in the following nucleotide sequence: ATCGACAAGAAAGGGACTGAAGCTGCTG, (SEQ ID NO: 593).

To assess if the PiM+Bystander protein was secreted from hepatocytes comparably to the PiM protein, the levels of each protein in serum (y-axis) was plotted against their respective allelic frequencies in the liver. If the PiM+Bystander protein was not secreted from hepatocytes as well as the PiM protein, there would have been less protein in the serum per % 7G+5G editing as compared to PiM protein per % 7G editing. However, the data indicated good correlation of both proteins being secreted to serum relative to their respective allelic frequencies, which suggested that both were secreted form the liver similarly.

### Example 6: PiM+Bystander Alpha-1 Antitrypsin (AAT) Protein was Active and Functionally Indistinguishable from PIM (WT) AAT In Vitro and Ex Vivo

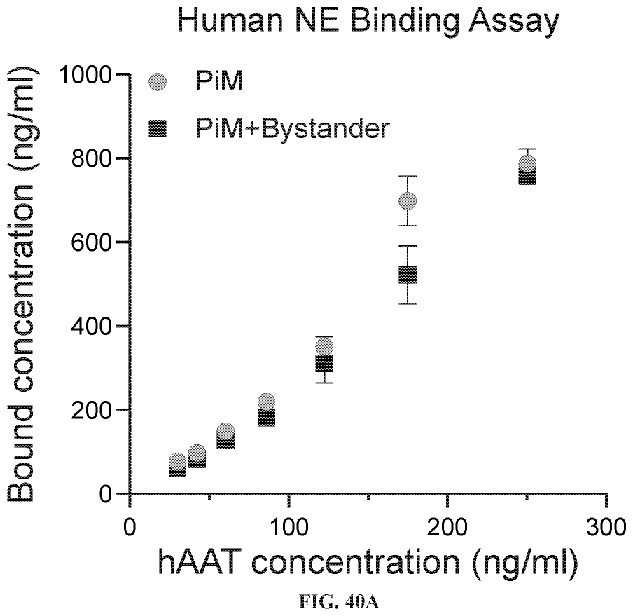

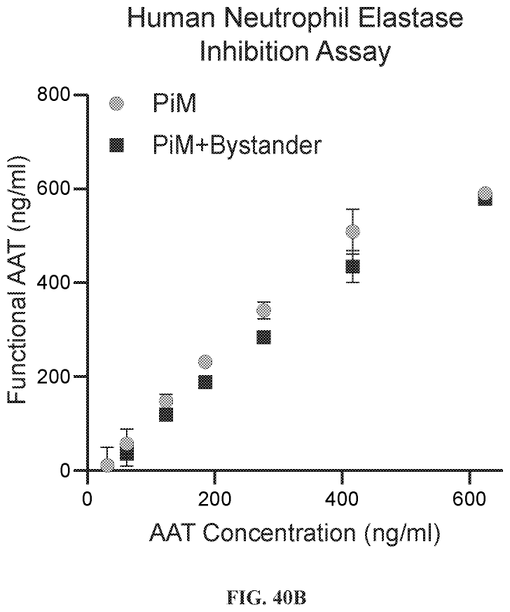

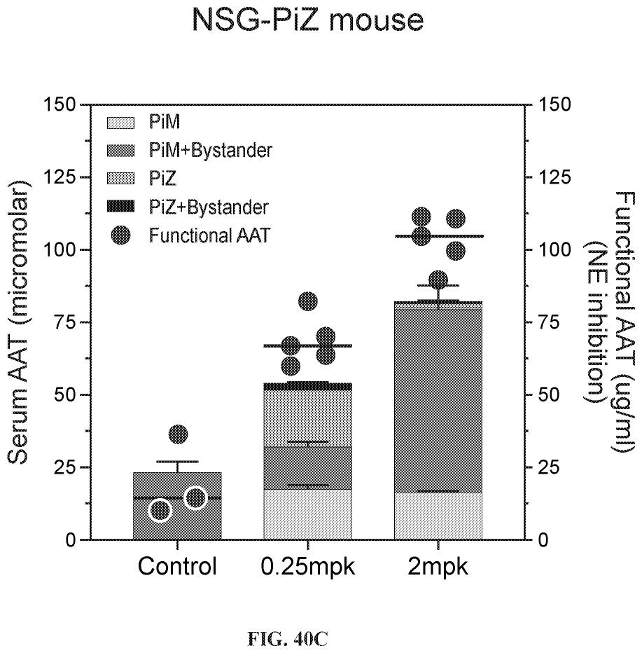

Bacterially expressed PiM and PiM+Bystander (D365G) proteins were tested in vitro for their ability to bind to or inhibit human neutrophil elastase (HNE). Both proteins demonstrated equivalent binding to elastase (FIG. 40A) and inhibition of elastase activity (FIG. 40B) in vitro.

In addition to the in vitro assays, sera from mice dosed once with lipid nanoparticles containing the guide RNA gRNA856 and mRNA encoding the base editor ABE Variant G (the “Variant G Formulation”) were tested in an ex vivo assay for their ability to inhibit HNE activity. An LC-MS assay performed on the same sera demonstrated that PiM+bystander (D365G) was the predominant form of AAT in mice administered a 2 mpk dose of the Variant G Formulation, while PiM and PiM+Bystander (D365G) AAT were present in relative equal amounts in mice administered a 0.25 mpk dose of the Variant G Formulation (stacked bars of FIG. 40C). Regardless of the ratio of PiM AAT to PiM+Bystander AAT, these sera showed a dose-dependent increase in inhibition of HNE activity that was represented as Functional AAT (dots in FIG. 40C). These in vitro and ex vivo data suggested a functional equivalence of PiM and PiM+Bystander AAT in their ability to bind and inhibit HNE.

### Example 7: Hepatocytes that Expressed PiM+Bystander AAT Demonstrated a Survival Advantage in NSG-PiZ Mice

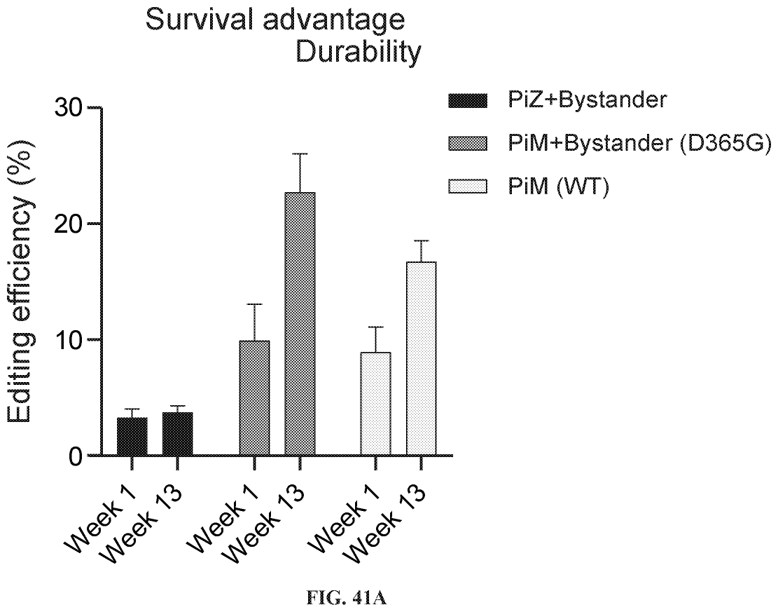

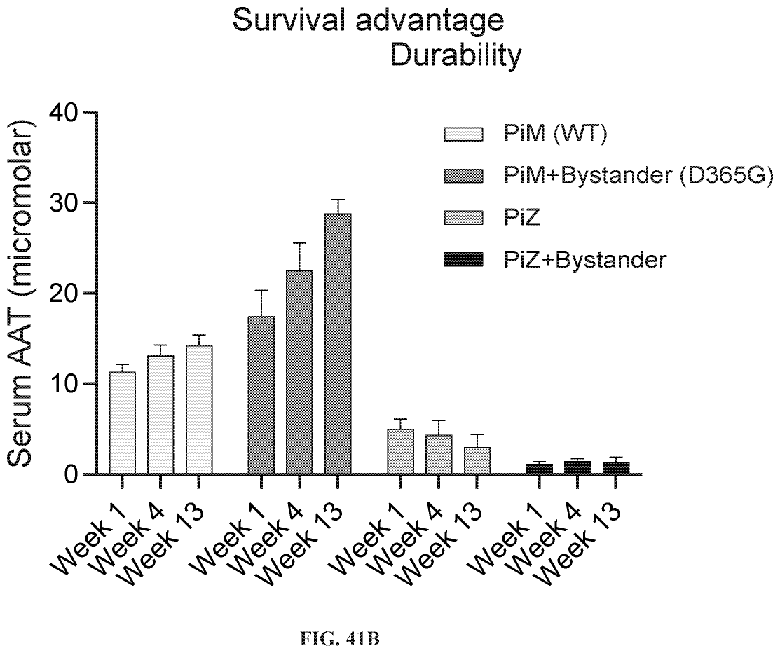

NSG-PiZ mice were administered a single dose of 0.25 mpk of the Variant G Formulation and liver editing was evaluated at week 1 and week 13 post-dose. Next gen sequencing results demonstrated that hepatocytes carrying either the precise correction (7G) or the precise correction paired with the bystander edit (7G+5G) both demonstrated a survival advantage relative to hepatocytes that remained uncorrected. This was evident from the increased editing efficiency observed at week 13 relative to week 1 for PiM and PiM+Bystander alleles but not for PiZ+Bystander alleles (FIG. 41A). The sequencing data were supported by serum LC-MS measurements (FIG. 41B), which confirmed increases in circulating PiM and PiM+Bystander protein levels secreted from hepatocytes carrying the corresponding correction edits at week 13 post-dose when compared to week 1. These data suggested that expression of PiM+Bystander AAT was not detrimental to hepatocyte health and provided a survival advantage similar to PiM (wild type) AAT.

### Example 8: Increased Liver Editing in PiZ-Rat with the Variant G Formulation Redose

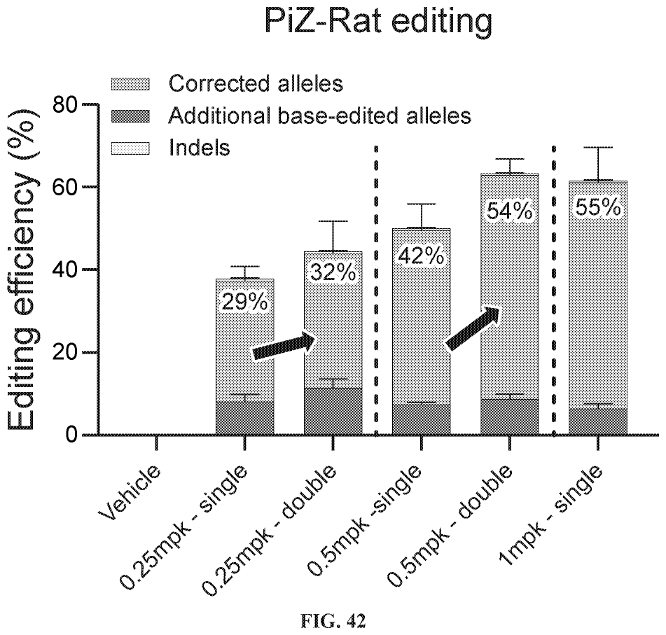

PiZ rats were dosed with a single or double dose (i.e., two separate doses) of the Variant G Formulation at 0.25 mpk or 0.5 mpk. The second dose was delivered 2-weeks after the first dose and the final/terminal takedown was 14 days after the second dose of the Variant G Formulation. A control group was also evaluated, which only received a single dose of 1 mpk of the Variant G Formulation. Next-generation sequencing (NGS) results indicated that repeat dosing increased correction editing relative to a single dose (FIG. 42). Furthermore, two Variant G Formulation doses of 0.5 mpk resulted in the same level of correction editing as a single dose of 1 mpk. Repeat dosing may be used to avoid LNP-associated toxicity observed at higher doses while still achieving optimal correction editing.

### Example 9: Durable Liver Editing and Biomarker Changes in PiZ Rats

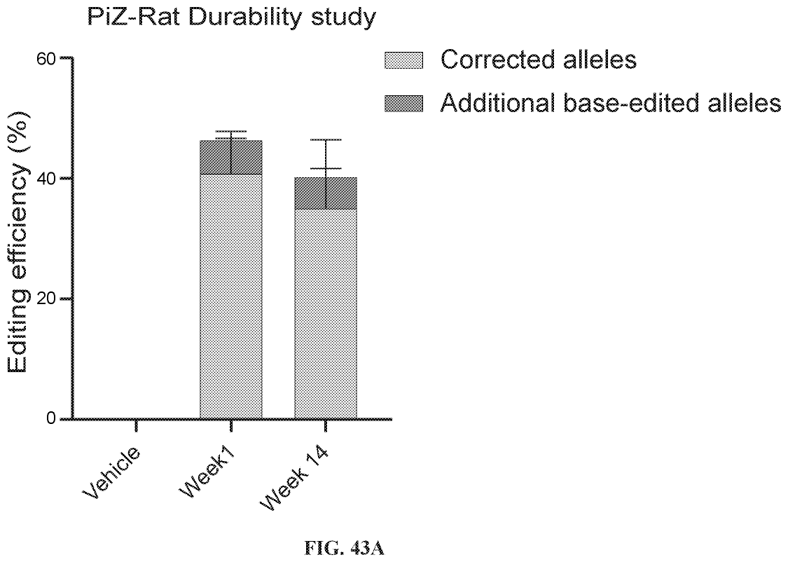

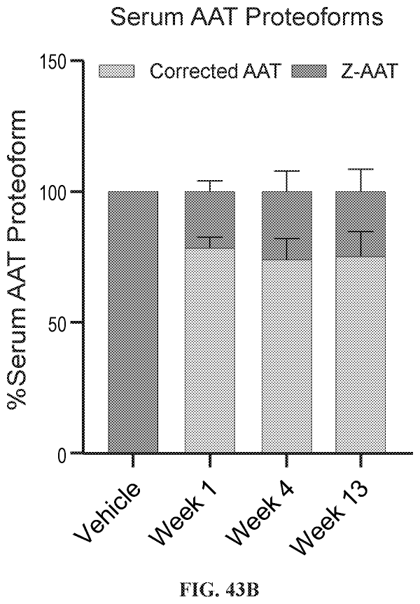

PiZ rat next-generation sequencing (NGS) data (FIG. 43A) demonstrated durable correction editing in PiZ rats 1 week or 14 weeks after being administered a single 0.5 mpk dose of the Variant G Formulation. Liquid chromatography-mass spectrometry (LC-MS) measurements of serum collected from these rats at week 1, week 4, and week 14 (FIG. 43B) demonstrated that the liver correction editing corresponded to a sustained increase in circulating corrected AAT accompanied by a decrease in PiZ AAT. This data confirmed durable editing in a second rodent species after Variant G Formulation-mediated base editing.

### Example 10: Decrease in Liver Z-AAT in PiZ Rats One-Week after Dosing with the Variant G Formulation

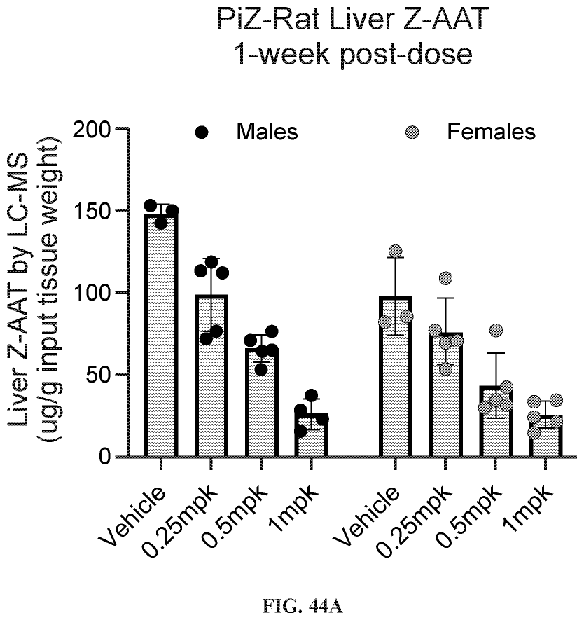

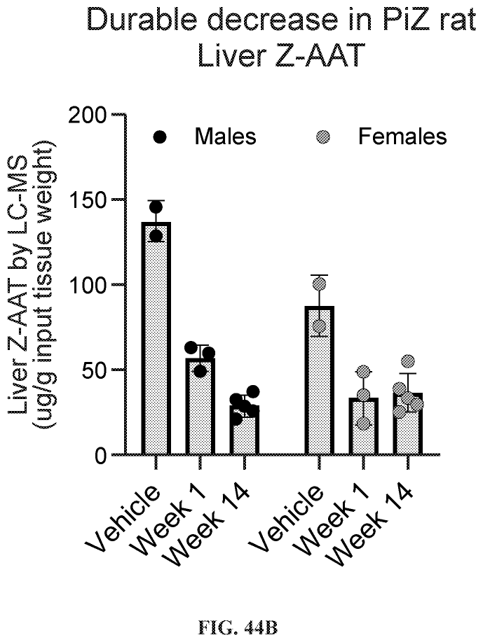

Livers from PiZ rats administered the Variant G Formulation in a dose response study where rats were given increasing dose levels of the Variant G Formulation, and livers from a durability study where rats were administered 0.5 mpk of the Variant G Formulation were evaluated by liquid chromatography-mass spectrometry (LC-MS) for protein levels of mutant Z-AAT. A dose responsive (FIG. 44A) decrease after 1 week of a single dose of Variant G Formulation was observed in PiZ rat livers. This decrease in liver Z-AAT was still observed (i.e., durable) as of 14 weeks after a single dose of the Variant G Formulation (FIG. 44B).

### Example 11: Improved Liver Editing in a PiZ Rat Model Dosed with the Variant G Formulation

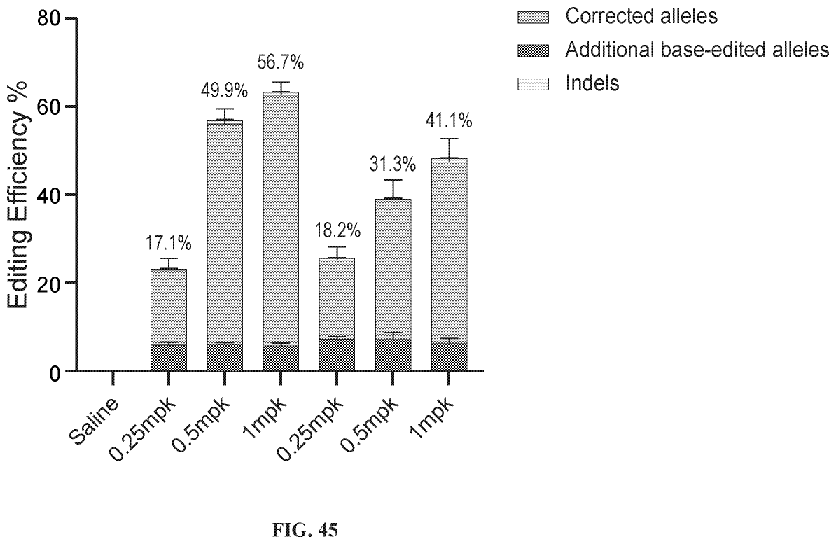

PiZ rats were administered the Variant G Formulation at doses of 0.25 mpk and 0.5 mpk. The rats were taken down 1-week post dose, and livers were collected to assess correction editing using next-generation sequencing (NGS). Improved liver editing was observed using the Variant G Formulation as compared to a formulation similar to the Variant G Formulation but prepared using lipid nanoparticles having a lipid composition that was different from that of the Variant G Formulation (FIG. 45).

### Example 12: Variant G Formulation

Experiments were undertaken to further characterize the lipid nanoparticle (LNP) formulation containing an mRNA that encodes a Variant G Adenosine Base Editor protein (SEQ ID NO: 557) and a gRNA (gRNA856; SEQ ID NO: 575) that targets the SERPINA1 g. 1096G>A. This LNP formulation is referred to as “Variant G Formulation” throughout the present disclosure. In-vivo pharmacology studies were conducted to evaluate the base-editing correction of the SERPINA1-g. 1096G>A allele (as measured by the relative frequency of on-target base-edited, corrected SERPINA1 alleles corresponding to PiM and PiM+Bystander alleles) and corresponding changes in clinically relevant biomarkers (human AAT and functional AAT in serum), both at 1 week after a single IV administration of the Variant G Formulation at a dose ranging from 0.05 to 2.0 mg/kg in NSG-PiZ mice or 0.10 to 3.0 mg/kg in PiZ rats, and up to approximately 3 months after a single IV administration of the Variant G Formulation at 0.25 mg/kg in NSG-PiZ mice or 0.50 mg/kg in PiZ rats. Dose-dependent increases in corrected SERPINA1 alleles were observed in both rodent models, with highest observed frequencies of 46% in NSG-PiZ mice at a total RNA dose of 0.75 mg/kg and 57% in PiZ rats at a total RNA dose of 3.0 mg/kg. A dose-dependent decrease in the abundance of pathologic PiZ alpha-1 antitrypsin (AAT) variants in liver was observed 1 week after administration of the Variant G Formulation, achieving up to 82% reduction at a dose level of 1 mg/kg in PiZ rats; the NSG-PiZ mouse may not be a suitable model for evaluation of this particular endpoint due to the constitutively high accumulation of human PiZ AAT in the liver (driven by the multicopy SERPINA1c. 1096G>A transgene) and high individual variability in hepatic PiZ AAT burden. In addition, a dose-dependent increase in total human AAT in serum (up to 4.1-fold change from baseline in NSG-PiZ mice and up to 2.6-fold change from baseline in PiZ rats), concomitant with a dose-dependent decrease in the levels of pathogenic PiZ AAT variants (relative to total AAT) in serum was observed in both rodent models 1 week after administration of the Variant G Formulation, the lowest levels (2%) of residual PiZ AAT (relative to total AAT) were observed at a total RNA dose of 2.0 mg/kg in NSG-PiZ mice and at a total RNA dose of 3.0 mg/kg in PiZ rats. The durability of base editing in liver and corresponding biomarker changes after the administration of a single dose of the Variant G Formulation at 0.25 mg/kg in NSG-PiZ mice and 0.50 mg/kg in PiZ rats was confirmed in both rodent models to be sustained for at least approximately 3 months.

### Pharmacodynamics

Pharmacodynamic studies of the Variant G Formulation were conducted in 2 rodent models of alpha-1 antitrypsin deficiency (AATD), NSG-PiZ mice and PiZ rats, to evaluate the pharmacodynamic dose response of the Variant G Formulation and the durability of response.

The NSG-PiZ mouse model used (NOD.Cg-Prkdoscid Il2rgtmlWilTg (SERPINA1*E342K) #Slcw/SzJ mouse; RRID: IMSR JAX: 028842) was a commercially available, humanized transgenic mouse strain on the immunodeficient NSG background that carries multiple copies (>10 copies; Carlson J A, Rogers B B, Sifers R N, et al. Accumulation of PiZ alpha 1-antitrypsin causes liver damage in transgenic mice. J Clin Invest. 1989; 83 (4): 1183-90. DOI: 10.1172/JCI113999) of the human SERPINA1-c. 1096G>A allele, which encodes the pathogenic human PiZ AAT variant. The PiZ rat model used was a newly developed, humanized knock-in/knock-out rat strain (SD-Serpina1^{em1(SERPINA1*)} strain, commonly named PiZ rat) that constitutes a unique immunocompetent animal model in which a single copy of the human SERPINA1-c.1096G>A allele is expressed under the endogenous Serpina1 promoter, resulting in the expression of the AAT-p.Glu366Lys, or PiZ, variant at levels relevant to human AATD disease. These complementary animal models of AATD enabled the characterization of the Variant G Formulation both in immunocompromised mice that overexpress the pathogenic human PiZ AAT protein and exhibit PiZ aggregates or globules in the liver (a histopathological hallmark of AATD liver disease observed in patients with AATD), and in immunocompetent rats that express the human PiZ AAT protein at physiologically relevant levels under the control of the endogenous Serpina1 promoter.

In vivo primary pharmacodynamic studies were conducted in both NSG-PiZ mice and PiZ rats to demonstrate the pharmacological activity of the Variant G Formulation up to approximately 3 months after a single IV (bolus) administration. The primary endpoints evaluated in these studies were the relative levels of on-target base-edited, corrected SERPINA1 alleles in liver and corresponding changes in potentially clinically relevant biomarkers (levels of human AAT protein in liver and serum, and levels of functional AAT in serum).

Dose-response studies were evaluated at 1 week after a single IV (bolus) administration of Variant G Formulation at doses ranging from 0.05 to 2.0 mg/kg in NSG-PiZ mice and from 0.10 to 3.0 mg/kg in PiZ rats. The duration selected for these studies represented an early time point at which the base-editing levels in the liver had nearly peaked. Noteworthy findings from these studies are described in the following paragraphs.

A dose-dependent increase in corrected SERPINA1 alleles was observed in the liver in both models. In NSG-PiZ mice, the maximum effect was observed at 0.75 mg/kg in males and at 1 mg/kg in females (mean relative frequencies of corrected SERPINA1 alleles of 46% in males, and 47% in females). In PiZ rats, the maximum effect was observed at 3.0 mg/kg (mean relative frequencies of corrected SERPINA1 alleles of 57% in males and 49% in females).

A dose-dependent decrease in the abundance of pathogenic PiZ AAT variants in liver was observed in PiZ rats; the maximum effect was observed at 1.0 mg/kg (mean decreases, relative to the vehicle control group, of 82% in males and 73% in females). The NSG-PiZ mouse was not a suitable model for the evaluation of this endpoint, due to the constitutively high accumulation of human PiZ AAT in the liver (driven by the multicopy SERPINA1c. 1096G>A transgene), and high individual variability in hepatic PiZ AAT burden.

A dose-dependent increase in total human AAT in serum, concomitant with a dose-dependent decrease in the levels of pathogenic PiZ AAT variants (relative to total AAT) was observed in both models. In NSG-PiZ mice, the mean levels of PiZ AAT, relative to total AAT, were 2% at 2.0 mg/kg in males, and 3% at 1.0 mg/kg in females. In PiZ rats, the mean levels of PiZ AAT, relative to total AAT, were 2% in males and 4% in females at 3.0 mg/kg.

An increase in functional AAT in serum was observed in both models. In NSG-PiZ mice, the maximum effect was observed at the highest doses evaluated in each group (mean changes, relative to baseline, of 5.5-fold at 2.0 mg/kg in males, and 3.8-fold at 1.0 mg/kg in females). In PiZ rats, the maximum effect was observed at 2.0 and 3.0 mg/kg (median changes, relative to baseline, of 2.6-fold in males at 2.0 and 3.0 mg/kg, and 2.8-fold in females at 3.0 mg/kg).

Durability of response studies were evaluated for up to approximately 3 months after a single IV (bolus) administration of the Variant G Formulation at 0.25 mg/kg in NSG-PiZ mice and 0.50 mg/kg in PiZ rats. The doses selected for each of these studies represented the lowest doses at which potentially clinically meaningful increases in total AAT and functional AAT above baseline levels in serum, and increase in corrected AAT above 50% relative to total AAT in serum were observed in association with correction of the pathogenic SERPINA1 g.1096G>A allele in liver, in the dose-response studies. Noteworthy findings from these studies are described in the following paragraphs.

Durable base-editing correction of the SERPINA1-g. 1096G>A allele in the liver was observed in both models. In NSG-PiZ mice, an increased frequency of corrected SERPINA1 alleles was observed between Weeks 1 and 13 (mean relative frequencies of corrected SERPINA1 alleles of 19% in both sexes at Week 1, and 36% in males and 43% in females at Week 13); this increase could be attributed to competitive survival advantage of hepatocytes in which wild-type AAT was expressed (Borel F, Tang Q, Gernoux G. Survival advantage of both human hepatocyte xenografts and genome-edited hepatocytes for treatment of α-1 antitrypsin deficiency. Mol Ther. 2017; 25 (11): 2477-2489. DOI: 10.1016/j.ymthe.2017.09.020) relative to hepatocytes expressing a high burden of PiZ AAT (Lindblad D, Blomenkamp K, Teckman J. Alpha-1-antitrypsin mutant Z protein content in individual hepatocytes correlates with cell death in a mouse model. Hepatology. 2007; 46 (4): 1228-1235. DOI: 10.1002/hep.21822). In PiZ rats, a generally sustained frequency of corrected SERPINA1 alleles was observed between Weeks 1 and 14 (mean relative frequencies of corrected SERPINA1 alleles of 42% in males and 37% in females at Week 1, and 46% in males and 27% in females at Week 14).

A durable decrease in the abundance of pathogenic PiZ AAT variants in liver was observed in both models (except for female NSG-PiZ mice). In NSG-PiZ mice, the mean decreases, relative to the vehicle control group, were 17% in males and 16% in females at Week 1, and 46% in males at Week 13. No effect of the Variant G Formulation was detectable in female mice at Week 13, due to high individual variability among female mice in the vehicle control group in this study. In PiZ rats, the mean decreases, relative to the vehicle control group, were 58% in males and 61% in females at Week 1, and 79% in males and 58% in females at Week 14.

A durable increase in total human AAT in serum, concomitant with a durable decrease in the levels of pathogenic PiZ AAT variants (relative to total AAT) in serum was observed in both models. In NSG-PiZ mice, PiZ AAT variants reached the lowest levels at the last time point evaluated (mean levels, relative to total AAT, of 12% in males and 7% in females at Week 13). In PiZ rats, the decrease in the levels of PiZ AAT variants was generally sustained between Weeks 1 and 14 (mean levels, relative to total AAT, of 20% in males and 23% in females at Week 1, and 17% in males and 32% in females at Week 14).

A durable increase in functional alpha-1 antitrypsin (AAT) in serum was observed in both models (except for female PiZ rats). In NSG-PiZ mice, the mean changes, relative to baseline, were 2.4-fold in both sexes at Week 1, and 3.8-fold in males and 3.1-fold in females at Week 13. In PiZ rats, the mean changes, relative to baseline, were 1.7-fold in both sexes at Week 1, and 1.4-fold in males at Week 14. No effect of the Variant G Formulation was detectable in female rats at Week 14, possibly due to the decrease with age in the levels of human AAT in serum that has been observed in the PiZ rat, which may have different temporal dynamics between sexes in this model.

### Dose-Response Pharmacology in NSG-PiZ Mice

The correction of the pathogenic SERPINA1 g. 1096G>A allele in liver and corresponding changes in biomarkers (levels of human AAT protein in liver and serum, and levels of functional AAT in serum) were evaluated in a dose-response study in NSG-PiZ mice, 1 week after a single IV (bolus) administration of the Variant G Formulation at a total RNA dose ranging from 0.05 to 2.0 mg/kg.

A total of 54 NSG-PiZ mice (3 or 4 male or female mice per group; 8 weeks of age) were administered the Variant G Formulation as a single IV (bolus) injection (lateral tail vein) at a total RNA dose of 0.05, 0.10, 0.25, 0.50, 0.75, 1.0, or 2.0 mg/kg, or normal saline as a control. One week after administration of test or control article, liver samples were collected for evaluation of corrected SERPINA1 alleles and human AAT protein. Both 2 days prior to and 1 week after administration of test or control article, serum samples were collected for evaluation of levels of human AAT and functional AAT.

All mice survived to scheduled euthanasia.

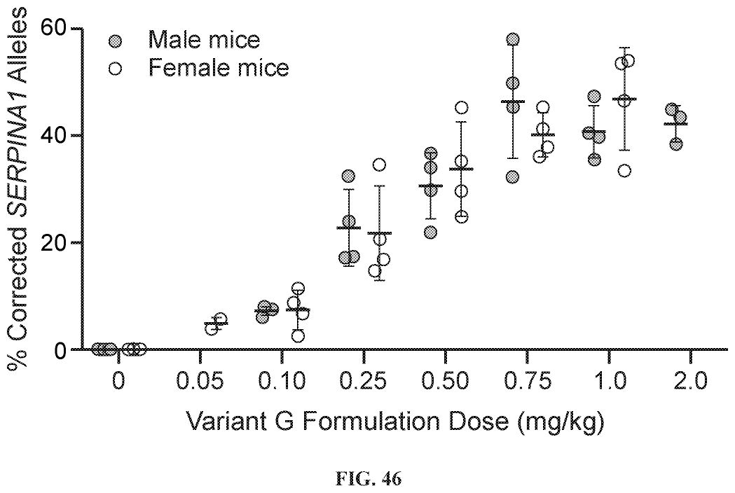

A Variant G Formulation dose-dependent increase in corrected SERPINA1 alleles in liver was observed in male and female NSG-PiZ mice at Day 7, with maximum effect below the top dose tested. Mean relative frequencies of corrected SERPINA1 alleles ranged from 7% (at 0.10 mg/kg, the lowest dose assessed) to 46% (at 0.75 mg/kg) in males, and from 5% (at 0.05 mg/kg) to 47% (at 1.0 mg/kg) in females (FIG. 46).

No statistically significant effect of the Variant G Formulation dose was observed on the abundance of PiZ AAT in liver, possibly due to the constitutively high accumulation of PiZ AAT in the liver of NSG-PiZ mice (driven by the multicopy SERPINA1-g. 1096G>A transgene) combined with high individual variability in hepatic PiZ AAT burden in this mouse model.

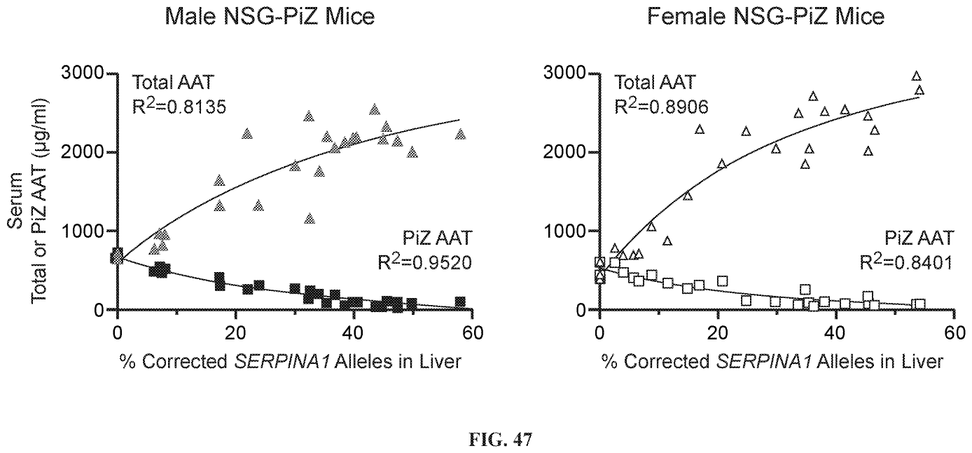

The Variant G Formulation dose-dependent increase in corrected SERPINA1 alleles in liver at Day 7 was associated with a dose-dependent increase in total human AAT in serum, concomitant with a dose-dependent decrease in the levels of PiZ AAT variants (relative to total AAT) in serum, in male and female NSG-PiZ mice (FIG. 47). The greatest increases in the levels of total AAT in serum were observed at Variant G Formulation total RNA doses between 0.50 and 2.0 mg/kg (mean changes of 3.1 to 3.6-fold in males and 3.7 to 4.1-fold in females, relative to baseline at Day-2). Corrected AAT variants were the predominant forms of AAT in serum at Variant G Formulation total RNA doses between 0.25 and 2.0 mg/kg (mean levels, relative to total AAT, of 77% to 98% in males and 83% to 97% in females). Pathogenic PiZ alpha-1 antitrypsin (AAT) variants reached the lowest levels in serum at the highest doses of Variant G Formulation evaluated (mean levels, relative to total AAT, of 2% at 2.0 mg/kg in males, and 3% at 1.0 mg/kg in females).

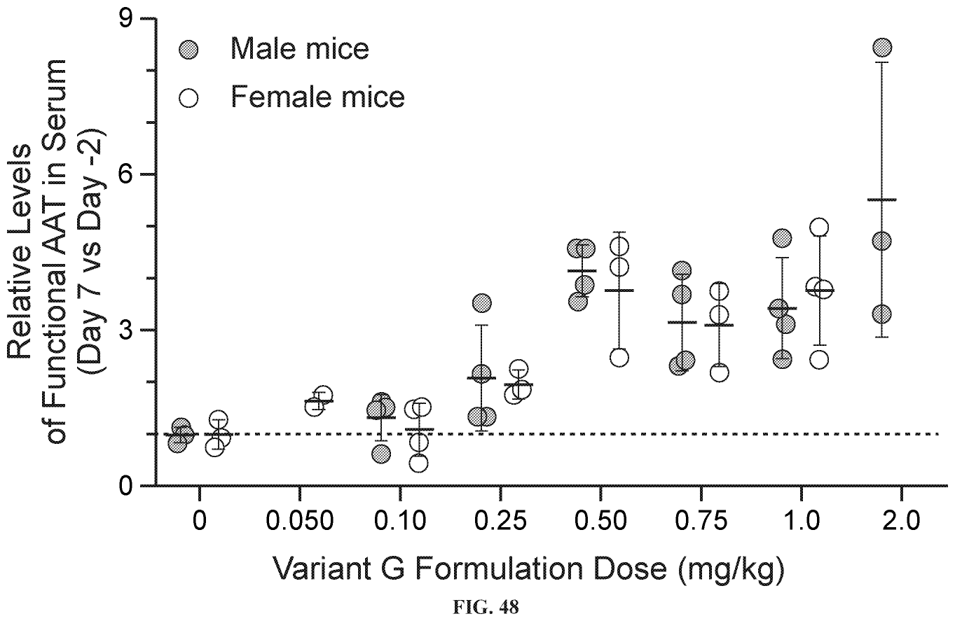

In addition, a Variant G Formulation dose-dependent increase in functional AAT in serum was observed at Day 7 in male and female NSG-PiZ mice; the greatest increases were observed at the highest doses of the Variant G Formulation evaluated (mean changes, relative to baseline at Day-2, of 5.5-fold at 2.0 mg/kg in males, and 3.8-fold at 1.0 mg/kg in females) (FIG. 48).

These results demonstrated that a single IV (bolus) administration of the Variant G Formulation to NSG-PiZ mice resulted in a dose-dependent increase in corrected SERPINA1 alleles in the liver at Day 7, which was associated with increased total human AAT protein and functional AAT in circulation, and with decreased pathogenic PiZ AAT variants in circulation.

### Durability Pharmacology NSG-PiZ Mice

The durability of correction of the pathogenic SERPINA1 g. 1096G>A allele in liver, and of the corresponding changes in biomarkers (levels of human AAT protein in liver and serum, and levels of functional AAT in serum) was evaluated in NSG-PiZ mice, up to 13 weeks after a single IV (bolus) administration of the Variant G Formulation at a dose of 0.25 mg/kg.

A total of 48 NSG-PiZ mice (3 or 5 male or female mice per group; 6 to 7 weeks of age) were administered the Variant G Formulation as a single IV (bolus) injection (lateral tail vein) at a total RNA dose of 0.25 mg/kg, or normal saline as a control. At 1 and 13 weeks after administration of test or control article, liver samples were collected for evaluation of corrected SERPINA1 alleles and human AAT protein. Both 3 days prior to and at Weeks 1, 4, and 13 after administration of test or control article, serum samples were collected for evaluation of levels of human AAT and functional AAT.

All mice survived to scheduled euthanasia.

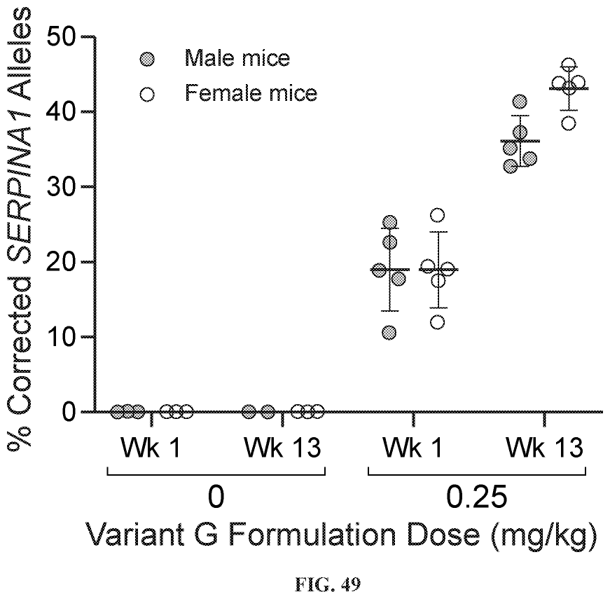

Durable base-editing correction of the SERPINA1-g. 1096G>A allele in liver was observed in male and female NSG-PiZ mice after administration of the Variant G Formulation. The mean relative frequencies of corrected SERPINA1 alleles were 19% at Week 1 (males and females) and approximately 2-fold higher at Week 13 (36% in males and 43% in females) (FIG. 49). The increased frequency of corrected SERPINA1 alleles observed between Weeks 1 and 13 can likely be attributed to competitive survival advantage of hepatocytes that carried corrected SERPINA1 alleles, possibly associated with apoptosis of hepatocytes that expressed high levels of the pathogenic PiZ variant, and consistent with observations in this mouse model (Borel F, Tang Q, Gernoux G. Survival advantage of both human hepatocyte xenografts and genome-edited hepatocytes for treatment of α-1 antitrypsin deficiency. Mol Ther. 2017; 25 (11): 2477-2489. DOI: 10.1016/j.ymthe.2017.09.020Borel, 2017; Rudnick D A, Liao Y, An J K, et al. Analyses of hepatocellular proliferation in a mouse model of alpha-1-antitrypsin deficiency. Hepatology. 2004; 39 (4): 1048-1055. DOI: 10.1002/hep.20118 Lindblad D, Blomenkamp K, Teckman J. Alpha-1-antitrypsin mutant Z protein content in individual hepatocytes correlates with cell death in a mouse model. Hepatology. 2007; 46 (4): 1228-1235. DOI: 10.1002/hep.21822Lindblad, 2007).

The durable increase in corrected SERPINA1 alleles in liver through Week 13 was associated with a decrease in the abundance of pathogenic PiZ AAT variants in liver at Week 1 (mean decreases, relative to the vehicle control group, of 17% in males and 16% in females) and at Week 13 after dosing (mean decrease, relative to the vehicle control group, of 46% in males). No effect of the Variant G Formulation was observed in NSG-PiZ female mice at Week 13, due to high individual variability among female mice in the vehicle control group.

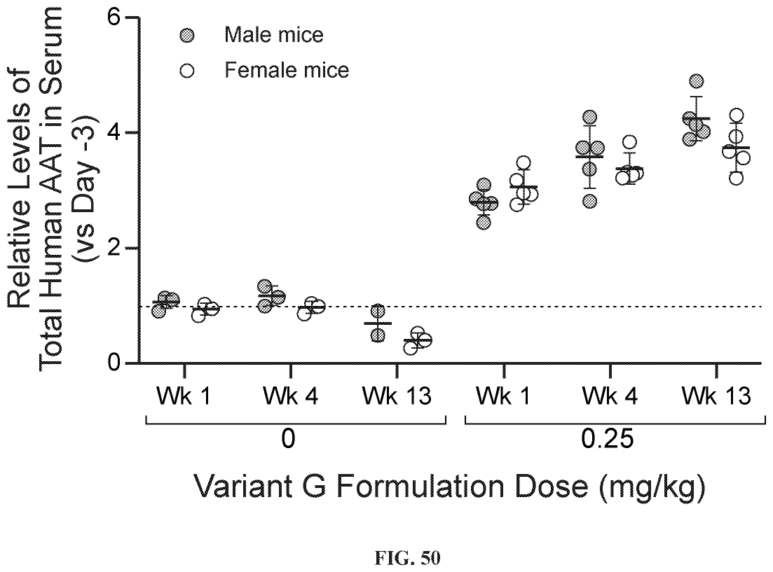

In addition, the durable increase in corrected SERPINA1 alleles in liver through Week 13 was associated with a durable increase in total human AAT in serum, concomitant with a sustained decrease in the levels of PiZ AAT variants (relative to total AAT) in serum, in male and female NSG-PiZ mice administered the Variant G Formulation. The mean increases in the levels of total human AAT in serum were approximately 3-, 3.5-, and 4-fold higher at Weeks 1, 4, and 13, respectively, than at baseline (Day 3) (both sexes) (FIG. 50). Corrected AAT variants were the predominant forms of AAT in serum during the period between Weeks 1 and 13 after administration of the Variant G Formulation (mean levels, relative to total AAT, of 78% to 88% in males and 86% to 94% in females). Pathogenic PiZ AAT variants reached the lowest levels in serum at the last time point evaluated (mean levels, relative to total AAT, of 12% in males and 7% in females at Week 13).

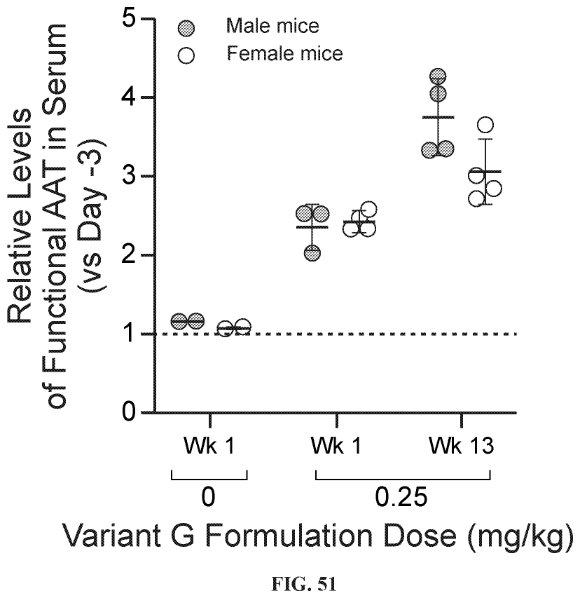

Finally, and consistent with the increase in corrected AAT, a durable increase in functional AAT in serum was observed through Week 13 in male and female NSG-PiZ mice administered the Variant G Formulation (mean changes, relative to baseline at Day 3, of 2.4-fold [both sexes] at Week 1, and 3.8-fold [males] and 3.1-fold [females] at Week 13) (FIG. 51).

These results demonstrate that a single IV (bolus) administration of Variant G Formulation to NSG-PiZ mice at a dose of 0.25 mg/kg resulted in a durable increase in corrected SERPINA1 alleles in the liver through Week 13, which was associated with a durable increase in total human AAT protein and functional AAT in circulation, and durable decrease of pathogenic PiZ AAT variants in circulation.

### Humanized PiZ Knock-In Rat Model of AATD

Experiments were undertaken to generate a rat strain in which expression of the rat AAT protein was replaced with expression of the human AAT-p.Glu366Lys, or PiZ, variant under the control of the endogenous Serpina1 promoter (SD-Serpina1^{em1(SERPINA1*)}, referred to as PiZ rat), and to assess the phenotype of this model up to 1 year of age.

A human SERPINA1-c.1096G>A full-length cDNA, which encodes the AAT-p.Glu366Lys, or PiZ, variant, was inserted using CRISPR/Cas9 technology at the location of the translation initiation codon of the endogenous Serpina1 gene in Sprague Dawley outbred rat embryos, resulting in disruption of the rat Serpina1 gene due to introduction of a poly(A) signal sequence upstream of the remainder of the Serpina1 gene. Founder rats were identified by PCR assay and were confirmed by Sanger sequencing to carry an error-free SERPINA1-c. 1096G>A sequence inserted at the target site in the endogenous Serpina1 locus. In homozygous mutant PiZ rats derived from the confirmed founders, the SERPINA1-c. 1096G>A copy number was estimated to be 2 copies per genome, confirming the absence of random integration of the SERPINA1-c. 1096G>A cDNA in the rat genome.

Validation of the PiZ rat strain at the protein level was conducted by assessment of rat and human AAT in serum through a combination of approaches. Western blot confirmed the absence of rat AAT protein in the serum from homozygous mutant PiZ rats. Meso Scale Discovery immunoassay confirmed the presence of antigenic levels of human AAT protein in the serum of heterozygous and homozygous mutant PiZ rats. Relative quantification of rat and human AAT, through an LC-MS peptide mapping assay that tracked panels of peptides specific for each of the 2 AAT orthologs, showed loss of endogenous rat AAT in the serum of homozygous mutant PiZ rats, and emergence of human AAT in heterozygous and homozygous mutant PiZ rats. As expected based on the sequence of the knock-in cDNA, only peptides corresponding to the human PiZ AAT variant, and not wild-type human AAT, were detected in the serum of heterozygous and homozygous mutant PiZ rats by LC-MS peptide mapping.

The phenotypic effect of expressing the human PiZ AAT variant was evaluated in cohorts of homozygous PiZ rats (2 independent founder lines, Lines 18428 and 18574) compared to age matched Sprague Dawley rats. A total of 27 PiZ Line 18428, 32 PiZ Line 18574, and 28 Sprague Dawley rats (5 to 10 male rats and 6 to 10 female rats per study group) were aged to 6 months (Week 26) or 1 year (Week 52); levels of human PiZ AAT protein and of the liver damage marker ALT were measured in serum longitudinally (between Weeks 16 and 52). In addition, microscopic evaluation of the liver (PAS-D and H&E stains) was conducted at Weeks 26 and 52.

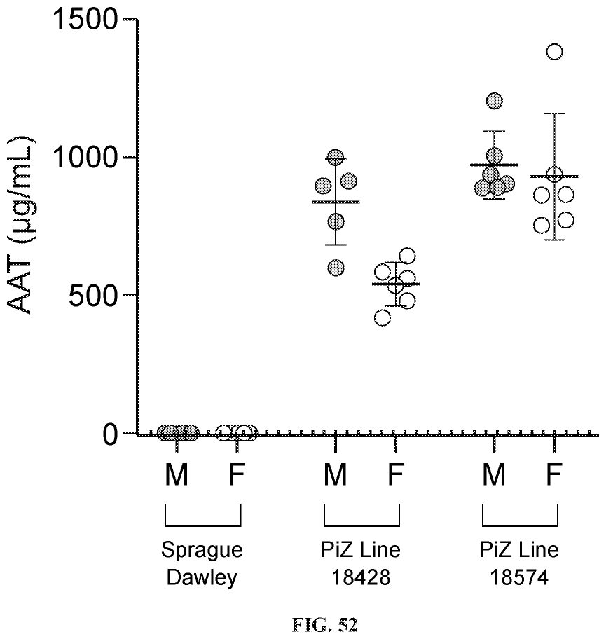

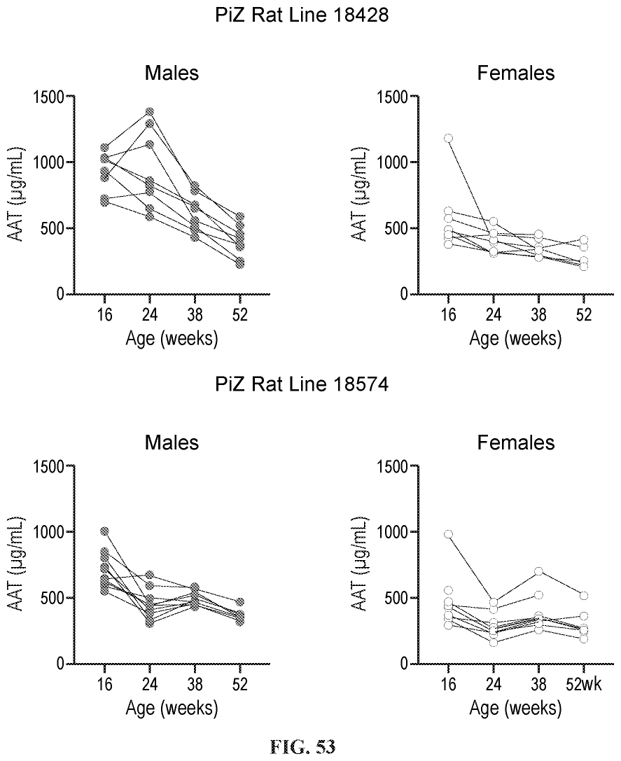

All PiZ rats appeared healthy throughout the study and had a normal lifespan. As expected, circulating human PiZ AAT protein was only detected in PiZ rats, with mean (SD) levels that ranged, across study groups, from 537 (80) to 973 (122) μg/mL at Week 26 (FIG. 52). This was consistent with the levels of AAT observed in serum from patients with Pi*ZZ genotype, which are rarely above 570 μg/mL (Franciosi N A, Fraughen D, Carroll T P, et al. Alpha-1 antitrypsin deficiency: clarifying the role of the putative protective threshold. Eur Respir J. 2022; 59 (2): 2101410. DOI: 10.1183/13993003.01410-2021). Longitudinal assessment of human PiZ AAT in the serum of PiZ rat cohorts aged to 1 year showed statistically significant effects of age and sex in both founder lines (FIG. 53). Increasing age was associated with decreased levels of PiZ AAT in serum; the mean decrease at Week 52 relative to Week 16 ranged between 32% in female rats (Line 18574) and 57% in male rats (Line 18428). Male sex was associated with higher levels of PiZ AAT in serum; the mean relative differences (male vs female) ranged between 1.6 and 2.3-fold (Line 18428), and 1.3- and 1.7-fold (Line 18574).

Longitudinal assessment of the liver damage marker ALT (Week 16 to Week 52) showed a statistically significant increase in the levels of ALT in serum with age in both Sprague Dawley and PiZ rats; no statistically significant differences between Sprague Dawley and PiZ rats (both founder lines) or between sexes were observed. In humans, only 7.8% of individuals with Pi*ZZ phenotype (n=638) have been found to have abnormally increased levels of ALT in serum, which did not differ significantly from healthy controls (Pi*MM phenotype; n=152) (Clark V C, Dhanasekaran R, Brantly M, et al. Liver test results do not identify liver disease in adults with α-1 antitrypsin deficiency. Clin Gastroenterol Hepatol. 2012; 10 (11): 1278-1283. DOI: 10.1016/j.cgh.2012.07.007).

Microscopic examination of the liver showed that PiZ rats did not exhibit PAS-D-stained globules (Week 52) but did have an increased incidence and severity of clear vacuoles in the midzonal region of the liver compared to wild-type control rats (12 of 12 PiZ rats compared to 3 of 12 Sprague Dawley rats at Week 26, and 11 of 15 PiZ rats compared to 3 of 14 Sprague Dawley rats at Week 52/53). There was a single, 52-week-old female PiZ rat with oval cell hyperplasia, hepatocellular anisokaryosis, multinucleated hepatocytes, and Kupffer cell pigmentation, which was not a background finding in wild-type rats of this strain and age. In humans, liver disease is found in only 10% of adult individuals with Pi*ZZ phenotype (n=1595), with a mean (SD) age at the onset of the disease of 61 (15) years (Tanash H A, Piitulainen E. Liver disease in adults with severe alpha-1-antitrypsin deficiency. J Gastroenterol. 2019; 54 (6): 541-548. DOI: 10.1007/s00535-019-01548-y). Furthermore, quantitative analysis by whole-slide morphometry of biopsy samples from individuals with alpha-1 antitrypsin deficiency (AATD) and Pi*ZZ phenotype (n=94) (i.e., homozygous for PiZ) showed that PAS D-stained globules occupy only a very small portion (<0.5%) of a given liver sample and that there is significant heterogeneity in accumulation of AAT polymer and globules polymer across biopsies despite the homogenous mutational background (Marek G, Collinsworth A, Liu C, et al. Quantitative measurement of the histological features of alpha-1 antitrypsin deficiency-associated liver disease in biopsy specimens. PLOS One. 2021; 16 (8): e0256117. DOI: 10.1371/journal.pone.0256117).

In conclusion, a genetically engineered rat strain, the PiZ rat, has been developed that constitutes an immunocompetent animal model in which a single copy of the human SERPINA1 c.1096G>A allele is expressed under the endogenous Serpina1 promoter, resulting in the expression of the AAT p.Glu366Lys variant (i.e., PiZ) at levels relevant to human AATD. Two independent founder lines (Lines 18428 and 18574) exhibited similar characteristics (ie, copy number and sequence of the SERPINA1 c.1096G>A knock-in gene, and levels of human AAT and rat ALT in serum) and, thus, either line would be representative of the PiZ rat model. Line 18574 was selected for use in further studies of PiZ rats.

### Dose-Response Pharmacology in PiZ Rats

Experiments were undertaken to measure correction of the pathogenic SERPINA1 g. 1096G>A allele in liver and corresponding changes in biomarkers (levels of human AAT protein in liver and serum, and levels of functional AAT in serum) using dose-response experiments in PiZ rats, 1 week after a single IV (bolus) administration of the Variant G Formulation at a total RNA dose ranging from 0.10 to 3.0 mg/kg.

A total of 66 PiZ rats (3 or 5 male or female rats per group; 8 weeks of age) were administered the Variant G Formulation as a single IV (bolus) injection (lateral tail vein) at a total RNA dose of 0.10, 0.25, 0.50, 1.0, 2.0, or 3.0 mg/kg, or normal saline as a control. One week after administration of test or control article, liver and ovary samples were collected for evaluation of corrected SERPINA1 alleles and human AAT protein (liver only). Both 4 days prior to and 1 week after administration of test or control article, serum samples were collected for evaluation of levels of human AAT and functional AAT.

All rats survived to scheduled euthanasia.

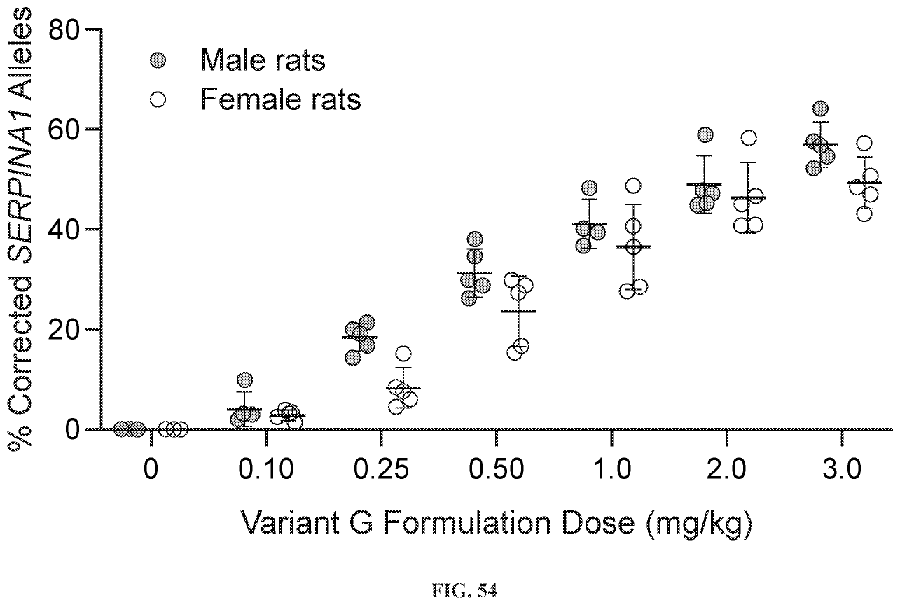

A Variant G Formulation dose-dependent increase in corrected SERPINA1 alleles in the liver was observed in male and female PiZ rats at Day 7, with no saturation in the dose range evaluated. Mean relative frequencies of corrected SERPINA1 alleles ranged from 4% (at a total RNA dose of 0.10 mg/kg) to 57% (at a total RNA dose of 3.0 mg/kg) in males, and from 3% (at a total RNA dose of 0.10 mg/kg) to 49% (at a total RNA dose of 3.0 mg/kg) in females (FIG. 54). The correction of the SERPINA1 g.1096G>A allele in ovaries, evaluated as an exploratory endpoint, was not dose responsive; the mean relative frequency of corrected SERPINA1 alleles was 2% at all doses evaluated (1.0, 2.0, and 3.0 mg/kg).

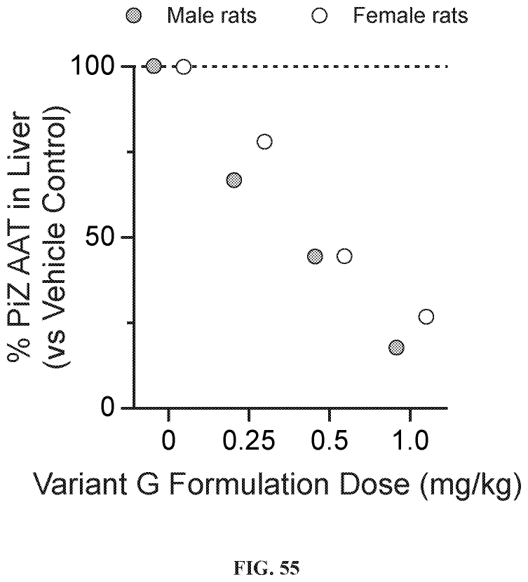

The Variant G Formulation dose-dependent increase in corrected SERPINA1 gene in liver at Day 7 was associated with a dose-dependent decrease in the abundance of pathogenic PiZ AAT variants in liver in male and female PiZ rats. The lowest abundances of PiZ AAT variants in liver were observed at the highest dose of the Variant G Formulation evaluated (mean decreases, relative to the vehicle control group, of 82% in males and 73% in females at 1.0 mg/kg) (FIG. 55).

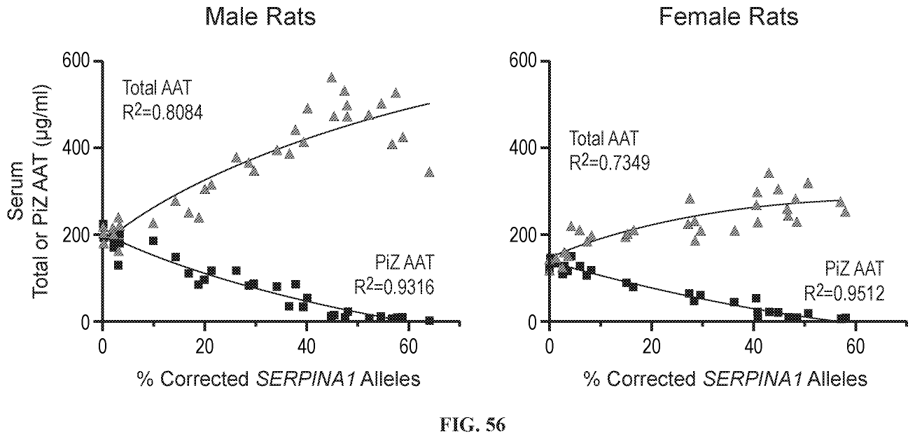

In addition, the Variant G Formulation dose-dependent increase in corrected SERPINA1 alleles in liver at Day 7 was associated with a dose-dependent increase in total human AAT in serum, concomitant with a dose-dependent decrease in the levels of PiZ AAT variants (relative to total AAT) in serum, in male and female PiZ rats (FIG. 56). The greatest increases in the levels of total AAT in serum were observed at a Variant G Formulation total RNA dose of 2.0 mg/kg (mean changes of 2.6-fold in males and 2.0-fold in females, relative to baseline at Day −4). Corrected AAT variants were the predominant forms of AAT in serum at Variant G Formulation total RNA doses between 0.50 and 3.0 mg/kg (mean levels, relative to total AAT, of 76% to 98% in males, and 66% to 96% in females). Pathogenic PiZ AAT variants reached the lowest levels in serum at the highest dose of Variant G Formulation tested (mean levels, relative to total AAT, of 2% in males and 4% in females at a total RNA dose of 3.0 mg/kg).

Finally, a Variant G Formulation dose-dependent increase in functional AAT in serum was observed at Day 7 in male and female PiZ rats; the greatest increases were observed at the top 2 doses of the Variant G Formulation tested (median changes, relative to baseline at Day −4, of 2.6-fold in males at total RNA doses of 2.0 and 3.0 mg/kg, and 2.8-fold in females at a total RNA dose of 3.0 mg/kg).

These results demonstrated that a single IV (bolus) administration of the Variant G Formulation to PiZ rats resulted in a dose-dependent increase in corrected SERPINA1 alleles in the liver at Day 7, which was associated with decreased abundance of pathogenic PiZ AAT variants in the liver, concomitant with increased total human AAT protein and functional AAT in circulation and decreased pathogenic PiZ AAT variants in circulation.

### Durability Pharmacology Study in PiZ Rats

Experiments were undertaken to evaluate the durability of correction of the pathogenic SERPINA1 g.1096G>A allele in liver, and of the corresponding changes in biomarkers (levels of human AAT protein in liver and serum, and levels of functional AAT in serum) in PiZ rats up to 14 weeks after a single IV (bolus) administration of the Variant G Formulation at a dose of 0.50 mg/kg.

A total of 42 PiZ rats (2 to 6 male or female rats per group; 13 weeks of age) were administered the Variant G Formulation as a single IV (bolus) injection (lateral tail vein) at a total RNA dose of 0.50 mg/kg, or normal saline as a control. At 1 and 14 weeks after administration of test or control article, liver samples were collected for evaluation of corrected SERPINA1 alleles and human AAT protein. Both 2 days prior to and at Weeks 1, 4, and 14 after administration of test or control article, serum samples were collected for evaluation of levels of human AAT and functional AAT.

All rats survived to scheduled euthanasia.

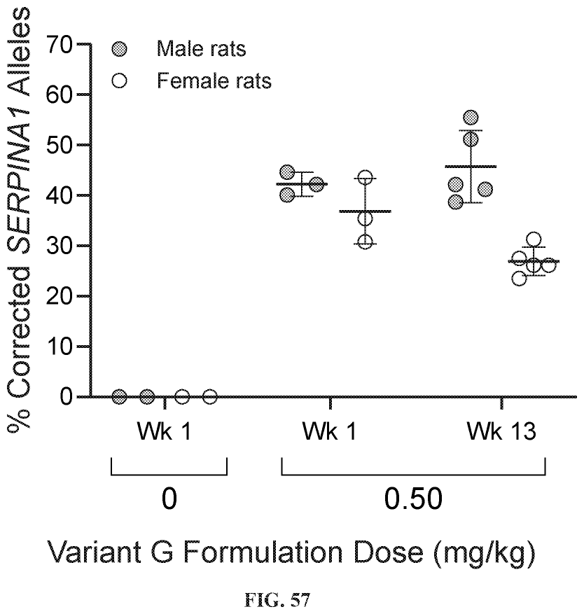

Durable base-editing correction of the SERPINA1-g. 1096G>A allele in liver was observed in male and female PiZ rats after administration of the Variant G Formulation. The mean relative frequencies of corrected SERPINA1 alleles at Weeks 1 and 14 were, respectively, 42% and 46% in males and 37% and 27% in females (FIG. 57).

The durable increase in corrected SERPINA1 alleles in liver through Week 14 was associated with a durable decrease in the abundance of pathogenic PiZ AAT variants in liver in male and female PiZ rats (mean decreases, relative to the vehicle control group [Week 1], of 58% in males and 61% in females at Week 1, and of 79% in males and 58% in females at Week 14).

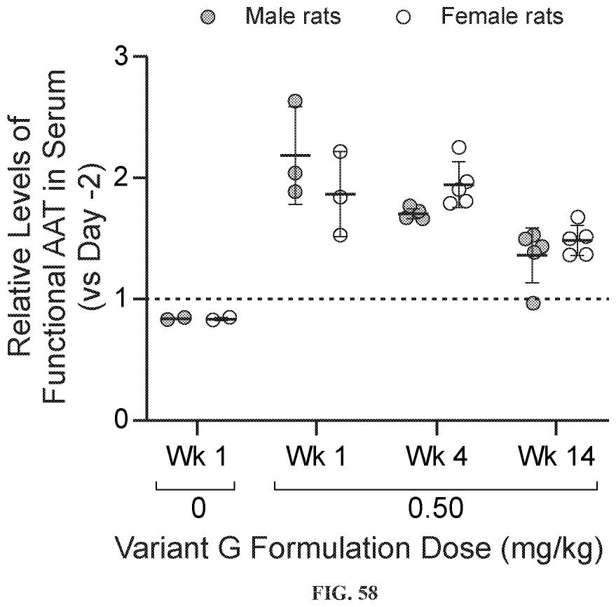

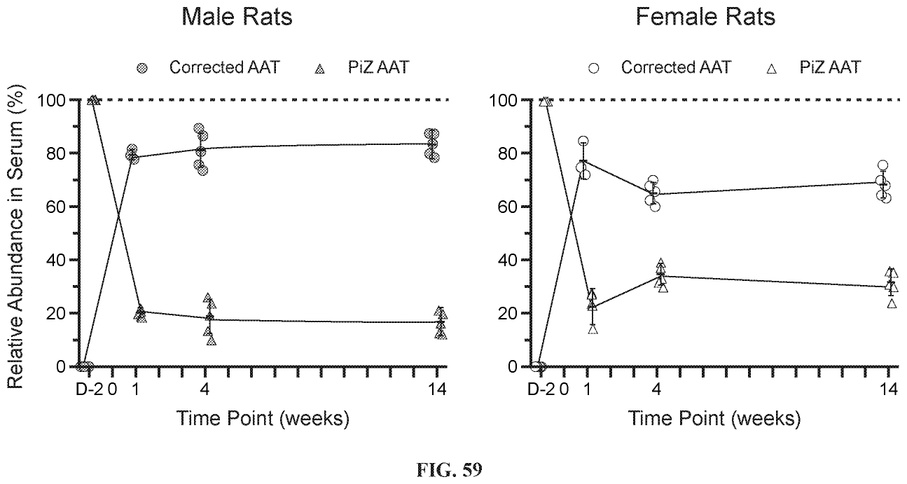

In addition, the durable increase in corrected SERPINA1 alleles in liver through Week 14 was associated with a durable increase in total human AAT in serum, concomitant with a durable decrease in the levels of PiZ AAT variants (relative to total AAT) in serum, in male and female PiZ rats administered the Variant G Formulation. The mean increases in the levels of total human AAT in serum were approximately 2-, 2-, and 1.5-fold higher at Weeks 1, 4, and 14, respectively, than at baseline (Day-2) (both sexes) (FIG. 58). Corrected AAT variants were the predominant forms of AAT in serum during the period between Weeks 1 and 14 after administration of the Variant G Formulation (mean levels, relative to total AAT, of 80% to 83% in males and 77% to 68% in females); the decrease in the levels of PiZ AAT variants in serum was sustained between Weeks 1 and 14 (mean levels, relative to total AAT, of 20% at Week 1 and 17% at Week 14 in males, and of 23% at Week 1 and 32% at Week 14 in females) (FIG. 59). The levels of total human AAT in serum was observed to decline with age in PiZ rats; this observation, together with the stability in the levels of corrected and PiZ AAT, relative to total human AAT, between Weeks 1 and 14, suggested that the small decline observed in relative levels of total human AAT at Week 14 compared to Weeks 1 and 4 in the serum of rats administered the Variant G Formulation may be attributable to temporal dynamics in the model system.

An increase in functional AAT in serum was observed through Week 14 in male, but not in female PiZ rats administered the Variant G Formulation (mean changes, relative to baseline at Day −2, of 1.7-fold [both sexes] at Week 1, and 1.4-fold [males] and 1.0-fold [females] at Week 14). The return of functional AAT activity to baseline levels in female PiZ rats, at Week 14 after administration of the Variant G Formulation, could be a result of the decrease with age in the levels of human AAT in serum that has been observed in the PiZ rat model, which may have a different temporal dynamics between sexes in this model.

These results demonstrated that a single IV (bolus) administration of the Variant G Formulation to PiZ rats at a total RNA dose of 0.50 mg/kg resulted in a durable increase in corrected SERPINA1 alleles in the liver through Week 14, which was associated with a durable increase in total human AAT protein (both sexes) and functional AAT (detected only in males for the time period assessed) in circulation, and durable decrease of pathogenic PiZ AAT variants in circulation.

### Other Embodiments

From the foregoing description, it will be apparent that variations and modifications may be made to the aspects or embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The present disclosure may be related to International Application No. PCT/US2020/018195, the full disclosure of which is incorporated herein by reference in its entirety for all purposes.

What is claimed:

1. A nucleic acid encoding a base editor comprising a nucleic acid programmable DNA binding protein (napDNAbp) domain and an adenosine deaminase domain, wherein the napDNAbp domain comprises an alteration selected from the group consisting of M1135L, E1250K, A1283D, Q1136Y, R1337K, R765A, and Q768A of an amino acid sequence, or a fragment thereof lacking an N-terminal methionine, that is at least 90% identical to:
(SEQ ID NO: 554)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMVKRYDEHHQDLTLLKALVRQQLPEKYKEI
FFDQSKNGYAGYIDGGASQEEFYKFIKPILEKMDGTEELLVKLNREDLLR
KQRTFDNGIIPHQIHLGELHAILRRQGDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRLRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGGHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKGNSDKLIARKKDWDPKKYGGFMQPTVAYSVLVVAKVE
KGKSKKLKSVKELLGITIMERSSFEKNPIDFLEAKGYKEVKKDLIIKLPK
YSLFELENGRKRMLASAKFLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPRAFKYFDTTIARKEYRSTKEVLDATLIHQ
SITGLYETRIDLSQLGGD.

2. The nucleic acid of claim 1, wherein the napDNAbp domain comprises a combination of alterations referenced to SEQ ID NO: 554 selected from the group consisting of:
a R1337K;
b. Q1136Y and R1337K;
C. M1135L, Q1136Y, and R1337K;
d. M1135L, Q1136Y, A1283D, and R1337K;
e M1135L, E1250K, A1283D, and R1337K;
f. Q1136Y, A1283D, and E1250K;
g. M1135L, Q1136Y, A1283D, and R1337K; and
h. R765A, Q768A, M1135L, Q1136Y, A1283D, and R1337K.

3. The nucleic acid of claim 1, wherein the adenosine deaminase domain comprises an alteration at an amino acid position selected from the group consisting of L36, I76, V82, Y147, Q154, and N157 of an amino acid sequence, or a fragment thereof lacking an N-terminal methionine, that is at least 85% identical to:
(SEQ ID NO: 1)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD.

4. The nucleic acid of claim 3, wherein the adenosine deaminase domain comprises an amino acid alteration selected from the group consisting of L36H, 176Y, V82T, Y147T, Q154S, and N157K referenced to SEQ ID NO: 1.

5. The nucleic acid sequence of claim 4, wherein the adenosine deaminase domain comprises the amino acid alterations L36H, I76Y, V82T, Y147T, Q154S, and N157K referenced to SEQ ID NO: 1

6. The nucleic acid of claim 5, wherein the adenosine deaminase domain comprises an amino acid sequence that is at least 90% identical to the following amino acid sequence:
(SEQ ID NO: 426)
SEVEFSHEYWMRHALTLAKRARDEREVPVGAVLVHNNRVIGEGWNRAIGL
HDPTAHAEIMALRQGGLVMQNYRLYDATLYTTFEPCVMCAGAMIHSRIGR
VVFGVRNAKTGAAGSLMDVLHYPGMNHRVEITEGILADECAALLCTFFRM
PRSVFKAQKKAQSSTD.

7. The nucleic acid of claim 1, wherein the adenosine deaminase domain comprises the amino acid sequence of SEQ ID NO: 426.

8. The nucleic acid of claim 1, further comprising a linker between the adenosine deaminase and the napDNAbp.

9. The nucleic acid of claim 8, wherein the linker comprises an amino acid sequence selected from the group consisting of: SGGSSGGSSGSETPGTSESATPESSGGSSGGS (SEQ ID NO: 357), EGGSEEEEESGS (SEQ ID NO: 542), and KGPKPKKEESEK (SEQ ID NO: 439).

10. The nucleic acid of claim 1, further comprising a nuclear localization sequence (NLS).

11. The nucleic acid of claim 10, wherein the NLS comprises the amino acid sequence
(SEQ ID NO: 438)
EGADKRTADGSEFESPKKKRKV.

12. The nucleic acid of claim 1, wherein the base editor comprises an amino acid sequence that is at least 90% identical to any one of SEQ ID NOs: 440-450.

13. The nucleic acid of claim 12, wherein the base editor comprises any one of SEQ ID NOs: 440-450.

14. The nucleic acid of claim 1, wherein the nucleic acid comprises RNA.

15. The nucleic acid of claim 14, wherein the nucleic acid is an mRNA molecule.

16. A base editor system comprising a nucleic acid encoding a base editor comprising (i) a nucleic acid programmable DNA binding protein (napDNAbp) domain and an adenosine deaminase domain and (ii) a guide polynucleotide,
wherein the napDNAbp domain comprises an alteration selected from the group consisting of M1135L, E1250K, A1283D, Q1136Y, R1337K, R765A, and Q768A of an amino acid sequence, or a fragment thereof lacking an N-terminal methionine, that is at least 90% identical to:
(SEQ ID NO: 554)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMVKRYDEHHQDLTLLKALVRQQLPEKYKEIFFDQSKNGYAGYIDGGASQEEF
YKFIKPILEKMDGTEELLVKLNREDLLRKQRTFDNGIIPHQIHLGELHAILRRQGDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRLRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGGHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKGNS
DKLIARKKDWDPKKYGGFMQPTVAYSVLVVAKVEKGKSKKLKSVKELLGITIMERSSFEKNP
IDFLEAKGYKEVKKDLIIKLPKYSLFELENGRKRMLASAKFLQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPRAFKYFDTTIARKEYRSTKEVLDATLIHQSITGLYETRIDLSQ
LGGD.

17. The base editor system of claim 16, wherein the guide polynucleotide is a single guide RNA (sgRNA).

18. The base editor system of claim 17, wherein the sgRNA comprises a spacer comprising a nucleotide sequence selected from the group consisting of: 5′-ACCAUCGACAAGAAAGGGACUGA-3′ (SEQ ID NO: 466); 5′-CCAUCGACAAGAAAGGGACUGA-3′ (SEQ ID NO: 559); 5′-CAUCGACAAGAAAGGGACUGA-3′ (SEQ ID NO: 560); 5′-AUCGACAAGAAAGGGACUGA-3′ (SEQ ID NO: 561); 5′-UCGACAAGAAAGGGACUGA-3′ (SEQ ID NO: 562); and 5′-CGACAAGAAAGGGACUGA-3′ (SEQ ID NO: 563).

19. The base editor system of claim 17, wherein the sgRNA comprises a nucleotide sequence selected from the group consisting of:
(SEQ ID NO: 558)
5′-ACCAUCGACAAGAAAGGGACUGA GUUUUAGAGC UAGAAAUAGC
AAGUUAAAAU AAGGCUAGUC CGUUAUCAAC UUGAAAAAGU
GGCACCGAGU CGGUGCUUUU-3′;
(SEQ ID NO: 564)
5′-CCAUCGACAAGAAAGGGACUGA GUUUUAGAGC UAGAAAUAGC
AAGUUAAAAU AAGGCUAGUC CGUUAUCAAC UUGAAAAAGU
GGCACCGAGU CGGUGCUUUU-3′;
(SEQ ID NO: 565)
5′-CAUCGACAAGAAAGGGACUGA GUUUUAGAGC UAGAAAUAGC
AAGUUAAAAU AAGGCUAGUC CGUUAUCAAC UUGAAAAAGU
GGCACCGAGU CGGUGCUUUU-3′;
(SEQ ID NO: 566)
5′-AUCGACAAGAAAGGGACUGA GUUUUAGAGC UAGAAAUAGC
AAGUUAAAAU AAGGCUAGUC CGUUAUCAAC UUGAAAAAGU
GGCACCGAGU CGGUGCUUUU-3′;
(SEQ ID NO: 567)
5′-UCGACAAGAAAGGGACUGA GUUUUAGAGC UAGAAAUAGC
AAGUUAAAAU AAGGCUAGUC CGUUAUCAAC UUGAAAAAGU
GGCACCGAGU CGGUGCUUUU-3′;
and
(SEQ ID NO: 568)
5′-CGACAAGAAAGGGACUGA GUUUUAGAGC UAGAAAUAGC
AAGUUAAAAU AAGGCUAGUC CGUUAUCAAC UUGAAAAAGU
GGCACCGAGU CGGUGCUUUU-3′.

20. The base editor system of claim 19, wherein the sgRNA comprises a nucleotide having a 2′-OMe modification, a 2′-fluoro (F) modification, and/or a phosphorothioate modification.

21. A guide RNA comprising a nucleotide sequence, from 5′ to 3′, selected from the group consisting of:
(SEQ ID NO: 569)
mAsmUsmCsGACAAGAAAGGGACUGAGUUUUAGAGCUAGAAAUAGCAAGU
UAAAAUAAGGCUAGUCCGUUAUCAACUUGAAAAAGUGGCACCGAGUCGGU
GCmUsmUsmUsU;
(SEQ ID NO: 570)
mAsmUsmCsGACAAGAAAGGGACUGAGUUUUAGAGCUAGAAAUAGCAAGU
UAAAAUAAGGCUAGUCCGUUAUCAACUUGAAAAAGUGGCACCGAGUCGGU
GCUsmUsmUsmU,
wherein the guide is covalently linked at the 3′ end to a peptide with the amino acid sequence CKRTADGSEFESPKKKRKV (SEQ ID NO: 543);
(SEQ ID NO: 571)
mAsmUsmCsmGmAmCmAmAmGmAfAfAfGIGGsAsfCfUGsmAmGUsUsUsfUfAmGmAmGm
CmUmAmGmAmAmAmUmAmGmCmAmAmGUUmAAmAmAUmAmAmGmGCUmAGUCmCGUUmAmUm
CmAmAmCmUmUmGmAmAmAmAmAmGmUmGmGmCmAmCmCmGmAmGmUmCmGmGmUmGmCsmU
smUsmU;
(SEQ ID NOs: 572)
mAsmUsmCsGACAAGAAAGGGACUGAmGUUUUAGmAmGmCmUmAmGmAmAmAmUmAmGmCmA
mAGUUmAAmAAmUAmAmGmGmCmUmAGUmCmCGUUAmUmCAAmCmUmUmGmAmAmAmAmAmG
mUmGGmCmAmCmCmGmAmGmUmCmGmGmUmGmCmUsmUsmUsmU;
(SEQ ID NOs: 573)
mCsmAsmUsCGACAAGAAAGGGACUGAGUUUUAGAGCUAGAAAUAGCAAGUUAAAAUAAGGC
UAGUCCGUUAUCAACUUGAAAAAGUGGCACCGAGUCGGUGCmUsmUsmUsU;
(SEQ ID NOs: 574)
mAsmUsmCsmGmAmCmAmAmGmAfAfAfGIGGsAsfCUGmAmGUSUUfUfAmGmAmGmCmUm
AmGmAmAmAmUmAmGmCmAmAmGUUmAAmAmAUmAmAmGmGCUmAGUCmCGUUmAmUmCmAm
AmCmUmUmGmAmAmAmAmAmGUGmGmCmAmCmCmGmAmGmUmCmGmGmUmGmCsmUsmUsmU;
(SEQ ID NOs: 575)
mAsmUsmCsGACAAGAAAGGGACUGAmGUUUUAGmAmGmCmUmAmGmAmAmAmUmAmGmCmA
mAGUUmAAmAAmUAmAmGmGmCmUmAGUmCmCGUUAmUmCAAmCmUmUmGmAmAmAmAmAmG
UGGmCmAmCmCmGmAmGmUmCmGmGmUmGmCmUsmUsmUsmU;
(SEQ ID NOs: 576)
mAsmUsmCsmGmAmCmAmAmGmAAAGGGACUGmAmGUUUUAmGmAmGmCmUmAmGmAmAmAm
UmAmGmCmAmAmGUUmAAmAmAUmAmAmGmGCUmAGUCmCGUUmAmUmCmAmAmCmUmUmGm
AmAmAmAmAmGUGmGmCmAmCmCmGmAmGmUmCmGmGmUmGmCsmUsmUsmU;
(SEQ ID NOs: 577)
mAsmUsmCsmGmAmCmAmAmGmAAAGGGACUGAmGUUUUAGmAmGmCmUmAmGmAmAmAmUm
AmGmCmAmAGUUmAAmAAmUAmAmGmGmCmUmAGUmCmCGUUAmUmCAAmCmUmUmGmAmAm
AmAmAmGUGGmCmAmCmCmGmAmGmUmCmGmGmUmGmCmUsmUsmUsmU;
(SEQ ID NOs: 578)
mCsmAsmUsmCmGmAmCmAmAmGmAAAGGGACUGAmGUUUUAGmAmGmCmUmAmGmAmAmAm
UmAmGmCmAmAGUUmAAmAAmUAmAmGmGmCmUmAGUmCmCGUUAmUmCAAmCmUmUmGmAm
AmAmAmAmGUGGmCmAmCmCmGmAmGmUmCmGmGmUmGmCmUsmUsmUsmU;
(SEQ ID NOs: 579)
mAsmUsmCsmGmAmCmAmAmGmAmAAmGmGGAmCUGmAmGUUUUAGmAmGmCmUmAmGmAmA
mAmUmAmGmCmAmAGUUmAAmAAmUAmAmGmGmCmUmAGUmCmCGUUAmUmCAAmCmUmUmG
mAmAmAmAmAmGUGGmCmAmCmCmGmAmGmUmCmGmGmUmGmCmUsmUsmUsmU;
(SEQ ID NOs: 580)
mCsmAsmUsmCmGmAmCmAmAmGmAmAAmGmGGAmCUGmAmGUUUUAGmAmGmCmUmAmGmA
mAmAmUmAmGmCmAmAGUUmAAmAAmUAmAmGmGmCmUmAGUmCmCGUUAmUmCAAmCmUmU
mGmAmAmAmAmAmGUGGmCmAmCmCmGmAmGmUmCmGmGmUmGmCmUsmUsmUsmU;
(SEQ ID NOs: 581)
mCsmAsmUsCGACAAGAAAGGGACUGAmGUUUUAGmAmGmCmUmAmGmAmAmAmUmAmGmCm
AmAGUUmAAmAAmUAmAmGmGmCmUmAGUmCmCGUUAmUmCAAmCmUmUmGmAmAmAmAmAm
GUGGmCmAmCmCmGmAmGmUmCmGmGmUmGmCmUsmUsmUsmU;
and
(SEQ ID NOs: 582)
mAsmUsmCsGACAAGAAAGGGACUGAGUUUUAGAmGmCmCmGmGmCmGmGmAmAmAmCmGmC
mCmGmGmCAAGUUAAAAUAAGGCUAGUCCGUUAUCAmAmCmUmUmGmAmAmAmAmAmGmUmG
mGmCmAmCmCmGmAmGmUmCmGmGmUmGmCmUsmUsmUsmU;
(SEQ ID NOs: 583)
mAsmUsmCsmGmAmCmAmAmGmAAAGGGACUGAGUUUUAGAmGmCmCmGmGmCmGmGmAmAm
AmCmGmCmCmGmGmCAAGUUAAAAUAAGGCUAGUCCGUUAUCAmAmCmUmUmGmAmAmAmAm
AmGmUmGmGmCmAmCmCmGmAmGmUmCmGmGmUmGmCmUsmUsmUsmU;
(SEQ ID NOs: 584)
mAsmUsmCsGACAAGAAAGGGACUGAGUUUUAGAmGmCmUmAmGmAmAmAmUmAmGmCAAGU
UAAAAUAAGGCUAGUCCGUUAmUmCAAmCmUmUGGACUUCGGUCCmAmAmGmUmGGmCmAmC
mCmGmAmGmUmCmGmGmUmGmCmUsmUsmUsmU;
or
(SEQ ID NO: 585)
mAsmUsmCsGACAAGAAAGGGACUGAGUUUUAGAmGmCmCmGmGmCmGmGmAmAmAmCmGmC
mCmGmGmCAAGUUAAAAUAAGGCUAGUCCGUUAmUmCAAmCmUmUGGACUUCGGUCCmAmAm
GmUmGGmCmAmCmCmGmAmGmUmCmGmGmUmGmCmUmUmUmU;
wherein “N” represents any nucleotide, “mN” indicates a 2′-OMe modification of the nucleotide “N”, fN indicates a 2′-fluoro (F) modification of the nucleotide “N,” and “Ns” indicates that the nucleotide “N” is linked to the following nucleotide by a phosphorothioate. sgRNA modified sequence here.

22. A pharmaceutical composition comprising the base editor system of claim 19 and a pharmaceutically acceptable carrier, vehicle, or excipient.

23. The pharmaceutical composition of claim 22 further comprising a lipid.

24. The pharmaceutical composition of claim 23, wherein the lipid is a cationic lipid.

25. The pharmaceutical composition of claim 22 further comprising a lipid nanoparticle.

26. The pharmaceutical composition of claim 25, wherein the nucleic acid encoding the base editor is an mRNA molecule, and wherein the mRNA molecule and the sgRNA are formulated in the lipid nanoparticle.

27. A method of editing an alpha-1 antitrypsin polynucleotide comprising a single nucleotide polymorphism (SNP) associated with alpha-1 antitrypsin deficiency, the method comprising contacting the polynucleotide with a guide RNA, and a base editor comprising a nucleic acid programmable DNA binding protein (napDNAbp) domain and an adenosine deaminase domain, wherein the napDNAbp domain comprises an alteration selected from the group consisting of M1135L, E1250K, A1283D, Q1136Y, R1337K, R765A, and Q768A of an amino acid sequence, or a fragment thereof lacking an N-terminal methionine, that is at least 90% identical to:
(SEQ ID NO: 554)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMVKRYDEHHQDLTLLKALVRQQLPEKYKEIFFDQSKNGYAGYIDGGASQEEF
YKFIKPILEKMDGTEELLVKLNREDLLRKQRTFDNGIIPHQIHLGELHAILRRQGDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRLRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGGHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKGNS
DKLIARKKDWDPKKYGGFMQPTVAYSVLVVAKVEKGKSKKLKSVKELLGITIMERSSFEKNP
IDFLEAKGYKEVKKDLIIKLPKYSLFELENGRKRMLASAKFLQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPRAFKYFDTTIARKEYRSTKEVLDATLIHQSITGLYETRIDLSQ
LGGD,
and wherein said guide RNA targets said base editor to effect an alteration of the SNP associated with alpha-1 antitrypsin deficiency.

28. A method of editing an alpha-1 antitrypsin polynucleotide comprising a single nucleotide polymorphism (SNP) associated with alpha-1 antitrypsin deficiency in a cell, the method comprising contacting the cell with the base editor system of claim 19.

29. A method for treating alpha-1 antitrypsin deficiency (A1AD) in a subject, the method comprising: administering to the subject the pharmaceutical composition of claim 22 to effect an A·T to G·C alteration of a single nucleotide polymorphism (SNP) associated with A1AD, thereby treating A1AD in the subject.

30. The method of claim 29, wherein the SNP associated with A1AD results in expression of an A1AT polypeptide comprising an amino acid alteration selected from the group consisting of E264V and E342K.
